(19) United States
(12) Patent Application Publication
(10) Pub. No.: US 2009/0104195 A1
(43) Pub. Date: Apr. 23, 2009

(54) TARGETED LIGANDS

(76) Inventor: William Herman, Thornhill (CA)

Correspondence Address: CHERYL H AGRIS PHD, PO BOX 806, PELHAM, NY, 10803, US

(21) Appl. No.: 12/337,610

(22) Filed: Dec. 17, 2008

Related U.S. Application Data
(63) Continuation of application No. 10/481,670, filed on Dec. 22, 2003, now abandoned.
(60) Provisional application No. 60/274,217, filed on Mar. 9, 2001.
(60) Provisional application No. 60/276,911, filed on Mar. 20, 2001.
(60) Provisional application No. 60/279,132, filed on Mar. 28, 2001.
(60) Provisional application No. 60/281,029, filed on Apr. 4, 2001.
(60) Provisional application No. 60/306,148, filed on Jul. 19, 2001.

(30) Foreign Application Priority Data
Jan. 14, 2002 (CA) 2368708

(51) Int. Cl. A61K 39/395; C07K 16/00; A61K 38/16; G01N 33/53; A61P 35/00; A61P 31/00; A61P 37/00; C07K 1/107; C07K 14/00

(52) U.S. Cl. 424/136.1; 530/387.3; 530/350; 514/12; 530/402; 435/7.2

(57) ABSTRACT

The invention contemplates a composition containing a multispecific ligand containing at least a first ligand binding moiety and a second ligand binding moiety. The first ligand binding moiety specifically binds with a pre-selected first affinity to at least a first ligand. The first ligand has a first biodistribution. The second ligand binding moiety specifically binds with a pre-selected affinity to at least a second ligand. The second ligand has a second biodistribution. The affinity of first and second ligand binding moieties are selected to bias the biodistribution of the multispecific ligand in favor of a selected location of one or both of the ligands.

### PRIORITY CLAIM

This application is a continuation of application Ser. No. 10/481,670, filed Dec. 19, 2008, the contents of which are incorporated herein by reference. Application Ser. No. 10/481,670 is a national stage application under 35 USC 371 of application no. PCT/CA02/00317, filed Mar. 11, 2002, the contents of which are incorporated herein by reference. PCT/CA02/00317 is a non-provisional application under 35 USC 119(e) of application Nos. 60/274,217, filed Mar. 11, 2002, the contents of which are incorporated herein by reference; 60/276,911, filed Mar. 9, 2001, the contents of which are incorporated herein by reference; 60/279,132, filed Mar. 28, 2001, the contents of which are incorporated herein by reference; 60/281,029, filed Apr. 7, 2001, the contents of which are incorporated herein by reference and application Ser. No. 60/306,148, filed Jul. 19, 2001, the contents of which are incorporated herein by reference. The application further claims priority to CA 2368708, filed Jan. 14, 2002, under 35 USC 119(a)-(d), the contents of which are incorporated herein by reference.

### FIELD OF THE INVENTION

The present invention relates to multispecific ligands, for example a heterofunctional ligand comprising at least first and second binding moieties which have cooperating functional affinities including a multispecific ligand, for example, a bispecific antibody, having at least a first portion which binds to a ‘lymphatic vessel associated’ antigen/receptor and a second portion having at least one immune-affecting functionality including, without limitation, functions related to antigen presentation, immune signaling, suppression or enhancement of immune tolerance or immune stimulation, or binding to a target molecule, for example a cell surface antigen, receptor etc.

### BACKGROUND OF THE INVENTION

Immunotherapy has gained wide acceptance as a promising measure to address several disease states including autoimmune disease, transplant rejection, infectious disease and cancer. Despite rapid and exciting progress in approaches to treatment, the disease burden attributable to such illnesses has not significantly abated. The complex nature of the normal and pathologic immunologic processes associated with such diseases, coupled with logistical problems in evaluating and implementing methods for immunotherapy in human subjects, continue to be some of the obstacles to successful advances in treatment.

Successful approaches to immunotherapy are predicated on the ability of the immunotherapeutic molecule to be delivered in a therapeutic, sub-toxic dose at the desired therapeutic frequency. In the process of selection of a suitable therapeutic molecule, it is recognized that sub-toxic doses may be insufficient for the desired therapeutic effect, especially where the antibody binds incidentally to cell populations other than the target population. In the case of an injectable preparation and especially an intravenous mode of delivery, in contrast to readily self-administered modes of delivery, the optimal dosing frequency for therapeutic purposes could impose an undesirable burden on the patient and care-giver, assuming that such optimal frequency is to begin with deemed convenient for clinical trials.

Numerous research efforts are underway to identify and test ligands including antibodies, biologic effector ligands (e.g. cytokines, chemokines, growth factors colony stimulating factors) receptor agonists or antagonists etc. which will bind to or otherwise interact with or trigger responses in or towards target entities, including pathogenic organisms, tissue specific cells, diseased cells, immune cells etc. A recent example is a renewed interest to find molecules and methods of triggering an interaction with CD45 (see for example Nature (2001) Vol. 409 p. 349-354). Evaluating the biological effect of interactions with such target ligands is often obfuscated and retarded by the biodistribution of such ligands on cells other than the target population which results in undesired and/or confusing pleiotropic effects.

The present invention facilitates scientific assessment, development, role evaluation, therapeutic evaluation, and delivery, particularly targeted delivery of molecules that exert biologic functions and particularly immune related functions. In particular, the targeting agents and methods which are the subject of the invention herein facilitate scientific evaluation of the biological effects of a more targeted biodistribution of such targeting agents, by limiting undesired or confusing side effects. In preferred aspects the invention contemplates compositions of matter and methods of delivery, in some cases using ligands that but for the targeting methods herein defined would be ineffective or have a broader effect than is desirable; or similarly, but for the severity of the disease or the absence of other therapeutic alternatives for which such ligands are useful, they would otherwise be inappropriate for therapeutic use. The present invention accommodates evaluation of the biological role and/or effects of such ligands for therapeutic or other scientific purposes using such targeting strategies. In particular, the present invention provides a vehicle to preferentially target, on a sub-population of cells for which there is a cell-associated marker, a receptor or receptor ligand which is present on a more heterogeneous population of cells.

### SUMMARY OF THE INVENTION

The invention contemplates a composition containing a multispecific ligand containing at least a first ligand binding moiety and a second ligand binding moiety. The first ligand binding moiety specifically binds with a pre-selected first affinity to at least a first ligand. The first ligand has a first biodistribution. The second ligand binding moiety specifically binds with a pre-selected affinity to at least a second ligand. The second ligand has a second biodistribution. The affinity of first and second ligand binding moieties are selected to bias the biodistribution of the multispecific ligand in favour of a selected location of one or both of the ligands.

The invention contemplates a composition containing a multispecific ligand. The multispecific ligand contains at least a first ligand binding moiety and a second ligand binding moiety. The first ligand binding moiety specifically binds to a first ligand having a first biodistribution. The second ligand binding moiety specifically binds to a second ligand having a second biodistribution. The second biodistribution is different from that of the first biodistribution, and the affinity of the first and second ligand binding moieties to their respective ligands are different and selected to bias the biodistribution of the multispecific ligand towards the first or second biodistribution.

The invention contemplates further, a composition containing a multispecific ligand. The multispecific ligand contains a first ligand binding moiety and a second ligand binding moiety. The first ligand binding moiety specifically binds with a pre-selected first affinity to a first ligand. The first ligand has a first biodistribution. The second ligand binding moiety specifically binds with a pre-selected affinity to a second ligand. The second ligand has a second biodistribution. In this embodiment of the multispecific ligand, the affinity of first and second ligand binding moieties are selected to bias the biodistribution of the multispecific ligand.

The invention further contemplates a composition containing a multispecific ligand. The multispecific ligand specifically binds to a target ligand. The target ligand is specific to a selected sub-population of a heterogeneous cell population. This embodiment of the multispecific ligand contains a first ligand binding moiety and a second ligand binding moiety. The first ligand binding moiety specifically binds to a cell sub-population associated ligand. The second ligand binding moiety binds to the target ligand. In this embodiment, the first ligand binding moiety has an affinity for the sub-population associated ligand higher than the affinity of the second ligand binding moiety for the target ligand.

The invention further contemplates a composition containing a bispecific ligand containing a first ligand and a second ligand. The first ligand binds to a first target ligand and the second ligand binds to a second target ligand. In this embodiment of the bispecific ligand, the affinity of the first ligand is selected to enable binding to the first target ligand independently of the ability of the second ligand to bind to the second target ligand. Further, the affinity of the second ligand is selected to substantially reduce the probability of its binding to the second target ligand without the first ligand binding first or substantially contemporaneously to the first target ligand.

The invention further contemplates a composition containing a bispecific antibody containing a first antibody component and a second antibody component. The first antibody component binds to a first target ligand and the second antibody component binds to a second target ligand. In this embodiment, the affinity or avidity or both the affinity and avidity of the first antibody component are selected to enable binding to the first target ligand independently of the ability of the second antibody component to bind to the second target ligand. The avidity or affinity or both the affinity and avidity of the second ligand are selected to substantially reduce the probability of its binding to the second target ligand without the first ligand binding first or substantially contemporaneously to the first target ligand.

The invention further contemplates a multispecific ligand containing a first moiety and a second moiety. The first moiety binds to a first target ligand. The second moiety binds to a second target ligand. The affinity or avidity or both the affinity and avidity of the first moiety are selected to enable the first moiety to bind to the first target ligand independently of the ability of the second moiety to bind to the second target ligand. The avidity or affinity or both the affinity and avidity of the second moiety are selected to substantially reduce the probability of its binding to the second target ligand without the first moiety, first or substantially contemporaneously, binding to the first target ligand.

The invention further contemplates a multispecific ligand containing a first moiety and a second moiety. The first moiety binds to a first target ligand. The second moiety binds to a second target ligand. The affinity or avidity or both the affinity and avidity of the first moiety are selected to enable the first moiety to bind to the first target ligand independently of the ability of the second moiety to bind to the second target ligand. The avidity or affinity or both the affinity and avidity of the second moiety are selected to substantially reduce the probability of either moiety binding for a sufficient duration or series of durations to its respective target ligand to accomplish a therapeutic function without the other moiety, first or substantially contemporaneously, binding to its respective target ligand.

The invention further contemplates a composition containing a multispecific ligand containing a first moiety and a second moiety. The first moiety binds to a first target ligand. The second moiety binds to a second target ligand. The affinity or avidity or both the affinity and avidity of the first moiety are selected to enable the first moiety to bind to the first target ligand independently of the ability of the second moiety to bind to the second target ligand. The avidity or affinity or both the affinity and avidity of the second moiety are selected to enable the second moiety to bind to the second entity in preference to the first moiety binding to the first entity when both first and second moieties are substantially contemporaneously bound to the respective first and second entities.

The invention contemplates a composition containing a multispecific ligand containing a first moiety, a second moiety and a third ligand binding moiety. The first moiety binds to a first target ligand and the second moiety binds to a second target ligand. In this embodiment, the affinity or avidity or both the affinity and avidity of the first moiety are selected to enable the first moiety to bind to the first target ligand in preference to the second moiety binding to the second entity when both first and second moieties are substantially contemporaneously bound to the respective first and second entities, and the avidity or affinity or both the affinity and avidity of the second moiety are selected to enable the third target ligand to bind to the second entity in preference to the second moiety binding to the second entity when both the third target ligand and the second moiety are substantially contemporaneously bound to the second entity.

The invention further contemplates a composition containing an antibody which specifically binds to an epitope on a ligand. The ligand recognized by the antibody exerts a biologic effect by binding to a target site on a target ligand. The epitope bound by the antibody is proximal to the binding site of the ligand for the target ligand, so that binding of the antibody reduces but does not prevent the affinity of the ligand for its target ligand.

The invention further contemplates a composition containing a multispecific ligand containing a first ligand binding moiety and a second moiety. The first ligand binding moiety specifically binds to a lymphatic endothelial cell associated marker. The second moiety contains an independent therapeutic function.

The invention further contemplates a composition containing an immunocytokine containing an anti-idiotypic antibody component and a cytokine component. The anti-idiotypic antibody component recognizes the paratope of an antibody which binds to a lymphatic vessel associated ligand.

The invention further contemplates a composition containing a bispecific antibody containing an anti-idiotypic antibody component and an anti-CD3 antibody or an anti-CD28 antibody component. The anti-idiotypic antibody recognizes the paratope of an antibody which binds specifically to a lymphatic vessel associated ligand.

The invention additionally contemplates physiologically acceptable compositions of the compositions encompassed by the invention.

The invention likewise contemplates methods of use of the compositions encompassed by the invention.

A composition comprising a multispecific ligand comprising at least a first ligand binding moiety which specifically binds to a first ligand having a first biodistribution and a second ligand binding moiety which specifically binds to a second ligand having a second biodistribution different from that of the first ligand, and wherein the affinity of the first and second ligand binding moieties are different and selected to bias the biodistribution of the multispecific ligand.

### DESCRIPTION OF THE PREFERRED EMBODIMENT

As exemplified above, the dual “affinity” based targeting strategy of the invention, may be understood in one aspect, in terms of a strategic allocation of the respective affinity properties of the multispecific ligand to at least one “targeting” function and at least one “effector” function. Accordingly, with respect to some embodiments of the invention, the term “multifunctional” ligand is used interchangeably.

Thus according to one preferred embodiment, at least one of the ligand binding moieties is a “targeting” arm in the sense that it at least preferentially recognizes a marker that is associated with one or more specific target entities e.g. cell populations, and the other ligand binding moiety is an “effector” arm which binds with relatively less affinity or functional affinity to a target ligand which has a more diverse biodistribution. In this case, the biodistribution of the multispecific ligand is biased in favour of the location(s) of both ligands relative to the location(s) of the target ligand so as to limit the big distribution to non-target entities.

Such binding or recognition is understood throughout to be specific, in contrast to non-specific binding.

The term “effector” is used to refer to the ability to effect a biological consequence through binding, for example effecting a signal transduction event by activating a receptor, or blocking the target ligand from associating with a complementary ligand, for example blocking a receptor from associating with a complementary ligand (e.g. its natural ligand) and thereby, for example, preventing a signal transduction, or for example in the case of a decoy receptor preventing the biological consequence (e.g. protective effect) associated with the function of such receptor, or blocking a ligand from associating with a complementary ligand e.g. receptor on another entity e.g., a cancer cell, infectious agent or immune cell.

A biased biodistribution is preferably accomplished by the multispecific ligand contemporaneously recognizing both ligands on the same entity e.g. cell, and may be accomplished by such contemporaneous recognition occurring on adjacent entities or by increasing the propensity of the multispecific ligand to locate in proximity to a target entity in virtue of the relatively high affinity targeting arm. The targeting arm may itself be an effector.

In another embodiment, the biological consequence accomplished by the effector arm is at least minimally retargeting, for example wherein the lesser affinity or functional affinity of the first binding moiety is selected to permit the multispecific ligand to preferentially bind to an adjacent entity, for example, a circulating entity which circulates in proximity to a lymphatic endothelial cell to which the multifunctional ligand is bound with lesser affinity. Again, the relatively high affinity first binding moiety may itself be an effector.

In another embodiment the biological consequence accomplished by the effector arm is minimally cooperative targeting, for example where the biodistributions of at least one of the first and second ligands extends to a diverse population of cells other than target cell population and where binding is only possible or consequential if both ligands are available for contemporaneous binding, in this case due to the affinities of the first and second ligand binding being individually insufficient for effective targeting (e.g. insufficient for other than ephemeral binding). In the context of this embodiment of the invention, the “cooperative targeting” is not simply ameliorated by the effector arm, it is predicated and reliant on this arm. One or both ligand binding moieties may exert additional effector properties.

It will also be appreciated that any multispecific ligand of the invention or any component thereof may be fused or conjugated to a separate effector as exemplified below, including toxins, cytokines, adhesion molecules etc.

The ligand binding moiety is preferably an antibody or a sequence or sequences of amino acids etc. which are the natural ligand for the target ligand, for example where the ligand is a cytokine or lymphokine receptor, such as IL-2 receptor, the ligand binding moiety may comprise a sequence of amino acids which is IL-2. The ligand binding moiety may also be a mutated or a newly developed form of the natural ligand (e.g. developed through combinatorial libraries) or a natural or synthetic chemical ligand (developed through combinatorial chemistry).

### DEFINITIONS

The term “associated” in relation to markers that are dominantly distributed on one or more particular entities is used to mean exclusively expressed, primarily expressed, or over-expressed to advantage from a targeting standpoint.

The term “receptor ligand” means a target ligand which is a ligand for a receptor, for example, a receptor on a cell or infectious agent or a receptor which circulates independently of another entity.

The term affinity is contrasted to functional affinity which may result from avidity.

The term epitope though technically understood to be specific for a given antibody, is used in a preferred embodiments to refer to antigenic determinants that are situated proximally to one another so that two antibodies will be considered to bind to the same epitope if one competitively inhibits the binding of the other through any probative competitive inhibition experiment known to those skilled in the art.

The invention contemplates that two antibodies with the same epitope specificity may have substantially the same amino acid composition i.e. with possible exception of one or more additions, deletions or substitutions including conservative amino acid substitutions which do not substantially affect the specificity and amino acid composition of the paratope

The term approximately in the context of orders of magnitude variations in affinity refers a variability that is up to a half an order or magnitude.

Without limiting the scope of the claims it is generally understood that biodistribution of a multispecific ligand in contrast to that of a ligand will be predicated on the bioavailability of its target ligand.

The term “overlap” and related terms connote that notwithstanding the difference in distributions of the first and second ligands the first and second ligands are bioavailable for recognition on the same entity. This term and related terms, exemplified below, are intended to exclude a situation where both ligands are preferentially expressed on substantially the same entity, for example two different tumor associated antigens associated differentially with a differentiated population of cells within a tumor, most particularly in the case where they are individually suitable targets for delivery of a toxic payload. Thus the terms “different” in regard to biodistributions and “heterogeneous” and “diverse” in reference to populations of entities are similarly understood to exclude such a common distribution, in the appreciation that the invention primarily represents an improved strategy for targeting two different ligands, in which one ligand has a broader distribution than the other or both have distributions that may overlap but are different from that of the target population. It will also be appreciated that the invention has particular application to a situation in which at least one of the non-target populations is one on which one of said first and second ligands is substantially represented (in contrast to one on which it simply enjoys limited expression).

The term “receptor ligand” means a target ligand which is a ligand for a receptor, for example, a receptor on a cell or infectious agent or a receptor which circulates independently of another entity.

The term “antigen binding fragment” refers to a polypeptide or a plurality of associated polypeptides comprising one or more portions of an antibody including at least one VH or VL or a functional fragment thereof.

A moiety that exerts a biologic function is understood to be a “biologic effector” in the sense that its intended interaction with an entity in the lymphatic system or elsewhere in the organism has a biological consequence.

The term neutralizing in regard to an immune function is used broadly to refer to any interposition, interference or impediment which affects the function of the target entity

The terms modulating, mediating, neutralizing function etc. are not intended to be mutually exclusive and are each used broadly, for example, without limiting the generality of the scope accorded herein or by those skilled in the art the term modulating preferably refers to effecting a change, and the term mediating preferably connotes an indirect effect achieved through the instrumentality of another entity, for example a cell, cytokine, chemokine etc.

The term “preferentially binds” recognizes that a given ligand binding moiety might have some non-defeating cross-reactivities.

The term biologic effector ligands is used to refer to any ligand for which there is a complementary target ligand on a target entity, and wherein binding of the biologic effector ligand to the target ligand exerts a biologic effect. For example the target ligand is typically a receptor and the biologic effector ligand may be any complementary ligand such as a cytokine, chemokine, hormone, colony stimulating factor, growth factor, receptor inhibitor, agonist or antagonist, which binds to the receptor with resulting biologic effect.

The term “pre-selected” in reference to the affinity of ligand binding moiety refers to any selection or choice of differential or cooperative affinities relative to a second ligand binding moiety which is generated as a result of a mental or physical process or both, preferably through a process of prediction or post-facto validation of the effects of the choice of the first and second affinities and/or more preferably through an empirical evaluation of different choices for at least one of the first and second affinities, and preferably both.

The term multi means at least two and the term ligand is used broadly to refer to any entity or part thereof which can participate in an intermolecular interaction that can result in specific binding of suitable affinity for the interaction in question.

The term entity includes without limitation any molecule including without limitation, antibodies, complex or association of molecules, drugs, drug carriers (e.g., vesicles, liposomes, nanoparticles, etc.) or any cell as well as any infectious agent or parasite (including, without limitation, spores, viruses, bacteria, fungi) as well as any other immune or therapeutic target.

The term “low affinity” means an affinity of approximately (this term is defined herein) 10⁻⁴ molar to micromolar affinity, preferably (subject to safety considerations), approximately, 10⁻⁵ molar affinity, more preferably (subject to safety considerations), approximately micromolar affinity, the term “medium affinity” means approximately 10⁻⁷ to nanomolar affinity, preferably approximately 10⁻⁸ molar affinity, more preferably approximately nanomolar affinity, and the term “high affinity” means approximately 10⁻¹⁰ affinity or greater. Thus is one embodiment the invention contemplates that the multispecific ligand comprises a “target-ligand” binding moiety which binds with low or medium affinity to a target ligand present on a diverse population of cells (preferably this moiety is an effector moiety i.e. one which exerts a biological effect attributable to its binding e.g. blocking or activating a receptor or blocking a cell membrane channel) and a “targeting”, ligand binding moiety, which binds with medium or high affinity to a ligand associated with a sub-population of those cells so as to bias the biodistribution of the multifunctional ligand in favor of said sub-population. Preferably the multispecific ligand is adapted to be bound contemporaneously to the same cell. In another embodiment the first and second ligands binding moieties each bind to ligands present on diverse overlapping populations of entities e.g. cells (i.e. neither ligand being preferentially associated with a target cell population) and are adapted to be bound contemporaneously and to both bind individually with low affinity, so as to bias the distribution of the multispecific ligand to the population of cells bearing both ligands.

As discussed elsewhere the term approximately, in reference to “order of magnitude” increments in affinity, refers to up to a half order of magnitude in affinity.

According to another embodiment, the invention is directed to an antibody termed a “coybody”. A “coybody” is an antibody in which the on-rate contribution to affinity of the antibody is proportionally less than the off-rate contribution relative to a reference antibody of the same specificity and a greater affinity of up to several orders of magnitude, preferably a reference antibody of approximately one to three orders of magnitude greater affinity, preferably a reference antibody of medium affinity or preferably high affinity. As discussed above the reference antibody preferably comprises cooperating light and heavy variable regions in which at least at least one of the CDRs of at least one of the chains, preferably at least the CDR3, preferably that of the heavy chain, is exclusively or primarily responsible for the binding affinity of the coybody preferably in conjunction with the contribution of at least one of the CDRs of the other chain, such that alterations in the length and or amino acid compositions of one or more other non-contributing CDRs can be leveraged to diminish the on-rate, for example due to steric and/or electrostatic hindrance. In one embodiment the on-rate is reduced by a factor of 2 to 100×. In one embodiment, the coybody binds to a ligand which is over-expressed on a target population of entities (e.g. cells) relative to a non-target population of entities such that the biodistribution of the coybody to the non-target population (and target population) is diminished in a given increment of time following administration. This targeting strategy is understandably adapted to situations where the resulting delay in biodistribution is preferable for diminished toxicity attributable to reduced non-target entity binding in a given unit of time especially where the effectiveness threshold in that same amount of time is not significantly if at all compromised or is preferable due to a sustained release effect (for example using a larger antibody format that is not readily cleared) As discussed below, advantages accrue particularly when this antibody is coupled to a higher affinity antibody (in the form of a multifunctional ligand) which binds to a different ligand associated with the target population. The invention contemplates that coybodies have multiple independent applications, including tempering the effects through antibody mediated neutralization of an over-production or sensitivity to biologic effector ligands (e.g. cytokines e.g. TNF alpha, chemokines e.g. IL-16 (Crohns disease) etc. which are over-produced and/or mediate or aggravate e.g. a chronic medical condition (which for example is not an acute phase) by binding to such ligands, over a prolonged periods, preferably using larger antibody formats which are not readily cleared, especially where such tempering has side effects which are better spread over time and/or where effectiveness is not a limiting factor and/or where a second therapeutic with different non-cumulative side-effects shares the therapeutic burden and/or where a the same antibody with a higher on-rate is used in combination.

The term “antibody” is used broadly, unless the context dictates otherwise, to refer without limitation, to a whole antibody of any class or biologic origin, or chimeric combinations of antibody regions or domains (e.g. FRs and CDRs) of different origins or species e.g. humanized, any combination of one or more antibody fragments or recombinant reconstructions (scFvs) of antibodies including dimers, diabodies, triabodies, a myriad of known bispecific, trispecific, tetraspecific antibody formats or monovalent, divalent, trivalent, tetravalent or other multivalent antibody formats (see for example review in Kriangkum J, et al. Bispecific and bifunctional single chain recombinant antibodies. Biomol Eng 2001 September; 18(2): 31-40 and others herein directly or otherwise referenced) or any fragment, portion, or reconstruction of one or more portions of an antibody (scFv) or any truncated form a ligand binding entity, such antibody typically comprising at least a VH or VL portion or both or a functional portion of same (e.g. microbodies), including single domain antibodies, F(ab′)₂, Fab, Fab′, Facb, Fc, etc. The term antibody also includes fusions of such an antibody so defined and other functional moieties (e.g. toxins, cytokines, chemokines, streptavidin, adhesion molecules).

According to one aspect, the invention is directed to a multispecific ligand with at least two different binding specificities for different target ligands on the same target entity e.g. a cell and which is preferably adapted to bind contemporaneously to (i.e. there are no geometric or other constraints which preclude both moieties from functionally interacting with their respective target ligands at the same time) the different target ligands, said multispecific ligand comprising a first target binding moiety which preferentially (some cross-reactivity(s) does not preclude the utility of the invention) recognizes a first target ligand and a second target binding moiety which preferentially recognizes a second target ligand, and wherein the ability of the second target binding moiety to bind to the second target ligand is diminished relative the ability of the first target binding moiety to bind to the first target ligand, the first target binding moiety having an ability to bind to the first target ligand which is at least sufficient for the first target moiety to bind to the first target ligand independently of the second target binding moiety binding to the second target ligand and an off-rate (with respect to the first target ligand) which at least sufficiently exceeds the on-rate of the second target binding moiety for the second target ligand to at least provide opportunity for the second target moiety to bind the second target ligand when the first target binding moiety is bound to first target ligand, the second target binding moiety having a relatively diminished ability to bind and/or stay bound to the second target ligand independently of the binding of the first target binding moiety to the first target ligand (such that a plurality of the multispecific ligand will bind to a population of cells bearing both target ligands in preference to a population of cells bearing only the second target ligand (i.e. at least in part due to the first target binding moiety assisting (i.e. providing opportunity) the second target binding moiety to bind to the second target ligand and preferably out of proportion to what could be statistically attributed to the presence of two targets ligands on the target cell e.g. the binding of the first target binding moiety providing necessary assistance for the second target moiety to bind is relatively increased (i.e. relative to the situation where both of are of comparable affinity)

It will be appreciated that relative number of bioavailable second target ligands relative to the number of the bioavailable first target ligands will influence the selection of affinities of the first and second target binding moieties. For example, from the standpoint of safety, the affinity of the first target binding moiety for the first target ligand may well be sufficient if initially approximating nanomolar affinity and the affinity of the second target binding moiety for the second target ligand will be selected to limit the number of effective binding events on the population of cells bearing only the second target moiety; an affinity which is inversely proportional to the number of bioavailable second target ligands on the population of cells bearing only the second target ligand i.e. the non-target population (relative to the number of first target ligands on the target population of cells). For example, this may be assessed by determining the amount of labeled multispecific ligand on the target and non-target populations of cells in vivo (or in vitro where the number of bioavailable first and second target ligands can be roughly estimated). This selected affinity, from a effectiveness point of view, will then be assessed as to whether it is sufficient for the second ligand binding moiety to bind to the second target ligand on the target population of cells, with the benefit of the first ligand binding moiety bound or having been bound to first target ligand. For example, where the binding of the second target binding moiety may be assessed through an in vitro assay (e.g. an assay in which the blocking or activating of a receptor is measurable e.g. through inhibition of binding of the natural ligand for a target receptor or through some measurable parameter associated with effective binding for example the release of cytokines or other biologic effector ligand. The effect of binding may be also be assessed by comparing the effects over time relative to a higher affinity second binding moiety which is not associated with a first ligand binding moiety. It will be appreciated that a more ubiquitous second target ligand may require selecting a higher initial affinity of the first target binding moiety for the first target ligand e.g. picomolar affinity, and selecting an affinity of the second target ligand which may for example be of micromolar affinity plus/minus approximately one order of magnitude. It will also be appreciated that the deleterious effects of non-target cell binding will vary as will the degree to which the first target ligand is uniquely found on the target population of cells. In the final analysis a suitable difference in affinity between the two binding affinities may well be at least, approximately, one, two, three, four, five, six, seven or eight orders of magnitude. In this connection the term approximately refers to +/−up to a half order of magnitude (<5×). As discussed below, the invention contemplates that variants of a dual affinity multispecific ligand may be assessed in a high throughput screen or series of such screens with a view to selecting a variant that has one or more predefined properties, alluded to above such as a) the ability to mediate a biologic effect on a target population relative to a negative control; b) the ability to mediate an improved or diminished biologic effect on a target population relative to a positive control. This ability may also be assessed in a competition experiment of any probative type well-known to those skilled in the art; c) the inability or diminished ability to mediate a biologic effect on a non-target population relative to negative and positive controls. Such diminished ability may be also assessed in a competition experiment of any probative type well known to those skilled in the art; d) the ability to target a target population through binding relative to controls and in a competition; e) the inability or diminished ability to target a non-target population relative to controls and in such competition experiment.

In one embodiment, said first target binding moiety recognizes an entity-associated ligand e.g. a target cell-associated* target ligand, for example a ligand which is exclusively expressed, primarily expressed or over-expressed to advantage on the target cell population and said second target binding moiety recognizes a non-target cell-associated target ligand which is present on target cells and non-target cells, for example a receptor, including a decoy receptor e.g. for TRAIL. The multispecific ligand is thereby adapted to block or activate the receptor primarily on the target population of cells. In this connection, the invention is also directed to methods of evaluating or implementing the effects of this enhanced selectivity for the receptor on the target cell population and can be employed to diminish the adverse consequences and evaluate the benefits associated with using a ligand binding moiety that would otherwise undesirably bind to receptors on non-target cells.

The invention contemplates that a variety of different strategies that can be used alone or in any variety of compatible permutations to differentiate between target cells and/or between target and non-target cells. The choice of strategies, may depend at least in part on the circumstances, including the nature of the fluid environment in question, including the rapidity and pressure of flow and the direction(s) of this pressure, the method of delivery, the medical condition for which the molecule is being evaluated, whether the target is moving or stationary, or both, the location or various locations of the target, the targeting venue or venues that is/are most effective and the importance of the size of the molecule for reaching the target as well as bioavailability, and the importance of creating immunoconjugates and immunofusions with other molecules (insofar as this affects the size and distribution of weight in the molecule). The invention contemplates that employing more than one than one type of construct may be desirable and the invention is therefore directed to the various combinations and permutation of constructs according to the invention, in combination with each other and other therapeutic molecules or modalities. One of constructs contemplated by the invention, is a multispecific antibody, for example a bispecific antibody having a configuration which allows for binding to two antigens on the same cell, for example a traditional four chain immunoglobulin configuration having a hinge region (including F(ab′)₂ minibodies etc.), a diabody configuration (depending on the relative positions of the target ligands) and others herein referenced and known to those skilled in the art. It will also be appreciated that the mode of action of the multifunctional ligand may be contributed to by fusing or conjugating the multifunctional ligand to another functional moiety, for example, as described in the literature referenced below. These supplementary strategies are set forth below:

### Additional Strategies for Modifying Targeting Capabilities

According to one embodiment, the intrinsic affinity of the first target binding moiety for the first target is greater than the intrinsic affinity of the second target binding moiety for the second target. The term “intrinsic” affinity connotes a measure of the affinity of a given target binding moiety for its target ligand which is independent of the affinity of the at least one other target binding moiety for its target ligand and as used herein could theoretically be evaluated in the context of the multispecific ligand as a whole, if the other target binding moiety had an irrelevant specificity and therefore could not bind to its target ligand. The invention contemplates that at least approximately one, two, three, four, five, six, seven or eight orders of magnitude differences in “intrinsic affinity” may be required to accomplish the targeting objectives of the invention.

According to another embodiment, the relative on-rate* of the first target binding moiety is greater than the relative on-rate of the second target binding moiety. The term relative on rate is used to connote an effective difference in on-rate that may be intrinsic to the individual target binding ligand or may attributable to its configuration or relationship vis-à-vis other parts of the molecule.

Where the intrinsic on-rateⁱ of the first target binding moiety is greater than the intrinsic on-rate of the second target binding moiety, the invention contemplates that the off-rate contribution to the affinity of the second target binding moiety may be proportionally greater than the off-rate contribution to the affinity of the first target binding moiety. The invention contemplates that the binding of the second target ligand binding moiety to its target ligand may be more effective if its lower affinity is attributable in part due its reduced on-rate. The invention contemplates methods for reducing the affinity a target binding moiety by reducing its on rate for example by mutating or adding amino acid residues in regions of the VH or VL that don't directly contribute to the off-rate (of a relatively high affinity binder for the target, forⁱⁱ example, as determined by modeling and structural analysis, for example, by evaluating x-ray crystal structure and evaluating NMR data of the binding, or by mutagenesis, preferably by introducing a diversity of changes in a high-throughput manner (e.g. phage display, ribosome display, microarray or other expression library) including substitutions, additions and deletions within various regions of the VH or VL and determining their effect. For example, the invention contemplates that the second target binding moiety is generated using a library characterized by members in which one of the regions of VH or VL, including particularly the CDR1 and CDR2, for example the CDR1 of the VH or CDR2 of the VL, is shortened and/or mutated in a manner to reduce the probability of its having any direct contribution to the affinity of the selected molecule (through molecular interaction), for example mutated to introduce amino acids that are least important for intermolecular interactions, for example by minimizing the occurrence of amino acids that are important for electrostatic interactions and optionally also hydrogen binding, generating a binder whose affinity will be postulated to be independent of the contribution of the modified CDR, and then optionally evaluating the success of this latter step through further mutagenesis (this step is most revealing if the CDR is shortened but not mutated or mutated to introduce amino acids important for intermolecular interactions) and then using the library to incrementally lengthen the region and/or introduce amino acids important for intermolecular interaction at a distance (e.g. electrostatic interactions and optionally also hydrogen binding) to introduce minimal steric hindrance or intermolecular repulsion. The invention also contemplates that introducing amino acids that have the greatest potential for hydrogen bonding may introduce an aqueous cushion into the interface region with the target ligand to diminish the on-rate contribution to affinity. The invention also contemplates modifying the amino acid composition of an existing binder by introducing or one or amino acids or mutations into a framework region at a location which is proximal to the binding region or a region which borders the interface of approach to the binding region or any interface between the target binding moiety and the target ligand. The invention contemplates that the on-rate and off-rate can be routinely measured using various technologies (e.g. Biacore) known to those skilled in the art, including various techniques of measuring these rates in real-time, for example those that measure the deflection pattern of an incident form of radiation (e.g. Biosite). In one embodiment of the method the antibodies each have unique preferably cleavable peptide tags that are generated for example through a random or partially random insertion of nucleotides into the DNA encoding the antibody and that serve to link them to their DNA e.g. a phage (as per techniques known to those skilled artisans or published in the art) and the antibodies are evaluated independently of a phage (e.g. they may even be cleavable from the phage) or other expression system linkage which allows a more accurate measure of their true on rates and off-rates. The invention also contemplates that FR1 could be lengthened in a relatively high affinity second target binding moiety to reduce its on rate. The cleanable peptide could be a unique identifying CDR. ⁱ The actual on-rate if the on-rate was to be measured independently of the on-rate of the other binding moiety

In another aspect the invention contemplates that the multispecific ligand may comprise an Fc portion and a hinge portion and that one or both of a) the length, amino acid composition or* molecular weight (or various combinations of these interrelated factors) of the Fab or Fc portion; and b) the amino acid composition (including length) of the hinge portion (e.g. any polypeptide segment that provides means for linking two typically heavy chains, e.g. through one or more disulfide bonds, leucine zipper fos-jun, optionally a flexible hinge typical of an IgG1 or having one to several more disulfide bonds e.g. IgG3) are selected to reduce the circumstantial (shear rate, presence of degrading enzymes) affinity of the second ligand binding moiety where the first ligand binding moiety is unbound relative to the circumstantial affinity of the second ligand binding moiety where the first ligand binding moiety is bound. The term circumstantial affinity broadly contemplates that the length and molecular weight of the Fc and the flexibility of the hinge region will individually and collectively contribute to the affinity of the molecule in proportion the shear rate of the fluid environment to a degree depending on whether the target is stationary or moving, once the multispecific ligand is bound. If bound via the second target binding moiety, any increase in the molecular weight especially a distribution of the molecular weight towards the Fc or first ligand binding moiety will serve as a lever in a moving fluid environment, to favor disengagement from binding especially since the off-rate of this binding arm is relatively low to begin with. This same lever effect will impinge on the binding of the first ligand binding moiety but to a lesser functional degree due to its higher affinity. To an extent depending on the context in which binding occurs, the invention also contemplates that the high affinity ligand binding moiety will draw the multispecific ligand from the circulation into a desired target tissue and that the low affinity binding arm will then have greater opportunity to bind even if it does not bind simultaneously with the high affinity binding arm. Where the hinge region is extra flexible or has several regions of flexibility (for example where the heavy chains are linked through several disulfide bonds with regions of flexible linker therebetween) the disengaging effect on the individual and paired binding of both the first and second ligand binding moieties will be less Similarly, using a truncated Fc portion (CH3 deleted, F(ab′)₂ or minibody format) will assist the first ligand binding moiety to remain bound or foster binding of the second ligand moiety and will assist the second ligand binding moiety to remain bound. This construct may be preferred from an effectiveness standpoint (getting both ligand binding moieties bound), where the affinity of the second ligand binding moiety is low to begin with. On the other hand, decreasing the flexibility of the hinge region by alteration to its length and/or amino acid composition and increasing the molecular weight distribution towards the “free” end of the Fc will affect all binding scenarios to a greater extent. The latter strategy may be less desirable where the Fab of the first ligand binding moiety is lengthened (e.g. has a longer hinge region at the N-terminus of the disulphide bond linking the heavy chains, than the low affinity binding arm) to increase its propensity for individual binding. For example, in a conventional four chain or heavy chain antibody (two heavy chains but no light chains) the hinge region could be lengthened or shortened on the amino terminus side of the disulfide bond linking the heavy chains to an extent that does interfere with the simultaneous binding to both the first and second target binding moieties. The invention also contemplates that the target cell environment, naturally or through intervention, is a fluid environment (low shear rate) or enzyme environment which will favor a greater impact on disengagement of the second ligand binding moiety, in the case of an enzyme, one which will cleave off an Fc into which a cleavage site has been introduced so that disengagement due to the lever effect will primarily impinge on binding of the second ligand moiety to the non-target cell population (e.g. low shear rate or presence of MMP type enzymes in a targeted solid tumor environment).

The invention also contemplates that second ligand binding moiety may be selected in an environment in which there is a selective pressure (moderate fluid flow e.g. using live cells or tissue, candidate ligand binding molecules or pairs of the target ligands on latex beads, where the substrate to which they are bound is on an incline or otherwise subject to fluid flow (optionally with rigid or high mol. weight Fc), for simultaneous binding so that the affinity of the second ligand binding moiety is selected on the basis of its ability to augment the binding affinity of a first ligand binding moiety of preselected affinity for the first target ligand (after or optionally before its affinity maturation, depending on the shear force and affinity in question) and thereby augment the affinity of the multispecific binding ligand as a whole, while the first ligand binding moiety is bound. In this way, the strength of the binding affinity of the second ligand may be predicated on the first ligand moiety being bound. The foregoing strategy may have accentuated or at least equal application where the first ligand binding moiety has a longer Fab or for example where both the first and second ligand binding moiety are devoid of a light chain i.e. where having the correct binding interface for the second target binding moiety might be more acute. The invention contemplates that the individual affinity of second ligand binding moiety selected in the above manner would be tested to ensure that its individual affinity was not sufficient for substantial independent targeting.

The invention also contemplates that engineering a suitable affinity antibody for solid tumor targeting in which the on-rate contribution to affinity is reduced (according to the strategy suggested above) may assist a dose of such antibody in achieving better tumor penetration. An antibody having a reduced on rate could be fused to a toxin such as a truncated version of PE or conjugated to a radionuclide, etc. the reduced on-rate contribution ensuring that the antibody will be less likely to bind at sites proximal to the point of entry to relieve congestion in that area and better ensure its diffusion throughout a tumor. The invention contemplates that the strategies described above will better permit the affinity to be more suitably apportioned between the on-rate. The invention contemplated that a higher on-rate lower off-rate Ab could be delivered in alternating days or other cycles of treatment. Thus the invention is directed to an antibody conjugated or fused to a functional moiety, wherein the on-rate contribution to the affinity of the antibody is anywhere between 3× and two order of magnitudes less than typical molecules having suitable properties for tumor penetration through diffusion, for example molecules having anywhere (any increments) between 10⁻⁷ and 10⁻¹⁰ molar affinities (e.g. 5×10⁻⁷, 3×10⁻⁸) preferably increments between 10⁻⁸ to 10⁻¹⁰ (molecules where the on rate is normally approx. 10⁻⁵) molar affinities, more preferably increments between 5×10⁻⁸ and 5×10⁻⁹.

It will be appreciated that the foregoing strategies could be employed for designing a multispecific ligand which will primarily target cells which have both the first and second target ligand (e.g. where the ligands together are present primarily on the target cell population) even where neither target ligand is individually found primarily on the target cell population, by employing a multispecific ligand in which neither target ligand is of sufficient affinity in the circumstances to effectively (with effect) bind or remain bound without the other target ligand being available for simultaneous binding. As suggested above, it will be appreciated that a relatively higher affinity ligand could initially be employed on one of the ligand binding arms to select a second ligand binding arm which improves the binding properties of the multispecific ligand under a suitable biologically relevant shear stress and which is selected or later modified so that it is individually insufficient for targeting its target on non-target cells in the circumstances in which it will be employed, and that the high affinity ligand binding arm can subsequently be reduced to moderate affinity with similar lack of individual effect. In one embodiment, this construct can be employed to evaluate the effect of blocking two receptors on the same cell, for example chemokine receptors e.g. CCR7 and CXCR4 on a breast cancer cell. In one embodiment, the off-rate of one or optionally both ligand binding moieties is sufficient in the circumstances to permit the moiety to remain bound for a sufficient duration for the other moiety to bind i.e. it exceeds its effective or intrinsic on-rate. In one embodiment, both arms of such multispecific ligand, bind to their respective ligands with low affinity. In one embodiment, one such arm is a “coybody”.

In connection with the foregoing and ensuing strategies it will also be appreciated that the hinge region may be lengthened on the N-terminal side of the most N-terminus linker between the heavy chains so as to permit greater flexibility in the binding of different antigens at different possible proximities to one another.

With respect to each of the preceding aspects of the invention, the invention also directed to a multispecific ligand comprising a first ligand moiety which recognizes a first target ligand that is over-expressed on a disease associated entity for example a diseased or disease-causing or mediating cell or infectious agent and a second ligand binding moiety that recognizes a target ligand and wherein the first target ligand is characterized in that it does not lend itself to facilitating or permitting internalization of the second ligand binding moiety.

The invention also contemplates that a target ligand can be distributed in various concentrations for testing purposes on cell sized latex beads, columnar packing materials or flat substrates having a high density dispersion of both target ligands.

The invention is also directed to combination therapies with the foregoing multispecific ligands including, without limitation, immunotoxins, drugs, therapies with other multispecific ligands herein described and particularly for cancer therapies directed at interfering with the integrity of tumor cell vasculature.

Delivering Biologic Effector Ligands To a target entity

With respect to each of the preceding aspects of the invention, the invention also contemplates that the second ligand binding moiety may be constituted in whole or in part by a ligand which binds to a biologic effector ligand (such as a cytokine, colony stimulating factor, chemokine, growth factor etc. or related extracellularly expressed regulatory molecules that control their expression such as inhibitors, agonists, antagonists of same, which may have corresponding biological receptors), the ligand optionally having a higher affinity for the biologic effector ligand than the affinity of that biologic effector ligand for its receptor, and wherein the ligand, combined with the biologic effector ligand (i.e. bound thereto), has a relatively diminished ability to bind and/or stay bound to the receptor (the second target ligand) independently of the binding of the first target binding moiety to the first target ligand e.g. a lower affinity of approximately one, two, three, four, five, six, seven or eight orders of magnitude. The invention contemplates that the foregoing construct can be used to deliver the biologic effector ligand more selectively to the target cell population recognized by the first ligand binding moiety. The second ligand binding moiety may be an antibody portion of a multispecific ligand of the invention and the invention contemplates that a library of second ligand binding moieties, recognizing multiple different epitopes on the biologic effector ligand, can be screened for their ability to bind to the biologic effector ligand, while it is bound in situ to its receptor, for example, using a microarray of such antibodies, and the affinities of the binders can be evaluated. The invention also contemplates that suitable antibodies could be generated by “panning” (with an expression library, e.g. phage display, ribosome display, or other similar display systems including yeast, bacterial, viral, cell based or cell-free display systems) or otherwise screening (e.g. using antibody microarrays) against the biologic effector ligand while bound to its receptor and screening for their ability to bind to the biologic effector ligand independently of its receptor. Again, the affinities of the antibody coupled to the biologic effector ligand for the target receptor could be evaluated. More generally, the invention contemplates that an array of antibodies which recognize all different epitopes on a given biologic effector ligand could be generated and tested for their ability to accommodate binding of a biologic effector ligand to a first but not a second in a related family of receptors. This could be accomplished by screening the array for one or more members that bind to the biologic effector ligand (BEL) while bound to its receptor, and testing the identified members for their ability to bind to the second receptor, preferably by loading the biologic effector ligand onto an array of those members pre-bound with BEL and detecting those BEL bound members for those which do and do not bind to the second receptor. Therefore the invention is also directed to an antibody which accommodates binding of the BEL to one receptor but hinders the binding to at least one second receptor, preferably by steric, charge or other inter-molecular hindrance, attributable to the proximity of the antibody epitope on the BEL to the BEL's receptor binding site and optionally also the amino acid composition of the antibody at that interface.

The invention contemplates that fluid flow can be simulated in a purification or immunoaffinity column packed with one or more known packing materials to simulate flow over a ligand coated substrate.

The invention also contemplates an apparatus and method for testing ligand binding in a circulating fluid environment in which the multispecific ligands of the invention can be tested and wherein a continuous flow of ligands, including target ligands, ligands of the invention and/or ligand bearing entities (e.g. cells or synthetic e.g. latex spheres which can be adjusted to a cell size) to which one or types of ligands have been affixedly associated accordingly to known methods) can be generated. The fluid contact interface of the apparatus has a generally circular shape and is convex or otherwise capable of containing the fluid and thereby preferably permits fluid to flow around the surface continuously. For example, this surface may be enclosed with a bagel-shaped cylinder which is optionally open at a location opposite the fluid contact surface for introducing and/or removing its contents, or it may completely enclosed with the exception of an access port, from which any air may optionally be displaced or evacuated. The invention contemplates that the apparatus (at least the fluid contact vessel) can be rotated or oscillated (e.g. in an elliptical, oval or similar shape well known to those skilled in the arts of fluid mechanics and related engineering arts) in a variety of different planes or with rocking-like motion in multiple planes or subject to peristaltic pressure (i.e. where flexible tubing is used) to generate a continuous, optionally turbulence free fluid flow over the fluid contact surface at selected rates simulating the various shear rates of arterial, venous, intra-lymphatic flow (including different diameters of such vessels) or interstitial flow. The invention also contemplates that the fluid contact surface may be provided with a 1) substrate for linking ligands of the invention or target ligands or ligand bearing entities to permit fluid flow across the substrate in a plane substantially parallel or conforming to the axis of flow.

In another aspect the invention is directed to methods of making a multispecific antibody in which:
a) the light chains are the same for both the VL domains. For example, the light chains (assuming the construct has two light chains) are generated for a first target binding moiety e.g. in one aspect of the invention, the relatively high affinity binder, optionally from a light chain germ line sequence, and this light chain is then coupled with a diversity of heavy chains to select a pair of chains which bind to the second target ligand, thereby constituting the second ligand binding moiety, which may be a relatively low affinity binder. An alternate or concomitant strategy to generate a lower affinity second ligand binding moiety would simply be to substitute the light chain of the first ligand binding moiety for that of the second ligand binding moiety and to test the affinity. In the case of a multispecific which target BELs to particular target cells, where for example, two high affinity binders are preferred, the heavy chain and light chain binding to the BEL can be truncated correspondingly at the CH1/CL region so that the VH/VL interfaces and cysteines pairing these heavy and light chains are similarly spaced but spaced differently from the other VH/VL chains. By linking the heavy chains as explained above, all chains will pair correctly. It will be appreciated that the foregoing production strategies could be applied to the production of heavy chain antibodies (two chains structures without associated light chains), wherein the heavy chains are from human or other species and that production in this case could be adapted to E. Coli. It will also be appreciated that deletion of a substantial part of the CH1 and CL domains can be measured to provide a space for the BEL to sit in line with the other Fab which can be lengthened in the linker or CH1 domain, as shown in Figure C. The invention contemplates that evaluation of a diversity of the first target binding moiety can be accomplished with the BEL place to best accommodate selection in the context of the entire structure as a whole.
b) With respect to other methods to make bispecific and bispecific fusions see Antibody Fusion Proteins Wiley-Liss 1999 (infra) e.g. particularly p 131 et seq., and Chapter 7 and the discussion, Methodologies improving the correct pairing of heavy chains are well-known in the art.

Such a construct could also be employed in conjunction with other functional moieties fused or conjugated thereto, for example toxins, cytokines, enzymes, prodrugs, radionuclides etc.

In one preferred embodiment, the invention is directed to a multispecific ligand* with at least two different binding specificities for different target ligands* on the same target cell* and adapted to bind contemporaneously to the different target ligands, said multispecific ligand comprising a first target binding moiety which preferentially* recognizes a first target ligand and a second target binding moiety which preferentially recognizes a second target ligand, and wherein said first target binding moiety recognizes a target cell-associated* target ligand and said second target binding moiety recognizes a non-cell-associated target ligand which is present on target cells and non-target cells; and wherein the ability of the second target binding moiety to bind to the second target is diminished relative the ability of the first target binding moiety to bind to the first target ligand, the first target binding moiety having an ability to bind to the first target ligand which is at least sufficient for the first target moiety to bind to the first target ligand independently of the second target binding moiety binding to the second target ligand and an off-rate which at least sufficiently exceeds the on-rate of the second target binding moiety for the second target ligand to provide opportunity for the second target moiety to bind the second target ligand when the first target binding moiety is bound to first target ligand, the second target binding moiety having a relatively diminished ability to bind or stay bound to the second target ligand independently of the binding of the first target binding moiety to the first target ligand, such that the multifunctional ligand will bind to the target population of cells in preference to the non-target population of cells. As suggested above, the strategy embodied in this preferred embodiment can also be employed in connection with any one or any combination of compatible strategies referred to above, to diminish in degree the requirement of using a low affinity second ligand binding moiety.

In another aspect the invention is directed to heterofunctional ligand comprising a first moiety which binds to a first target ligand and a second moiety which binds to a second target ligand, and wherein the affinity or avidity or both the affinity and avidity of said first moiety are selected to enable the first moiety to bind to the first target ligand independently of the ability of said second moiety to bind to the second target ligand and wherein the relative avidity or affinity or both the affinity and avidity of said second moiety are selected or adjusted to substantially reduce the probability of the second moiety binding to the second target ligand without the first moiety, first or substantially contemporaneously, binding to the first target ligand. For example, in one embodiment the first moiety is divalent and the second moiety is monovalent. In one embodiment the affinity of the first moiety for its target ligand is for example up to several orders of magnitude greater than the affinity of the second moiety for its target ligand, as discussed below. In a preferred embodiment both moieties are capable of binding to different target ligands on the same cell, for example as hereinafter specified, although in the case of tumor cell targeting, particularly with respect to cells that are growing adjacent to another the invention contemplates that the first moiety may bind to one cell and the second moiety may bind to a neighbouring cell. Accordingly, in the case of receptors requiring cross-linking for biological activity the invention contemplates that such same cell interactions and adjacent cell interactions are optionally accomplished when the second moiety is bivalent. In one embodiment, at least one of said first and second moieties comprise one or more antibody components. In another embodiment, said first moiety binds to at least one cell-surface ligand which differentiates between cells of the same population or sub-population, for example, at least one ligand which differentiates which between populations or sub-populations of immune cells (e.g. see WO 01/21641, U.S. Pat. No. 6,156,878), for example, activated vs. non-activated, disease-associated or non-disease-associated (over-expressing or uniquely expressing certain receptors or other ligands [for example cytokine or growth factor receptors, particular immunoglobulin like molecules or MHC peptide complexes] or other differentiating markers hereinafter exemplified or apparent to those skilled in the art), and said second moiety, in virtue of its binding to the second target ligand, directly or indirectly exerts a biologic effect e.g. a therapeutic effect, for example an immune modulating effect. In a further preferred embodiment said second moiety has a broader target cell population than said first moiety e.g. see Wiley H. et al. Expression of CC Chemokine Receptor-7 and Lymph Node Metastasis, J. Natl. Cancer Inst. 93:1638-1643; Moore M A Bioessays 2001 August; 23(8):674-6. (The invention contemplates that by targeting CCR7 receptor selectively on tumor cells, for example using a relatively high affinity binding moiety for a tumor associated antigen and a relatively low affinity moiety which binds to and blocks CCR7 receptor, e.g. when combined in therapy with a chemotherapeutic agent or an immunotoxin for the same tumor, metastasis can be inhibited). For example, in one embodiment said first moiety binds to a tumor associated antigen on a tumor cell and said second moiety binds to a receptor which is found on the tumor cell but also on a broader population of cells. In another embodiment said first moiety binds to an antigen associated with particular population of leukocytes and said second moiety binds to a receptor which is found on that population of cells but also on a broader population of cells (e.g. apoptosis mediating receptors Journal of Immunology 1998 160:3-6, Nat Med 2001 August; 7(8)954-960, WO 01/85782; ICAM-R WO 00/29020; see also WO 01/85768, WO 01/85908; WO 01/83755, WO 01/83560, WO 01/29020; Vitale et al. Proc. Nat. Acad. Sci. 2001 May 8; 98(10):5754-5769; CCR2 see also U.S. Pat. No. 6,312,689; U.S. Pat. No. 6,294,655 Anti-interleukin-1 receptor antagonist antibodies and uses thereof; U.S. Pat. No. 6,262,239; U.S. Pat. No. 6,268,477). In another embodiment the second moiety does not necessarily bind with lower affinity to its target however it may bind to a first ligand which in turn binds to a second ligand on a target cell (e.g. a receptor on the target cell e.g. a cytokine, chemokine or growth factor receptor), for example the receptor being on the same cell to which the first moiety binds, and it binds in a manner in which it partially interferes with the binding of the first ligand to the second ligand and thereby directs or retargets that first ligand to the second ligand in a manner which accomplishes the intended interaction of the first with the second ligand (e.g. a signal transduction or blocking interaction i.e. the second moiety causes the e.g. cytokine to bind to its receptor without engendering the biological effects attributable to receptor binding e.g. signal transduction, which may be assessed by assaying for effects of e.g. signal transduction according to well established techniques in the art) but less competitively relative to the first moiety so that the first moiety exerts a targeting function i.e. where the first ligand bound by the second moiety binds to a broader than desired population of cells. The binding of the second moiety may also be compatible with the first ligand binding to one cell surface ligand but not another e.g. see WO 00/64946 the contents of which are hereby incorporated by reference. The ability to identify ligand residues of importance to binding or residues other these, the alteration of which might interfere with binding is well established in the art. The invention contemplates varying, by high throughput techniques e.g. phage display, residues of an antibody that are not involved in first ligand binding to create variants which can be tested for partial interference with first ligand binding to the second ligand e.g. receptor binding.

Examples of receptors for blocking or activation by the targeting methods described herein include tyrosine kinase type receptors, serine kinase type receptors, heterotrimeric G-protein coupled receptors, receptors bound to tyrosine kinase, TNF family receptors, notch family receptors, guanylate cyclase types, tyrosine phosphatase types, adhesion receptors etc. (for example receptors see those discussed in Cancer: Principles and Practice of Oncology 6^{th} Ed. De Vita et al. Eds Lippincott 2001, including particularly Chapter 3, 7 and 18, The Autoimmune Diseases, Academic Press Third Edition, Rose/Mackay ISBN: 0125969236, Immunology 6^{th} Edition, Mosby 2001 Roitt et al. Eds; Molecular Mimicry, Microbes & Autoimmunity by Madeleine W. Cunningham (Editor), Robert S. Fujinami (Editor) December 2000, among other references hereinbelow identified). Further mention may also be made of interleukin and interferon type receptors, HGF receptor (see for example U.S. Pat. No. 6,214,344), CD45, CXC family receptors including CXCR1 and CXCR2 receptors including IL-8 receptor, EGFRs, receptors for molecules with functions in apoptosis or homeostasis, receptors such as FGF which sensitize tumor cells to chemotherapeutic agents, etc. It is known for example to modify receptor ligands in a way which does not interfere with a signaling function (the residues important for signaling may be known or can be readily ascertained e.g. see Retargeting interleukin 13 for radioimmunodetection and radioimmunotherapy of human high-grade gliomas. Debinski W, Thompson J P. Clin Cancer Res 1999 October; 5(10 Suppl): 3143s-3147s) but reduces the affinity of the ligand for this receptor (see also WO 01/19861). Alternatively, the second moiety may be an antibody which is agonistic or antagonistic and used to block, activate, neutralize etc the receptor. With respect to EGFR family, TNF family and other receptor targeting antibodies which are capable of causing apoptosis directly or indirectly, see U.S. Pat. No. 5,876,158, WO 00/20576, WO96/08515, WO 01/44808 (P75AIRM1), WO 00/29020 (ICAM-R), WO 99/12973, CA 2236913 etc. The invention also contemplates that the second moiety may also be targeted to a specific portion of a receptor which differentiates it from other receptors of its class and more generally contemplates that the second moiety may contribute to the targeting ability of the multifunctional ligand.

In another aspect, the invention also contemplates that the first moiety binds to a target cell and said second moiety binds to a ligand, for example a natural ligand, (e.g. a cytokine or chemokine circulating at normal levels or at higher levels attributable to a disease or treatment of a disease with another therapeutic molecule) and retargets that ligand (for example, the ligand may be retargeted from circulation) to a targeted cell. For example the invention contemplates that IL-2 may be retargeted to LAK cells or CTLs via a high affinity Leu-19 binding first moiety. For example, antibodies including fragments thereof which bind to cytokines or other natural ligands for retargeting purposes (e.g. single domain antibodies) can be made by phage display against the cytokine or ligand while bound in situ to its receptor. The invention also contemplates that the affinity for the cytokine may be adjusted to regulate the degree of targeting and that serum samples may be evaluated to assess the degree of bound cytokine and the relative degree of bound and unbound cytokine. Among other methods, for example, the invention contemplates that a radiolabeled multifunctional ligand may be used assess the amount of label associated with the multifunctional ligand when bound to the cytokine, by capturing the ‘complex’ with an antibody that recognizes both antigenic determinants on both the cytokine and an adjacent portion of the ligand binding thereto i.e. forming a composite epitope), such as may be generated by phage display and assessing the amount of label relative to the amount of captured complex. The invention also contemplates administration of supplemental amounts of natural ligand to compensate for the degree in which the ligand is retargeted insofar as such retargeting might impact negatively on immune or other physiological processes.

In another aspect the invention contemplates that patients treated with antibodies to a particular biologic effector ligand e.g. a natural ligand e.g. a cytokine, for example TNFα, may preferably be treated with a multifunctional ligand having a first moiety which binds to at least one cell type and a second moiety which binds to a natural ligand such as a cytokine for retargeting that cytokine to that cell type, as in a preventative method for treating a disease, e.g. cancer. In this respect the invention contemplates that the antibody is capable of binding to the cytokine but once bound the cytokine, the cytokine is incapable and/or only weakly capable of binding to its receptor and/or that the multifunctional ligand also comprises a higher affinity receptor blocking moiety to minimize retargeting of the primary disease site. In one embodiment, the first moiety binds with relatively higher functional affinity (i.e. avidity, affinity, and/or relatively advantageous binding capacity in virtue of multiple ligand binding arms, each binding to different ligands on the target cell) to ensure binding to the retarget cell. In another embodiment the bound cytokine is capable of binding to the cytokine receptor at the retarget site but incapable of binding to the receptor at the disease site owing to differences in the receptors at the two sites. The invention also contemplates using antibodies which interfere but do not preclude binding of the biologic effector to provide a less toxic effect.

For example, patients with Crohn's disease that are treated with anti-TNFα (see for example, Expert Opin Pharmacother 2000 May; 1(4): 615-22 and references cited therein) may be treated according to the invention with a bispecific antibody having, in addition to an anti-TNFα binding moiety, which reduces the affinity of the bound TNF for the receptor, but also an antibody moiety which binds to tumor antigen which is expressed on many different tumor types or optionally a trispecific antibody which additionally binds to a second multi-carcinomic antigen, preferably one which broadens the range of targeting against prevalent cancers. With respect to tumor antigens mention may be made of EGFR, EPCAM, MUCINs, TAG-72, CEA, H11 among other known multicarcinomic antigens (see also Cancer: Principles and Practice of Oncology 6^{th} Ed. De Vita et al. Eds Lippincott 2001 Chapters 18 and 20.5). In another embodiment, the second moiety differentially retargets a cytokine to one receptor in preference to another, for example, to a TNF receptor over-expressed on tumor cells in preference to a TNF receptor associated with Crohns disease. In a related but also independent aspect, the invention contemplates a method of screening for an antibody which preferentially binds to a ligand when bound to a first receptor relative to another second receptor by screening for antibodies (e.g. by phage display, ribosome display, etc.) which bind to the ligand e.g. a cytokine, when bound in situ to the first receptor, and selecting among them those that bind to the ligand e.g. cytokine but do not bind (substractive screening) or bind with lesser affinity to the cytokine when bound to the second receptor, as well as to antibodies and multifunctional ligands created by this method (see also U.S. Pat. No. 6,046,048 and WO 99/12973 and references cited therein with respect to TNF family of receptors). Variations in the extracellular domains of such receptors are known and can be ascertained by methods known to those skilled in the art.

Further with respect to multifunctional ligands having a higher affinity targeting moiety relative to the second i.e. effector moiety, the second moiety may be for example an antibody or other ligand which interferes with the binding of the regular ligand for this receptor. For example, the invention contemplates a first ligand binding moiety which recognizes activated T-cells and a second ligand binding moiety which blocks the IL-16 receptor for testing the effect on Crohns disease (or alternatively activates an IL-16 receptor on those cells e.g. by using a high affinity IL-16 bound second moiety which becomes relatively low affinity IL-16 receptor ligand when bound to the antibody, again to test the effect on Crohn's disease (see Gut 2001 Dec. 49(6) 795-803) For example, in one embodiment, the invention contemplates that the second moiety blocks a receptor that are found on cells other than the target cell, the blockage of which leads to the apoptosis of or destruction of the cell e.g. CD95 (e.g. see Jung G. et al., Target cell-restricted triggering of the CD95 (APO-1/Fas) death receptor with bispecific antibody fragments Cancer Res 2001 Mar. 1; 61(5):1846-8). With respect to blocking insulin like growth factor receptor, insulin receptor etc. see The IGF system in thyroid cancer: new concepts. Vella V., Mol Pathol 2001 June; 54(3): 121-4; Insulin receptor isoform A, a newly recognized, high-affinity insulin-like growth factor II receptor in fetal and cancer cells. Mol Cell Biol 1999 May; 19(5): 3278-88; Expression of the insulin-like growth factors and their receptors in adenocarcinoma of the colon. Freier S Gut 1999 May; 44(5): 704-8; Pandini G., Insulin and insulin-like growth factor-I (IGF-I) receptor overexpression in breast cancers leads to insulin/IGF-I hybrid receptor overexpression: evidence for a second mechanism of IGF-I signaling. Clin Cancer Res 1999 July; 5(7): 1935-44. With respect to targeting beta-1 integrins, see e.g. Masumoto A, et al Role of beta1 integrins in adhesion and invasion of hepatocellular carcinoma cells. Hepatology. 1999 January; 29(1): 68-74. Arao S, et al. Beta1 integrins play an essential role in adhesion and invasion of pancreatic carcinoma cells. Pancreas. 2000 March; 20(2): 129-37. Xi.e. Y, Xi.e. H. Characterization of a novel monoclonal antibody raised against human hepatocellular carcinoma. Hybridoma. 1998 October; 17(5): 437-44. Peng H, et al Production and characterization of anti-human hepatocellular carcinoma monoclonal antibodies. Hua Xi Yi Ke Da Xue Bao. 1990 September; 21(3): 259-62; Whittard J D, Akiyama S K. Activation of beta1 integrins induces cell-cell adhesion. Exp Cell Res. 2001 Feb. 1; 263(1): 65-76 Nejari M, et al. alpha6beta1 integrin expression in hepatocarcinoma cells: regulation and role in cell adhesion and migration. Int J. Cancer. 1999 Nov. 12; 83(4): 518-25; Yao M et al Expression of the integrin alpha5 subunit and its mediated cell adhesion in hepatocellular carcinoma. J Cancer Res Clin Oncol. 1997; 123(8): 435-40.

The invention also contemplates a method of optimizing the cooperative affinities of respective binding ligands of a multifunctional ligand described herein and the length of a linker therebetween for the above and applications described below by phage or ribosome display etc. in which the multifunctional ligand is a single polypeptide chain, for example, two single chain Fvs or single domain antibodies linked in sequence, or a diabody (see U.S. Pat. No. 5,837,242), by varying the DNA sequence corresponding to amino acids that represent linker and/or for example CDR regions that are postulated to impact on affinity according to methods and strategies that well known in the art for affinity maturation. These same strategies can be employed for engineering lower affinity molecules. Accordingly, more generally the invention is directed to a phage display or similar library (e.g. a ribosome display library or a microarray) in which the population of variants is a multispecific ligand, including a multispecific ligand according to the invention herein defined.

In another embodiment blockage of a receptor does not necessarily lead to cell death but may lead only to decreased or increased release of certain cytokines etc, for example as mediated via the IL-6 receptor. In another embodiment the second moiety may achieve the desired therapeutic effect by constituting the normal ligand for that receptor or a functional substitute. The multispecific ligand may also be fused or conjugated to a toxic moiety or other effector. In another or further preferred embodiment, said first moiety comprises two binding ligands (e.g. one or both of which may be an antibody) which respectively bind to two different target ligands each of which contributes to its total binding capacity and neither of which are sufficient to efficiently target the cell, for example a ligand which binds to a specific MHC peptide complex and a second reduced affinity ligand which binds to a ligand on an APC. This approach also obviates the need to create high affinity ligand for a particular MHC peptide complex, although this can been accomplished. In another or further preferred embodiment the target cell is an immune cell and the second moiety binds to a molecule involved in cellular adhesion, a cytokine receptor, a ligand which stimulates the activity of said immune cell, a ligand which inhibits the activity of said immune cell, a ligand which causes one or more cytokines to be released, a ligand which prevent one or more cytokines from being released, a ligand which causes or facilitates apoptosis of said immune cell or a ligand which permits internalization of said multispecific ligand. In another preferred embodiment the heterofunctional ligand is fused or conjugated to a therapeutic agent or a moiety that binds to a therapeutic agent (exemplified below) or a ligand which effects binding to another immune cell, for example a T cell. In another preferred embodiment, the multispecific ligand is a bispecific antibody, a trispecific antibody or a tetraspecific antibody. In a further preferred embodiment the first moiety binds to but is incapable of modulating the activity of said immune cell and said second moiety modulates the activity of said immune cell independently of said first moiety. In another preferred embodiment the multispecific ligand further comprises a moiety that binds to at least one ligand located on the intraluminal surface of a lymphatic vessel, preferably a lymphatic vessel associated ligand, as hereinafter defined. In other aspects the invention is directed to a pharmaceutical composition comprising such a multispecific ligand and a pharmaceutically acceptable carrier, a method of using the heterofunctional ligand in the preparation of a pharmaceutical composition for treating a disease, and to a method of treating a subject by administering same in a therapeutically effective amount.

The term heterofunctional is used broadly to refer to a ligand: 1) comprising at least two functional moieties that have different functions or different capacities to perform the same function and 2) which is typically and preferably heterospecific (having two binding specificities).

Unless the context dictates otherwise the term avidity when used in a comparative, quantifiable or controllable sense is used to refer the valency of the binding entity or moiety. The term functional affinity is used as a composite term referring to a quantitative and controllable (though not necessarily quantifiable, especially when its consists of both avidity and affinity components) propensity to specific binding attributable to one or both of avidity and affinity effects.

In another aspect, the invention contemplates that cells, particularly immune cells, that are expected to be present at or proximal to a disease site (e.g. at the site where an immune cell crosses the vascular endothelial cell wall), in virtue of the disease or a therapeutic modality which is employed in relation to the disease or a concurrent disease, including cells that directly mediate the disease, may be targeted in virtue of a marker associated with such cells, e.g. markers associated with activated immune cells or disease mediating immune cells e.g. LEU-19, a marker associated with activated or killer T-cells, etc for example with an antibody, which is linked to a moiety that is capable of exerting a therapeutic effect in relation to the disease, for example, an immunoliposome or an antibody linked to another therapeutic delivery system (for example streptavidin or biotin fused, coated or conjugated entities or other payload carrying entities (see for example U.S. Pat. Nos. 5,439,686, 6,007,845, 5,879,712, 5,456,917, 6,165,502, 5,079,005, 5,888,500, 5,861,159, 6,193,970, 6,190,692, 6,077,499, WO 00/69413, WO 01/07084, etc.). For example, an immunoliposome may carry one of or a combination of cytokines, chemokines, toxins or other therapeutic molecules suitable for treating the disease directly or indirectly, for example by attracting or preventing the attraction, activating, anergizing or otherwise modulating the activity of immune cells for therapeutic or related purposes. Thus according to another aspect, the invention is directed to a multifunctional ligand characterized in that it exerts an independent biologic function said multifunctional ligand comprising a ligand which binds to a non-diseased disease associated cell and: a) a therapeutic entity; b) a ligand which binds to a therapeutic entity; or c) a ligand which binds to a disease mediating entity e.g. a biologic effector molecule which is released by the disease mediating entity or the diseased cell e.g. a cytokine or other BEL which mediates or aggravates a disease process. Preferably said multifunctional ligand comprises at least two of a), b) or c) and preferably all three.

The term “independent” refers to a function which is primarily exerted in relation to an entity other than the entity that is targeted (save for possible entity associated side effects). The invention contemplates that targeting a cell which localizes to a disease site will better localize the independent effect of the targeting ligand to that locale. For example, an antibody which binds to and neutralizes a cytokine or other BEL associated with Crohn's disease e.g. TNF alpha at the disease locale if targeted to an activated CD4+ T-cell using a marker which identifies activated T-cells.

In another aspect the invention is directed to a heterofunctional ligand comprising a first moiety which specifically binds to at least a first target ligand on a first entity and a second moiety which specifically binds to at least a second target ligand on a second entity, and wherein the affinity or avidity or both the affinity and avidity of said first moiety are selected to enable the first moiety to bind to the at least one first target ligand independently of the ability of said second moiety to bind to the at least one second target ligand and wherein the avidity or affinity or both the affinity and avidity of said second moiety are selected to enable the second moiety to bind to the second entity in preference to the first moiety binding to the first entity when both first and second moieties are substantially contemporaneously bound to the respective first and second entities. In one embodiment the first moiety comprises at least one ligand preferably at least one antibody which binds to a first cell, for example an intraluminal lymphatic endothelial cell and the second moiety comprises a ligand, preferably at least one antibody which binds to a different cell, for example a disease associated cell (hereinafter exemplified and meaning, unless the context implies otherwise, diseased cells or disease causing, mediating (i.e. having a role which is known to be intermediary or indirectly facilitating e.g. antigen presenting cells) or mitigating cells (cells, typically immune cells, which directly or indirectly counteract the diseased or disease causing or mediating cells). In other aspects the invention is directed to a pharmaceutical composition comprising such a heterofunctional ligand and a pharmaceutically acceptable carrier, a method of using the heterofunctional ligand in the preparation of a pharmaceutical composition for treating a disease, and to a method of treating a subject by administering same in a therapeutically effective amount.

In another aspect the invention is directed to a multispecific ligand comprising a first moiety which specifically binds to at least one first target ligand on a first entity (e.g. a lymphatic endothelial cell, a diseased cell or a cell proximal to a site of disease) and a second moiety which specifically binds to a second target ligand or site on a second entity, and wherein the second entity binds to a third target ligand, and wherein the first and third target ligands may be on the same or different entities e.g. the same or different cells, and wherein preferably the affinity or avidity or both the affinity and avidity of said first moiety are selected to enable the first moiety to bind to the first target ligand independently of the ability of said second moiety to bind to the second target ligand and independently of the ability of the second moiety to bind to the third target ligand (the first moiety optionally comprising more than one ligand (which may be the same ligand or a different ligand) one or more of which are necessary for binding and optionally each of which is sufficient for specific binding) to corresponding first target ligands) and preferably wherein 1) the avidity or affinity or both the affinity and avidity of said first moiety is/are selected to enable it to bind to the at least first target ligand in preference to the second moiety binding to the third target ligand when both said first and second moieties and the second entity are substantially contemporaneously bound to their respective target ligands e.g. to effect a transfer or 2) wherein the avidity or affinity or both the affinity and avidity of said second moiety for the second entity are selected to enable the first moiety to bind to the first entity in preference to the second moiety binding to the second entity and/or 3) wherein the avidity or affinity or both the affinity and avidity of said second moiety for the second entity are selected to enable the second moiety to bind to the third target ligand in preference to the second moiety binding the second entity when both first and second moieties are substantially contemporaneously bound to the respective first and second entities and the second moiety is substantially contemporaneously bound to the third target ligand), or 4) wherein both 1) and 2) above are both operative conditions. In one embodiment, the first entity is a diseased or disease causing, mediating or mitigating cell, for example an immune cell (the first moiety preferably binding to a particular population or sub-population of the first target entity e.g. the immune cell, for example activated T cells), the first moiety optionally comprising two or more ligands which may be the same or different and which bind to two or more respective first target cell surface ligands (though not necessarily to any particular effect (and in one embodiment to no effect at all) other than to better bind to and thereby target the cell, preferably in competition with the second entity, which in a preferred embodiment targets a broader population of cells), and the second entity e.g. a biologic effector ligand is an entity that binds to a third target ligand, the third target ligand preferably being expressed on the surface of a cell for example the same immune cell, for example a natural cell surface ligand, to which binding yields a desired effect, for example a therapeutic advantage, the second moiety being, for example, the natural ligand for the cell surface ligand or functional mimitope or antagonist or agonist thereof, for example a cytokine, the third target ligand in this case being a cytokine receptor on the immune cell. The invention is also directed to a method of “targeted delivery” of a therapeutic entity to a cell in need of such therapy by administering said heterofunctional ligand. In this respect numerous therapeutic entities will be apparent to those skilled in the art, only some of which are mentioned herein by referring to the therapeutic entity itself or by referring to the third target ligand for which such entity is known and available or readily made by routine skill in the art. Optionally the heterofunctional ligand (and similarly in the case of other multispecific ligands of the invention described above which are adapted to deliver a BEL) is delivered with the second entity, preferably in the same composition (preferably bound). In the case where the second entity is a natural ligand circulating in the path of delivery of the heterofunctional ligand, some proportion (0-100%) of the heterofunctional ligand may be delivered without supplied second entity, particularly if the treatment or the disease generates an abundance of the natural ligand. In another embodiment the first moiety binds to a target ligand on a stationary cell (for example a vascular endothelial cell or a lymphatic endothelial cell), preferably a tissue or cell type “associated” ligand (more abundantly expressed uniquely expressed on target cells relative to non-target cells), and the third target ligand and the second moiety are cell-surface target and ligand therefore as stated above, for example the second moiety binds to a cytokine and the third target ligand is a cytokine receptor, for example on an immune cell. In one embodiment the first moiety binds to at least one target ligand which differentiates between populations or sub-populations of immune cells and the second entity in virtue of its binding to the third target ligand, directly or indirectly exerts a therapeutic effect, for example by modulating the activity of said immune cell. In another or further preferred embodiment the first moiety is incapable of modulating the activity of said immune cell and said second entity modulates the activity of said immune cell independently of said first moiety. In another or further preferred embodiment the second entity binds to a molecule involved in cellular adhesion, a cytokine receptor, a ligand which stimulates the activity of said immune cell, a ligand which inhibits the activity of said immune cell (e.g. via anergy or tolerance mechanisms), a ligand which causes one or more cytokines to be released, a ligand which prevent one or more cytokines from being released, a ligand which causes or facilitates apoptosis of said immune cell, a ligand which permits internalization of said heterofunctional ligand. In another preferred embodiment the heterofunctional ligand is fused or conjugated to a therapeutic agent or a moiety (e.g. biotin, avidin) that binds to a therapeutic agent (exemplified below) or a ligand which effects binding to another immune cell, for example a T cell. In another preferred embodiment, the heterofunctional ligand is a bispecific antibody, a trispecific antibody or a tetraspecific antibody. In another preferred embodiment the heterofunctional ligand further comprises a moiety that binds to at least one ligand located on the intraluminal surface of a lymphatic vessel, preferably a lymphatic vessel associated ligand, as hereinafter defined.

In other aspects the invention is directed to a pharmaceutical composition comprising such aforementioned heterofunctional ligand and a pharmaceutically acceptable carrier, a method of using the heterofunctional ligand in the preparation of a pharmaceutical composition for treating a disease, and to a method of treating a subject by administering same in a therapeutically effective amount. As suggested below, the foregoing strategy could be used in combination with other targeting strategies herein mentioned or known in the art. The invention contemplates making antibodies to second entities, for example, while bound to their natural receptor, by phage or ribosome display, by methods as hereinafter disclosed.

In another aspect the invention is directed to a heterofunctional ligand comprising at least a first moiety which specifically binds to a first target ligand on a cell and a second moiety which specifically binds to at least a second target ligand on the same cell, and wherein the affinity or avidity or both the affinity and avidity of said first moiety and the affinity or avidity or both the affinity and avidity of the second moiety are selected to substantially reduce the probability of the either moiety singly binding to its respective ligand for a sufficient duration or series of durations to accomplish the function of said heterofunctional ligand unless both first and second moieties are substantially contemporaneously bound to the cell. In a preferred embodiment the first moiety binds to at least one target ligand which differentiates between populations or sub-populations of immune cells and the second moiety in virtue of its binding to the second target ligand, directly or indirectly exerts a therapeutic effect, for example by modulating the activity of said immune cell. In another or further preferred embodiment the first moiety is incapable of modulating the activity of said immune cell and said second moiety modulates the activity of said immune cell independently of said first moiety. In another or further preferred embodiment the second moiety binds to a BEL, for example a molecule involved in cellular adhesion, a cytokine receptor, a ligand which stimulates the activity of said immune cell, a ligand which inhibits the activity of said immune cell (e.g. via anergy or tolerance mechanisms), a ligand which causes one or more cytokines to be released, a ligand which prevent one or more cytokines from being released, a ligand which causes or facilitates apoptosis of said immune cell, a ligand which permits internalization of said heterofunctional ligand. In another preferred embodiment the heterofunctional ligand is fused or conjugated to a therapeutic agent or a moiety (e.g. biotin, avidin) that binds to a therapeutic agent (exemplified below) or a ligand which effects binding to another immune cell, for example a T cell. In another preferred embodiment, the heterofunctional ligand is a bispecific antibody, a trispecific antibody or a tetraspecific antibody. In another preferred embodiment the heterofunctional ligand further comprises a moiety that binds to at least one ligand located on the intraluminal surface of a lymphatic vessel, preferably a lymphatic vessel associated ligand, as hereinafter defined. In other aspects the invention is directed to a pharmaceutical composition comprising such a heterofunctional ligand and a pharmaceutically acceptable carrier, a method of using the heterofunctional ligand in the preparation of a pharmaceutical composition for treating a disease, and to a method of treating a subject by administering same in a therapeutically effective amount.

In other aspects the invention is directed to a method of in vivo modeling or testing using one or more foregoing targeting strategies by administering a heterofunctional/multifunctional ligand as hereinbelow disclosed as well as a method of intra-lymphatic drug delivery employing such ligand and such strategies including adaptations thereof for such purposes, as hereinafter described. In related aspects the invention is directed to a test ligand in the form of such a heterofunctional/multifunctional ligand and compositions thereof.

In one aspect, the invention is directed to a heterofunctional ligand, comprising a first moiety which specifically binds to at least one ligand located on the intraluminal surface of a lymphatic vessel and a second moiety which specifically binds to a disease associated cell and the use of such heterofunctional ligand in treating or preparing a pharmaceutical composition for treating disease associated cells, including diseased cells or disease causing, mediating (i.e. having a role which is known to be intermediary or indirectly facilitating e.g. antigen presenting cells) or mitigating cells (cells, typically immune cells, which directly or indirectly counteract the diseased or disease causing or mediating cells), within a lymphatic vessel. Preferably, the ligand located on the intraluminal surface of a lymphatic vessel is a lymphatic vessel associated ligand.

In another aspect, the invention is directed to a pharmaceutical composition comprising a pharmaceutically acceptable carrier and a heterofunctional ligand comprising a first moiety which specifically binds to a ligand located on the intraluminal surface of a lymphatic vessel and a second moiety which specifically binds to said disease associated cell and the use of such ligand in treating disease associated cells, including diseased cells or disease causing or mediating cells, within a lymphatic vessel. Preferably, the ligand located on the intraluminal surface of a lymphatic vessel is a lymphatic vessel associated ligand.

In another aspect, the invention is directed to a method of treating disease associated cells, including diseased cells or disease causing or mediating cells, within a lymphatic vessel comprising administering to a subject a heterofunctional ligand comprising a first moiety which specifically binds to a ligand located on the intraluminal surface of a lymphatic vessel and a second moiety which specifically binds to said disease associated cell.

It is to be understood that disease-causing cells as used herein includes diseased cells and pathogens, including micro-organisms and viruses.

In another aspect, the invention is directed to a heterofunctional ligand, comprising a first moiety which specifically binds to at least one ligand located on the intraluminal surface of a lymphatic vessel and a second moiety which specifically binds to a therapeutic entity for example a cytotoxin or cytotoxin-linked-entity or a non-toxic entity which is present in toxic amounts and to a method of reducing the toxic effect of such entity in a subject by administering said heterofunctional ligand to said subject.

In another embodiment the invention is directed to a method of therapeutic evaluation and/or targeting/intervention in which such heterofunctional ligand is administered substantially contemporaneously with a cytotoxic substance for example a cytotoxic substance useful for treatment of cancer. The term substantially contemporaneously is used in this connection to mean in a time frame that permits both to exert their respective effects, preferably one or both exerting their respective effect optimally, or one exerting its effect dominantly. It will be appreciated that this might entail that one such entity is advanced in its delivery over the other. Optionally, one or both of these cooperating entities are delivered proximally to their respective target cells, for example by cannulating one or more blood vessels as proximally as possible to the site(s) of a tumor and/or actual or anticipated site(s) of metastases (as discerned by using one or more tumor and vascular imaging agents, for example, one or a combination two or more agents selected from a vascular opaquing agent, a radionuclide conjugated anti-angiogenic antibody, and a radionuclide conjugated anti-vascular endothelial cell marker antibody, which cannulation may occur for example in the course of initial surgical intervention with respect to the primary tumor site) and/or at the same time cannulating one or more lymphatic vessels (which may optionally be located which the help of a radionuclide conjugated anti-lymphatic vessel marker antibody) leading to or from such tumor sites or metastases. The invention contemplates that small sections of vascular prostheses, well known to those skilled the art (e.g. Dacron types) may be grafted into those locations to permit a prolonged and secure attachment of such prosthesis to an intra-vascular cannula for secure delivery to such vascular or lymphatic locations for repeated and/or prolonged administration, optionally while the patient is mobile, optionally using one or more portable infusion devices, including micropumps designed for such purpose (see for example J Neurosci Methods 1997 March; 72(1):35-8, U.S. Pat. No. 5,180,365: Implantable infusion device. See also Cancer: Principles and Practice of Oncology (infra). Numerous embodiments and improvements in vascular prosthesis and in such portable infusion devices and micropumps are described in the relevant scientific and patent literature known to those skilled in the art. The invention also contemplates delivering any multifunctional ligand herein disclosed in the above manner.

It is to be understood that targeting strategies employing the cooperative action of ligands with different affinities for their targets exemplified above, may preferably have affinities which differ, depending on the application and their avidity, by a factor of 20% up to a number of orders of magnitude which may one, two, three, four, five, six and even seven or eight order of magnitude, in order to achieve substantial advantage, as hereafter detailed in connection with one such strategy.

In another aspect the invention is directed to a heterospecific ligand comprising a first moiety which specifically binds to at least a first disease associated ligand located on a diseased or disease causing, mediating or mitigating cell for example a cancer cell or an immune cell, as well as on non-diseased or disease causing, mediating or mitigating cells (non-target cells) and at least a second moiety which specifically binds to a second different disease associated ligand on the same cell and wherein each ligand is expressed on a substantially (see definition below) different, n-overlapping, subset of non-target cells, so that functional binding to a non-target tissue is substantially (see definition below) precluded. In another embodiment the functional affinities of the respective ligands may be selected in accordance with the strategies suggested above, to further facilitate targeting. In another embodiment, both different ligands are required for internalization. In other related embodiments, the heterofunctional ligand comprises at least two different pairs of binding moieties (e.g. a trispecific or tetraspecific antibody which depending on its construction will permit 2, 3 or 4 such different pairs e.g. a tetraspecific single domain type antibody (i.e. consisting primarily of the heavy or light chain variable region or a functional fragment thereof) (see discussion below regarding its construction) allowing the greatest variation in such geometries and preferably simultaneous binding of more than one pair), wherein 1) at least three such ligands are expressed on a substantially (see definition below) different, preferably non-overlapping, subset of non-target cells, so as to further limit binding to non-target cells and/or 2) wherein at least two different pairs of ligands target a substantially different subset of cells within the same target population e.g. different cells within the same tumor (e.g. proliferating vs. non-proliferating cell—the respective amounts of the different types of cells will dictate the percentage of the dose that will be targeted to one population or another). In other aspects the invention is directed to a pharmaceutical composition comprising such a heterospecific ligand and a pharmaceutically acceptable carrier, a method of using the heterospecific ligand in the preparation of a pharmaceutical composition for treating a disease, and to a method of treating a subject by administering same in a therapeutically effective amount. It will be appreciated that the foregoing general strategy can be accomplished with two or more different antibodies have differing and preferably non-overlapping normal i.e. non-targeted cell distributions, preferably administered in the same composition and preferably cross-linked by biotin-avidin like complementary pairs to facilitate cross-linking for internalization or targeting of therapeutic agents. In a preferred embodiment, each such independent antibody carries a different complimentary aspect of a toxic payload e.g. a different liposome (or other payload carrying entity for example a micro or nano particle or sphere or albumin) which complement each other in virtue of their respective contents (e.g. one carries the prodrug and the other the necessary converting enzyme).

In another aspect, the invention is directed to a multifunctional (“multi” meaning at least two) ligand having, at least, a first portion which binds to a lymphatic vessel associated ligand and a second portion comprising an immune function-exerting moiety.

The term lymphatic vessel is used to facilitate broader reference to ligands (e.g. antigens/receptors) present on cells bordering the intra-luminal pathway through the lymphatic system including preferably the lymphatic vessels and optionally also parts of the lymph nodes, and refers in the case of the lymphatic vessels, primarily (from a functional standpoint) to the intra-luminal cell surfaces (not necessarily to the exclusion of non-luminal surfaces) on the intra-luminal endothelial cells (not necessarily to the exclusion of non-luminal lymphatic endothelial cells) of those vessels.

The term ‘associated’ with reference to lymphatic vessels, is used to mean differentially expressed on the surface of endothelial cells of those vessels for targeting purposes, such as to accomplish an object of the invention, but unless otherwise expressly indicated in a particular instance, it is used limitatively, to reference ligands that are predominantly, if not exclusively, found on the aforementioned endothelial cell surface (as well as in lymph nodes), such that the first portion of the multifunctional ligand is for all intents and purposes functionally targeted to the intra-luminal surface of the lymphatic system. For instance, it is appreciated that the ligand in question may be targeted to a limited extent elsewhere e.g. in the case of preferred LYVE-1 ligand discussed below, to parts of the spleen (which also provides a venue for immune cell interactions).

The invention is not concerned with imparting effects to or simply blocking a receptor on the intraluminal lymphatic endothelial cell. In this context, the multifunctional ligand of the invention is intended to exclude only, unless otherwise specifically stated in the claims, only those embodiments disclosed in WO 98/06839 or other references describing ligands, antagonists or antibodies which bind to a lymphatic vessel associated ligand or receptor (see examples of such references below), insofar as such embodiments comprise lymphatic vessel associated ligands as hereinabove limitatively defined, and to this limited extent only, the term therapeutic function exerting moiety or immune function exerting moiety preferably excludes: 1) an antibody F_{c} receptor, insofar as such limitation excludes from the scope of the multifunctional ligand (per se) aspects of the invention, substantially intact naked antibodies which simply bind to a lymphatic vessel associated ligands, as well as preferably excluding 2) cytotoxins or drugs, insofar as this excludes from the scope of the multifunctional ligands of the invention an antibody or fragment thereof which is fused or conjugated etc. exclusively to a cytotoxic molecule (including an atom) or drug (i.e. an antibody linked to a cytotoxin or drug only, which is not per se an or is not integrated with an independent biologic or immune function exerting component) so as to accomplish a function in relation to cells or other entities (including other multifunctional ligands) within the lymphatic system other than the cell or ligand to which the multifunctional ligand is anchored. Similarly, the invention is not concerned with multifunctional ligands which are adapted to be internalized into a lymphatic endothelial cell and the invention is specifically concerned with targeting a lymphatic vessel associated marker which does not promote internalization and/or in which the first portion has an affinity (high or medium) which limits this effect (i.e. to a side effect)

In the same vein, the term immune function is broad in intent (as discussed below, and includes particularly any function, including binding, capable of being exerted by an ligand preferably an antibody (e.g. multifunctional ligands which are bispecific antibodies) however it is to be understood that the invention and particularly the immune function exerting moiety does not have as an object (despite possible incidental effects) evaluating or exerting a disease responsive or immune function vis-à-vis ligands/cells lining the intra-luminal surface of the lymphatic system i.e. insofar as such ligands have a role in disease (other than simple binding exclusively for anchoring purposes which is attributable not the immune function exerting moiety but to the first portion) but rather, as evident in preferred aspects of the invention, preferably an independent biologic or immune function which is not predicated on blocking the lymphatic endothelial receptor or treating cells bearing the receptor i.e. exerted vis-à-vis targets other than the lymphatic endothelium target, for example 1) in the case of stationary diseased cells or disease causing cells or molecules, targets at the site of the disease (which may optionally be effected, for example, in case of immunization or other immune cell stimulation, inhibition etc. in the lymphatic system); and 2) in the case of non-stationary diseased or disease causing cells or molecules, at the site of those cells/molecules including, preferably, within the lymphatic system, for example by binding to or signaling those cells in the lymphatic system.
1. In one embodiment, the first portion of the multifunctional ligand is an antibody.
2. In another embodiment, the immune function exerting moiety binds to a target ligand and thereby directly or indirectly accomplishes its effect (in whole or part). For example, the target ligand may be a cytokine, for example in order to target immune cells to the lymphatic system to assist in, diseased, disease causing or other target cell ablation or phagocytic type activity (e.g. by the cytokine in turn binding to a ligand, for example on an immune cell having phagocytic activity) or exerting a chemotactic effect within the lymphatic system, or to mop up cytokines, for example, when released in toxic amounts due, for example due to effects of a disease or particular immunotherapy (such as anti-CD3 therapy; see for example U.S. Pat. No. 6,193,969, Kummer U. et al., Immunol Lett 2001 Jan. 1; 75(2):153-158) (with respect to removing disease associated antibodies from circulation see for example a bispecific dsDNAx monoclonal antibody construct for clearance of anti-dsDNA IgG in systemic lupus erythematosus. J Immunol Methods. 2001 Feb. 1; 248(1-2):125-138). (See also, for example, U.S. Pat. No. 5,968,510 with respect to antibody-CTLA-4 fusion proteins for use in binding to various target ligands).
3. In another embodiment, said immune function exerting moiety comprises an antibody and optionally both the first portion and the immune function exerting moiety are antibodies (with respect to bispecific antibodies, and a recent review of some of the technologies referred to or applicable to various aspects of the invention (see particularly, Journal of Immunological Methods February 2001 Vol. 248(1-2) page 1-200)
4. In another embodiment, said immune function exerting moiety binds to an immune cell, a diseased host cell or a disease causing cell or entity (e.g. see U.S. Pat. No. 6,193,968). The term disease is used broadly to refer to any undesirable condition. The term diseased host cell includes but is not limited to a cancerous (in the broadest sense of that term) cell and a virally infected cell (these examples are given inasmuch as the invention in a preferred embodiment involves targeting such cells for destruction) and the term disease causing cell includes but is not limited to a virus or other infectious agent and as well as immune cell which is directly or indirectly involved in mediating or causing a undesired, deleterious or pathologic consequence, including but not limited to autoimmune disorders, transplant rejection, and other immune system linked diseases. The term disease causing entity is used to refer, without limitation, to any molecule, atom, peptide, ligand, complex, chemical, component, epitope etc. that is directly or indirectly involved or associated in mediating or causing a disease or disease causing event including an antibody. Such binding to the entity may be effected through the instrumentality of one or more (same or different) multifunctional ligands and through binding to any ligand or set of ligands, including receptors, multi-component epitopes etc, including for example, tumor “associated” (i.e. differentially expressed to advantage for targeting purposes) epitopes which may or may not or may only be partially present on tumor associated antigens, or commonly, for example antigens/epitopes/ligands/receptors etc. which are over-expressed in association with cancer cells; or for example, antigens/epitopes/ligands/receptors etc. involved in immune signaling, stimulatory, co-stimulatory, inhibitory, adhesion or other interactions, including without limitation, cytokine receptors, ligands associated with immune cell adhesion (see for example U.S. Pat. No. 5,747,035), ligands to which binding results in stimulation, activation, apoptosis, anergy or costimulation, or ligands which differentiate between different populations or subpopulations or immune cells, including sub-populations of B cells and T cells, activated versus non-activated lymphocytes, diseased or disease-causing cells versus non-diseased/disease causing lymphocytes and specific immune cell clones for example those having specific Ig type and MHC-peptide type ligands/and correlative ligands. Examples of such ligands include CCR5, CTLA-4, LFA-1, and LFA-3. ICAMs e.g. ICAM-1, ELAM-1, CD2, CD3, CD4 (e.g. see U.S. Pat. No. 6,136,310), CD5, CD6, CD18, CD22, CD40, CD44; CD80, CD86, CD134 and CD154, to name only a few (see also U.S. Pat. No. 6,087,475: PF4A receptor, U.S. Pat. No. 6,135,941, WO 01/13945). Such ligand may also selectively be targeted using any dual affinity strategy according to the invention.
5. The invention is also directed to a multifunctional ligand and a method which comprises using the multifunctional ligand to assess the toxicity of directly or indirectly targeting, for example, primarily within the lymphatic vessel system (see discussion below), cells having well known markers that are associated with immune cells, for example, those exclusively associated with activated immune cells, in-so-far as such targeting has a role in prolonging or counteracting the activated state, destroying the cell (e.g. where the multifunctional ligand is a immunotoxin) causing the cell to be destroyed (e.g. through apoptosis (e.g. see WO 01/19861, fas-fasL, U.S. Pat. No. 6,046,048) or assisting another molecule or cell for example a T-cell or other killing or immune modulating cell to do the modulation or killing (markers such as CD23, CD25, CD26, CD28, CD30, CD38, CD49a, CD69, CD70, are just some of the markers associated with activated immune cells) etc. (for a complete listing of marker associated with activated immune cells see for example Roitt I et al. Immunology, sixth edition, Mosby 2001 referenced below and Encyclopedia of Immunology (1998), Abbas et al. Cellular and Molecular Immunology 2000, Harcourt & Brace, the contents of which are incorporated by reference herein). Antibodies for many such ligands are known or could be readily made by e.g. phage display (see references herein including J Immunol Methods 1999 Dec. 10; 231(1-2): 65-81), and natural ligands for such markers or functional analogues thereof are in some cases known or could be made by recombinant DNA technologies referenced herein (see also Cellular & Molecular Immunology 4th Edition, Abbas Ak et al. WB Saunders and Company 2000, Antibody Fusion Proteins, Steven M Chamow, Avi Ashkenazi Eds. ISBN 047118358X May 1999 Wiley; Kontermann, R., et al. (Eds.) Antibody Engineering, Springer 2001. ISBN 3-540-41354-5; Antibody Engineering, Carl A. Borrebaeck Oxford University Press, 1995; Antibody Engineering: A Practical Approach David J. Chiswell, Hennie. R. Hoogenboom, John McCafferty Oxford University Press, 1996; Antibody Engineering Protocols, Sudhir Paul (1995) Humana Press; Antibody Expression & Engineering (1998) Henry Y. Wang, Tadayuki Imanaka, American Chemical Society). The term modulation is used broadly to refer to any change, directly or indirectly, in an immune function or effect, as broadly understood. Many such forms of modulation are well known in the art (some are exemplified herein), and therefore these need not be specifically recited (for a review of such effects see for example Roitt I et al. Immunology, sixth Edition, Mosby 2001; Encyclopedia of Immunology; (1998) Morgan Kaufmann Publishers, ISBN: 0122267656).
6. In one aspect the invention contemplates that the multifunctional ligand exerts its function substantially (i.e. upon gaining entry into lymphatic system and when bound to the lymphatic endothelial cells, which is dependant on the mode of administration) within the lymphatic system, on cells and/or molecules circulating through the lymphatic system, for example with respect to some embodiments, for greatest effect, to avoid an undesired degree of immunosuppression (for example, embodiments where immune cells are targeted for ablation and/or apoptosis). Preferably, such effect, is exerted at least in part, and preferably substantially to the exclusion of regions within lymphatic system that house at the time of administration non-circulating cells (e.g. thymus, bone marrow, and various parts of the secondary lymphoid tissues) or/and with respect to some embodiments (excluding for example those related to immunization or mopping up toxins or antibodies) preferably, non-activated cells. This specificity of targeting can be accomplished in part to the natural distribution of the lymphatic endothelium associated marker of choice, the mode of administration and various targeting strategies herein described.
7. For example, the invention contemplates modes of delivery that to varying degrees ensure a greater degree of lymphatic system targeting, for example administration directly within the lymphatics, administration in tissues that drain to the lymphatics or parts thereof, intravenous delivery, as are well known to those skilled in the art, preferably in each individual case at strategic sites of administration that are most pertinent or selective for the disease in question, to the extent that selectivity is desired. The invention contemplates a variety of different size multifunctional ligands (MRU, single domain, scFv, Fab, minibodies, F(ab)₂, F(ab′)₂, substantially whole antibodies etc. and known or obvious multimers thereof referenced herein and in the referenced literature) that are most suitable (e.g. for small enough or, for example, having longest half life in circulation) for particular modes of administration to the extent that this is a limitation (e.g. size, where drainage into the lymphatic system is sought to be increased or optimized).
8. In a preferred embodiment the invention contemplates that the immune function exerting moiety of the multifunctional ligand comprises (e.g. by way of recombinant fusion, conjugation etc.), or binds to (such antibodies are known or may be made by phage, ribosome or other such ‘display’ methods), so as to present the functional part of an adhesion molecule (molecule involved in cellular adhesion), for example an endothelial adhesion molecule such as a selectins, ICAMs (e.g. ICAM-1, ICAM-2) V-CAM, MAdCAM-1 or functional analogues or portions thereof (see for example U.S. Pat. Nos. 6,143,298, 5,512,660, 5,861,151, 5,489,533, 5,538,725, 6,037,454, 5,565,550, Circulation 2001 Feb. 27; 103(8):1128-1134, and specific examples/references recited below) in order to control cell traffic including facilitating cell anchoring within the lymphatic system, including for example to facilitate interaction with another “arm” (functional moiety) of the multifunctional ligand or a second etc. multifunctional ligand or an immune cell (or a cell-sized latex sphere as described herein—for this purpose the adhesion molecule may be on the surface of another, preferably multifunctional-ligand-anchored latex sphere or on a similarly anchored cell) as well as combination therapies, for example, with therapeutic entities that enhance or inhibit leucocyte adhesion, or multifunctional ligands or antibodies that bind to one of their corresponding ligands on immune cells (e.g. integrins) or other ligands e.g. CD44, to facilitate control and/or some selectivity of cell entry into the lymphatic system, for example, for reactivity with the multifunctional ligands of the invention. The invention also contemplates that such adhesion molecules may be the subject of targeting with dual affinity ligands of the invention and that such ligands may include a moiety which binds to a lymphatic endothelial cell.
9. The invention also contemplates that one or more multifunctional ligands in which the immune function exerting moiety comprises an antibody type molecule targeted to a particular cell surface ligand may be able to mimic effect of such adhesion molecules, as discussed below (any such discussion of an antibody mimicking this function is unless otherwise stated not intended to limit the broader concept of utilizing any class of molecule that would facilitate anchoring or controlling, e.g. slowing the passage of cells through the lymphatic vessels). It is to be understood that there may be limitations in the number of cells that can be targeted for ablation in the lymphatic system by slowing the passage of cells, particularly for the purpose herein specified of allowing them the requisite period of residence within the lymphatic system for immune cell targeting or interaction or prolonged interaction with multifunctional ligands of the invention for binding purposes while bound to the lymphatic system endothelium, for example, certain end stage lymphomas/leukemias. In this particular context it is to be understood that: 1) the invention may have greatest application when the multifunctional ligand is administered so as to primarily target cells within the circulatory system, or as an adjunct therapy, or for remission or near remission conditions, or when combined with hyaluronic acid therapy. For example, the invention contemplates that an effective amount of hyaluronic acid is pre-administered to tissues draining to the lymphatic system so as to initially occupy binding sites on LYVE-1 primarily in the smallest lymphatic vessels and thereby minimize excessive binding within the narrowest vessels.
10. In a preferred embodiment said first portion binds to LYVE-1 or podoplantin described below.
11. In a preferred embodiment, said first portion is fused, conjugated or otherwise linked directly or indirectly to an immunizing moiety, for example an antigen, epitope, mimitope or peptide etc. presenting/incorporating entity/scaffold that generates by itself or with the help of one or more cytokines, costimulatory molecules and/or adjuvants etc. an immune response to a desired antigenic determinant (this term is used broadly to correspond at least in scope to the overlapping groupings: antigen, epitope, mimitope or peptide), for example an anti-idiotypic antibody, an antibody component which is capable of binding to a T cell activating entity for example a cell (e.g. an APC see Int Immunol 2000 January; 12(1):57-66 or other cell having e.g. immune modulating activity e.g. see U.S. Pat. No. 6,004,811) which is for example genetically engineered to express relevant ligands for activating (or with respect to functions not necessarily related to immunizing, anergizing, tolerizing or otherwise modulating the activity of), an immune cell for example a B cell or T-cell, for example an MHC-peptide and B7 co-stimulatory molecules for activation of T-cells (see for example Proc Natl Acad Sci U S A 2001 Jan. 2; 98(1):241-246 see also Tham E L et al. J of Immunological Methods Vol. 249(1-2)(2001) p 111-119 with respect to latex spheres that can be used for this purpose), or for example a CTLA-4 scaffold, a peptide fused to an Fc domain (see WO 01/18203) a HSP-peptide complex/conjugate, an MHC protein or peptide complex etc. Antibody-MHC complex fusions and antibody-B7 costimulatory fusion molecules are known and the invention contemplates that fusion molecules with anti-lymphatic marker antibodies could be made and used together for immunization purposes. It is also contemplated that the absence of costimulatory molecules for presentation in a co-stimulatory fashion with an MHC peptide complex will cause a tolerizing effect. Accordingly, the invention is also directed to a multifunctional ligand comprising an immune function exerting moiety which comprises an MHC, preferably complexed or otherwise linked to a peptide. Peptide linking may for example be effected independently, naturally or for example through causing release of peptides from an MHC peptide or HSP peptide complex by injecting a weak acidic solution into tumor e.g. just prior to excision. Suitable such solutions which may for example be combined with a cytokine, e.g. IL-12 and/or adjuvant are known in the art.
12. In a preferred embodiment said immune function exerting moiety comprises an anti-idiotypic antibody, for example an antibody that a) mimics, for example, a cell surface expressed tumor associated epitope, a virus or other infectious agent associated surface epitope, a toxin, an immune stimulatory, costimulatory, inhibitory, or otherwise interactive ligand; or b) serves to bind to the idiotype (i.e. paratope) bearing antibody to which it binds as an anti-idiotype, for example an autoimmune antibody, etc. or an antibody bearing a toxic moiety for removing such antibody from passage into the circulation.

13. In a preferred embodiment, the invention contemplates that the first multifunctional ligand is used for development, therapeutic evaluation or combination therapy in conjunction with a second different multifunctional ligand of the invention, to achieve a cooperative effect (for example, in the same composition or substantially contemporaneously administered (i.e. to reach the same or an interrelated destination in a cooperative time frame) or in necessary or desired sequence/interval, etc.). An example of such cooperative effect is an interaction (not necessarily simultaneously) with two different immune cell surface ligands (for example via an antibody binding interaction), or to deliver different payloads e.g. toxins, to a diseased cell see (U.S. Pat. No. 6,077,499). The invention also contemplates a method of effecting substantially coordinated interactions of differing temporal and spatial complexities, ranging from a somewhat proximal and contemporaneous delivery (e.g. in the same composition) of a first multifunctional ligand having, for example, a cancer cell binding second portion, and a second multifunctional ligand having, for example, a cytokine binding Ab, e.g. to reduce any toxic effects associated with toxic levels of cytokine release, a cytokine component (for example to harness the effect of such component as a means to attract one or more immune cells to kill a diseased cell or to harness the inhibitory effect of such component (e.g. using one or more cytokines employed by cancer cells to evade immune cell targeting) e.g. on undesired immune cell elimination or immune cell elimination of the multifunctional ligand, or a T-cell binding component (e.g. anti-CD3) to harness the effects of such component on cancer cell killing optionally with a concomitant object of assessing possible counterproductive immune cell elimination (e.g. as would be enabled by using a radiolabeled multifunctional ligand and determining the disposition of the label over time) of the multifunctional ligand.
14. Also contemplated are methods to implement more spatially and/or temporally sensitive interactions. For example, when administered in empirically determined suitable proportions and in empirically determined sufficient total amounts for, at least, partial and/or local lymphatic-vessel-associated-ligand saturation or partial saturation to achieve proximal binding of a first to second multifunctional ligand (having regard to the route of administration e.g. local saturation can be more readily accomplished by administration into the lumen of the lymphatic vessel). Two different such multifunctional ligands may be used, for example, to deliver two different immune function exerting moieties in substantial proximity to one another for contemporaneous interaction with two different ligands on an immune cell (i.e. when it approaches the luminal wall of a lymphatic vessel). For example, this approach may be used to implement one or more effects including increased avidity to the cell for prolonged cell anchoring, which may positively impact on desired (in some embodiments) transfer of the multifunctional ligand from the lymphatic vessel wall to the target cell e.g. for achieving an inhibitory effect via ligand binding (e.g. assessed via duration of multifunctional ligand binding e.g. quantitative or radioimage approximated label elimination) (N.B. this effect may be assessed with multiple copies of the same multifunctional ligand), delivery of a cooperative payload e.g. different entities which contribute to the same or a different mechanism of cell killing, counterparts in a two component interaction (biotin-avidin), which preferably yields evidence (preferably quantifiable evidence) of the interaction, for example an enzyme-substrate interaction to quantitatively assay the amount of an enzyme converted substrate (e.g. using a conjugated prodrug and pro-drug conversion akin to ADEPT and assessing the extent of prodrug conversion e.g. by labeled anti-drug specific antibody). For example, the invention contemplates the use of a respectively linked catalytic antibody component (see for example U.S. Pat. No. 5,658,753: Catalytic antibody components) and labeled substrate or RNAase and labeled RNA etc. for this purpose. Another example, discussed in more detail below is the use of one multifunctional ligand for targeting (selectivity) purposes and another for implementing directly or indirectly a desired therapeutic effect, both ligands optionally being required to give rise to a substantial probability of binding (the invention also contemplates that this strategy could be used with a single multifunctional ligand having two intra-luminally directed binding moieties).
15. The invention contemplates that such interactive entities may be conjugated fused or otherwise linked to a respective first and second multifunctional ligand for achieving a cooperative interaction between adjacently bound such ligands.
16. The invention contemplates that adjacently interacting multifunctional ligands yielding detectable evidence of the interaction, could be use in a method to assess e.g. a) luminal ligand saturation for dosing, b) multiple simultaneous binding interactions, and c) perhaps most spatially sensitive, development of a process to achieve cross-linked binding with multiple e.g. immune cell ligands e.g. a costimulatory immune effect (i.e. the effect of different simultaneous interactions e.g. on stimulation, inhibition etc. of e.g. an immune cell for example combining a first multifunctional ligand capable of selectively binding to, conjugated to or fused to a B7 component (see J Immunother 2001 January-February; 24(1):27-36; J Immunol 2001 Feb. 15; 166(4):2505-2513; Chalitta P M et al. J. Immunol. 160:3419-3426) and a second multifunctional ligand capable of selectively binding to, conjugated to or fused to an MHC molecule delivered initially with or without peptide. For example, the invention contemplates using various amounts/proportions of multifunctional ligands having antibody components fused or conjugated to or capable of binding selectively to, for example an MHC class I or II peptide complex and recombinant B7-1-Fc and/or B7-2-Fc respectively (see Eur J Immunol 2001 January; 31(1): 32-38; Eur J Immunol 2001 February; 31(2): 440-449) (for tumor reactive peptides see for example J Immunother 2001 January-February; 24(1): 1-9). In this latter connection (cross-linking type interaction), and/or for permanence of binding or ease of attaching other cooperative entities (for example biotin coated or conjugated radionuclides, liposomes or other payload carrying entities (e.g. see for example U.S. Pat. Nos. 5,439,686, 6,007,845, 5,879,712, 5,456,917, 6,165,502, 5,079,005, 5,888,500, 5,861,159, 6,193,970, 6,190,692, WO 00/69413, WO 01/07084) the invention contemplates biotinylating the two multifunctional ligands and linking the two biotinylated cooperative multifunctional ligands with avidin, streptavidin (or other modified forms thereof e.g. deglycosylated avidin or using other complementary linking components—see e.g. U.S. Pat. No. 6,077,499).
17. The invention also contemplates enhancing the cross-linking of the multifunctional ligands of the invention through complementary components such as biotin and avidin.
18. Preferably, with respect to, for example, increasing selectivity of targeting certain cells (e.g. to induce immune tolerance), the invention also contemplates that a first multifunctional ligand is used to bind to a marker specific to a particular kind of cell (e.g. activated immune cells) and a second multifunctional ligand (which may not be specific for activated immune cells) is used to modulate the activity of the immune cell (for example inactivate it or reduce its disease causing capability directly or indirectly by binding to it). For example, where the marker is used to determine the selectivity of the targeting but cannot be used for modulating its activity, it is contemplated that the functional affinity of one or both the first portion and second portions of one or both of the cooperating multifunctional ligands can be selected to at least partially control the selective modulating effect of the pair, for example both interactions would be required for the second multifunctional ligand to have an optimal opportunity to bind. For example, the functional affinity for the target cell is relatively weak for the purpose of attaching to the e.g. immune cell for a sufficient duration (e.g. so as to yield the effect of becoming attached to the immune cell in preference to the lymphatic vessel), compared with that of the first multifunctional ligand (i.e. the one that accomplishes the selective recognition through binding) to reduce the likelihood that the second moiety will bind in the absence of binding of the first moiety (notably a similar type of coordinated interaction i.e. two binding interactions, is naturally used for cell adhesion). (NB. this type of coordination has application i.e. both specificities are optimally required for binding, to a single multifunctional ligand, having a divalent immune function exerting moiety e.g. a triabody or tetrabody or for cross-linking and other types of coordinated interactions). In a preferred embodiment, if transfer of binding of the first multifunctional ligand to the immune cell is not desired its functional affinity of the first portion to the lymph vessel can be greater than that of its second portion, while the reverse could be true for the second multifunctional ligand. It will also be appreciated that antibodies which cross-link for example an integrin and a marker of immune cell activation could be used to limit the number of activated immune cells that migrate through the lymphatic system. For example bispecific d Abs, diabodies, etc. in which the functional affinity of each specific binding portion individually does not strongly favour binding, could be used to selectively target specific sub-populations of immune cells or even specifically activated immune cells (for example antibodies that recognize particular antigen/peptide specific T cell or B cells).
19. Accordingly, more generally speaking, the invention is directed a bispecific ligand, preferably a bispecific antibody, having a first portion which binds to a ligand which differentiates between members of the same immune cell population (e.g. a particular type of T cell) and a second portion which binds to a second ligand on the same cell, which binding exerts directly or indirectly a desired effect, wherein the functional affinity of said first and second portions are selected so as to substantially increase amount of immune cells in which both such portions are bound to their respective ligands relative to those which a single such portion is bound to a single ligand and preferably wherein the amount of immune cells to which the bispecific ligand is not bound is substantially greater than the number of immune cells that are not bound when compared to using a bispecific ligand having the same specificity and for example a 10¹ to 10⁷ (preferably 10¹ to 10⁶, preferably 10³ to 10⁶, preferably 10³ to 10⁵) increase in affinity of one or both portions. This invention also contemplates that binding to the ligand which differentiates between members of the same population (a particular type of T cell) does not have a negative consequence other than to cause the molecule to be ineffectual unless both of its portions are bound and that its binding is itself sufficient for binding and/or stronger relative to the second portion by two fold to 5 orders of magnitude, preferably 1 to 3 orders of magnitude. The term substantially greater imports medical significance and may preferably be 15%-10000% greater. The foregoing examples are not meant to be limitative.
20. In a preferred embodiment, the invention more broadly speaking contemplates a two ligand interaction (using one or more multifunctional ligands) wherein for example both are required or increase the likelihood of interaction and wherein the interaction of at least one contributes to specificity, though not necessarily to modulation, thus permitting a broader selection of modulators including those that but for the selectivity enhancing effect of the cooperating ligand and the lymphatic system venue, would be toxic in the desired therapeutic dose. Examples of markers that could assist in selectivity include those are unique to, for example, activated B cells or T cells or those having particular specificities in virtue of unique Ig type receptors. Examples of ligands on, for example immune cells, through which modulation/inhibition/stimulation etc. (including, for example apoptosis), for example by antibody binding or supply of a natural interactive ligand, are well known. Some examples are provided herein. Combinations and permutations of markers and ligands for selectivity and exerting an immune effect such as modulation/inhibition/stimulation referred to herein or in the literature incorporated herein by reference or well known in the art are contemplated to be within the scope of the invention.
21. It will be appreciated that a combination of factors, such as dose, using additional molecules that increase or decrease migration or adhesion optionally in a tissue targeted manner, route of administration (e.g. within tissue that best drain to lymphatic vessels or a portion thereof), use of cytokines, etc. and immune modulating drugs, as well combination therapies with known entities, can be employed in various combinations for strategies of harnessing the unique properties of the multifunctional ligand of the invention, to achieve a selectivity enhancing and/or modulatory/inhibitory/stimulatory etc or otherwise cooperating effects with respect to the desired target population of cells. Unless their function are self-evidently conflicting the invention contemplates all permutations of the multifunctional ligands disclosed herein or in the literature incorporated by reference herein as well as those evident to persons skilled in the art whose mention is omitted.
22. In a preferred embodiment, the immune function exerting moiety binds with greater functional affinity to its target ligand than said first portion binds to its target ligand. For example said immune function exerting moiety may bind with greater avidity (preferably at least 2 times greater (divalent vs. monovalent) and lesser or greater affinity (e.g. within a range of 1×10⁻⁶ to 1×10⁶ fold) or with the same avidity and greater affinity (e.g. up to 1×10⁶ fold). In applicable aspects, the invention contemplates that this increased functional affinity can be employed to effect transfer of a lymphatic vessel bound multifunctional ligand (e.g. a bispecific antibody) to a cell passing through lymphatic system. The invention also contemplates a method comprising radiolabeling the multifunctional ligand to assess, for example, the degree to which immune cells at a disease site have passed through the lymphatic system. Certain aspects of the invention, discussed herein, relate to a multifunctional ligand based system of targeting a particular immune cell ligand for stimulation, inhibition etc. predominantly within select portions of the lymphatic system that contain migrating cells (although some general targeting can controllably occur before the multifunctional ligand binds to the lymphatic system or when the multifunctional ligand releases from the lymphatic system without having found its target within the lymphatic system) will have at least a partially selective effect on targeting disease causing/mediating immune cells (e.g. activated with a specificity that causes the disease) as opposed to non-disease causing/mediating cells, in the case where such ligand is also expressed on such other immune cells e.g. of the same type e.g. T cells. This permits targeting of immune cells primarily within the portions of the lymphatic system that contain migrating cells particularly disease causing/mediating cells while minimizing immune system dysfunction. This effect can be even more selectively accomplished, for example, by delivering the multifunctional ligand directly into the lymphatic system and within a time frame which is shorter that the normal duration of binding of the multifunctional ligand determining the degree to which the multifunctional ligand is bound to such diseased related cells at the disease site and similarly the degree to which it is bound to the cells unrelated to the same disease e.g. via radiolabel. As discussed more fully below, the invention also contemplates a multifunctional ligand based system of assessing the effects of certain types of immune stimulation e.g. how stimulating enhanced migration or adhesion, will differentially affect disease activated cell migration through the lymphatic system to enhance such disease cell targeting within the lymphatic system. For example, for tumor cell targeting and stimulation of disease-activated immune cells the invention contemplates evaluating cytokine (e.g. TNFα) linked anti-angiogenic marker antibodies, optionally, preferably in combination with anti-tumor vaccination strategies, to direct disease activated immune cells to tumor site and the lymphatic system for further immune stimulation. Based on a “bait and trap” type approach, ligands such as OX40L and CD44 may also be assessed for this purpose.
23. In this connection and more generally the invention also contemplates using a bi-specific antibody, for example having a lymphatic endothelial binding first portion and for example a cytokine binding second portion, wherein the cytokine binding portion has a lower functional affinity for the cytokine (for example 1×10—6 to 0.9 fold) compared with that of its natural receptor on an immune cell. It is contemplated that a multifunctional ligand of the invention could be used optionally in conjunction with a multifunctional ligand which displays a functional adhesion molecule (a selectin, ICAM, etc.) to assess the optimal parameters for transfer of the cytokine, for example, as is known to occur by monitoring the effects of cytokine release attributable to such cytokine transfer. It will be appreciated that this information or approach could be used to optimize the binding parameters for other ligands as well (e.g. anti CD3) and could be employed not only in lymphatic system but to locally deliver inhibitory or stimulatory cytokines or other ligands to certain tissue targets, for example new blood vessels forming within tumors or other tissue specific markers.
24. The foregoing strategies could be used as part of a primary, adjunct or low disease burden therapy.
25. In a preferred embodiment, the second portion comprises a ligand which is capable of binding to an immune cell for example B cells, T cells etc, preferably in one embodiment to assist in cell killing or immune modulation of a target cell (re NK cells see for example U.S. Pat. No. 5,770,387)(see also U.S. Pat. No. 6,071,517: Bispecific heteroantibodies with dual effector functions; Bispecific antibody-mediated destruction of Hodgkin's lymphoma cells. J Immunol Methods 2001 Feb. 1; 248(1-2): 113-123; Bispecific antibody-targeted phagocytosis of HER-2/neu expressing tumor cells by myeloid cells activated in vivo. J Immunol Methods. 2001 Feb. 1; 248(1-2): 167-182 as well as J Immunol Methods 2001 Feb. 1; 248(1-2): 103-111).
26. With respect to avidity, affinity and other elements of design including size, blood clearance, additional functionality etc. the multifunctional ligand may be, for example, a bispecific antibody having a monovalent first portion and a monovalent second portion, a bispecific antibody having a divalent first portion and a divalent second portion, a trivalent trispecific antibody having a monovalent first portion and a second portion comprising a monovalent immune function exerting moiety which binds, for example, to a target ligand on a target diseased, disease causing or immune cell, and for example, a monovalent portion which binds to an immune cell which assists in killing or modulation for example anti-CD3 or anti-CD28 antibody component, a tetravalent trispecific antibody having a monovalent first portion and a second portion comprising a divalent immune function exerting moiety which binds, for example, to a target ligand on a target diseased, disease causing or immune cell, and for example, a monovalent anti-CD3 or anti-CD28 antibody component (it is contemplated that this orientation might advantageously position the anti-CD3 component for interaction with a T-cell almost exclusively when the first portion is not bound to the luminal wall of a lymphatic vessel), a trivalent bispecific antibody having a monovalent first portion and a second portion comprising a divalent immune function exerting moiety, for example, one which binds, for example, to a target ligand on a target diseased, disease causing or immune cell. The antibody subunit may be for example, a Fab, a substantially intact antibody, a single domain antibody (see also Hufton S E. Dis Markers 2000; 16(1,2): 37 Single domain human immunoglobulin fold-based biomolecules; Antigen specificity and high affinity binding provided by one single loop of a camel single-domain antibody. J Biol. Chem. 2001 Jul. 13; 276(28): 26285-90. Optimal Design Features of Camelized Human Single-domain Antibody Libraries. J Biol Chem. 2001 Jul. 6; 276(27):24774-24780; Recognition of antigens by single-domain antibody fragments: the superfluous luxury of paired domains. Trends Biochem Sci. 2001 April; 26(4): 230-5; Llama heavy-chain V regions consist of at least four distinct subfamilies revealing novel sequence features. Mol. Immunol. 2000 August; 37(10): 579-90) a minibody an scFv or a minimal recognition unit (MRU e.g. see U.S. Pat. No. 6,174,691: Minimum recognition unit of a PEM mucin tandem repeat specific monoclonal antibody).
27. In a preferred embodiment, the multifunctional ligand binds to an immune cell which is associated with an autoimmune reaction, for example a CCR5-expressing cell. (See also Apoptosis genes and autoimmunity. Curr Opin Immunol. 2000 December; 12(6): 719-24. for application herein)
28. In a preferred embodiment, the second portion comprises a cytokine component.
29. In a preferred embodiment, the second portion comprises a cytotoxic component.
30. In a preferred embodiment, the second portion of the multifunctional ligand comprises an internalizing antibody and a cytotoxic component.
31. In a preferred embodiment, the second portion consists of an antibody which binds to T cells, for example, an anti-CD3 antibody or an anti-CD28 antibody.
32. In a preferred embodiment, the second portion consists of a cytokine component.
33. In a preferred embodiment, the second portion comprises an antibody which binds to a target diseased, disease causing or immune cell and further comprises one or more components selected from the group consisting of a cytokine component, a cytotoxic component and an anti-CD3/CD28 component.
34. In another aspect the invention is directed to a composition comprising a multifunctional ligand and a pharmaceutically acceptable excipient.
35. In another aspect the invention is directed to a composition comprising a plurality of different multifunctional ligands.
36. In another aspect the invention is directed to methods and compositions for developing and evaluating the therapeutic value of stimulators, mediators, inhibitors etc. of immune cell signaling (e.g. stimulatory, inhibitory, costimulatory), adhesion, migration, etc. including the effects of ligand/receptor blocking and supply of specific cooperative ligands, using the multifunctional ligands of the invention.
37. In a preferred aspect, the multifunctional ligands of the invention may be used to assess the effects of such compositions on the sub-population of cells that migrates into lymphatic vessels. In particular, the invention is directed to assessing the expectation that some disease causing, mediating or otherwise disease active immune cells have an enhanced ability/opportunity (and/or can be enhanced in their ability/opportunity to make their way into the lymphatic system) so that targeting of relevant ligands on that sub-population of cells within the lymphatic system will cause at least a partial selective targeting effect, preferably with positive effect on the dosing capability and choice of ligands i.e. in terms of limiting more universal and/or deleterious consequences. The invention is also directed to a method of reducing the toxic side effects of a pharmaceutical composition comprising a multifunctional ligand in which the immune function exerting moiety is targeted to a ligand that is not found exclusively on disease causing, mediating or otherwise disease active immune cells, by administering said composition in a manner in which it enter more directly into the lumen of a lymphatic vessel. (It contemplated that immunization within the lymphatic system can also be enhanced in virtue of such selective targeting.) In particular, the invention is directed to a multifunctional ligand, a pharmaceutically acceptable composition thereof and method of using same for assessing enhanced migration or enhancing migration of disease-active immune cells, said multifunctional ligand comprising an immune function effecting moiety which has an immune effect on an immune cell surface ligand i.e. effects including signaling (e.g. stimulatory, inhibitory, costimulatory, antagonistic, agonistic), including for adhesion and migration effects, etc. This may be accomplished practically, for example through ligand/receptor blocking e.g. via antibody, or by antibody fusions/conjugates etc. that supply the natural ligand or a functional fragment or chemical/biological mimotope thereof. In a preferred embodiment the invention is directed to a multifunctional ligand in which the immune function exerting moiety is an antibody that binds to a ligand selected, for example from the group consisting of CTLA-4, IL-2 receptor, CCR5, CD44, CD134, CD3, CD28, CD2.
38. In another aspect the invention is directed to a composition comprising a plurality of different multifunctional ligands which exert a potentially cooperative immune effect with respect to an immune cell, for example binding to two or more different ligands on an immune cell, wherein said ligands are selected, for example from the group consisting of CTLA-4, IL-2 receptor, CCR5, CD44, CD134, from any of the ligands herein mentioned or referenced or preferably CD3, CD28, CD2.
39. The invention is also directed to a method of inhibiting metastasis during the course of surgical removal of a tumor comprising administering to a patient prior to surgical treatment of the tumor site, a pharmaceutical composition comprising a multifunctional ligand in which the immune function effecting moiety binds to a tumor associated epitope on a cancer cell.
40. In another aspect the invention is directed to an immunocytokine having an anti-idiotypic antibody component which recognizes the paratope of an antibody which binds to a lymphatic vessel associated ligand and a cytokine component fused therewith or conjugated thereto. For example the cytokine component comprises IL-2 or a functional fragment thereof and/or IL-12 or a functional fragment thereof. In addition to their individual use in fusion proteins for tumor cell killing, combinations of II-2 and IL-12 have been used successfully for this purpose. It is contemplated that such cytokine fusion could be used to target T-cells or phagocytic cells to a multifunctional ligand that has bound to its disease causing or diseased cell target, preferably having left the lymphatic vessel endothelium in preference for binding its target. In this connection it is contemplated that the functional affinity of the anti-idiotypic Ab for the first portion would be less than that of the first portion to the lymphatic endothelium, so as to minimize competition between the two. It is also contemplated that the delivery of the immunocytokine occur substantially contemporaneously but separately and after that of the multifunctional ligand, optionally by a different route of administration.
41. Similarly the invention contemplates for the same purpose, a bispecific antibody having an anti-idiotypic antibody component which recognizes the paratope of an antibody which binds specifically to a lymphatic vessel associated ligand (preferably with lower affinity than that of the Ab for its target) and for example an immune cell binding portion e.g. an anti-CD3 antibody or an anti-CD28 antibody component.
42. Thus the invention is directed to a method of targeting a diseased or disease causing cell for destruction by the immune system comprising administering separately but substantially contemporaneously to a subject hosting the diseased or disease causing cell, preferably in sequence with an interposed interval and/or by different routes of administration, first a multifunctional ligand in which the immune function effecting moiety binds to a diseased or disease causing cell surface associated epitope, and an immunocytokine or bispecific antibody as described in the immediately preceding two paragraphs.
43. In a preferred embodiment, the invention contemplates modification of the multi-functional ligand to substitute one or more amino acids which reduce without functional impact on the molecule the number of immunogenic MHC II class peptide sequences within the molecule. This can be accomplished through procedures available to those skilled in the art, for example through the Delmmunisation services of Biovation Limited (see also U.S. Pat. No. 5,821,123 and related Xoma patents).
44. Inasmuch as the invention is predicated on intraluminal lymphatic system targeting such lymph association may be alternatively implemented, in suitable circumstances by the method of delivering the multifunctional ligand, for example into the lumen of a lymphatic system vessel or (where the multifunctional ligand is not of an unsuitable size (see for example Ikomi, F. et al. Lymphology 32 (1999) 90-122, within a portion of body that drains to the lymphatic system (i.e. a portion of the lymphatic system), for eventual migration to the lymphatic system. Particularly, with respect to embodiments of the invention in which the immune function exerting moiety is targeted with greater functional affinity to a therapeutic target (i.e. not the lymphatic system target), such lymphatic system oriented modes of delivery coupled with preferred targeting to the therapeutic target may combine, absent saturated binding to the therapeutic target, to better accomplish functional lymphatic targeting. Accordingly, in a broader aspect the invention is directed a lymphatic system targeted multifunctional ligand in which the second portion is as described herein and in which the specificity of the first portion exclusively for a lymphatic system is inessential. In this connection, the invention contemplates targeting markers on lymphatic vessels that are also present, for example on blood vessel endothelial cells (e.g. VEGF2). (With respect to lymph specific markers see also Birner P. et al. Clin Cancer Res 2001 January; 7(1): 93-7 “Selective immunohistochemical staining of blood and lymphatic vessels reveals independent prognostic influence of blood and lymphatic vessel invasion in early-stage cervical cancer” and published references to the markers therein mentioned.)
45. In the case of purely sustained release aspects of the invention where the first portion is temporarily anchoring a second portion for eventual release back into the circulation, the use of term immune function affecting moiety with reference to the role of the second portion does not adequately accommodate the breadth of the invention since any form of disease palliating active moiety or entity which exerts its effect elsewhere than at the lymphatic endothelial cell may gain advantage from this form of delayed delivery (depot effect) or anchoring.
46. Furthermore, in another preferred aspect, the second portion is capable of binding directly or indirectly (e.g. binding to an entity which in turn binds to a target entity) to a target entity, for example a therapeutic entity (for example to mop up excess such entity that does not immediately reach its target (e.g. an entity that is toxic elsewhere in the body), a toxic entity including an entity which is not per se toxic but the presence of which is undesirable at a particular time or in particular amount or concentration (e.g. a cytokine, for example when released as a result of anti-CD3 therapy), to redirect an entity to a target, for example a therapeutic entity, for example through the instrumentality of an antibody portion that is directed to that target (e.g. a multifunctional ligand in which the second portion comprises an anti-tumor antibody portion that is conjugated to streptavidin, to retarget biotin conjugated radionuclide back to the tumor (see Martin J. et al. (1997) Cancer Chemother, Pharmacol. 40:189-201), to temporarily anchor liposomes or other carriers of entities (e.g. drugs) having an direct or indirect beneficial effect elsewhere.

In a preferred embodiment, the invention provides a multifunctional ligand having, at least, a first portion which binds to a lymphatic vessel associated antigen/receptor (and thereby exerts, not necessarily to the exclusion of other effects) at least an anchoring function, and a second portion having at least one independent immune function. The term “immune function” is broad in intent including but not limited to direct or indirect and primary or corollary effects related to simple targeting, tolerance, immunization, stimulation, inhibition, modulation or various other immune related effects (other than simply forming part of the entity which blocks the lymphatic endothelial associated ligand). The term independent is used to exclude only an effect specifically targeted towards the ligand (blocking) or cell bearing the ligand to which the first portion of the multifunctional ligand is bound, which is not contemplated as an object of the invention. The invention contemplates rather that the immune function is exerted, for example, vis-à-vis immune cells or molecules or against cancer or infected cells to affect an immune function that relates to assessment, diagnosis, therapeutic modeling, or treatment of various disease states such as autoimmune disease, transplant rejection, cancer and infectious disease. In a preferred embodiment, the invention contemplates that the independent immune function is exerted through a physical ligand-ligand interaction. In a preferred embodiment the multifunctional ligand has an ability to bind in the manner of an antibody in virtue of at least one of the first or second portions, and preferably at least the first portion. The lymphatic system directed first portion may in some embodiments (LYVE-1) be hyaluronic acid or analogues thereof that have the appropriate binding capacity. In a further preferred embodiment, the second portion binds to a target ligand on a cell or molecule (e.g. a cytokine or autoimmune antibody) which passes through the lymphatic system. In a more preferred embodiment the multifunctional ligand is a bispecific antibody. The term antibody is used to refer to any antigen binding fragment of an antibody that substantially has the binding capability of an antibody including anti-idiotypic antibodies, and therefore the term bispecific antibody is used (unless the context implies a more specific usage) in a functional sense to refer to at least two different specificities (including trispecific antibodies etc.) and includes well known entities which are diabodies, triabodies, tetrabodies, minibodies, scFv dimers, etc., and entities in which one or both binding moieties are scFv or single domain type antibody fragments or dimers etc of such fragments (with respect to single domain antibodies see for example Camel single-domain antibodies as modular building units in J Biol Chem. 2000 Oct. 25, & Mulligan-Kehoe U.S. patents).

The term “anchoring function” is used broadly to refer to physical attachment for a period which renders the second portion of the multi-functional ligand capable of exerting its immune function. For example where the function of the second portion is to interact with a cell passing through the lymphatic vessels, for at least a period which permits sufficient interaction for the desired effect.

The term ligand is used very broadly herein to refer to any moiety, preferably in some cases, a specifically interacting moiety including binding moieties (e.g. antibodies, receptors etc.) and bound moieties (e.g. antigens, epitopes etc) and/including otherwise interacting moieties (e.g. chemotactic interactions or interactions that require multiple points of interface e.g. cross-linking or multi-component epitopes). In other words, the term ligand is used broadly to refer to any entity or part thereof which can be subject to an intermolecular interaction that can result in binding. The term moiety is used broadly and non-limitatively to refer primarily to a functional part of an entity, and may refer to even the whole of the entity depending on the context in light of the broadest concept of the invention.

Optionally, depending on the mode of delivery and the relative functional affinity of the respective first and second portions, the multi-functional ligands of the present invention, may exert their immune function primarily in lymphatic system and also significantly before and optionally after entry into the lymphatic system. In a preferred embodiment the multifunctional ligand is capable of simulating a depot effect by binding for a prolonged period to the intra-luminal lymphatic endothelium for later release over time back into the circulation. The choice (avidity effect resulting from multiple binding “arms”) and affinity of the binding molecule as well as various, preferably controllable factors impacting on any “undulating” movements of the lymphatic vessels (e.g. water consumption) or competitive binding is contemplated to impact the binding time.

With respect to the depot and delivery aspects of the invention discussed herein, it is contemplated the second portion of the multi-functional ligand of the invention may have at least primary medicinal effects that are not immune function related as broadly understood.

It is to be understood that a use of a slash (/) means the broader of “or” or “and/or” unless to the context dictates otherwise.

Some immune interactions require, prefer or are capable of being enhanced via coordinated ligand interactions, for example for optimal immune stimulation, for example, specific costimulatory ligand interactions e.g. CD80/CD86 interactions with CD28, or for example, interactions aimed at tolerizing or otherwise inhibiting or reducing immune effects or preventing such inhibition (for example using anti-CTLA-4/CD152 see related U.S. patents, for example U.S. Pat. Nos. 6,051,227, 5,844,095) (see also Hodge J W et al. Ernst Schering Res Found Workshop 2000 (30): 23-52 and Immunological Reviews Vol 172 December 1999, Entire Issue).

The invention contemplates modeling, evaluating and/or effecting these interactions for therapeutic intervention within the lymphatic system through the substantially contemporaneous use of different multifunctional ligands of the invention. Furthermore, control of the relative proportion of each of the different ligands permits different spatial interspersion of these ligands on the intraluminal surface of the lymphatic system (primarily) so as to provide controlled variability of spatial configurations appropriate for optimizing the coordinate interaction with multiple ligands on another entity, for example immune cells or cancer cells. This strategy also permits controls on avidity that extend beyond the choice of valency for a given single multifunctional ligand for controlling the nature and duration of the coordinate interactions including the duration of temporary anchoring, for example to allow cancer cells to be killed by immune cells, as well delivery of, for example, cytokines (through cytokine antibody fusions), superantigens etc. to the site of interaction. Such coordinate interactions may be substantially contemporaneous or sequential, for example the effect of a first interaction with a first multifunctional ligand slowing the progression of a cell or infectious agent through the lymphatic system for eventual reaction with another first multifunctional ligand (i.e. of the same type) or reaction with a second type of multifunctional ligand. The invention also contemplates as a strategy, alone or in combination with other strategies: 1) delivery of a multifunctional ligand of the invention to a particular site of action for the purpose of exerting, for example a local effect, with the result of causing the multifunctional ligand (whether or not it has exerted its effect, provided that or to the extent that it remains functional in at least one aspect) to subsequently be targeted to the lymphatic system for exerting a second effect (be it the same or a different disease counteracting effect) including simply elimination, or return back to the circulation (i.e. where the ligand is selected (e.g. based on size, immunogenicity etc.) to be preferably minimally eliminated (at least not maximally eliminated) by the body in the course of its circulation, having regard to competing design considerations) for example, in the case of multifunctional ligand which is an anti-tumor ligand that has some residual binding to normal tissues, to set up, in effect, a site of competitive binding that advantageously impacts (i.e. reduces) undesired binding more than desired target binding; 2) delivery of a multifunctional ligand of the invention or an entity that binds to a multifunctional ligand of the invention to a particular site of action e.g., local disease mediating immune cells, for the purpose of simple binding with the expectation that a delayed immune or other effect will be exerted within the lymphatic system. Accordingly, the invention is also directed to a composition comprising at least one and optionally a plurality of different multi-functional ligands of the invention. The invention is also directed to such a composition when combined with a pharmaceutically acceptable carrier for example those that may be suitable for one or more of the various well known and heretofore used routes of administration including intravenous, intradermal etc which (for present purposes) are preferably not incompatible with delivering a multifunctional ligand of the invention to the lymphatic system. The invention is also directed to therapeutic compositions comprising a multifunctional ligand of the invention and to methods of treatment using such compositions. The invention is also directed to method of: 1) evaluating the therapeutic effect of a particular therapeutic entity against a particular target with reduced effect on undesired targets; 2) facilitating elimination a therapeutic entity;—by administering the therapeutic entity as part of or in circumstances which permit interaction with, a multifunctional ligand of the invention.

The invention also contemplates cannulating particular portions of the lymphatic system to localize the delivery of a multifunctional ligand (see U.S. Pat. No. 4,911,690), for example 1) to accommodate or further accommodate the treatment of conditions in which the immune affecting molecule has an undesirable systemic or localized side-effect if delivered otherwise; 2) for the localized delivery, as required, of larger molecules, complexes (e.g. for temporarily anchoring MHC-peptide complexes) or otherwise associated (at least temporarily) entities (i.e. associated other than through complex formation) etc. and/or 3) for the localized delivery of additional compositional elements e.g. adjuvants, cytokines (see Immunological Reviews 2000 Vol 177 p. 5-246; Nature Immunology February 2001 Vol 2 No. 2 page 89), or for affecting only subsets of populations of cells or molecules that pass through the lymphatic system or a desired portion of the lymphatic system or are found with greater concentration within the lymphatic system. The invention also contemplates methods of selective, enhanced or localized, targeting/delivery by administering multifunctional ligands of the invention as well as methods (including methods directly or indirectly employing the multifunctional ligands of the invention) of enhancing/inducing entry of cells or molecules, particularly immune cells (i.e. cells having an immune system function as broadly understood) or subsets thereof, to the lymphatic system or a portion of the lymphatic system, for example for the purpose of direct or indirect interaction with the multifunctional ligands of the invention (in order to be acted on directly or indirectly, by multifunctional ligands of the invention) or for recruiting cells that will for example kill or modulate the activity of other cells, for example kill cancer cells or infected cells that will have, are having or have had direct or indirect interaction with the multifunctional ligands of the invention, as discussed further below, for example in the case of cancer, by targeting immunocytokines to the disease affected tissue e.g. using cytokines e.g. TNFα fused to antibody that binds specifically to tumor cell markers or markers for angiogenesis. Similarly tissue targeted as opposed to disease targeted immunocytokines could be used selectively recruit immune cells within that tissue for example a diseased tissue to enter the lymphatic system for such purposes including for example interaction with a multifunctional ligand of the invention.

It is also contemplated that a single multifunctional ligand can have multiple requisite interactive functionalities for example to stimulate, attract, anergize (or otherwise inactivate) sub-populations of B-cells of T cells via the use, for example, of trivalent or tetravalent antibodies and antibody conjugates/fusions thereof having multiple ligand interactive capabilities (see also for example technologies being developed for selection of successful binders by phage or ribosome display (see for example WO 01/00866; Adv Protein Chem 2000; 55:367-403). A particular application of this technology for application to this invention are antibodies which retarget T-cells to tumor cells (see for example Manzke O. et al. Int. J. Cancer 82, 700-708 (1999); Br J Cancer 2000 January; 82(2): 472-9; J Control Release 2000 Feb. 14; 64(1-3): 229-39 as well as related references, cited therein or citing these publications.

The present invention accommodates such technology through multispecific antibodies or alternatively obviates the need for combining a T-cell receptor type molecule with the primary immune function effecting moiety (e.g. a cancer cell binding moiety) by using a separate multifunctional ligand which combines, for example, a first portion and a second portion comprising a T-cell interacting moiety (e.g. anti-CD3). This is accomplished by administering in the same composition or substantially contemporaneously an amount of the second multifunctional ligand that provides, as may empirically predicted by assessing the dispersion of the marker on the endothelial cell, a strong probability (e.g. 0.001-100%, optionally 1-100%, optionally 5-100%, optionally 10-100%, etc) that the T cell will be targeted in the vicinity of a given lymphatic endothelial cell that happens be proximal to the cell sought be targeted e.g. the cancer cell. It is self-evident that a 50/50 proportion of the first and second multifunctional ligand will yield a strong chance that a second multifunctional ligand will be immediately adjacent on a particular given side (assuming for the sake of argument that there are sides when in reality the dispersion of the lymphatic endothelial marker is governing). It is also contemplated that adjacent multifunctional ligands may be linked for example through linkage effective pairs of ligands (avidin-biotin), the second portions having an antibody component which binds to a common ligand (e.g. on a liposome (see U.S. Pat. No. 6,197,333 and refs. therein cited) or other pharmaceutically acceptable micro/nano particle/sphere of preferably selectable size for optimal spacer or endothelial cell protective purposes) and that such entities could optionally also be employed to house and deliver a payload to a given target vicinity.

In one aspect, the multi-functional ligands of the present invention provide for a method and preferably a means for evaluating and/or inducing immune tolerance (with respect to B cells see strategies discussed in Immunological Reviews 2000 Vol. 176 pp. 5-247).

It is believed that immune tolerance is enhanced or prolonged through prolonged/strategic exposure to tolerance inducing and/or enhancing molecules for example prolonged antigen exposure (see Wang Y et al. Eur. J. Immunol. 2000; 30(18): 2226-2234; Encyclopedia of Immunology; (1998) Morgan Kaufmann Publishers, ISBN: 0122267656; Hoyne G F et al. Immunology 2000 July; 100(3): 281-8; Lerner C G et al. J. Immunol. 2000 Apr. 15; 164(8): 3996-4002; Grossman Z. et al. Semin Immunol 2000 June; 12(3): 197-203; discussion 257-344 Textbook of the Autoimmune Diseases by Lahita R. et al. ISBN: 0781715059 Lippincott Williams & Wilkins; Multi-Systemic Auto-Immune Diseases: An Integrated Approach Dermatological & Internal Aspects ISBN: 0444818960 Elsevier Science; Arthritis and Allied Conditions—A Textbook of Rheumatology, Thirteenth and Fourteenth Editions, William J. Koopman, MD 14^{th}: ISBN: 0-7817-2240-3, November 2000; Principles of Drug Development in Transplantation & Autoimmunity Landes Bioscience, ISBN: 0412100614; Cancer & Autoimmunity by Gershwin M. et al., ISBN: 0444503315 Elsevier Science; J Autoimmun 2000 June; 14(4):278-82; The multi-functional ligands of the present invention, depending on their mode of administration (direct application by cannulating a lymphatic vessel or conventionally e.g. intradermally or intravenously), can be advantageously employed to provide prolonged/strategic exposure to tolerance enhancing molecules (for example by employing a multivalent e.g. bi-specific Ab fragment or diabody which has a first portion which binds to a lymph associated antigen and second portion which optionally comprises anti-idiotypic Ab portion mimicking the desired Ag or the antigen itself or a suitable portion thereof fused or conjugated to the first portion) on the intra-luminal surface of the lymphatic vessels, optionally, in addition to its conventional effects, when administered intradermally or intravenously, etc. It is anticipated that the multi-functional ligands of the present invention would be useful to assess and/or effect tolerance induction (see Bassadona G P et al. Proc Nat/Acad Sci U S A 1998 Mar. 31; 95(7): 3821-6; U.S. Pat. No. 6,106,834; U.S. Pat. No. 6,099,838; U.S. Pat. No. 6,010,902: Antibody heteroconjugates and bispecific antibodies for use in regulation of lymphocyte activity; as well as additional examples cited below with reference to examples of suitable anti-idiotypic antibodies).

It is also contemplated that a multispecific construct as described in WO99/37791 could be used with respect to various aspects of the invention.

### Additional Applications of Various Aspects of the Invention

It is contemplated that the present invention could be used to strategically mediate, CD45 (or variants/other PTPs) related “cell signaling”, for example through signaling molecules (e.g. inhibitors) using multifunctional ligands of the invention including but not limited to bispecific antibodies, antibody fusions/conjugates e.g. where the immune affecting antibody portion or other moiety is conjugated, fused etc. to an antibody or fragment that binds to an entity associated marker e.g. LYVE-1 (1999) Journal of Cell Biology Vol 144 No 4 p. 789-801) (see for example U.S. Pat. No. 5,914,111 Sievers E L, Cancer Chemother Pharmacol 2000 46 Suppl s18-22 WO9946268, Neel B G Curr Opin Immunol 1997 Jan. 9(3) 405-420; Front Biosci 1998 Nov. 13: D-1060-96, Slifka M K et al. J. Mol. Med. 2000 78(2) 74-80 Goodnow C C Ciba Found Symp 1997 204: 190-202; Mustelin T. et al. Front Biosci. 1998 Nov. 1; 3: D1060-96; Gaya A, Leuk Lymphoma, 1999 Oct. 35 (3-4): 237-43; Sievers E L, Curr Opin Oncol. 2000 Jan. 12(1): 30-5; Thomas M L, et al. Immunol. Today 1999 Sep. 20(9): 406-411; Appelbaum F R, Semin. Hematol. 1999 October; 36 (4 suppl. 6): 2-8; Ulyanova T; Immul. Res 1997 February; 16(1): 101-13; re PP32 for example U.S. Pat. No. 5,846,822 and Brody J R, et al. J Biol Chem. 1999 Jul. 16; 274(29): 20053-5 regarding the functional moiety of PP32 which is necessary for interaction with CD45, and for example U.S. Pat. No. 5,981,251 with respect to methods of identifying such molecules).

In preferred embodiments the invention is directed to multifunctional ligands that comprise immune function exerting moieties having functionalities of molecules currently in clinical trials or proposed for clinical trials (see for example Glennie M J et al. August 2000, Immunology Today 408 Vol 21(8); see also Journal of Immunological Methods 237 (2000) 131-145; Mol Immunol 2000 June; 37(9) 515-526; Annu Rev Med 2001; 52:125-145; Annu Rev Med 2001 52:63-78; Q J Nucl Med 2000 September; 44(3) 268-83) including those that have an anti-CD2 functionality (see U.S. Pat. No. 5,795,572) anti-CD4 functionality (see for example U.S. Pat. No. 6,136,310 Herzyk D, J Infect Immun 2000 February; 69(2): 1032-1043) anti-CD3 functionality (for example WO 00/41474; WO 98139363; U.S. Pat. No. 6,113,901; Transplantation 2000 Dec. 27 70(12) 1707-12); Anti-CD44 functionality see for example Weiss L, et al., Proc Nat Acad Sci USA 2000; Jan. 4 97(1)285-290; Sugiyama K, Immunol Invest (1999) March-May 28(2-3)185-200; Brocke S. et al. Proc Nat Acad Sci USA 1999 Jun. 8 96(12) 6896; Mickecz K et al. Nat Med 11995 June; 1(6); 558-63; Ahrens T et al., J Invest Dermatol. 2001 January 116(1) 93-101); with respect to control of migration of T-cell lymphocytes see Nohara C, et al. J. Immunol. 2001 Feb. 1; 166(3) 2108-2115), anti-CD20 functionality (see Crit Rev Oncol Hematol 2001 Jan. 37(1): 13-25) etc. anti-CD22 functionality see for example Newton D L, et al. Blood 2001 Jan. 15; 97 (2): 528-535, U.S. Pat. No. 5,184,892; Anti-CD40/CTLA-4 see for example J Immunol 2000 Oct. 1; 165(7): 3612-9; Microsurgery 2000; 2c (8); 448-452; U.S. Pat. No. 5,874,082; U.S. Pat. No. 6,056,959; U.S. Pat. No. 5,801,227; U.S. Pat. No. 6,004,552; U.S. Pat. No. 5,677,165; U.S. Pat. No. 6,087,329; U.S. Pat. No. 5,961,974; U.S. Pat. No. 6,051,228; White C A, et al. Annu Rev Med. 2001; 52: 63-78 (see also reviews and specific applications referred to in Ditzel et al., Immunol Res. 2000; 21(2-3): 185-93; U.S. Pat. No. 6,010,902, U.S. Pat. No. 5,876,950; U.S. Pat. No. 5,876,718; U.S. Pat. No. 5,601,819, U.S. Pat. No. 5,981,251, U.S. Pat. Nos. 5,885,579 and 5885796; Cancer Immunol Immunother 2000 June; 49(3): 173-80; Omar K, J Neuroimmunol 2001 Feb. 1, 113(1) 129-141; Bellido M, Eur J. Haematol 2001 Feb. 66(2) 100-106; Broeren et al. J Immunol (2000) Dec. 15 165(12) 6908-14; Alexandroff A B et al Mol Immunol 2000 June 37(9) 515-526; Werkerle T J. Immunol. 2001 Feb. 15 166(4) 2311-2316; Howard L M J Immunol 2001 February; 116(3) 1547-53 anti-CD154; J Pharmacokinet Biopharm 1999 August; 27(4):397-420, J Clin Oncol 2000 April; 18(8):1622-36, Leukemia 2000 March; 14(3):474-5, Clin Cancer Res 2000 February; 6(2):372-80, Leukemia 2000 January; 14(1):129-35, J Nucl Med 1999 November; 40(11):1935-46, Blood 1999 Nov. 15; 94(10): 3340-8, Blood 1999 Aug. 15; 94(4):1237-47, Cancer Res 1999 May 1; 59(9):2096-101, Vaccine 1999 Apr. 9; 17(15-16):1837-45, Blood 1998 Dec. 1; 92(11):4066-71, J Rheumatol 1998 November; 25(11):2065-76, Clin Pharmacol Ther 1998 September; 64(3):339-46, Mult Scler 1996 July; 1 (6):339-42, Cancer Immunol Immunother 1997 July; 44(5):265-72, Transplant Proc 1996 December; 28(6):3210-1, Arthritis Rheum. 1996 July; 39(7):1102-8, Immunology 1996 May; 88(1):13-9 and U.S. Pat. No. 5,876,718).

The invention contemplates assessment and therapeutic benefit of lymphatic localization in the case of antibodies and multispecific ligands which are toxic to non-target cell populations which express the targeted ligand to a limited extent or in the case of toxic cross-reactivity of the second portion e.g. antibody for its desired target with an undesired target (see e.g. Lancet 1999 Nov. 13; 354(9191): 1691-5). It is contemplated that the toxic effect of a given effector moiety of a multifunctional ligand of the invention could be alternated using an additional binding arm for a lymphatic marker.

### Antibody Structure and Function

Antibody structure and function has bee extensively described in the literature. For example, see Antibody Engineering 2^{nd} ed. Carl A. K. Borrebaeck, Oxford University Press 1995 p 3-44.

### Production of Bispecific Antibodies

A variety of different constructs have been developed for the production of bispecific antibodies including conventional four chain antibodies (including truncated version thereof such minibodies (see U.S. Pat. No. 5,837,821), F(ab′)₂ (see Antibody Fusion Proteins, Steven M Chamow, Avi Ashkenazi Eds. ISBN 047118358X May 1999 Wiley p. 136-144; or using CH3-truncated heavy chains), diabodies (see U.S. Pat. No. 5,837,242 Multivalent and multispecific binding proteins, their manufacture and use) constructs in which of one or two diabody molecules are heterodimerized by creating a fusion protein with the CL and CH1 immunoglobulin constant domains (see WO 02/02781).

In recent years, a variety of chemical and recombinant methods has been developed for the production of bispecific and/or multivalent antibody fragments. For review, see: Kriangkum J, et al. Bispecific and bifunctional single chain recombinant antibodies. Biomol Eng 2001 September; 18(2): 31-40, Holliger P. and Winter, G., Curr. Opin. Biotechnol. 4, 446-499 (1993); Carter, P. et al., J. Hematotherapy 4, 463-470 (1995); Pluckthan, A. and Pack, P., Immunotechnology 3, 83-105 (1997). Bispecificity and/or bivalency has been accomplished by fusing two scFv molecules via flexible linkers, leucine zipper motifs, C_{H}C_{L}-heterodimerization, and by association of scFv molecules to form bivalent monospecific diabodies and related structures. Multivalency has been achieved by the addition of multimerization sequences at the carboxy or amino terminus of the scFv or Fab fragments, by using for example, p53, streptavidin and helix-turn-helix motifs. For example, by dimerization via the helix-turn-helix motif of an scFv fusion protein of the form (scFv1)-hinge-helix-turn-helix-(scFv2), a tetravalent bispecific is produced having two scFv binding sites for each of two target antigens.

Production of IgG type bispecific antibodies, which resemble IgG antibodies in that they posses a more or less complete IgG constant domain structure, has been achieved by chemical cross-linking of two different IgG molecules or by co-expression of two antibodies from the same cell. Both methods result in production of significant amounts of undesired and non-functional species due to mispairing among the component heavy and light chains. Methods have been employed to reduce or eliminate mispairing.

One strategy developed to overcome unwanted pairings between two different sets of IgG heavy and light chains co-expressed in transfected cells in modification of the Cₕ3 domains of two heavy chains to reduce homodimerization between like antibody heavy chains. Merchant, A. M., et al., (1998) Nat. Biotechnology 16, 677-681. In that method, light chain mispairing was eliminated by requiring the use of identical light chains for each binding site of those bispecific antibodies.

To produce bispecific antibodies, Kostelny et al (J. Immunology 148:1547 (1992)) fused Fab fragments of antibodies to the leucine zipper portions of fos and jun proteins in the absence of a single chain construct for the antigen combining region. These methods are well described in the literature and summarized with references in Antibody Fusion Proteins, Steven M Chamow, Avi Ashkenazi Eds. ISBN 047118358X May 1999 Wiley; Kontermann, R., et al. (Eds.) particularly at pages 139-145. Pack and Pluckthun, fused a single chain antibody to amphipathic helices from a four helix bundle or from leucine zipper proteins.

Bispecific antibodies that are in a conventional IgG-like and Fab-like format have been developed by Zhu as tetravalent or bivalent molecules, respectively with each of the chains serving to anchor a binding moiety (see WO 01/90192 and FIG. 1 therein), preferably consisting of a scFv. In the bispecific IgG-like construct, each side of the molecule comprises a CH1 domain and a CL domain and each CH and CL domain is linked through its N-terminus to a scFv of different specificity. The invention herein contemplates that this construct can readily be adapted to have each half of the molecule associated with a polypeptide e.g. a scFv of the same specificity so that each half of the molecule is monospecific (or to have each half of the molecule associated with different pairings of scFvs) so that each half of the molecule is effectively monospecific. The invention herein contemplates that a bivalent relatively low affinity second ligand binding moiety is used to activate receptors that require cross-linking for activity. The invention also contemplates that numerous permutations in which the functional affinity of the first ligand binding moiety whether monospecific or bispecific can be accentuated relative the functional affinity of the second ligand binding moiety including employing a first ligand high affinity scFvs in which the second ligand binding moiety is effectively monovalent (has one, or one useful binding moiety). The invention also contemplates that this construct can have a truncated Fc portion and various known methods in the art for improving the pairing efficiency of the heavy chains. The invention also contemplates that the CH1 and CL domains of the second ligand binding moiety can be truncated as in camelid antibodies for efficient delivery e.g. of biologic effector ligands.

Methods of Generating Antibodies that Bind to Selected Target Ligands

A variety of technologies for generating antibodies with desired specificity have been extensively developed and become well known to and routinely practiced by those skilled in the art including phage display (see review in Basic Methods in Antibody Production & Characterization G. C. Howard et al. eds. CRC Press 2001 p. 105) and other display systems (ribosome display, display on the surface of various cells), immunizing mice, including particularly mice having human Ig genes, and antibody microarray technologies. These methods have also been extended to making antibodies with dual specificities such as diabodies (U.S. Pat. No. 5,837,242 Multivalent and multispecific binding proteins, their manufacture and use) and are the subject of extensive scientific and patent literature. For example, see U.S. Pat. Nos. of Winter et al. 6,291,650; 6,291,161; 6,291,158; 6,017,732; 6,225,447; 6,172,197; 6,140,471, 6,010,884 5,969,108, 5,871,907, 5,858,657; 5,733,743, 5,723,287 and those of Dyax, Morphosys, and Cambridge Antibody Technology.

### Affinity Maturation

Methods of codon based mutagenesis have been extensively developed for engineering the antibody binding site. For example, the use of such methods in a filamentous phage display system is described in Antibody Engineering 2^{nd} ed. Carl A. K. Borrebaeck, Oxford University Press 1995 p 117-128 see also pp. 53-84 with respect to techniques of phage display of antibodies (see also Kontermann, R, Dubel, S., (Eds.): (2001) Antibody Engineering ISBN: 3-540-41354-5)

### Methods of Generating Single Domain Ligands

The ability of a single variable fragment of an antibody to bind with specificity and suitable selected affinities in the nanomolar+ range has been extensively demonstrated using camelid and human VH fragments. Methods of generating VHs with the desired specificity have been extensively described (see U.S. Pat. No. 6,248,516 Single domain ligands, receptors comprising said ligands methods for their production, and use of said ligands and receptors). (See also literature referenced herein on this subject).

### Methods of Making Antibodies in E. Coli

The expression of recombinant antibodies, including diabodies in E. Coli has become routine. General precepts, and methods are discussed in Antibody Engineering 2^{nd} ed. Carl A. K. Borrebaeck, Oxford University Press 1995 p 229-266 see also Antibody Therapeutics W J Harris et al. eds. CRC Press 1997 p. 221; see also review in Biotechnology, Volume 5A, Recombinant Proteins, Monoclonal Antibodies, and Therapeutic Genes A. Mountain, U. Ney, Dietmar Schomburg ISBN: 3-527-28315-3, January 1999, Antibody Production Essential Techniques Peter J. Delves ISBN: 0-471-97010-7 Wiley June 1997 and Antibody Therapeutics Production, Clinical Trials, and Strategic Issues, By Rathin C. Das, Ph.D., M.B.A. & K. John Morrow, Jr., Ph.D., D&MD Publications October 2001 Chapter 3.

### Eukaryotic & Other Expression & Production Systems

Approaches for the eukaryotic expression of antibodies and antibody fusion proteins and the preparation of vectors for use in such methods are well known and extensively described in the literature. General precepts, and methods are discussed is Antibody Engineering 2^{nd} ed. Carl A. K. Borrebaeck, Oxford University Press 1995 p 267-293 (see also Antibody Therapeutics W J Harris et al. Eds. CRC Press 1997 p. 183-220; see also review in Biotechnology, Volume 5A, Recombinant Proteins, Monoclonal Antibodies, and Therapeutic Genes A. Mountain, U. Ney, Dietmar Schomburg ISBN: 3-527-28315-3, Wiley, January 1999 and Antibody Production: Essential Techniques Peter J. Delves ISBN: 0-471-97010-7 Wiley June 1997 and Antibody Therapeutics Production, Clinical Trials, and Strategic Issues, By Rathin C. Das, Ph.D., M.B.A. & K. John Morrow, Jr., Ph.D., D&MD Publications October 2001 Chapter 3.

With respect to a review of immunotoxins, see also Antibody Therapeutics W J Harris et al. Eds. CRC Press 1997 p 33

With respect to Methods for producing recombinant vectors, see also U.S. Pat. No. 5,962,255 Methods for producing recombinant vectors

Formulation, purification and analytic methods involving antibodies are well known to those skilled in the art and have been extensively reviewed. With respect to formulation, purification and analytic methods see for example, reviews in Antibody Therapeutics Production, Clinical Trials, and Strategic Issues, By Rathin C. Das, Ph.D., M.B.A. & K. John Morrow, Jr., Ph.D., D&MD Publications October 2001, Chapter 4.

With respect to methods of generating antibodies against self-antibodies, see U.S. Pat. No. 5,885,793 Production of anti-self antibodies from antibody segment repertoires and displayed on phage

### Antibody Conjugates

Methods of chemical manipulation of antibodies for attachment of ligands (e.g. biotin), radionuclides etc. are well known in the art and have been extensively reviewed (for example see review in Basic Methods in Antibody Production & Characterization G. C. Howard et al. eds. CRC Press 2001, p. 199; with respect to therapeutic principles see for example, Antibody Therapeutics W J Harris et al. eds. CRC Press 1997 p 53-88).

The applications of bispecific antibodies, including methods of making and using them have been extensively reviewed (see for example van Spriel A B, van Ojik H H, van De Winkel J G. Immunotherapeutic perspective for bispecific antibodies. Immunol Today. 2000 August; 21(8): 391-7; Weiner L M. Bispecific antibodies in cancer therapy. Cancer J Sci Am. 2000 May; 6 Suppl 3:S265-71. Barbet J, et al. Pretargeting with the affinity enhancement system for radioimmunotherapy. Cancer Biother Radiopharm. 1999 June; 14(3): 153-66. de Wolf F A, Brett G M. Ligand-binding proteins: their potential for application in systems for controlled delivery and uptake of ligands. Pharmacol Rev. 2000 June; 52(2): 207-36. Wang H, Liu Y, Wei L, Guo Y. Bi-specific antibodies in cancer therapy Adv Exp Med. Biol. 2000; 465:369-80; Staerz U D, Lee D S, Qi Y. Induction of specific immune tolerance with hybrid antibodies. Immuno Today. 2000 April; 21(4): 172-6: 1999 December; 43(4): 336-43. Elsasser D, Stadick H, van de Winkel J G, Valerius T. GM-CSF as adjuvant for immunotherapy with bispecific antibodies. Eur J. Cancer. 1999 August; 35 Suppl 3:S25-8. Molema G, Kroesen B J, Helfrich W, Meijer D K, de Leij L F. The use of bispecific antibodies in tumor cell and tumor vasculature directed immunotherapy. J Control Release. 2000 Feb. 14; 64(1-3): 229-39. Bodey B, Bodey B, Siegel S E, Kaiser H E. Genetically engineered monoclonal antibodies for direct anti-neoplastic treatment and cancer cell specific delivery of chemotherapeutic agents. Curr Pharm Des. 2000 February; 6(3): 261-76. Kudo T, Suzuki M, Katayose Y, Shinoda M, Sakurai N, Kodama H, Ichiyama M, Takemura S, Yoshida H, Saeki H, Saijyo S, Takahashi J, Tominaga T, Matsuno S. Specific targeting immunotherapy of cancer with bispecific antibodies. Tohoku J Exp Med. 1999 August; 188(4): 275-88. Koelemij R, et al. Bispecific antibodies in cancer therapy, from the laboratory to the clinic. J. Immunother. 1999 November; 22(6): 514-24. Segal D M, Weiner G J, Weiner L M Bispecific antibodies in cancer therapy Curr Opin Immunol. 1999 October; 11(5): 558-62. Hudson P J. Recombinant antibody constructs in cancer therapy. Curr Opin Immunol. 1999 October; 11(5): 548-57. Barth R F et al, Boron neutron capture therapy of brain tumors: an emerging therapeutic modality. Neurosurgery. 1999 March; 44(3): 433-50; Fleckenstein G, Osmers R, Puchta J. Monoclonal antibodies in solid tumours: approaches to therapy with emphasis on gynecological cancer, Med. Oncol. 1998 December; 15(4): 212-21. Guyre C A, Fanger M W. Macrophage-targeted killing and vaccines. Res Immunol. 1998 September-October; 149(7-8): 655-60 Cao Y, Suresh M R. Bispecific antibodies as novel bioconjugates. Bioconjug Chem. 1998 November-December; 9(6): 635-44. Farad R A, et al, The development of monoclonal antibodies for the therapy of cancer. Crypt Rev Eukaryote Gene Expert. 1998; 8(3-4): 321-56.: Volt M. Multiorgan resistance and its reversal. Anticancer Res. 1998 July-August; 18(4C): 2905-17. Rouard H, et al, Fc receptors as targets for immunotherapy. Int Rev Immunol. 1997; 16(1-2): 147-85. Fan Z et al. Therapeutic application of anti-growth factor receptor antibodies; Curr Opin Oncol. 1998 January; 10(1): 67-73. de Gast G C, et al, Clinical perspectives of bispecific antibodies in cancer. Cancer Immunol Immunother. 1997 November-December; 45(3-4): 121-3. Carter P, Merchant A M. Engineering antibodies for imaging and therapy. Curr Opin Biotechnol. 1997 August; 8(4): 449-54. Pluckthun A, et al, New protein engineering approaches to multivalent and bispecific antibody fragments. Immunotechnology. 1997 June; 3(2): 83-105. Rihova B. Targeting of drugs to cell surface receptors. Crit Rev Biotechnol. 1997; 17(2): 149-69. Molema G et al, Tumor vascular endothelium: barrier or target in tumor directed drug delivery and immunotherapy. Pharm Res. 1997 January; 14(1): 2-10. Bodey B, et al, Human cancer detection and immunotherapy with conjugated and non-conjugated monoclonal antibodies. Anticancer Res. 1996 March-April; 16(2): 661-74 Hartmann F et al, Treatment of Hodgkin's disease with bispecific antibodies. Ann Oncol. 1996; 7 Suppl 4:143-6. Wels W, et al, Intervention in receptor tyrosine kinase-mediated pathways: recombinant antibody fusion proteins targeted to ErbB2. Curr Top Microbiol Immunol. 1996; 213 (Pt 3): 113-28.: Kairemo K J. Radioimmunotherapy of solid cancers: Acta Oncol. 1996; 35(3): 343-55.; Verhoeyen M E, et al, Antibody fragments for controlled delivery of therapeutic agents. Biochem Soc Trans. 1995 November; 23(4): 1067-73. Haagen I A. Performance of CD3xCD19 bispecific monoclonal antibodies in B cell malignancy. Leuk Lymphoma. 1995 November; 19(5-6): 381-93.

In another aspect the invention is directed to presenting antigen within the lymphatic system (e.g. in the form of an anti-idiotype antibody) such as to facilitate a desired immune response e.g. vaccination type responses). Optionally, adjuvants can be conventionally employed to assist initial immune stimulation e.g. intradermally when appropriately delivered. Activating cytokines for example as specified above, can also be employed to enhance the immune response. Examples of antibodies having an anti-idiotypic counterpart or for which an anti-idiotypic counterpart could made by well known techniques in the art (and that are capable of exerting the desired anti-idiotypic effect) are numerous and numerous such antiidiotypic antibodies have application to immunization as well as applications relating to tolerance (see for example U.S. Pat. Nos. 6,146,627 Method for reducing T cell-mediated cytotoxicity in HIV using anti-idiotypic antibody; 6,063,679 Anti-idiotypic monoclonal antibodies and compositions including the anti-idiotypic monoclonal antibodies; 6,060,049 Surrogate tolerogenesis for the development of tolerance to xenografts; 6,042,827 Anti-idiotypic antibody induction of anti-tumor response; 6,007,815 Anti-idiotype vaccination against diseases resulting from pathogenic responses by specific T cell populations; 5,981,502 Methods and compositions for inducing apoptosis in tumor cells; 5,766,588 Tumor immunotherapy using anti-idiotypic antibodies; 5,728,812 Anti-idiotypic antibody composition for inhibiting acute complement-mediated cytotoxicity.

According to another aspect of the invention the multi-functional ligand comprises a first portion which binds to a lymph associated antigen and a second portion which binds to a tumor cell infected cell or infectious agent. This embodiment of the invention can be used for example, to assess and affect the ability of the tumor-binding portion to more advantageously inhibit metastasis. Optionally, for example, the portion which binds to a lymph associated antigen has a lower affinity and/or avidity so that the tumor cell binding portion preferentially binds to the tumor cell and is therefore more likely to accompany its passage through the lymphatic system. This strategy also has application to bi-specific antibodies of the invention in which the second portion is for example targeted to an immune cell. Optionally, multiple such multi-functional ligands may permit sufficient tumor cell anchoring to permit the tumor cell to be killed within the lymphatic system via a toxic payload carried by the multifunctional ligand or through the recruitment of immune cells which accomplish this end (e.g. using the same or a different multifunctional ligand fused or conjugated to a suitable cytokine (e.g. IL-2, IL-12). The prolonged presence of these cells could be advantageously used to assess methods of immunization directly against the tumor cell using, for example, cytokines including cytokines fused or conjugated in whole or functional part to a lymph targeted Ab on the same, or a different multifunctional ligand delivered in a suitable dose (with respect to generation of anti-tumor antibodies and other antibody fragments for application herein as well as important related technologies see also WO 00/50008; WO 01/01137; WO 97/37791; WO 99/37791; WO 97/10003; Hoogenboom et al. Nat. Biotechnology 15(2) February 1997 p 125-126; Fell H. et al. Journal Of Immunology Vol 146(7) April 1991 p 2446-2452; Anderson D. et al Bioconjugate Chemistry 14(1) January 1993 p 10-18; U.S. Pat. No. 6,172,197; U.S. Pat. No. 6,171,782; Immunological Investigations 2000 29(2) entire issue). Optionally the tumor binding portion internalizes and/or delivers a toxic payload, for example a radionuclide, or other toxin, or a cytokine to the tumor cell (with respect to selection of tumor internalizing human antibodies see for example Pool M et al. J Mol. Biol. 2000 Sep. 1; 301(5): 1149-61, see also Kohl M D et al. J. Mol. Biol. Biotechniques (2000) Vol 28(1) p 162 In this way the multi-functional ligands of the invention, for example, when provided in a sufficient dose to both target the tumor and line a portion of the lymphatic system to which the target tumor is likely to drain, acts as a cancer treatment as well as a sentry system for assessing/augmenting (for example as an adjunct therapy) the ability of the tumor binding portion with/without payload to inhibit metastasis. There are numerous examples of functional cytokine and toxin fusions used for example in cancer therapy that may have application to the invention herein (for examples and reviews see references herein cited as well as WO 99/37791; WO99 WO00/06605; WO 99/52562 WO 99/37791 MULTIPURPOSE ANTIBODY; Proceeding of the IBC's 11^{th} Annual International Conference on Antibody Engineering State of the Art Science, Technology and Applications, Dec. 3-6, 2000; Amplification of T cell-mediated immune responses by antibody-cytokine fusion proteins. Immunol Invest. 2000 May; 29(2): 117-20; Cancer Res. 1999 May 1; 59(9): 2159-66.; Pharmacokinetics and stability of the ch14.18-interleukin-2 fusion protein in mice. Cancer Immunol Immunother. 1999 August; 48(5): 219-29. Phase I study of single, escalating doses of a superantigen-antibody fusion protein (PNU-214565) in patients with advanced colorectal or pancreatic carcinoma. J. Immunother. 2000 January; 23(1): 146-53. Targeted toxin therapy for malignant astrocytoma. Neurosurgery. 2000 March; 46(3): 544-51; Targeting cytokines to tumors to induce active antitumor immune responses by recombinant fusion proteins. Hum Antibodies. 1999; 9(1): 23-36; Lode H N, et al. Tumor-targeted IL-2 amplifies T cell-mediated immune response induced by gene therapy with single-chain IL-12. Proc Natl Acad Sci USA. 1999 Jul. 20; 96(15): 8591-6; Cancer Vaccines and Immunotherapy 2000 (textbook); Immunotherapy With Intravenous Immunoglobulins P. Imbach (1991) Academic Press; Molecular Approaches to Tumor Immunotherapy (1997) World Scientific Publishing Company, Incorporated; Vaccines & Immunotherapy S. J. Cryz (1991) McGraw-Hill Ryerson, Limited

With respect to internalizing antibodies, see e.g. Biological Effects of Anti-ErbB2 Single Chain Antibodies Selected for Internalizing Function. Biochem Biophys Res Commun. 2001 Jan. 12; 280(1): 274-279 and references cited therein, Immunoconjugates of geldanamycin and anti-HER2 monoclonal antibodies: antiproliferative activity on human breast carcinoma cell lines J Natl Cancer Inst. 2000 Oct. 4; 92(19): 1573-81; Foulon C F, et al., Radioiodination via D-amino acid peptide enhances cellular retention and tumor xenograft targeting of an internalizing anti-epidermal growth factor receptor variant III monoclonal antibody. Cancer Res. 2000 Aug. 15; 60(16): 4453-60. Poul M A, Becerril B, Nielsen U B, Morisson P, Marks Selection of tumor-specific internalizing human antibodies from phage libraries J Mol. Biol. 2000 Sep. 1; 301(5): 1149-61. Vrouenraets M B, et al., Targeting of a hydrophilic photosensitizer by use of internalizing monoclonal antibodies: A new possibility for use in photodynamic therapy. Int J. Cancer. 2000 Oct. 1; 88(1): 108-14.

In yet another aspect, the invention contemplates that the passage of tumor cells can be inhibited within the tumor vasculature using a bispecific ligand, optionally a bispecific antibody, which targets on the one hand a well known vascular endothelial marker and one the other hand binds to a ligand on the surface of the tumor. Other aspects of the invention related to tumor cell targeting are understood to described in reference to this aspect of the invention as well. It is also contemplated that markers which are present on both the lymphatic endothelium and the tumor vasculature can be simultaneously targeted with bispecific ligands of the invention to inhibit tumor metastasis and/or immunize a subject against tumor cells.

It is contemplated that the multifunctional ligands of the invention when used to inhibit metastasis, for example, in the manner described above, could be advantageously employed in combination with other well known therapies for example cytotoxic drugs, other tumor targeted antibodies and conjugate/fusions therewith used or currently being evaluate for immunotherapies, angiogenesis targeted drugs etc. (re angiogenesis see for example Angiogenesis in cancer and other diseases. Nature. 2000 Sep. 14; 407(6801): 249-57).

Similarly, a bi-specific antibody of the invention could be used to bind to antigens/ligands on lymphocytes which are known or become known to inhibit or enhance immune function or mediate a disease e.g. CD45.

With respect to target receptors related to the inventions defined herein, see also U.S. Pat. No. 6,277,962.

As discussed above, as used herein the term “lymph associated antigen” refers to antigens that are expressed significantly on lymphatic endothelial cells but not significantly expressed, if at all, on other tissues. Examples of such antigen include LYVE-1a CD44 receptor analogue which binds to HA (Feb. 22, 1999, Banerji et. al., Journal of Cell Biology Vol. 144, #4, p 789-801) and which is expressed primarily on lymphatic endothelial cells. LYVE-1 specific antisera have been shown to inhibit binding of HA. The invention contemplates research and treatments using multi-functional ligands of the invention with respect to non-human mammals, including preferably agricultural animals, canine species, primates and mice having similar receptors/antigens. For example, a murine counterpart to LYVE-1 (published in Prevo R. et al. 2001 Feb. 20, J. Biol. Chem.; Manuscript M011004200) can be employed to implement the various methods and embodiments described herein in a mouse model, for example to assess the extent of inhibition of metastasis effected by a multifunctional ligand (optionally comprising for example to a toxin, cytokine T cell receptor etc) which has a first portion which binds to LYVE-1 and a second portion which binds to, for example to GI-101, a breast tumor which is known to metastasize to the lung (see U.S. Pat. Nos. 6,037,520 and 5, 693, 533 see also U.S. Pat. Nos. 5,643,551, 5,491,284, 5,569,812, 5,917,124 and 6,107,540 and references cited in these patents, particularly with respect to other metastatic models and methods of evaluating anticancer drugs in mice). LYVE-1 counterparts in other mammals can be identified in the manner described by Prevo R. et al. (see also Skobe M. et al. Induction of tumor lymphangiogenesis by VEGF-C promotes breast cancer metastasis Nat. Med. February; 7(2) 192-8.)

Other models of metastasis in animals are well known in the art (see for example Chirgwin J M, Guise T A. Molecular mechanisms of tumor-bone interactions in osteolytic metastases. Crit Rev Eukaryot Gene Expr. 2000; 10(2): 159-78. 3: Kobaek-Larsen M, et al. Review of colorectal cancer and its metastases in rodent models: comparative aspects with those in humans. Comp Med. 2000 February; 50(1): 16-26. 5: Magnano M, et al. A physical-based model for the simulation of neoplastic growth and metastasis. J Surg Oncol. 2000 June; 74(2): 122-9. 6: Hoffman R M. Orthotopic metastatic mouse models for anticancer drug discovery and evaluation: a bridge to the clinic. Invest New Drugs. 1999; 17(4): 343-59. Russo J, Russo I H. The pathway of neoplastic transformation of human breast epithelial cells. Radiat Res. 2001 January; 155(1 Pt 2): 151-154. Duffy M J, McCarthy K. Matrix metalloproteinases in cancer: prognostic markers and targets for therapy (review). Int J. Oncol. 1998 June; 12(6): 1343-8. 22: Banerjee A, Quirke P. Experimental models of colorectal cancer. Dis Colon Rectum. 1998 April; 41(4): 490-505. Wu T T et al. Establishing human prostate cancer cell xenografts in bone: induction of osteoblastic reaction by prostate-specific antigen-producing tumors in athymic and SCID/bg mice using LNCaP and lineage-derived metastatic sublines. Int J. Cancer. 1998 Sep. 11; 77(6): 887-94.61: Molpus K L, et al. Characterization of a xenograft model of human ovarian carcinoma which produces intraperitoneal carcinomatosis and metastases in mice. Int J. Cancer. 1996 Nov. 27; 68(5): 588-95.65: Pages J C, Sordat B, Bautista D, Costa J, Benhattar J. Detection of rare circulating human colon tumor cells in a nude mouse xenograft model. Cancer Lett. 1996 Aug. 23; 106(1): 139-44.66: Sakakibara T, et al. Doxorubicin encapsulated in sterically stabilized liposomes is superior to free drug or drug-containing conventional liposomes at suppressing growth and metastases of human lung tumor xenografts. Cancer Res. 1996 Aug. 15; 56(16): 3743-6.

With respect to modifying an antibody to increase its affinity see also Crystal structure of Fab198, an efficient protector of the acetylcholine receptor against myasthenogenic antibodies. Eur J. Biochem. 2001 July; 268(13): 3685-3693.

For example, in one embodiment the invention contemplates a bispecific antibody comprising an antigen binding component specific for a tumor cell associated antigen and a relatively low affinity anti-IL-6 receptor antibody component. With respect to the anti-tumor role of IL-6 see Wei L H et al. Interleukin-6 in cervical cancer: the relationship with vascular endothelial growth factor. Gynecol Oncol. 2001 July; 82(1): 49-56.

The invention contemplates that TCRs and modified TCRs (see for example, WO 01/48145) may be used as ligands, in place of antibody fragments for binding to target ligands such as peptide/MHC ligands.

Techniques for generating antibodies, and methods, for example of subtractive screening useful to identify other lymphatic vessel associated antibodies, including those optionally having smaller scFv, Fab and dAb (single domain antibody or functional fragment thereof) component (more easily passaging to lymphatic vessels from tissues particularly when constructed in the form of bispecific antibodies e.g. diabodies etc.) by phage or ribosome display are well known in the art (see for example Hoogenbom H R et al. Immunol. Today (August 2000) Vol 8 p 371; Schaffitzel C. et al. J. Immunol. Methods (Dec. 10, 1999) 231(1-2) p. 119; Roberts R W et al. Curr Opin Chem. Biol. 1999 June; 3(3): 268-73; Winter G. et al. Annu Rev Immunol 1994 12:433-55; Kontermann R E et al. Nat Biotechnol. 1997 July; 15(7): 629-31; Phage Display of Peptides and Proteins, A Laboratory Manual Kay B K et al. Eds 1996 Academic Press; Immunology Methods Manual Lefkovits, I ed. 1997 Academic Press; Hoogenboom et al. Immunotechnology 4 (1998) 1-20;

**With respect to making single domain antibodies see for example U.S. Pat. No. 5,824,520, U.S. Pat. No. 5,622,836, U.S. Pat. No. 5,702,892, U.S. Pat. No. 5,959,087, Unique single-domain antigen binding fragments derived from naturally occurring camel heavy-chain antibodies. J Mol Recognit. 1999 March-April; 12(2): 131-40. An antibody single-domain phage display library of a native heavy chain variable region: isolation of functional single-domain VH molecules with a unique interface. J Mol Biol. 1999 Jul. 16; 290(3): 685-98 and references cited in these references.

Methods for making antibody fusion proteins and bi-specific antibodies including diabodies etc. and fusion proteins thereof are well established in the art (for reviews and particular applications see for example Adams G P et al. Journal of Immunological Methods 231 (1999) 249-260; U.S. Pat. Nos. 6,121,424, 6,027,725 and 6,025,165; EP 0654085; Hudson P. Exp. Opin. Invest. Drugs (2000) 9(6): 1231-1242; Antibody Fusion Proteins Steven M Chamow, Avi Ashkenazi Eds. ISBN 047118358X May 1999 Wiley; Antibody Engineering, Carl A. Borrebaeck Oxford University Press, 1995; Antibody Engineering: A Practical Approach David J. Chiswell, Hennie. R. Hoogenboom, John McCafferty Oxford University Press, 1996; Antibody Engineering Protocols, Sudhir Paul (1995) Humana Press; Antibody Expression & Engineering (1998) Henry Y. Wang, Tadayuki Imanaka, American Chemical Society; Zhu Z. Biotechnology (NY) 1996 February; 14(2): 192-6; Nielsen U B et al. Cancer Res. 2000 Nov. 15; 60(22):6434-40; Lawrence L J. Et al Febs Lett. 1998 Apr. 3; 425(3) 479-84; Hollinger et al., Cancer Immunol Immunother 1997 November-December 45 (3-4) 128-30; Immunotargeting of tumors: state of the art and prospects in 2000 Bull Cancer. 2000 November; 87(11): 777-91; Hellfrich W et al Int. J. cancer 1998 Apr. 13 76(2): 232-9; Wu A M, Q J Nuc Med. 2000 September; 44(3): 268-83 Krebs B. Et al. J Interferon cytokine Res 1998September 18(9): 783-91; Takemura Si, et al. Protein Eng. 2000 August; 13(8): 583-8; Cochlovius B et al. J. Immunol. 2000 Jul. 15; 165(2): 888-95; Atwell J L et al. Protein Eng. 1999 July; 12(7): 597-604; Kiprivanov S M et al. J. Mol. Biol. 1999 Oct. 15, 293 (1): 41-56; Alt M. et al FEBS Lett. Jul. 2 454 01-2) 90-4. Hudson P J et al. J Immunol Methods 1999 Dec. 10; 231(1-2): 177-89 Ardnt M A et al. Blood 1999 Oct. 15 94(8): 2562-8; Lu D. et al. J. Immunol. Methods 1999 Nov. 19; 230(1-2): 159-171; Santos A D et al, Clin Cancer Res 1999 Oct. 5 (10 suppl): 31185-31235 Kontermann R E et al. Nat Biotechnol. 1997 July; 15(7): 629-31; Doles et al. Protein eng. (2000) Aug. 13 (8): 565-74; Adams G P et al. Nucl. Med. Biol (2000) May 27 (4); 339-46; Williams L E et al. Med phys 2000 may 27(5) 988-94; Fitzgerald K. Protein Eng 1997 October 10(10): 1221-5 and the various references cited therein) as are various methods for identifying internalizing antibodies and creating toxin, radionuclide and cytokine fusions/conjugates (see also Y et al Bioconj. Chem. 1998 November-December; 9(6): 635-44) for fully exploiting various aspects of the invention herein defined (see for example Becerril B et al. Biochem Biophys Res Comm 1999 Feb. 16; 255(2): 386-93 see also additional references below.

Triabodies and other known multivalent antibodies etc. (see for example Iliades P et al. FEBS Lett. 1997 June 16; 409(3): 437-41) etc. could advantageously be employed to provide additional functionalities, as well as variation in avidity etc. for the purposes of variously exploiting the invention herein.

Methods of expressing and identifying new molecules like LYVE-1 are also well known in the art (see WO 98/06839)

Technologies for rendering the multifunctional ligands of the invention less immunogenic (e.g. such as employed by Biovation) are preferably applied to the multifunctional ligands of the invention.

For recent progress in the treatment of lupus nephritis, see Zimmerman R. Annu Rev. Med. 2001; 52:63-78.

With respect to targeting Fas-L see U.S. Pat. No. 6,068,841: Antibodies to Fas-L for treatment of hepatitis.

The invention also contemplates using chemokines and variously targeted antibodies and fragments thereof fused or conjugated to chemokines or other molecules with for example, lymphocyte or other immune cell attractant properties (see for example Sun J. et al. Lymphology 32 (1999) 166-170; and Gerard C. et al. Nature Immunology (2001, Feb.) 2(2): p 108; Immunological Reviews 1999 Vol 170 p 5-197) to attract immune cells into target tissues for eventual penetration into the lymphatic vessels for activation, signaling, binding to, inhibition, etc. For example, for cancer treatment antibodies that bind to angiogenesis markers fused to such type such molecules e.g. TNF-α can be advantageously employed optionally in conjunction with various vaccination strategies (including the use of the multi-functional ligands of the present invention) to attract immune cells including, optionally, vaccination-activated tumor targeting lymphocytes to the tumor site. In an indirectly related aspect (having independent applications as well as for combination therapy with a multifuctional ligand, the invention is also directed to an antibody that targets an angiogenesis marker fused/conjugated to a cytokine or antibody (i.e. a bispecific antibody) which binds to a cytokine, which cytokine augments adhesion of immune cells to blood vessels and method of using same (by administration to a subject), alone, in combination with multifunctional ligands of the invention or with other vaccination strategies to increase immune cell targeting to a solid tumor. In the case of a bispecific antibody it is contemplated that the cytokine binding portion has a relatively low functional affinity to the cytokine so as to compete unfavourably for its binding to its natural receptor.

With reference to modulating binding of leucocytes to endothelial adhesion molecules see for example U.S. Pat. No. 6,123,915 and the references therein cited.

It is well known to those in the art to make bispecific antibodies which are adapted to bind two different ligands on the same cell, for example so called antigen-forks as disclosed in U.S. Pat. No. 5,705,614 (see also Shi T et al. Murine bispecific antibody 1A10 directed to human transferrin receptor and a 42-kDa tumor-associated glycoprotein also Clin Immunol Immunopathol 1996 February; 78(2): 188-95; Amoroso A R et al., Binding characteristics and antitumor properties of 1A10 bispecific antibody recognizing gp40 and human transferrin receptor Cancer Res 1996 Jan. 1; 56(1):113-20; Ring D B et al., Antigen forks: bispecific reagents that inhibit cell growth by binding selected pairs of tumor antigens, Cancer Immunol Immunother 1994 July; 39(1):41-8; Lu D et al., Complete inhibition of vascular endothelial growth factor (VEGF) activities with a bifunctional diabody directed against both VEGF kinase receptors, fms-like tyrosine kinase receptor and kinase insert domain-containing receptor. Cancer Res 2001 Oct. 1; 61(19): 7002-8; Schmiedl A, Breitling F, Dubel S. Expression of a bispecific dsFv-dsFv′ antibody fragment in Escherichia coli. Protein Eng 2000 October; 13(10): 725-34 see also Park S S, et al., Generation and characterization of a novel tetravalent bispecific antibody that binds to hepatitis B virus surface antigens Mol Immunol 2000 December; 37(18): 1123-30; Kriangkum J et al., Bispecific and bifunctional single chain recombinant antibodies Biomol Eng 2001 September; 18(2): 31-40; U.S. Pat. Nos. 4,474,893, 5,989,830; WO 00/29431).

With respect to antibodies to autoantigens, ADEPT, use of anti-eotaxin antibodies, Delmmunization, antibody-cytokine fusions, ribosome display, xenomouse technology; cutting edge phage display techniques, construction of human antibody fragment based phage display libraries, selection of internalizing antibodies by phage-display, cancer targeting antibodies, antibody arrays, plantibodies, design of mutant IGSF domains of CD2, CD58 and TCR; oligopeptide e.g. paratope mimetics, diabodies, minibodies, triabodies, tetrabodies and related size/kinetics issues, caspase activatable pro-drugs, delivery of Bismuth-213 via scFv and diabodies, anti-angiogenesis marker strategies, immunoenzyme therapy of cancer (e.g. with RNases) pancarcinomic antigens like CEA (TAG″)-72; and related technologies see the papers and references in Proceedings of IBC's 11^{th} Annual International Conference on Antibody Engineering, State of the Art, Science, Technology and Applications Dec. 3-6 2000 La Jolla, Calif.

With respect to biology of the lymphatic system having practical application herein see Ikomi, F. Lymphology (1999) 32:90-102; Shield J W. Lymphology 1999 32: 118-122 and Lymphology 33 (2000) 144-147, as well as the references cited therein.

The invention also contemplates control of such migration by inhibition of receptors that mediate such migration (see for example Sun J. et al. Lymphology 32 (1999) 166-170) for controlled application of the multifunctional ligands of the invention.

With respect to recent developments with respect to target ligands and/or immunotherapy having application herein see also WO 01/12224, WO 01/14550, WO 01/11059, WO 01/10205, WO 01/00679, WO029445 WO 01/14885, WO/14564, WO 01/14558, WO 01/14224, WO 01/13945, WO 01/12840, WO 01/12781, WO 01/12674, WO 01/12670, WO 01/12224, WO 01/12646; WO 01/12223, WO 01/12218, WO 01/12217, WO 01/12216, WO 01/12154, WO 01/14557, WO 01/11059, WO 01/10912, WO 01/11040, WO 01/10888, WO 01/10460, WO 01/10205, WO 01/09611, WO 01/09328, WO 01/09186, WO 01/09192, WO 01/08635, WO 01/07481, WO 01/07082, WO 01/07084, WO 01/07081, WO 01/07484, WO 01/07466, Triggering Fc alpha-receptor I (CD89) recruits neutrophils as effector cells for CD20-directed antibody therapy. J. Immunol. 2000 Nov. 15; 165(10): 5954-61. CD47 engagement inhibits cytokine production and maturation of human dendritic cells. J. Immunol. 2000 Feb. 15; 164(4): 2193-9.

The invention also contemplates that a multifunctional ligand that recognizes an immune cell as a target in virtue of a particular cell marker and will be able to deliver a toxic payload to the cell, for example, in virtue of its second portion comprising such toxic component fused or conjugated thereto. The invention also contemplates attracting or supplying other immune cells or molecules to kill, or otherwise inactivate the target immune cell (e.g. lymphocytes e.g. by TH cell modulation or CD4 cell modulation or using antibodies including anti-idiotypic antibodies. The invention therefore contemplates that treatment of such immune cells can be accomplished by a combination of different mechanisms or drugs depending on the disease so as to reduce immunosuppression due to immune cell ablation where this is the dominant consideration. Such interactions may require interaction with one or more ligands on the surface of the targeted immune cell, as facilitated via anchoring interactions of varying affinity/avidity/duration. The invention also contemplates using multifunctional ligands comprising or bound to selectins and ICAMs etc. to facilitate such targeting, for example co-administering same in a proportion which is for example 0.01% to 25% of the targeting multifunctional ligand. The relative amounts of the selectin/ICAM etc. (including antibody mimics) bearing multifunctional ligand as compared with the targeting multifunctional ligand can be determined empirically by varying the proportions and assessing any objective indicator of successful targeting in a disease related or purely experimental context. For example successful targeting (e.g. antibody binding to e.g. CD3, CD28, CD2) using multifunctional ligands of the invention could be monitored by evaluating levels of cytokines normally attributable to such binding (see for example CD8 T cell activation after intravenous administration of CD3 x CD19 bispecific antibody in patients with non-Hodgkin lymphoma. Cancer Immunol Immunother. 1995 June; 40(6): 390-6. Definition of a lamina propria T cell responsive state. Enhanced cytokine responsiveness of T cells stimulated through the CD2 pathway. J. Immunol. 1995 Jan. 15; 154(2): 664-75.

With respect to multifunctional ligands that are used to directly or indirectly exert an immunization function, other examples of disease associated peptides that can be presented as immunogens or inhibitor/modulators of immune activity or disease progression in one of the fashions suggested above include, examples as well as technologies referenced in, for example, Knuth A, Cancer Chemother Pharmacol (2000); 46 suppl: 546-51; Engelhard V H, Cancer J Sci Am 2000 May; 6 Suppl 3: S272-80; Pietersz G A et al, Cell Mol Life Sci. 2000 February; 57(2): 290-310; Algarra I et al, Hum Immunol. 2000 January; 61(1): 65-73; Tumour vaccines: a new immunotherapeutic approach in oncology. Ann Hematol. 2000 December; 79(12): 651-9; Human tumor-rejection antigens and peptides from genes to clinical research Nippon Geka Gakkai Zasshi. 2000 September; 101(9): 612-7. Pinilla-Ibarz J, et al CML vaccines as a paradigm of the specific immunotherapy of cancer. Blood Rev. 2000 June; 14(2): 111-20).

In order to present an MHC-peptide complex in proximity to a B7 co-stimulatory molecule, the invention contemplates using, in addition to varying amounts (varying from a 50/50 proportion) of adjacent multifunctional ligands (which may be a dAb, diabody etc.) preferably cross-linked by an avidin component,—as a different strategy—cross-linking with avidin or the like adjacent arms of a single diabody, triabody or tetrabody etc. which binds to or has been fused or conjugated individually to respective B7 and MHC peptide components (with respect to recombinant B7 and MHC molecules and fusion proteins thereof including antibody fusions and related technologies see references above and EP 99/97477 WO 99/42597, WO 97 28191, U.S. Pat. No. 6,197,302, U.S. Pat. No. 6,015,884 U.S. Pat. No. 6,140,113, U.S. Pat. No. 6,045,796, U.S. Pat. No. 5,580,756, EP0935607, WO 9806749 WO9803552, EP 1054984, U.S. Pat. No. 5,869,270, Construction and characterization of bispecific costimulatory molecules containing a minimized CD86 (B7-2) domain and single-chain antibody fragments for tumor targeting; method is useful for cancer therapy Rohrbach F et al., Clin. Cancer Res.; (2000) 6, 11, 4314-22 WO 00/008057 17 Feb. 2000; WO 9921572 6 May 1999; WO 9913095 18 Mar. 1999; WO 9742329 13 Nov. 1997; WO 9720048 5 Jun. 1997; WO 9640915 19 Dec. 1996; WO 00/023087; EP 610046 10 Aug. 1994, U.S. Pat. No. 6,056,952 as well as references therein cited).

In a related aspect, the invention similarly contemplates using or more antibodies (optionally biotinylated and cross-linked by an avidin component) that bind to the same or different epitopes on a tumor including, where two such antibodies are used different proportions of MHC and B7 linked (i.e. fused, conjugated or capable of binding to) antibodies as well as different proportions of different epitope-specific antibodies to optimize the distribution of such cross-linked B7 and MHC peptide complexes for T-cell recognition. In this way, any strongly immunogenic peptide may be used in conjunction with suitable vaccination strategies to create a universal cancer antigen. Using a tumor unrelated peptide is advantageous to avoid any tolerization effects resulting from T-cell binding to the MHC-peptide alone and does not preclude immune system recognition of a different epitope or other therapies. In a preferred embodiment, a single multifunctional ligand or pair of multifunctional ligands optionally biotinylated and cross-linked by an avidin (or variants), is used to bind to both the lumen of the lymphatic system and to a tumor cell (using for example a trispecific antibody with monovalent linkage to both the cancer cell and lymphatic endothelial cell and a third antibody component having respective fusions to one of MHC-peptide and B7 on heavy and light chain, or a trispecific or tetraspecific tetrabody having an antibody component devoted to each or the B7 and MHC linkages). This permits a single molecule to be used for both the immunization within the lymphatic system and the tumor targeted antigen display. However, It will be appreciated that presentation of MHC-peptide complex on a tumor does not necessarily require costimulatory B7 presentation to induce a cytotoxic T cell response which is specific for the peptide and that multiple such presentations, preferably in a cross-linkable fashion may be preferable. Accordingly, strategies herein for costimulatory presentation of MHC-peptide and B7 may be differently applied to a lymphatic endothelial cell surface for immunization purposes and a tumor cell surface (primarily for recognition purposes), for example by using avidin facilitated cross-linking of in the former but not the latter (tumor) context or using different sets of molecules in each case or using modularly reconstructing the tumor cell surface with a bispecific antibody that binds to a separately administered MHC and/or B7 component.

Subject to the latter proviso, in preferred embodiments, the invention contemplates using as separate counterparts 1) separate trispecific Abs, each including for example, one antibody component which binds to the each of the respective B7 and MHC molecules which are preferably together, separately administered. Such multifunctional ligands are preferably biotinylated for cross-linking—both between adjacent trispecific Abs and adjacent T-cell stimulatory/co-stimulatory arms; or 2) separate bispecific pairs of Abs each respectively having 1) either a B7 and lymphatic vessel or B7 and tumor binding portion or 2) a MHC peptide complex and a lymphatic vessel or MHC complex and tumor binding, portions which again are preferably cross-linked by an avidin, streptavidin or a variant (i.e. using biotinylated antibodies) This latter embodiment permits smaller size antibody molecules to be used for better tumor targeting. Antibody components which recognize the non-T cell interactive portion of the B7 or MHC molecule can be readily generated by phage display, for example in the case of a known peptide specific antibody to an MHC peptide complex (see Chames et al. Proc Natl Acad Sci USA 97, 7969 and Chames et. al. “Affinity Maturation of TCR-Like MHC-peptide specific antibody: peptide specificity is possible over a wide affinity range” Proceedings of IBC Conference on Antibody Engineering December 2000) e.g. by first causing binding of the “peptide specific” antibody and then doing the phage display e.g. using an array of multiple (e.g. repeats of the same antibody) such peptide specific MHC antibodies, applying the MHC peptide complex to effect binding and then performing the phage or ribosome display. Alternatively, a TCR (e.g. cell bound) or analogue/mimitope could be used for the orientation. Similarly, antibodies could be generated which in effect do not compete with CD28 or a mimitope thereof to create suitable anti-B7 type antibodies. Anti-B7 antibodies are known in the art. The invention also contemplates that the MHC-peptide binding function may be supplied using a linked superantigen (U.S. Pat. No. 6,197,299, WO 9601650 25 Jan. 1996; Proc. Natl. Acad. Sci. U.S.A.; (1994) 91, 19, 8945-49) in both the tumor and lymphatic system binding sites. Optionally, the tumor antigen or one or both of the antigens are a pan-carcinomic antigen like TAG-72, CEA, H11 (WO 97/44461). The invention also contemplates using one or more phage display libraries to optimize the development of MHC/B7 costimulatory bispecific antibodies, by using cell sized latex spheres coated with an antigen e.g. CEA in various surface dispersions (or a cell) and using a array of preferably biotinylated antibodies which recognize the antigen and have a “oppositely located” portion fused, conjugated or capable of binding to one or both of MHC and B7, the library optionally also presenting also variations and combinations of lengths (truncations) of one or more constant regions or for example the CDR2 generated by phage display, depending on the choice of antibody, and with microarray technology, using a signaling means to detect T-cell recognition and evaluating cytotoxicity with for example a Cr51 release assay (with respect to protein chip or microarray technology see WO 00/63701 references, for example in the Proceedings of IBC's conference on Protein Microarray Technology March 19-21 Santiago Calif.

The invention also contemplates use of recently published antibodies in the context of the invention (see WO 01/19861, WO 01/19990, WO 01/19860, WO 01/19987, WO 01/19990, WO 99/58678, WO 00/59943, WO 01/18014, WO 01/18016, WO 01/18204, WO 01/18042, WO 01/18021, WO 01/18014, WO 01/18046, WO 01/16166, WO 01/15731, WO 01/15728, WO 01/16183, WO 01/16170, WO 01/15732.

The invention is also directed to a method of evaluating dosing, ligand saturation, avidity effects of simultaneous ligand binding on prolonged anchoring and associated benefits (e.g. to delay a cancer cell for targeted killing or facilitate transfer of the multifunctional ligand to the targeted cell), cooperative interactions, cross-linking interactions (see J Immunol 2001 Mar. 1; 166(5): 3256-3265; Nippon Rinsho. 1999 December; 57 Suppl: 428-32; Harefuah. 2000 Jun. 15; 138(12): 1046-50. Leuk Lymphoma. 1998 March; 29(1-2): 1-15 and costimulatory interactions by administering to a test subject two different multifunctional ligands of the invention with cooperating second portions.

With respect to the display of functional peptides on an antibody type scaffold see Nuttal S D; et al., Proteins (1999) 36: 217-227; see also Skerra A., J. Mol. Recognition. 2000 July-August 13(4): 167-187. The invention also contemplates bispecific multifunctional ligands in which the immune function exerting moiety exerts its function through binding to an immunogenic component or carrier for such component as discussed above, for example an Fc domain fused to a peptide, a heat shock protein (see for example Wang X Y, Immunol Invest 2000 May 29(2): 131-7 and references cited therein as well as U.S. Pat. No. 6,168,793; U.S. Pat. No. 6,071,956; U.S. Pat. No. 5,981,706; U.S. Pat. No. 5,948,646 Methods for preparation of vaccines against cancer comprising heat shock protein-peptide complexes; U.S. Pat. No. 5,830,464 Compositions and methods for the treatment and growth inhibition of cancer using heat shock/stress protein-peptide complexes in combination with adoptive immunotherapy as well as patents, scientific articles and patent applications referenced in these patents; with respect to MHC peptide complexes (see for example WO 99/64597, WO 98/03552, WO 98/06749 and references cited therein).

As described above, the invention also contemplates that the lower affinity ligand binding arm of the aforementioned multifunctional ligand (i.e. having a high affinity targeting arm and a lower affinity effector arm) is constituted by a high affinity ligand, for example an high affinity antibody or functional fragment thereof, which binds to a target biological effector (e.g. a cytokine, chemokine, growth factor, hormone or other biological response modifier or drug) with high affinity, in a manner which permits the effector to continue to bind to its desired target receptor while bound to the antibody (i.e. the antibody binds to a portion of the effector which is not critically involved in the effector binding to its receptor) provided that when bound to the effector the antibody or fragment thereof has, when combined with the effector, a suitably lower affinity for the receptor than the ligand binding arm which functions as the high affinity binder has for its target cell marker. In one embodiment, the binding moiety which binds to the biological effector binds to it with higher affinity than the affinity that the effector has for the effector receptor. The invention also contemplates that this binding arm can bind to biological effector in a manner which permits it to bind to one receptor but not a related receptor to which the effector would otherwise bind (see examples below). The invention also contemplates that antibody arrays are used to screen for antibodies which are capable of binding to such biological effectors, while bind in situ to their receptors. The invention also contemplates that such binders, when bound to the biological effector, can be used to test their ability to bind to related receptors, such as those within the same family e.g. within the same family of TNF like receptors. With respect to antibody microarrays, see for example Cahill D J. Protein and antibody arrays and their medical applications. J Immunol Methods. 2001 April; 250(1-2): 81-91. MacBeath G. Proteomics comes to the surface. Nat Biotechnol. 2001 September; 19(9): 828-9. Clewley J P. Recombinant protein arrays. Commun Dis Public Health. 2000 December; 3(4): 311-2; Holt L J, Enever C, de Wildt R M, Tomlinson I M. The use of recombinant antibodies in proteomics. Curr Opin Biotechnol. 2000 October; 11(5): 445-9. Walter G, et al. Protein arrays for gene expression and molecular interaction screening. Curr Opin Microbiol. 2000 June; 3(3): 298-302. de Wildt R M, Mundy C R, Gorick B D, Tomlinson I M. Antibody arrays for high-throughput screening of antibody-antigen interactions. Nat. Biotechnol. 2000 September; 18(9): 989-94. Holt L J, et al. Bypassing selection: direct screening for antibody-antigen interactions using protein arrays. Nucleic Acids Res. 2000 Aug. 1; 28(15): E72 and the references cited therein. The term receptor as used herein for greater certainty includes decoy receptors. Examples of decoy receptors include TRAIL decoy receptors (APO-2L), CD44 decoy like receptors (hyaluronan), interleukin receptor like protein (IL-17) (see J Biol Chem 2001 Nov. 12), CD95-Fc decoy receptor, TRAMP, IL-1 RII receptor, osteoprotegerin (OPG), IL13Ralpha2.

### Affinity Maturation

Techniques for affinity maturation using high throughput screening techniques to evaluate mutants are well known in the art. Femtomolar affinities have been achieved and it is quite common to obtain nanomolar to picomolar affinities as a result of an affinity maturation process. For example it well known to use techniques of parsimonious mutagenesis to engineer amino acid change at selected “hotspots”. With respect to affinity maturation, see for example Coia G, Hudson P J, Irving R A. Protein affinity maturation in vivo using E. coli mutator cells. J Immunol Methods. 2001 May 1; 251(1-2): 187-93. Manivel V, Sahoo N C, Salunke D M, Rao K V. Maturation of an antibody response is governed by modulations in flexibility of the antigen-combining site. Immunity. 2000 November; 13(5): 611-20. Boder E T, Midelfort K S, Wittrup K D. Directed evolution of antibody fragments with monovalent femtomolar antigen-binding affinity. Proc Natl Acad Sci USA. 2000 Sep. 26; 97(20): 10701-5. Holler P D, Holman P O, Shusta E V, O'Herrin S, Wittrup K D, Kranz D M. In vitro evolution of a T cell receptor with high affinity for peptide/MHC. Proc Natl Acad Sci USA. 2000 May 9; 97(10): 5387-92. Daugherty P S, Chen G, Iverson B L, Georgiou G. Quantitative analysis of the effect of the mutation frequency on the affinity maturation of single chain Fv antibodies. Proc Natl Acad Sci USA. 2000 Feb. 29; 97(5): 2029-34. Van Antwerp J J, Wittrup K D. Fine affinity discrimination by yeast surface display and flow cytometry. Biotechnol Prog. 2000 January-February; 16(1): 31-7. Adams G P, Schier R. Generating improved single-chain Fv molecules for tumor targeting. J Immunol Methods. 1999 Dec. 10; 231(1-2): 249-60. Daugherty P S, Chen G, Olsen M J, Iverson B L, Georgiou G. Antibody affinity maturation using bacterial surface display. Protein Eng. 1998 September; 11(9): 825-32. Wong Y W, Kussie. P H, Parhami-Seren B, Margolies M N. Modulation of antibody affinity by an engineered amino acid substitution. J. Immunol. 1995 Apr. 1; 154(7): 3351-8. Balint R F, Larrick J W. Antibody engineering by parsimonious mutagenesis. Gene. 993 December 27; 137(1): 109-18. Schillbach J F, Near R I, Bruccoleri R E, Haber E, Jeffrey P D, Novotny J, Sheriff S, Margolies M N. Modulation of antibody affinity by a non-contact residue. Protein Sci. 1993 February; 2(2): 206-14. Chames P, Baty D. Engineering of an anti-steroid antibody: amino acid substitutions change antibody fine specificity from cortisol to estradiol. Clin Chem Lab Med. 1998 June; 36(6): 355-9. Kussie. PH, Parhami-Seren B, Wysocki L J, Margolies M N. A single engineered amino acid substitution changes antibody fine specificity. J. Immunol. 1994 Jan. 1; 152(1): 146-52, as well as references cited therein. With respect to generation of high affinity antibodies and affinity maturation of antibodies, see also Hanes J. Nat. Biotechnol. 2000 December; 18(12): 1287-92; references in Hudson P J Exp. Opin. Invest. Drugs (2000) 9(6) 1231-1242; Toran J L et al Evr. J. Immunol. 2001 January; 31(1) 128-137. Nielson V B et al. Cancer Res 2000 Nov. 15; 60 (22) 6434-40 Adams G P, Journal of Immunological Methods (1999) 249-260; Chowdhury P S et al (June 1999) Nature Biotechnology Vol 17 p. 568. With respect to strategies and recent technologies which have application to the invention see references in Hudson P J Exp. Opin. Invest. Drugs (2000) 9(6) 1231-1242 and in particular references relating to strategies to achieve multivalency and multispecificity; recruitment of viruses, ADEPT, photoactivation of cytotoxic radionuclides; surface receptor cross-linking; (see also Eur. J. Immunol. 2000; 30(10) 3006), use of anti-B antibodies; immunocytokines (see also Lode H N Immunol. Res. 2000, 21 (2-3) 279-88; Gillies S D Cancer Research 59 2159-2166 May 1999; Lode H N et al Drugs of Today 2000 36(5) 3221-336).

With respect to practical size limitations and pharmacokinetics of various types of antibodies and fragments, see Colcher D. et al. G. J. Nucl. Med. (1999) 43: 132-139; Wu A M et al G. J. Nucl. Med. 2000 September; 44(3): 268:83; Williams L E et al Med Phys 2000 May 27(5): 988-941 Ikomi F. Lymphology 32 (1999) 90-102.

With respect to the construction of diabodies see also Takemura S I et al. Protein Eng. 2000 August; 13(8) 583-8; Biomol. Eng. 2001 September; 18(2): 31-40.

With respect to anti-cancer antibodies, see also U.S. Pat. No. 6,180,357.

With respect to technologies to produce multivalent and/or multispecific antibodies see also U.S. Pat. No. 6,172,197; WO 92/01047; WO 93/11161; WO 94/07921; WO 94/13804; Helfrich W. et al. Journal of Immunological Methods 237 (2000) 131-145. Proceedings of 11^{th} IBC Conference on Antibody Engineering; WO 01/85795

Monoclonal antibodies may be routinely produced as taught by Harlow, E. and D. Lane, (1988) ANTIBODIES: A Laboratory Manual, Cold Spring Harbor Laboratory, Cold Spring Harbor N.Y. Humanized antibodies may be routinely produced as taught, for example, by U.S. Pat. No. 5,585,089 and U.S. Pat. No. 5,530,101. Techniques for engineering antibodies are well known and described in Winter and Millstein (1991) Nature 349:293, and Larrich and Fry (1991) Hum. Antibod. and Hybridomas 2:17. One having ordinary skill in the art may use well-known techniques and starting materials and/or commercially available expression vectors and systems that are readily available and known in the art. See e.g., Sambrook et al., Molecular Cloning a Laboratory Manual, Second Ed. Cold Spring Harbor Press (1989).

Examples of radionuclides useful as toxins in radiation therapy are well known. Some examples are referred to below. Auger emitters may be preferred for internalizing antibodies. As suggested above, the term antibody is used interchangeably with antibody fragment and antigen binding fragment and includes a whole antibody; antibody fragment a portion of an antibody such as a scFV F(ab′)₂ F(ab)₂. Fab′, Fab, dAb, microbodies (WO0029004) or the like or multivalent such fragments, including those itemized or referenced herein. Regardless of structure, an antibody fragment can be made to bind with the same antigen that is recognized by the intact antibody. More particularly, in addition to fragments formed by enzymatic digestion of an intact Ab the term antibody or “antibody fragment” unless otherwise stated also includes any synthetic or genetically engineered protein that acts like an antibody by binding to a specific antigen to form a complex including/as applicable, cysteine noose peptides and minimal recognition units consisting of the amino acid residues that mimic the hypervariable region. Although fully human antibodies, for example, antibodies generated via human-human hybridomas or through phage display using human antibody based libraries, are preferred, the invention does not preclude other strategies to avoid a HAMA type response.

A chimeric antibody is a recombinant protein that contains the variable domains and complementary determining regions derived from, for example, a rodent antibody, while the remainder of the antibody molecule is derived from a human antibody.

With respect to stability engineering of scFv fragments for enhanced multifunctional ligands comprising scFvs see J Mol Biol 2001 Feb. 2; 305(5): 989-1010.

Humanized antibodies are recombinant proteins in which murine LDR's of a monoclonal antibody have been transferred from heavy and light variable chains of the murine immunoglobulin into a human variable domain.

The term therapeutic agent as used herein, is a molecule or atom which is conjugated etc. to an antibody moiety to produce combination including a conjugate which is useful for therapy. Examples of therapeutic agents include drugs, toxins, immunomodulators, chelators, boron compounds, photoactive agents or dyes, and radioisotopes.

The term “a naked antibody” may be used to refer specifically to an entire antibody, as opposed to an antibody fragment, which is not conjugated with a therapeutic agent. Naked antibodies include both polyclonal and monoclonal antibodies, as well as certain recombinant antibodies, such as chimeric and humanized antibodies.

The term immunoconjugate may be used to refer a conjugate of an antibody component with a therapeutic agent.

As used herein, the term antibody fusion protein refers to a recombinant molecule that comprises an antibody component and a second functional component for example a therapeutic agent. Examples of therapeutic agents suitable for such fusion proteins include immunomodulators (“antibody-immunomodulator fusion protein”) and toxins (“antibody-toxin fusion protein”).

Production of Antigen—Specific Monoclonal Antibodies, Rodent monoclonal antibodies to antigen can be obtained by methods known to those skilled in the art. See generally, for example, Kohler and Milstein, Nature 256:495 (1975), and Coligan et al. (eds.), Current Protocols in Immunology, Vol. 1, pages 2.5.1-2.6.7 (John Wiley & Sons 1991) [“Coligan”]. Briefly, monoclonal antibodies can be obtained by injecting mice with a composition comprising the antigen in a question (Ag), verifying the presence of antibody production by removing a serum sample, removing the spleen to obtain B-lymphocytes, fusing the B-lymphocytes with myeloma cells to produce hybridomas, cloning the hybridomas, selecting positive clones which produce anti-Ag antibodies, culturing the clones that produce antibodies to the antigen, and isolating the antibodies from the hybridoma cultures. Transgenic mice having for example engineered immune systems to create human antibodies such those used by Medarex and Abgenix are also contemplated for use herein to create suitably targeted antibodies.

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, for example, Coligan at pages 2.7.1-2.7.12 and pages 2.9.1-2.9.3. Also, see Baines et al., “Purification of Immunoglobulin G (IgG),” in Methods in Molecular Biology, Vol. 10, pages 79-104 (The Humana Press, Inc. 1992).

With respect to relevant molecular biology techniques See also, for example, Ausubel et al., (eds.), CURRENT PROTOCOLS IN MOLECULAR BIOLOGY, pages 8.2.8 to 8.2.13 (1990) [“Ausubel”]. Also, see Wosnick et al., Gene 60:115 (1987); and Ausubel et al. (eds.), Short Protocols in Molecular Biology, 3rd Edition, pages 8-8 to 8-9 (John Wiley & Sons, Inc. 1995). Established techniques using the polymerase chain reaction provide the ability to synthesize genes as large as 1.8 kilobases in length. Adang et al., Plant Molec. Biol. 21:1131 (1993) Bambot et al., PCR Methods and Applications 2:266 (1993); Dillon et al., “Use of the Polymerase Chain Reaction for the Rapid Construction of Synthetic Genes,” in Methods in Molecular Biology, Vol. 15: PCR Protocols: Current Methods and Applications, White (ed.), pages 263-268, (Humana Press, Inc. 1993).

Techniques for constructing chimeric antibodies are well known to those of skill in the art. As an example, Leung et al., Hybridoma 13:469 (1994).

In yet another embodiment, an antibody of the present invention is a “humanized” monoclonal antibody. That is, mouse complementarity determining regions are transferred from heavy and light variable chains of the mouse immunoglobulin into a human variable domain, followed by the replacement of some human residues in the framework regions of their murine counterparts. Humanized monoclonal antibodies in accordance with this invention are suitable for use in therapeutic methods. General techniques for cloning murine immunoglobulin variable domains are described, for example, by the publication of Orlandi et al., Proc. Nat'l Acad. Sci. USA 86: 3833 (1989). Techniques for producing humanized monoclonal antibodies are described, for example, by Jones et al., Nature 321:522 (1986), Riechmann et al., Nature 332:323 (1988), Verhoeyen et al., Science 239:1534 (1988), Carter et al., Proc. Nat'l Acad. Sci. USA 89:4285 (1992), Sandhu, Crit. Rev. Biotech. 12:437 (1992), and Singer et al., J. Immunol. 150:2844 (1993). The publication of Leung et al., Mol. Immunol. 32:1413 (1995) describes the construction of humanized LL2 antibody.

In a preferred embodiment of the invention, the multifunctional ligand has a unique portion which differentiates it from other antibodies and preferably other co-administered different multifunctional ligands, which unique portion, allows the multifunctional ligand to be efficiently segregated on an immunoaffinity column. In the case of differentiating a single multifunctional ligand, an anti-idiotype (assuming the first portion consists of an antibody) or other antibody uniquely recognizing the first portion could be employed. Modifying a portion of the first portion, for example in the case where it is antibody component and creating a antibody thereto, for example by phage display, is a matter of routine skill in the arts of antibody engineering and phage display.

In another embodiment, an antibody of the present invention is a human monoclonal antibody. Such antibodies are obtained from transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994).

### Examples of Production of Antibody Fragments

Antibody fragments can be prepared, for example, by proteolytic hydrolysis of an antibody or by expression in E. coli of the DNA coding for the fragment.

Antibody fragments can be obtained by pepsin or papain digestion of whole antibodies by conventional methods. For example, antibody fragments can be produced by enzymatic cleavage of antibodies with pepsin to provide a 5 S fragment denoted F(ab′)₂. This fragment can be further cleaved using a thiol reducing agent, and optionally a blocking group for the sulfhydryl groups resulting from cleavage of disulfide linkages, to produce 3.5 S Fab′ monovalent fragments. Alternatively, an enzymatic cleavage using pepsin produces two monovalent Fab fragments and an Fc fragment directly. These methods are described, for example, by Goldenberg, U.S. Pat. Nos. 4,036,945 and 4,331,647 and references contained therein. Also, see Nisonoff et al., Arch Biochem. Biophys. 89:230 (1960); Porter, Biochem. J. 73:119 (1959), Edelman et al., in Methods in Enzymology Vol 1, page 422 (Academic Press 1967), and Coligan at pages 2.8.1-2.8.10 and 2.10-2.10.4.

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

For example, Fv fragments comprise an association of V_{H} and V_{L} chains. This association can be noncovalent, as described in Inbar et al., Proc. Nat'l Acad. Sci. USA 69:2659 (1972). Alternatively, the variable chains can be linked by an intermolecular disulfide bond or cross-linked by chemicals such as glutaraldehyde. See, for example, Sandhu, supra.

Preferably, the Fv fragments comprise V_{H} and V_{L} chains which are connected by a peptide linker. These single-chain antigen binding proteins (scFv) are prepared by constructing a structural gene comprising DNA sequences encoding the V_{H} and V_{L} domains which are connected by an oligonucleotide. The structural gene is inserted into an expression vector which is subsequently introduced into a host cell, such as E. coli. The recombinant host cells synthesize a single polypeptide chain with a linker peptide bridging the two V domains. Methods for producing scFvs are described, for example, by Whitlow et al., Methods: A Companion to Methods in Enzymology 2:97 (1991). Also, see Bird et al., Science 242:423 (1988), Ladner et al., U.S. Pat. No. 4,946,778, Pack et al., Bio/Technology 11:1271 (1993), and Sandhu, supra.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2:106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

### Preparation of Immunoconjugates

The present invention contemplates immunoconjugates to assess and effect treatment of various disease conditions. Such immunoconjugates can be prepared by indirectly conjugating a therapeutic agent to an antibody component. For example, general techniques are described in Shih et al., Int. J. Cancer 41:832-839 (1988); Shih et al., Int. J. Cancer 46:1101-1106 (1990); and Shih et al., U.S. Pat. No. 5,057,313. The general method involves reacting an antibody component having an oxidized carbohydrate portion with a carrier polymer that has at least one free amine function and that is loaded with a plurality of drug, toxin, chelator, boron addends, or other therapeutic agent. This reaction results in an initial Schiff base (imine) linkage, which can be stabilized by reduction to a secondary amine to form the final conjugate.

The carrier polymer is preferably an aminodextran or polypeptide of at least 50 amino acid residues, although other substantially equivalent polymer carriers can also be used. Preferably, the final immunoconjugate is soluble in an aqueous solution, such as mammalian serum, for ease of administration and effective targeting for use in therapy. Thus, solubilizing functions on the carrier polymer will enhance the serum solubility of the final immunoconjugate. In particular, an aminodextran will be preferred.

The process for preparing an immunoconjugate with an aminodextran carrier typically begins with a dextran polymer, advantageously a dextran of average molecular weight of about 10,000-100,000. The dextran is reacted with an oxidizing agent to effect a controlled oxidation of a portion of its carbohydrate rings to generate aldehyde groups. The oxidation is conveniently effected with glycolytic chemical reagents such as NalO₄, according to conventional procedures.

The oxidized dextran is then reacted with a polyamine, preferably a diamine, and more preferably, a mono- or polyhydroxy diamine. Suitable amines include ethylene diamine, propylene diamine, or other like polymethylene diamines, diethylene triamine or like polyamines, 1,3-diamino-2-hydroxypropane, or other like hydroxylated diamines or polyamines, and the like. An excess of the amine relative to the aldehyde groups of the dextran is used to insure substantially complete conversion of the aldehyde functions to Schiff base groups.

A reducing agent, such as NaBH₄, NaBH₃ CN or the like, is used to effect reductive stabilization of the resultant Schiff base intermediate. The resultant adduct can be purified by passage through a conventional sizing column to remove cross-linked dextrans.

Other conventional methods of derivatizing a dextran to introduce amine functions can also be used, e.g., reaction with cyanogen bromide, followed by reaction with a diamine.

The aminodextran is then reacted with a derivative of the particular drug, toxin, chelator, immunomodulator, boron addend, or other therapeutic agent to be loaded, in an activated form, preferably, a carboxyl-activated derivative, prepared by conventional means, e.g., using dicyclohexylcarbodiimide (DCC) or a water soluble variant thereof, to form an intermediate adduct.

Alternatively, polypeptide toxins such as pokeweed antiviral protein or ricin A-chain, and the like, can be coupled to aminodextran by glutaraldehyde condensation or by reaction of activated carboxyl groups on the protein with amines on the aminodextran.

Chelators for radiometals or magnetic resonance enhancers are well-known in the art. Typical are derivatives of ethylenediaminetetraacetic acid (EDTA) and diethylenetriaminepentaacetic acid (DTPA). These chelators typically have groups on the side chain by which the chelator can be attached to a carrier. Such groups include, e.g., benzylisothiocyanate, by which the DTPA or EDTA can be coupled to the amine group of a carrier. Alternatively, carboxyl groups or amine groups on a chelator can be coupled to a carrier by activation or prior derivatization and then coupling, all by well-known means.

Boron addends, such as carboranes, can be attached to antibody components by conventional methods. For example, carboranes can be prepared with carboxyl functions on pendant side chains, as is well known in the art. Attachment of such carboranes to a carrier, e.g., aminodextran, can be achieved by activation of the carboxyl groups of the carboranes and condensation with amines on the carrier to produce an intermediate conjugate. Such intermediate conjugates are then attached to antibody components to produce therapeutically useful immunoconjugates, as described below.

A polypeptide carrier can be used instead of aminodextran, but the polypeptide carrier must have at least 50 amino acid residues in the chain, preferably 100-5000 amino acid residues. At least some of the amino acids should be lysine residues or glutamate or aspartate residues. The pendant amines of lysine residues and pendant carboxylates of glutamine and aspartate are convenient for attaching a drug, toxin, immunomodulator, chelator, boron addend or other therapeutic agent. Examples of suitable polypeptide carriers include polylysine, polyglutamic acid, polyaspartic acid, copolymers thereof, and mixed polymers of these amino acids and others, e.g., serines, to confer desirable solubility properties on the resultant loaded carrier and immunoconjugate.

Conjugation of the intermediate conjugate with the antibody component is effected by oxidizing the carbohydrate portion of the antibody component and reacting the resulting aldehyde (and ketone) carbonyls with amine groups remaining on the carrier after loading with a drug, toxin, chelator, immunomodulator, boron addend, or other therapeutic agent. Alternatively, an intermediate conjugate can be attached to an oxidized antibody component via amine groups that have been introduced in the intermediate conjugate after loading with the therapeutic agent. Oxidation is conveniently effected either chemically, e.g., with NalO₄ or other glycolytic reagent, or enzymatically, e.g., with neuraminidase and galactose oxidase. In the case of an aminodextran carrier, not all of the amines of the aminodextran are typically used for loading a therapeutic agent. The remaining amines of aminodextran condense with the oxidized antibody component to form Schiff base adducts, which are then reductively stabilized, normally with a borohydride reducing agent.

Analogous procedures are used to produce other immunoconjugates according to the invention. Loaded polypeptide carriers preferably have free lysine residues remaining for condensation with the oxidized carbohydrate portion of an antibody component. Carboxyls on the polypeptide carrier can, if necessary, be converted to amines by, e.g., activation with DCC and reaction with an excess of a diamine.

The final immunoconjugate is purified using conventional techniques, such as sizing chromatography on Sephacryl S-300.

Alternatively, immunoconjugates can be prepared by directly conjugating an antibody component with a therapeutic agent. The general procedure is analogous to the indirect method of conjugation except that a therapeutic agent is directly attached to an oxidized antibody component.

For application to linking MHC I/II peptide/B7 molecules to a latex which has previously conjugated to biotin, for avidin assisted linking to a multifunctional ligand, it will be appreciated that biotin can be conjugated to a part of a latex sphere which is then linked to MHC peptide and B7 molecules by placing the spheres in a confluent layer or in the spheres in a microwells such that only part of the sphere is exposed for conjugation and then coating the spheres onto avidin coated plates for the B7 and MHC linkage.

It will be appreciated that other therapeutic agents can be substituted for the chelators described herein. Those of skill in the art will be able to devise conjugation schemes without undue experimentation.

As a further illustration, a therapeutic agent can be attached at the hinge region of a reduced antibody component via disulfide bond formation. For example, the tetanus toxoid peptides can be constructed with a single cysteine residue that is used to attach the peptide to an antibody component. As an alternative, such peptides can be attached to the antibody component using a heterobifunctional cross-linker, such as N-succinyl 3-(2-pyridyldithio)propionate (SPDP). Yu et al., Int. J. Cancer 56:244 (1994). General techniques for such conjugation are well known in the art. See, for example, Wong, CHEMISTRY OF PROTEIN CONJUGATION AND CROSS-LINKING (CRC Press 1991); Upeslacis et al., “Modification of Antibodies by Chemical Methods,” in MONOCLONAL ANTIBODIES: PRINCIPLES AND APPLICATIONS, Birch et al. (eds.), pages 187-230 (Wiley-Liss, Inc. 1995); Price, “Production and Characterization of Synthetic Peptide-Derived Antibodies,” in MONOCLONAL ANTIBODIES: PRODUCTION, ENGINEERING AND CLINICAL APPLICATION, Ritter et al. (eds.), pages 60-84 (Cambridge University Press 1995).

As described above, carbohydrate moieties in the Fc region of an antibody can be used to conjugate a therapeutic agent. However, the Fc region is absent if an antibody fragment is used as the antibody component of the immunoconjugate. Nevertheless, it is possible to introduce a carbohydrate moiety into the light chain variable region of an antibody or antibody fragment. See, for example, Leung et al., J. Immunol. 154:5919 (1995); Hansen et al., U.S. Pat. No. 5,443,953 (1995). The engineered carbohydrate moiety is then used to attach a therapeutic agent.

In addition, those of skill in the art will recognize numerous possible variations of the conjugation methods. For example, the carbohydrate moiety can be used to attach polyethylene glycol in order to extend the half-life of an intact antibody, or antigen-binding fragment thereof, in blood, lymph, or other extracellular fluids. Moreover, it is possible to construct a “divalent immunoconjugate” by attaching therapeutic agents to a carbohydrate moiety and to a free sulfhydryl group. Such a free sulfhydryl group may be located in the hinge region of the antibody component.

Methods for determining the binding specificity of an antibody are well known to those of skill in the art. General methods are provided, for example, by Mole, “Epitope Mapping,” in METHODS IN MOLECULAR BIOLOGY, VOLUME 10: IMMUNOCHEMICAL PROTOCOLS, Manson (ed.), pages 105-116 (The Humana Press, Inc. 1992). More specifically, competitive blocking assays for example to determine CD22 epitope specificity are described by Stein et al., Cancer Immunol. Immunother. 37:293 (1993), and by Tedder et al., U.S. Pat. No. 5,484,892 (1996).

In another aspect the invention is directed to a bispecific ligand, preferably a bispecific antibody, comprising at least a first ligand, preferably an antibody component, which binds specifically to a first cell surface associated ligand and at least a second ligand, preferably a second antibody component which binds specifically to a second cell surface associated ligand on the same cell, and wherein the functional affinity of at least one and preferably both of said antibody components is selected so as to substantially limit functional binding unless both of said first and second antibody components are substantially contemporaneously bound to said cell. It is known to provide bifunctional ligands wherein functional binding, for example, to accomplish signal transduction, is predicated on both ligands being bound or cross-linking. However, this effect is not contemplated to be predicated on differentially controlling the functional affinity of the respective ligands. According to a broad aspect of this invention (in which inclusion of a ligand which binds to a lymphatic vessel associated marker is optional), the invention excludes known such bispecific ligands which inherently have a suitable differential functional affinity. Such bispecific ligands are mentioned herein. By controlling the affinity of at least one of said ligands, for example where the functional affinity of one said ligands is substantially less than that of the other ligand the invention contemplates that a substantially greater percentage of the administered dose of the bispecific ligand will affect cells in which only both ligands are present, and/or that a reduced percentage of the dose administered will functionally bind to the cells in virtue only of the reduced functional affinity ligand. The invention also contemplates that functional affinity of one ligand is greatly increased to establish the functional affinity differential and that the functional affinity of both ligands is reduced relative to that of a standard, for example relative to that of a comparable ligands in hand or known in the art or identified by phage display, ribosome display or other comparable techniques using a single such ligand. The invention also contemplates that a microarray (or library) of bispecific ligands in which for example, the bispecific ligand is “tethered” (i.e. immobilized) directly or indirectly in virtue of one or more amino acid residues which are positioned within the molecule to preferably minimally interfere with any binding, and in which the signal (e.g. its intensity) associated with a single ligand binding interaction can be differentiated from a two or more ligand interactions, for example cell surface binding (alternatively the ligands or cell may be immobilized) and that ribosome and phage display could be adapted to bispecific single domain antibodies constituting a single chain (see references herein) by elongating the end of the chain from which the molecule is tethered. The invention contemplates that the affinity of one such ligand may be fixed and that the variability in members of the library lies in the permutations of certain key residues to which binding is attributable which can readily be identified by persons skilled in the art. The invention also contemplates assessing single ligand binding capability of successful bi-ligand binders for example by blocking the other (non-assessed at that time) ligand (e.g. with correlative ligand or a mimitope thereof) and for example determining limited or non-existent such binding to as well as using inclined ligand testing surfaces for washing over the correlative ligand, for example of defined surface area, including preferably defined lengths and widths and concentrations/distributions/amounts of the bound ligand, where the degree of incline is selected to roughly simulate the micro-environment of the comparable in vivo target, be it a stationary cell with a roughly defined average shear force of bathing fluids e.g. within a tumor or in the lymphatic system, or a mobile cell within a vein, artery, or lymphatic vessel, including those of different sizes. The invention is also directed to a method of generating a target ligand or improving the target specificity of any ligand by using a population of variants of that ligand within a micro-environment simulated microarray system in which the at least one of the following factors is simulated: concentration or amount or distribution of correlative ligand, shear force and shape using length and width parameters to simulates intraluminal diameter and length. The invention also contemplates in the case of a multifunctional ligand or in the case of a bispecific or multispecific ligand (as herein described) that the affinity of its component binding ligands may be selected for venous or arterial targeting or to accommodate lymphatic system targeting or targeting within or through tissues or combinations of the aforementioned e.g. median, average or weighted compromises to improve desired targeting. In a preferred embodiment the first ligand is selected on the basis of its ability to at least partially discriminate between a target population of cells (e.g. a ligand that is “associated” with a target population of cells) and a non-target population of cells (in one embodiment it is selected so as to have no other effect other than binding for targeting purposes) and the second ligand is selected for its ability to modulate the activity of the targeted cell, optionally in virtue of binding alone e.g. without delivering a payload (the term modulate referring broadly to any desired effect on the cell or its functionality) In this case the functional affinity for the ligand which is targeted for modulating the activity of the cell is selected so as to reduce the likelihood of binding unless binding has first or contemporaneously occurred to the first ligand targeted for selectivity (e.g. the second ligand would have monovalent as opposed to divalent binding to the ligand required for selectivity and/or from 0.20 to 10⁻⁹ fold reduction in affinity (for example as measured by Biacore) relative to the binding affinity for the first ligand, preferably a greater than 20000000% reduction in affinity preferably a greater than 3000000% reduction in affinity, preferably a greater than 40,000,000% reduction in affinity, preferably a greater than 50000000% reduction in affinity, preferably a greater than 60000000% reduction in affinity, preferably a greater than 70000000% reduction in affinity, preferably a greater than 80000000% reduction in affinity, preferably a greater than 90000000% reduction in affinity preferably a greater than 100,000,000% reduction in affinity, preferably a reduction in affinity of between one and two orders of magnitude, preferably a reduction in affinity of between two and three orders of magnitude, preferably a reduction in affinity of between three and four orders of magnitude, preferably a reduction in affinity of between four and five orders of magnitude, preferably a reduction in affinity of between five and six orders of magnitude, preferably a reduction in affinity of between six and seven orders of magnitude preferably a reduction in affinity of between seven and eight orders of magnitude, preferably a reduction in affinity of between eight and nine orders of magnitude, preferably a reduction in affinity of between nine and ten orders of magnitude.

It will be appreciated that a suitable reduction in affinity, if any, will depend on the valency of the respective first and second ligands and the selected affinity of the first ligand, which for example may have been augmented. The invention also contemplates a trispecific (and trivalent) ligand in which two ligands differently define its specificity to reduce the likelihood of an undesired effect attributable to the function exerting moiety binding alone. In terms of the physical constitution of a ligand having a trispecific binding capability, the invention also contemplates linking three monovalent dabs, MRUs or the like or mixed combinations thereof or two bivalent dabs, MRUs or the like or mixed combinations thereof (see WO 99/42077, U.S. Pat. No. 6,174,691, WO0029004, Camel single-domain antibodies as modular building units in J Biol Chem. 2000 Oct. 25, & Mulligan-Kehoe U.S. patents including U.S. Pat. No. 5,702,892, U.S. Pat. No. 5,824,520; see also U.S. Pat. No. 6,040,136) (in the latter case optionally one or both may be bispecific and linked by well known methods in the art (see WO 99/42077, Celltech's TFM, leucine zippers, U.S. Pat. No. 5,910,573, U.S. Pat. No. 5,892,020, EP 0654085B, see also EP 0318554B). The term functional binding is used to refer to binding which yields the desired effect, for example a therapeutic effect on a target cell population attributable to the binding to one or both ligands. Using the previous example, one ligand, e.g. the first ligand, may be used to target activated immune cells, and the second ligand may be different and may upon being bound to, for example result in inactivation, anergy, apoptosis or reduced capacity for endothelial adhesion of the immune cell. In this case, the invention contemplates that the functional affinity of the antibody component which binds to the second ligand is selected such that binding is unlikely to occur without binding to the specificity dictating ligand, for example the ratio of targeted relative non-targeted cells affected by the dose administered is approximately 1.10 to 1, preferably approximately 1.15 to 1, more preferably approximately 1.20 to 1, more preferably approximately 1.25 to 1, more preferably approximately 1.30 to 1, more preferably approximately 1.35 to 1, more preferably approximately 1.40 to 1, more preferably approximately 1.45 to 1, more preferably approximately 1.50 to 1, more preferably approximately 1.55 to 1, more preferably approximately 1.60 to 1, more preferably approximately 1.60 to 1, more preferably approximately 1.65 to 1, more preferably approximately 1.70 to 1, more preferably approximately 1.75 to 1, more preferably approximately 1.80 to 1, more preferably approximately 1.85 to 1, more preferably approximately 1.90 to 1, more preferably approximately 1.95 to 1, more preferably approximately 2 to 1, more preferably greater than 2 to 1, more preferably approximately greater than 3 to 1, more preferably approximately greater than 4 to 1, more preferably greater than 5 to 1, more preferably greater than 6 to 1, more preferably greater than 7 to 1, more preferably greater than 8 to 1, more preferably greater than 9 to 1, more preferably greater than 10 to 1, more preferably greater than 20 to 1, more preferably greater than 30 to 1, more preferably greater than 40 to 1, more preferably greater than 50 to 1, more preferably greater than 60 to 1, more preferably greater than 70 to 1, more preferably greater than 80 to 1, more preferably greater than 90 to 1, more preferably greater than 100 to 1, more preferably greater than 500 to 1, more preferably greater than 1000 to 1, more preferably greater than 10,000 to 1, more preferably greater than 100,000 to 1, more preferably greater than 500,000 to 1 more preferably greater than 1,000,000 to 1.

It will be appreciated by persons skilled in the art that the foregoing aspects of the invention apply to a variety of different combinations of immune function or other therapeutic function exerting ligands and specificity dictating ligands including those involved in immune signaling, stimulatory, co-stimulatory, inhibitory, adhesion or other interactions, including without limitation, cytokine receptors, ligands associated with immune cell adhesion, ligands to which binding results in stimulation, activation, apoptosis, anergy or costimulation, or ligands which differentiate between different populations or subpopulations or immune cells (see e.g. U.S. Pat. No. 6,135,941, WO 00/63251, WO 00/61132, U.S. Pat. No. 6,120,767), including sub-populations of B cells and T cells (see for example U.S. Pat. No. 6,197,524) activated versus non-activated lymphocytes, diseased or disease-causing cells versus non-diseased/disease causing lymphocytes (see for example WO 01/13945A1, U.S. Pat. No. 6,132,980) and specific immune cell clones for example those having specific Ig type and MHC-peptide type ligands/and correlative ligands. Examples of such ligands include CCR5, CTLA-4, LFA-1, LFA-3. ICAMs e.g. ICAM-1, CD2, CD3, CD4 (e.g. see U.S. Pat. No. 6,136,310), CD18, CD22, CD40, CD44; CD80, CD86, CD134 and CD154, to name only a few (see also U.S. Pat. No. 6,087,475: PF4A receptor) (see also Glennie M J et al. Clinical Trial of Antibody Therapy. Immunology Today August 2000, Vol. 21 (no. 8) p. 403).

The invention also contemplates that the therapeutic function or immune function effecting ligand is also a specificity imparting ligand, which in the case of for example, an antigen presenting cell may be an antibody which recognizes and binds to a specific MHC peptide complex, as is established in the art (see pertinent Chames et al. references herein, see also WO 97/02342, Direct selection of a human antibody fragment directed against the tumor T-cell epitope HLA-A1-MAGE-A1 from a nonimmunized phage-Fab library. Proc Natl Acad Sci USA. 2000 Jul. 5; 97(14): 7969-74). In this case it will be appreciated that the APC targeting ligand assist the particular MHC peptide binding antibody to bind to its target.

See also WO 97/07819 which is hereby disclaimed with respect to all relevant aspects of the invention herein insofar as inherently disclosed therein. See also U.S. Pat. No. 5,770,403 with respect to antibodies which bind to cytokines.

In one embodiment, the respective antibody components of the multispecific ligand recognize a substantially different subset of non-targeted tissues so that functional binding to a non-targeted tissue is substantially precluded. It will be appreciated that this strategy can be accomplished with two different antibodies have differing and preferably non-overlapping normal i.e. non-targeted tissue distributions. In a preferred embodiment the target cell is a cancer cell and the respective first and second cell surface associated ligands are expressed on different subsets of normal cells, which are non-overlapping subsets, so as to minimize deleterious normal cell targeting and distribute the undesired effects or normal cell targeting (e.g. with a toxin), to different cell populations. For example in the case of tumor cell targeting one or both ligands may be expressed exclusively on a single tumor type (e.g. a human sarcoma or carcinoma, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (mycloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease) or a particular category of tumor types (e.g. adenocarcinomas, tumors of neuroectodermal origin, or on multiple different tumor types or categories of tumor. One or both components (they may be the same or different) may be a dAb, a scFv, an Fab, a minibody moiety or a substantially intact antibody, for example both may be scFvs and the resulting product may be a diabody, triabody, or tetrabody. For example in a preferred embodiment the bispecific antibody comprises two dAb components comprising linked via a linker (see above) and having at least at least part of a constant region for fusion for example to a toxin (e.g. at least a partial hinge region, and preferably also at least a partial CH2 domain (optionally also at least a partial CH3 domain). In another embodiment, a trispecific antibody or tetraspecific antibody with at least two different and preferably 3 or 4 subsets (preferably at least one or more of such subsets being non-overlapping subsets) of non-targeted cell reactivities may be employed in the form of a trispecific or tetraspecific antibody respectively whereby up to three or four different pairs of ligands are targeted, so as further minimize normal cell targeting and also preferably target a heterogeneous population of cells within the same tumor. Ligands with distributions on normal tissues are well known, some being referenced herein, for example CEA, CD-20, P53, epidermal growth factor, including known multicarcinomic and pancarcinomic ligands (e.g. see U.S. Pat. No. 5,171,665, U.S. Pat. No. 4,349,528.

The term functional binding is used to mean binding for the purpose of accomplishing the object of the binding, for example binding for a sufficient duration to inhibit or enhance a particular effect, such as cell killing, for example in the case where one both antibody components are selected for their ability to internalize, binding for a sufficient duration to permit internalization, for example to deliver a toxic payload. As discussed above, the term substantially in reference to therapeutic advantage is used to refer to a degree which provides a significant benefit from a therapeutic standpoint.

Examples of tumor associated antigens (e.g. WO 01/21828) and targets and related antibodies are referenced throughout the disclosure and the foregoing aspect of the invention is for greater certainty directed to bispecific antibodies (including trispecific and tetraspecific antibodies, optionally including a component which also binds to a lymphatic vessel associated ligand), which target each of the combinations and permutations of the target cell (diseased, disease causing or immune) associated antigens, ligands, epitopes or receptors well known to those skilled in the art, herein directly or indirectly referenced or referenced in the materials herein incorporated by reference (i.e. permutations and combinations of pairs or where a tri- or tetra-specific antibody is used possibly permutations of (3 or 4) groups of pairs including for example pairs in which one member is used for targeting and the second is used for modulation purposes such modulation including without limitation, simple binding e.g. to deliver a payload, apoptosis inducing (e.g. anti-fas), modified vascular adhesion properties (e.g. anti-CD44), modified cytokine binding (anti-CCR5) etc. (re: relevant ligands/markers see also U.S. Pat. No. 6,010,902 and the references cited therein, Samter's Immunologic Diseases, Fifth and Sixth Edition, Lippincott, Frank Austen, MD Michael M. Frank, MD John P. Atkinson, MD Harvey I. Cantor, MD (6^{th}-ISBN: 0-7817-2120-2); Fundamental Virology, Third and Fourth Edition, Lippincott David M. Knipe, PhD Peter M. Howley, MD Diane E. Griffin, MD, PhD Robert A. Lamb, PhD, ScD Malcolm A. Martin, MD Bernard Roizman, ScD Stephen E. Straus, MD (4^{th}-ISBN: 0-7817-1833-3); Arthritis and Allied Conditions—A Textbook of Rheumatology, Thirteenth and Fourteenth Editions, William J. Koopman, MD 14^{th}:ISBN: 0-7817-2240-3, November 2000; Cancer—Principles and Practice of Oncology, Fifth and Sixth Editions, Lippincott, Vincent T. DeVita, Jr., MD Samuel Hellman, MD Steven A. Rosenberg, MD, PhD ISBN: 0-7817-2229-2; Dubois' Lupus Erythematosus, Fifth Edition, Daniel J. Wallace, MD ISBN: 0-683-08665-0, December 1996; Cytokine Therapeutics in Infectious Diseases, Steven M. Holland, MD, PhD, Lippincott, ISBN: 0-7817-1625-X, U.S. Pat. No. 6,054,561), in each of their permutations of size/valency (i.e. dabs, scFv, diabodies etc herein referenced) as applied to each of the applicable disease conditions herein referenced or otherwise known to those skilled in the art.

With respect to recombinant techniques for producing Fv fragments see also WO 88/01649, WO 88/06630, WO 88/07085, WO 88/07086, and WO 88/09344.

With respect to preparing ligands for specific MHC peptide complexes see also WO 01/22083: Direct selection of a human antibody fragment directed against the tumor T-cell epitope HLA-A1-MAGE-A1 from a nonimmunized phage-Fab library. Proc Natl Acad Sci USA. 2000 Jul. 5; 97(14): 7969-74.

With respect to bispecific antigen binding constructs that are suitable for binding to more than one antigen on the same cell see also Schmiedl A et al. Protein Eng 2000 October 13(10): 725-34.

Preferred immunoconjugates include radiolabeled antibody components and conjugates of an anti-Lyve-1 antibody component and an antibody component which comprises an immunomodulator.

A radiolabeled immunoconjugate may comprise an alpha-emitting radioisotope, a beta-emitting radioisotope, a gamma emitting radioisotope, an Auger electron emitter, a neutron capturing agent that emits alpha-particles or a radioisotope that decays by electron capture. Suitable radioisotopes include ¹⁹⁸Au, ³²p, ¹²⁵I, ¹³¹I, ⁹⁰Y, ¹⁸⁶Re, ¹⁸⁸Re, ⁶⁷Cu, ²¹¹At, and the like.

As discussed above, a radioisotope can be attached to an antibody component directly or indirectly, via a chelating agent. For example, ⁶⁷CU, considered one of the more promising radioisotopes for radioimmunotherapy due to its 61.5 hour half-life and abundant supply of beta particles and gamma rays, can be conjugated to an antibody component using the chelating agent, p-bromoacetamido-benzyl-tetraethylaminetetraacetic acid (TETA). Chase, “Medical Applications of Radioisotopes,” in Remington's Pharmaceutical Sciences, 18th Edition, Gennaro et al. (eds.), pages 624-652 (Mack Publishing Co. 1990) (see also 19^{th} edition of Reminton's). Alternatively, ⁹⁰Y, which emits an energetic beta particle, can be coupled to an antibody component using diethylenetriaminepentaacetic acid (DTPA). Moreover, a method for the direct radiolabeling of the antibody component with ¹³¹I is described by Stein et al., Antibody Immunoconj. Radiopharm. 4: 703 (1991) (see also U.S. Pat. No. 6,080,384).

Alternatively, boron addends such as carboranes can be attached to antibody components, as discussed above.

In addition, therapeutic immunoconjugates can comprise an immunomodulator moiety suitable for application for the purposes herein. Broadly speaking, the term “immunomodulator” includes cytokines, stem cell growth factors, lymphotoxins, such as tumor necrosis factor (TNF), and hematopoietic factors, such as interleukins (e.g., interleukin-1 (IL-1), IL-2, IL-3, IL6, IL-10 and IL-12), colony stimulating factors (e.g., granulocyte-colony stimulating factor (G-CSF) and granulocyte macrophage-colony stimulating factor (GM-CSF)), interferons (e.g., interferons alpha, beta and gamma), the stem cell growth factor designated “S1 factor,” erythropoietin and thrombopoietin. Examples of suitable immunomodulator moieties include IL-2, IL-6, IL-10, IL12, interferon-gamma, TNF-alpha. and the like.

A related form of therapeutic protein is a fusion protein comprising an antibody moiety and an immunomodulator moiety.

Methods of making antibody-immunomodulator fusion proteins are known to those of skill in the art as discussed herein. For example, antibody fusion proteins comprising an interleukin-2 moiety are described by Boleti et al., Ann. Oncol. 6:945 (1995), Nicolet et al., Cancer Gene Ther. 2:161 (1995), Becker et al., Proc. Nat'l Acad. Sci. USA 93:7826 (1996), Hank et al., Clin. Cancer Res. 2:1951 (1996), and Hu et al., Cancer Res. 56:4998 (1996). In addition, Yang et al., Hum. Antibodies Hybridomas 6:129 (1995), describe a fusion protein that includes an F(ab′)₂ fragment and a tumor necrosis factor alpha moiety.

Such immunoconjugates and antibody-immunomodulator fusion proteins provide a means to deliver an immunomodulator to a target cell and are particularly useful against tumor cells. The cytotoxic effects of immunomodulators are well known to those of skill in the art. See, for example, Kle et al., “Lymphokines and Monokines,” in Biotechnology and Pharmacy, Pessuto et al. (eds.), pages 53-70 (Chapman & Hall 1993) as well as other references herein cited. As an illustration, interferons can inhibit cell proliferation by inducing increased expression of class I histocompatibility antigens on the surface of various cells and thus, enhance the rate of destruction of cells by cytotoxic T lymphocytes. Furthermore, tumor necrosis factors, such as TNF-alpha, are believed to produce cytotoxic effects by inducing DNA fragmentation.

Moreover, therapeutically useful immunoconjugates can be prepared in which an antibody component is conjugated to a toxin or a chemotherapeutic drug. Illustrative of toxins which are suitably employed in the preparation of such conjugates are ricin, abrin, ribonuclease, DNase I, Staphylococcal enterotoxin-A, pokeweed antiviral protein, gelonin, diphtheria toxin, Pseudomonas exotoxin, and Pseudomonas endotoxin. See references herein as well as for example, Pastan et al., Cell 47:641 (1986), and Goldenberg, CA-A Cancer Journal for Clinicians 44:43 (1994). Other suitable toxins are known to those of skill in the art.

With respect to bispecific antibody constructs which are capable of binding simultaneously to two ligands on the same cell see also WO96/32841. Various such constructs are known in the art. An alternative approach to introducing the combination of therapeutic antibody and toxin is provided by antibody-toxin fusion proteins. An antibody-toxin fusion protein is a fusion protein that comprises an antibody moiety and a toxin moiety. Methods for making antibody-toxin fusion proteins are known to those of skill in the art (see references cited herein); antibody-Pseudomonas exotoxin A fusion proteins have been described by Chaudhary et al., Nature 339:394 (1989), Brinkmann et al., Proc. Nat'l Acad. Sci. USA 88:8616 (1991), Batra et al., Proc. Nat'l Acad. Sci. USA 89:5867 (1992), Friedman et al., J. Immunol. 150:3054 (1993), Wels et al., Int. J. Can. 60:137 (1995), Fominaya et al., J. Biol. Chem. 271:10560 (1996), Kuan et al., Biochemistry 35:2872 (1996), and Schmidt et al., Int. J. Can. 65:538 (1996). Antibody-toxin fusion proteins containing a diphtheria toxin moiety have been described by Kreitman et al., Leukemia 7:553 (1993), Nicholls et al., J. Biol. Chem. 268:5302 (1993), Thompson et al., J. Biol. Chem. 270:28037 (1995), and Vallera et al., Blood 88:2342 (1996). Deonarain et al., Tumor Targeting 1:177 (1995), have described an antibody-toxin fusion protein having an RNase moiety, while Linardou et al., Cell Biophys. 24-25:243 (1994) produced an antibody-toxin fusion protein comprising a DNase I component. Gelonin was used as the toxin moiety in the antibody-toxin fusion protein of Wang et al., Abstracts of the 209th ACS National Meeting, Anaheim, Calif., Apr. 2-6, 1995, Part 1, BIOT005. As a further example, Dohlsten et al., Proc. Nat'l Acad. Sci. USA 91:8945 (1994), reported an antibody-toxin fusion protein comprising Staphylococcal enterotoxin-A. Numerous other examples have been reported in the literature.

Useful cancer chemotherapeutic drugs for the preparation of immunoconjugates include nitrogen mustards, alkyl sulfonates, nitrosoureas, triazenes, folic acid analogs, pyrimidine analogs, purine analogs, antibiotics, epipodophyllotoxins, platinum coordination complexes, hormones, and the like. Suitable chemotherapeutic agents are described in Remington's Pharmaceutical Sciences, 19th Ed. (Mack Publishing Co. 1995), and in Goodman and Gilman's The Pharmacological Basis of Therapeutics, 7th Ed. (MacMillan Publishing Co. 1985). Other suitable chemotherapeutic agents, such as experimental drugs, are known to those of skill in the art.

In addition, therapeutically useful immunoconjugates can be obtained by conjugating photoactive agents or dyes to an antibody composite. Fluorescent and other chromogens, or dyes, such as porphyrins sensitive to visible light, have been used to detect and to treat lesions by directing the suitable light to the lesion. In therapy, this has been termed photoradiation, phototherapy, or photodynamic therapy (Jori et al. (eds.), Photodynamic Therapy of Tumors and Other Diseases (Libreria Progetto 1985); van den Bergh, Chem. Britain 22:430 (1986)). Moreover, monoclonal antibodies have been coupled with photoactivated dyes for achieving phototherapy. Mew et al., J. Immunol. 130:1473 (1983); Mew et al., Cancer Res. 45:4380 (1985); Oseroff et al., Proc. Natl. Acad. Sci. USA 83:8744 (1986); Oseroff et al., Photochem. Photobiol. 46:83 (1987); Hasan et al., Prog. Clin. Biol. Res. 288:471 (1989); Tatsuta et al., Lasers Surg. Med. 9:422 (1989); Pelegrin et al., Cancer 67:2529 (1991). However, these earlier studies did not include use of endoscopic therapy applications, especially with the use of antibody fragments or subfragments. Thus, the present invention contemplates the therapeutic use of immunoconjugates comprising photoactive agents or dyes.

With respect to a multifunctional ligand having a first portion that binds to both lymphatic endothelial cells and tumor vasculature, the invention contemplates using phage display or ribosome display to generate an antibody that binds to vefgr-3 as well as one or both of vegfr-2 or vegfr-1, having regard to the sequences of those respective receptors (see U.S. Pat. Nos. 5,776,755, 5,877,020, 5,952,199, 6,107,046, 6,130,071, 6,221,839, 6,235,713, 6,245,530; see also WO 00/21560, WO 95/33772, WO 97/05250, WO 98/33917). Preferably the antibody does not internalize, particularly in the case where the multifunctional ligand is fused or conjugated to a toxic moiety. The invention also contemplates, for example, fusing the binding domain of VEGF-C or VEGF-D to antitumor antibody. The invention also contemplates that the risk of retargeting cancer cells to non-tumor sites of angiogenesis, can be minimized by employing one or more of the following strategies pre- and/or co-treatment with inhibitors of angiogenesis, providing the multifunctional ligand with an effector function, such as a toxic moiety, cytokine or antibody component which retargets immune cells capable of killing such cancer cells. The invention also contemplates using in combination or alone a multifunctional ligand having a second portion that comprises an anti-VEGF antibody portion which binds to one or more of the VEGF family of ligands in order to inhibit lymphangiogenesis and/or angiogenesis. (see also for example, WO 00/37025, WO 98/33917, U.S. Pat. No. 6,130,071, WO 01/12669). With respect to angiogenesis and particularly lymphangiogenesis see also: 1: Shibuya M. Structure and function of VEGF/VEGF-receptor system involved in angiogenesis. Cell Struct Funct. 2001 February; 26(1): 25-35. Yonemura Y, et al. Lymphangiogenesis and the vascular endothelial growth factor receptor (VEGFR)-3 in gastric cancer. Eur J. Cancer. 2001 May; 37(7): 918-23; lljin K, et al VEGFR3 gene structure, regulatory region, and sequence polymorphisms FASEB J. 2001 April; 15(6): 1028-36. Tang R F, et al. Overexpression of lymphangiogenic growth factor VEGF-C in human pancreatic cancer. Pancreas. 2001 April; 22(3): 285-92: Kadambi A, Carreira C M, Yun C O, Padera T P, Dolmans D E, Carmeliet P, Fukumura D, Jain R K. Vascular endothelial growth factor (VEGF)-C differentially affects tumor vascular function and leukocyte recruitment: role of VEGF-receptor 2 and host VEGF-A. Cancer Res. 2001 Mar. 15; 61(6): 2404-8. Karpanen T, et al., Vascular endothelial growth factor C promotes tumor lymphangiogenesis and intralymphatic tumor growth. Cancer Res. 2001 Mar. 1; 61(5): 1786-90: Baldwin M E, et al., The Specificity of Receptor Binding by Vascular Endothelial Growth Factor-D Is Different in Mouse and Man. Biol. Chem. 2001 Jun. 1; 276(22): 19166-19171. Niki T, et al J. Pathol. 2001 April; 193(4): 450-7. Veikkola T, et al Signaling via vascular endothelial growth factor receptor-3 is sufficient for lymphangiogenesis in transgenic mice. EMBO J. 2001 Mar. 15; 20(6): 1223-31. Achen M G, et al., Localization of vascular endothelial growth factor-D in malignant melanoma suggests a role in tumour angiogenesis. J. Pathol. 2001 February; 193(2): 147-54 Stacker S A, et al., VEGF-D promotes the metastatic spread of tumor cells via the lymphatics. Nat. Med. 2001 February; 7(2): 186-91. Plate K. From angiogenesis to lymphangiogenesis. Nat. Med. 2001 February; 7(2): 151-2. Joukov V, et al; A novel vascular endothelial growth factor, VEGF-C, is a ligand for the Flt4 (VEGFR-3) and KDR (VEGFR-2) receptor tyrosine kinases. EMBO J. 1996 Apr. 1; 15(7): 1751. Lee J, et al, Proc Natl Acad Sci USA. 1996 Mar. 5; 93(5): 1988-92.

Multimodal therapies are also contemplated within the present invention, including particularly for cancer, therapies which can be determined to be useful complementary therapies for the anti-metastatic embodiments of this invention such as anti-angiogenic Ab conjugates

In another form of multimodal therapy, subjects receive the multifunctional ligands of the present invention and standard cancer chemotherapy. For example, “CVB” (1.5 g/m.sup.2 cyclophosphamide, 200-400 mg/m² etoposide, and 150-200 mg/m² carmustine) is a regimen used to treat non-Hodgkin's lymphoma. Patti et al., Eur. J. Haematol. 51:18 (1993). Other suitable combination chemotherapeutic regimens are well known to those of skill in the art. See, for example, Freedman et al., “Non-Hodgkin's Lymphomas,” in Cancer Medicine, Volume 2, 3rd Edition, Holland et al. (eds.), pages 2028-2068 (Lea & Febiger 1993). As an illustration, first generation chemotherapeutic regimens for treatment of intermediate-grade non-Hodgkin's lymphoma include C-MOPP (cyclophosphamide, vincristine, procarbazine and prednisone) and CHOP (cyclophosphamide, doxorubicin, vincristine, and prednisone). A useful second generation chemotherapeutic regimen is m-BACOD (methotrexate, bleomycin, doxorubicin, cyclophosphamide, vincristine, dexamethasone and leucovorin), while a suitable third generation regimen is MACOP-B (methotrexate, doxorubicin, cyclophosphamide, vincristine, prednisone, bleomycin and leucovorin). Additional useful drugs include phenyl butyrate and brostatin-1.

In general, the dosage of administered multifunctional ligands, immunoconjugates, and fusion proteins will vary depending upon such factors as the patient's age, weight, height, sex, general medical condition and previous medical history. Typically, it is desirable to provide the recipient with a dosage of antibody component, immunoconjugate or fusion protein which is generally at least in the range of from about 1 pg/kg to 10 mg/kg (amount of agent/body weight of patient), although a lower or higher dosage also may be administered as circumstances dictate, particularly to take advantage of the depot effect of the invention.

Administration of the invention including, immunoconjugates or fusion proteins to a patient can be intravenous, intraarterial, intraperitoneal, intramuscular, subcutaneous, intrapleural, intrathecal, by perfusion through a regional catheter, or by direct intralesional injection. When administering therapeutic proteins by injection, the administration may be by continuous infusion or by single or multiple boluses.

Those of skill in the art are aware that intravenous injection provides a useful mode of administration due to the thoroughness of the circulation in rapidly distributing antibodies. Intravenous administration, however, is subject to limitation by a vascular barrier comprising endothelial cells of the vasculature and the subendothelial matrix. Still, the vascular barrier is a more notable problem for the uptake of therapeutic antibodies by solid tumors. Lymphomas have relatively high blood flow rates, contributing to effective antibody delivery. Intralymphatic routes of administration, such as subcutaneous or intramuscular injection, or by catheterization of lymphatic vessels, also provide a useful means of treating lymphomas.

With regard to “low doses” of ¹³¹I-labeled immunoconjugates, the invention includes a dosage is in the range of 15 to 40 mCi, 20 to 30 mCi. In contrast, a preferred dosage of ⁹⁰Y-labeled immunoconjugates is in the range from 10 to 30 mCi, while the more preferable range is 10 to 20 mCi.

Immunoconjugates having a boron addend-loaded carrier for thermal neutron activation therapy will normally be affected in similar ways. However, it will be advantageous to wait until non-targeted immunoconjugate clears before neutron irradiation is performed. Clearance can be accelerated using an antibody that binds to the immunoconjugate. See U.S. Pat. No. 4,624,846 for a description of this general principle.

The immunoconjugates, and fusion proteins of the present invention can be formulated according to known methods to prepare pharmaceutically useful compositions, whereby the therapeutic proteins are combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well known to those in the art. See, for example, REMINGTON'S PHARMACEUTICAL SCIENCES, 19th Ed. (1995).

For purposes of therapy, antibody components (or immunoconjugates/fusion proteins) and a pharmaceutically acceptable carrier are administered to a patient in a therapeutically effective amount. A combination of an antibody component, optionally with an immunoconjugate/fusion protein, and a pharmaceutically acceptable carrier is said to be administered in a “therapeutically effective amount” if the amount administered is physiologically significant. An agent is physiologically significant if its presence results in a detectable change in the physiology of a recipient patient. In one aspect, an agent is physiologically significant if its presence results in the inhibition of the growth of target tumor cells.

Yet another therapeutic method included in the invention is a method of treating cancer by administering to an animal suffering from cancer a pharmaceutically effective amount of one or more multifunctional ligands capable of binding to cancer cells, wherein the compound is associated with a substance capable of damaging cancer cells.

Pharmaceutical compositions herein described or alluded to include multifunctional ligands of the invention or therapeutics used in combination therapy which may be administered by a variety of routes of administration.

By administration of an “effective amount” is intended an amount of the compound that is sufficient to enhance or inhibit a response, is some embodiments particularly an immune response or cellular response to a multifunctional ligand. One of ordinary skill will appreciate that effective amounts of a multifunctional ligand can be determined empirically and may be employed in pure form or, where such forms exist, in pharmaceutically acceptable salt, ester or prodrug form. The multifunctional ligand may be administered in compositions in combination with one or more pharmaceutically acceptable excipients. It will be understood that, when administered to a human patient, the total daily usage of the compounds and compositions of the present invention will be decided by the attending physician within the scope of sound medical judgment. The specific therapeutically effective dose level for any particular patient will depend upon a variety of factors including the type and degree of the cellular response to be achieved; activity of the specific multifunctional ligand employed; the specific composition employed; the age, body weight, general health, sex and diet of the patient; the time of administration, route of administration, and rate of excretion of the agonist or antagonist; the duration of the treatment; drugs used in combination or coincidental with the specific agonist or antagonist; and like factors well known in the medical arts.

On administration parenterally, for example by i.v. drip or infusion, dosages optionally at least on the order of from 0.01 to 5 mg/kg/day, optionally 0.05 to 1.0 mg/kg/day and more preferably 0.1 to 1.0 mg/kg/day can be used. Suitable daily dosages for patients are thus on the order of from 2.5 to 500 mg p.o., optionally 5 to 250 mg p.o., optionally 5 to 100 mg p.o., or on the order of from 0.5 to 250 mg i.v., optionally 2.5 to 125 mg i.v. and optionally 2.5 to 50 mg i.v.

Dosaging may also be arranged in a patient specific manner to provide a predetermined concentration of an agonist or antagonist in the blood, as determined by the RIA technique. Thus patient dosaging may be adjusted to achieve regular on-going trough blood levels, as measured by RIA, optionally on the order of at least from 50 to 1000 ng/ml, preferably 150 to 500 ng/ml.

From above, pharmaceutical compositions are provided comprising an agonist or antagonist and a pharmaceutically acceptable carrier or excipient, which may be administered orally, rectally, parenterally, intracisternally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intracisternal, subcutaneous and intraarticular injection and infusion.

Optionally a composition for parenteral injection can comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylceullulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

Some compositions herein described may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In some cases, in order to prolong the effect of one or therapeutic components herein described, it is desirable to slow the absorption from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

The multifunctional ligand can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolically lipid capable of forming liposomes can be used. The present compositions in liposome form can contain, in addition to the agonist or antagonist, stabilizers, preservatives, excipients, and the like. The preferred lipids are the phospholipids and the phosphatidyl choaes (lecithins), both natural and synthetic. Methods to form liposomes are known in the art. See, for example, Prescott, Ed., Methods in Cell Biology, Volume XIV, Academic Press, New York, N.Y. (1976), p. 33 et seq.

The present invention also contemplates a method of treatment in which immunomodulators are administered to prevent, mitigate or reverse radiation-induced or drug-induced toxicity of normal cells, and especially hematopoietic cells. Adjunct immunomodulator therapy allows the administration of higher doses of cytotoxic agents due to increased tolerance of the recipient mammal. Moreover, adjunct immunomodulator therapy can prevent, palliate, or reverse dose-limiting marrow toxicity. Examples of suitable immunomodulators for adjunct therapy include G-CSF, GM-CSF, thrombopoietin, IL-1, IL-3, IL-12, and the like. The method of adjunct immunomodulator therapy is disclosed by Goldenberg, U.S. Pat. No. 5,120,525.

For example, recombinant IL-2 may be administered intravenously as a bolus at 6×10⁵ IU/kg or as a continuous infusion at a dose of 18×10⁶ IU/m²/d. Weiss et al., J. Clin. Oncol. 10:275 (1992). Alternatively, recombinant IL-2 may be administered subcutaneously at a dose of 12×10⁶ IU. Vogelzang et al., J. Clin. Oncol. 11:1809 (1993). Moreover, IFN-gamma may be administered subcutaneously at a dose of 1.5×10⁶ U. Lienard et al., J. Clin. Oncol. 10:52 (1992). Furthermore, Nadeau et al., J. Pharmacol. Exp. Ther. 274:78 (1995) have shown that a single intravenous dose of recombinant IL-12 (42.5.mu.g/kilogram) elevated IFN-.gamma levels in rhesus monkeys.

Suitable IL-2 formulations include PROLEUKIN (Chiron Corp./Cetus Oncology Corp.; Emeryville, Calif.) and TECELEUKIN (Hoffmann-La Roche, Inc.; Nutley, N.J.). ACTIMMUNE (Genentech, Inc.; South San Francisco, Calif.) is a suitable INF-.gamma preparation.

In the preceding detailed description, reference was made to various methodologies known to those of skill in the art of molecular biology and immunology. Publications and other materials setting forth such known methodologies to which reference was made or is made below are incorporated herein by reference in their entireties along with references cited therein as though set forth in full.

Standard reference works setting forth the general principles of recombinant DNA technology include Watson, J. D. et al, Molecular Biology of the Gene, Volumes I and II, the Benjamin/Cummings Publishing Company, Inc., publisher, Menlo Park, Calif. (1987), Darnell, J. E. et al., Molecular Cell Biology, Scientific American Books, Inc., Publisher, New York, N.Y. (1986); Lewin, B. M. Genes II, John Wiley & Sons, publishers, New York, N.Y. (1985); Old, R. W., et al., Principles of Gene Manipulation: An Introduction to Genetic Engineering, 2d edition, University of California Press, publisher, Berkeley, Calif. (1981); Maniatis, T., et al., Molecular Cloning: A Laboratory Manual, 2nd Ed. Cold Spring Harbor Laboratory, publisher, Cold Spring Harbor, N.Y. (1989), and Current Protocols in Molecular Biology, Ausubel et al., Wiley Press, New York, N.Y. (1989). Standard reference works setting forth general principles and techniques of immunology include Handbook of Experimental Immunology Blackwell Science, Incorporated, ISBN: 0632009756; Antibody Engineering Blackwell Science, Incorporated, ISBN: 0632009756; Therapeutic Immunology ISBN: 086542375X Blackwell Science, Incorporated; Encyclopedia of Immunology (1998) Morgan Kaufmann Publishers, ISBN: 0122267656; Immunology Mosby, Incorporated, ISBN: 0723429189; Abbas A K. et al. Cellular & Molecular Immunology 4^{th} Ed. 2000 ISBN 0721650023; Breitling F. et al. Recombinant Antibodies 1999 ISBN 0-471-17847-0; Masseyeff R. et al. Methods of Immunological Analysis Wiley-VCH ISBN 3-527-27906-7, 1992; Mountain et al. eds, Biotechnology 2^{nd} ed. Vol 5A 1998 ISBN 3-527-28315-3 Wiley-VCH; Campbell, A., “Monoclonal Antibody Technology,” in, Burdon, R., et al., eds, Laboratory Techniques in Biochemistry and Molecular Biology, Volume 13, Elsevier, Publisher, Amsterdam (1984);

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All publications referred to herein are indicative of the level of skill of those in the art to which the invention pertains. All publications are herein (as well as references cited therein) are incorporated by reference to the same extent as if each individual publications were specifically and individually indicated to be incorporated by reference in its entirety.

The present invention, thus generally described, will be understood more readily by reference to the preceding and following examples, which are provided by way of illustration and are not intended to be limiting of the present invention.

With respect to making applications, methods of using, target cell-associated markers etc. of multispecific ligands and bispecific antibodies see also Methods Mol Biol 2001; 166:177-92; U.S. Pat. No. 6,071,517; U.S. Pat. No. 5,897,861; U.S. Pat. No. 6,096,311; U.S. Pat. No. 5,922,845, Journal of Immunological Methods February 2001 Vol. 248(1-2) page 1-200; Mol Immunol 1999 May; 36(7): 433-45; U.S. Pat. No. 6,051,227 U.S. Pat. No. 6,143,297 U.S. Pat. No. 5,977,318 U.S. Pat. No. 5,968,510 U.S. Pat. No. 5,885,796 U.S. Pat. No. 5,885,579 U.S. Pat. No. 5,869,050 Methods of blocking T-cell activation using anti-B7 monoclonal antibodies; U.S. Pat. No. 5,851,795 U.S. Pat. No. 5,747,034 U.S. Pat. No. 6,113,901 U.S. Pat. No. 5,877,021 B7-1 targeted ribozymes; U.S. Pat. No. 5,844,095 U.S. Pat. No. 6,090,914 U.S. Pat. No. 5,718,883 Transgenic animal model for autoimmune diseases; U.S. Pat. No. 5,855,887 U.S. Pat. No. 5,811,097 U.S. Pat. No. 5,770,197 Methods for regulating the immune response using B7 binding molecules and IL4-binding molecules; U.S. Pat. No. 6,084,067 EP01073741A2 U.S. 6,130,316 U.S. Pat. No. 6,068,984 Antibodies to lymphocyte activation antigens uses therefore; U.S. Pat. No. 5,766,570 Lymphocyte activation antigens and thereto; U.S. Pat. No. 5,434,131 U.S. Pat. No. 5,316,920 Lymphocyte activation antigen HB15, a member of the immunoglobulin superfamily; U.S. Pat. No. 6,111,090 Mammalian cell surface antigens; U.S. Pat. No. 6,083,751 Chimeric receptors for the generation of selectively-activatable TH-independent cytotoxic T cells; U.S. Pat. No. 5,977,303 Mammalian cell surface antigens; U.S. Pat. No. 5,738,852 Methods of enhancing antigen-specific T cell responses U.S. Pat. No. 5,714,667

Bruhl H, et al. Depletion of CCR5-Expressing Cells with Bispecific Antibodies and Chemokine Toxins: A New Strategy in the Treatment of Chronic Inflammatory Diseases and HIV. J. Immunol. 2001 Feb. 15; 166(4): 2420-2426; Tesch H, et al treatment of patients with malignant lymphomas with monoclonal antibodies. Bone Marrow Transplant. 2000 May; 26 Suppl 2:S50-3; Jung G, Brandl M, Eisner W, Fraunberger P, Reifenberger G, Schlegel U, Wiestler O D, Reulen H J, Wilmanns W. Local immunotherapy of glioma patients with a combination of 2 bispecific antibody fragments and resting autologous lymphocytes: Evidence for in situ t-cell activation and therapeutic efficacy. Int J. Cancer. 2001 Jan. 15; 91(2): 225-230; Kan K S, Anderson V A, Leong W S, Smith A M, Worth A T, Stevenson G T. Thioether-Bonded Constructs of Fab′-gamma and Fc-gamma Modules Utilizing Differential Reduction of Interchain Disulfide Bonds J. Immunol. 2001 Jan. 15; 166(2): 1320-1326; Cao Y, Suresh M R. Bispecific MAb aided liposomal drug delivery. J Drug Target. 2000; 8(4): 257-66; Schoonjans R, et al., Fab chains as an efficient heterodimerization scaffold for the production of recombinant bispecific and trispecific antibody derivatives. J. Immunol. 2000 Dec. 15; 165(12): 7050-7. Schmiedl A, et al., Expression of a bispecific dsFv-dsFv′ antibody fragment in Escherichia coli. Protein Eng. 2000 October; 13(10): 725-34. Rohrbach F, et al., Construction and characterization of bispecific costimulatory molecules containing a minimized CD86 (B7-2) domain and single-chain antibody fragments for tumor targeting. Clin Cancer Res. 2000 November; 6(11): 4314-22. Schoonjans R, Efficient heterodimerization of recombinant bi- and trispecific antibodies. Bioseparation. 2000; 9(3): 179-83. Wallace P K, et al., Production of macrophage-activated killer cells for targeting of glioblastoma cells with bispecific antibody to FcgammaRI and the epidermal growth factor receptor. Cancer Immunol Immunother. 2000 November; 49(9): 493-503. Bioconjug Chem. 2000 November; 11(6): 842-54. Conrath K E, et al., Camel single-domain antibodies as modular building units. J Biol. Chem. 2000 Oct. 25-[Cancer Immunol Immunother. 2000 October; 49(8):441-8. Talac R, Nelson H. Current perspectives of bispecific antibody-based immunotherapy. Biol Regul Homeost Agents. 2000 July; 14(3): 175-81. Tomlinson I, Holliger P. Methods for generating multivalent and bispecific antibody fragments. Methods Enzymol. 2000; 326: 461-79. Morimoto K, Inouye K., Application of bispecific F(ab′) fragments prepared from IgMs against carcinoembryonic antigen and alkaline phosphatase. Clin Chem. 2000 September; 46(9): 1492-3. Cochlovius B, et al., Cure of Burkitt's lymphoma in severe combined immunodeficiency mice by T cells, tetravalent CD3 x CD19 tandem diabody, and CD28 costimulation. Cancer Res. 2000 Aug. 15; 60(16): 4336-41. Takemura Si, et al., Construction of a diabody (small recombinant bispecific antibody) using a refolding system. Protein Eng. 2000 August; 13(8): 583-8. Katzenwadel A, et al., Construction and in vivo evaluation of an anti-PSA x anti-CD3 bispecific antibody for the immunotherapy of prostate cancer. Anticancer Res. 2000 May-June; 20(3A): 1551-5. Yoshida H, Katayose Y, Suzuki M, Matsuno S, Kudo T, Kumagai I. Functional Fv fragment of an antibody specific for CD28: Fv-mediated co-stimulation of T cells. FEBS Lett 2:000 Jul. 7; 476(3): 266-71. 6 38, Zeidler R, et al., The Fc-region of a new class of intact bispecific antibody mediates activation of accessory cells and NK cells and induces direct phagocytosis of tumour cells. Br J Cancer. 2000July; 83(2):261-6. 0 39: Hilairet et al., Enhanced targeting specificity to tumor cells by simultaneous recognition of two antigens. Bioconjug Chem. 2000 July-August; 11(4): 452-60. Weiner L M. Bispecific antibodies in cancer therapy. Cancer J Sci Am. 2000 May; 6 Suppl 3:S265-71. Zangemeister-Wittke U, Leech S H, Oli.e. R A, Zuo Z, et al., An efficient route to the production of an IgG-like bispecific antibody. Protein Eng. 2000 May; 13(5): 361-7. Helfrich W, et al A rapid and versatile method for harnessing scFv antibody fragments with various biological effector functions. J Immunol Methods. 2000 Apr. 3; 237(1-2): 131-45 Lu D, et al., Acquired antagonistic activity of a bispecific diabody directed against two different epitopes on vascular endothelial growth factor receptor 2. J Immunol Methods. 1999 Nov. 19; 230(1-2): 159-71. Ohmi Y, et al., Tumor-specific targeting of T helper type 1 (Th1) cells by anti-CD3 x anti-c-ErbB-2 bispecific antibody. Cancer Immunol Immunother. 1999 November; 48(8): 456-62. Kipriaanov S M, et al G, Schuhmacher J, Cochlovius B, Von der Lieth C W, Matys E R, Little M. Bispecific tandem diabody for tumor therapy with improved antigen binding and pharmacokinetics. J Mol Biol. 1999 Oct. 15; 293(1): 41-56. Segal D M, Weiner G J, Weiner L M. Bispecific antibodies in cancer therapy. Curr Opin Immunol. 1999 October; 11(5) 558-62 4 88: Hudson P J. Recombinant antibody constructs in cancer therapy. Curr Opin Immunol. 1999 October; 11(5): 548-57. Alt M, et al. Novel tetravalent and bispecific IgG-like antibody molecules combining single-chain diabodies with the immunoglobulin gamma1 Fc or CH3 region. FEBS Lett. 1999 Jul. 2; 454(1-2): 90-4. Kontermann R E, Muller R. Intracellular and cell surface displayed single-chain diabodies. J Immunol Methods. 1999 Jun. 24; 226(1-2): 179-88. Morimoto K, Inouye K. Method for the preparation of bispecific F(ab′)2mu fragments from mouse monoclonal antibodies of the immunoglobulin M class and characterization of the fragments. J Immunol Methods. 1999 Apr. 22; 224(1-2): 43-50. Chaudri Z N, et al., Dual specificity antibodies using a double-stranded oligonucleotide bridge. FEBS Lett. 1999 Apr. 30; 450(1-2): 23-6. Somasundaram C, et al., Development of a trispecific antibody conjugate that directs two distinct tumor-associated antigens to CD64 on myeloid effector cells. Hum Antibodies. 1999; 9(1): 47-54. Robert B, et al. Tumor targeting with newly designed biparatopic antibodies directed against two different epitopes of the carcinoembryonic antigen (CEA). Int J. Cancer. 1999 Apr. 12; 81(2): 285-91. Kreutz F T, et al., Efficient bispecific monoclonal antibody purification using gradient thiophilic affinity chromatography. J Chromatogr B Biomed Sci Appl. 1998 Sep. 4; 714(2): 161-70. Muller K M, et al. A dimeric bispecific miniantibody combines two specificities with avidity. FEBS Lett. 1998 Jul. 31; 432(1-2): 45-9. Kreutz F T, et al., A new method to generate quadromas by electrofusion and FACS sorting. Hybridoma. 1998 June; 17(3): 267-73. Production of bispecific and trispecific F(ab)₂ and F(ab)₃ antibody derivatives. Methods Mol. Biol. 1998; 80: 121-34. Merchant A M. et al., Protein, Nucleotide An efficient route to human bispecific IgG. Nat. Biotechnol. 1998 July; 16(7): 677-81. Zhu Z, et al., High level secretion of a humanized bispecific diabody from Escherichia coli. Biotechnology (N Y). 1996 February; 14(2): 192-6. Rapid assay of phage-derived recombinant human fabs as bispecific antibodies. Biotechnology (N Y). 1995 November; 13(11): 1221-4. McGuinness B T, et al., Phage diabody repertoires for selection of large numbers of bispecific antibody fragments. Nat Biotechnol. 1996 September; 14(9): 1149-54. Helfrich W, et al., Construction and characterization of a bispecific diabody for retargeting T cells to human carcinomas. Int J. Cancer. 1998 Apr. 13; 76(2): 232-9. Muller K M, et al., The first constant domain CH1 and CL of an antibody used as heterodimerization domain for bispecific miniantibodies. FEBS Lett. 1998 Jan. 30; 422(2): 259-64. 223: FitzGerald K, et al. Improved tumour targeting by disulphide stabilized diabodies expressed in Pichia pastoris. Protein Eng. 1997 October; 10(10): 1221-5. Coloma M J, Morrison S L. Design and production of novel tetravalent bispecific antibodies. Nat Biotechnol. 1997 February; 15(2): 159-63. Hoogenboom H R. Mix and match: building manifold binding sites. Nat Biotechnol. 1997 February; 15(2): 125-6. Ridgway J B, et al., ‘Knobs-into-holes’ engineering of antibody CH3 domains for heavy chain heterodimerization. Protein Eng. 1996 July; 9(7): 617-21. Reinartz H W, et al., Bispecific multivalent antibody studied by real-time interaction analysis for the development of an antigen-inhibition enzyme-linked immunosorbent assay. Analyst. 1996 June; 121(6): 767-71. Tibben J G, et al., Pharmacokinetics, biodistribution and biological effects of intravenously administered bispecific monoclonal antibody OC/TR F(ab′)2 in ovarian carcinoma patients. Int J. Cancer. 1996 May 16; 66(4): 477-83. de Kruif J, Logtenberg T. Leucine zipper dimerized bivalent and bispecific scFv antibodies from a semi-synthetic antibody phage display library. J Biol. Chem. 1996 Mar. 29; 271(13): 7630-4. Ruf J, et al., The molecular recognition theory applied to bispecific antibodies. Nat. Med. 1995 December; 1(12): 1222; Grant S D, et al Expression of monovalent and bivalent antibody fragments in Escherichia coli. J. Hematother. 1995 October; 4(5): 383-8. Dubel S, et al., Protein, Nucleotide Bifunctional and multimeric complexes of streptavidin fused to single chain antibodies (scFv). J Immunol Methods. 1995 Jan. 27; 178(2): 201-9. Kostelny S A, et al Protein Nucleotide Formation of a bispecific antibody by the use of leucine zippers. J. Immunol. 1992 Mar. 1; 148(5): 1547-53. Pack P, Pluckthun A. Miniantibodies: use of amphipathic helices to produce functional flexibly linked dimeric FV fragments with high avidity in Escherichia coli. Biochemistry. 1992 Feb. 18; 31(6): 1579-84. Smith W, et al., Immunoglobulins secreted by a hybrid-hybridoma: analysis of chain assemblies. Hybridoma. 1992 February; 11(1): 87-98. Suresh M R, et al., Bispecific monoclonal antibodies from hybrid hybridomas. Methods Enzymol. 1986; 121: 210-28. Paulus H. Preparation and biomedical applications of bispecific antibodies. Behring Inst Mitt. 1985 December; (78): 118-32. Brennan M, et al., Preparation of bispecific antibodies by chemical recombination of monoclonal immunoglobulin G1 fragments. Science. 1985 Jul. 5; 229(4708): 81-3.

With respect to anti-CCR5 antibodies used to kill CCR5-expressing cells, with for example, bi-specific antibody chemokine fusions see Bruhl H. et al. J. Immunol. 2001 Feb. 15 166(4): 2420-2426.

With respect to targeting IKAP proteins see for example U.S. Pat. No. 6,172,195.

With respect to pertinent diseased cells, disease causing cells and other suitable targets for immunotoxins, as well as optional toxins and methods of making and using immunotoxins and related technologies see for example U.S. Pat. No. 5,980,895 Immunotoxin containing a disulfide-stabilized antibody fragment joined to a Pseudomonas exotoxin that does not require proteolytic activation; U.S. Pat. No. 5,686,072 Epitope-specific monoclonal antibodies and immunotoxins and uses thereof; U.S. Pat. No. 4,956,453 Antihuman ovarian cancer immunotoxins and methods of thereof; U.S. Pat. No. 6,146,631 Immunotoxins comprising ribosome-inactivating proteins; U.S. Pat. No. 5,756,699 Immunotoxins comprising ribosome-inactivating Proteins; U.S. Pat. No. 5,744,580 Immunotoxins comprising ribosome-inactivating Proteins; U.S. Pat. No. 6,146,850 Proteins encoding gelonin sequences; U.S. Pat. No. 5,837,491 Polynucleotides encoding gelonin sequences; U.S. Pat. No. 5,578,706 Methods and compositions concerning homogenous immunotoxin preparations; U.S. Pat. No. 5,185,434 Prolonged-action immunotoxins containing a glycopeptide constituent which inactivates ribosomes, modified on its polysaccharide units; U.S. Pat. No. 4,958,009 Anti-human ovarian cancer immunotoxins and methods of use thereof; U.S. Pat. No. 5,980,896 Antibodies reactive with human carcinomas; U.S. Pat. No. 6,074,644 Nucleic acids encoding immunotoxins containing a disulfide-stabilized antibody fragment replacing half or more of domain IB of pseudomonas exotoxin, and methods of use of the encoded immunotoxins; U.S. Pat. No. 4,981,953 Immunotoxins, process for their preparation and pharmaceutical compositions in which they are present; U.S. Pat. No. 4,980,457 Cytotoxic conjugates which can be used in therapy and process or their preparation; U.S. Pat. No. 4,545,985 Pseudomonas exotoxin conjugate immunotoxins; U.S. Pat. No. 6,020,145 Methods for determining the presence of carcinoma using the antigen binding region of monoclonal antibody BR96; U.S. Pat. No. 5,792,458 Mutant diphtheria toxin conjugates; U.S. Pat. No. 5,338,542; U.S. Pat. No. 6,051,230 Compositions for targeting the vasculature of solid tumors; B3 antibody fusion proteins and their uses; U.S. Pat. No. 5,990,275 Linker and linked fusion polypeptides; U.S. Pat. No. 5,981,726 Chimeric and mutationally stabilized tumor-specific B1, B3 and B5 antibody fragments; immunotoxic fusion proteins; and uses thereof; U.S. Pat. No. 5,965,132 Methods and compositions for targeting the vasculature of solid tumors; U.S. Pat. No. 5,889,157 Humanized B3 antibody fragments, fusion proteins, and uses thereof; U.S. Pat. No. 6,027,725 Multivalent antigen-binding proteins; EP00861091A1 IMMUNOTOXIN CONTAINING A DISULFIDE-STABILIZED ANTIBODY FRAGMENT; U.S. Pat. No. 5,776,427 Methods for targeting the vasculature of solid tumors; U.S. Pat. No. 5,772,997 Monoclonal antibodies directed to the HER2 receptor; U.S. Pat. No. 5,665,357 Antibodies recognizing tumor associated antigen CA 55.1; U.S. Pat. No. 5,660,827 Friedrich K, et al A two-step selection approach for the identification of ligand-binding determinants in cytokine receptors. Anal Biochem. 1999 Mar. 15; 268(2): 179-86. Krebs B, et al Recombinant human single chain Fv antibodies recognizing human interleukin-6. Specific targeting of cytokine-secreting cells. J Biol. Chem. 1998 Jan. 30; 273(5): 2858-65. Wilbur D S, et al, Biotin reagents for antibody pretargeting. 2. Synthesis and in vitro evaluation of biotin dimers and trimers for cross-linking of streptavidin. Bioconjug Chem. 1997 November-December; 8(6): 819-32. Ring D B, et al. Antigen forks: bispecific reagents that inhibit cell growth by binding selected pairs of tumor antigens. Cancer Immunol Immunother. 1994 July; 39(1): 41-8. WO09942597A1 MONOVALENT, MULTIVALENT, AND MULTIMERIC MHC BINDING DOMAIN FUSION PROTEINS AND CONJUGATES, AND USES THEREFOR; EP00935607A2 SOLUBLE MONOVALENT AND MULTIVALENT MHC CLASS II FUSION PROTEINS, AND USES THEREFOR; WO09811914A1 TARGETING ANTIGENS TO THE MHC CLASS I PROCESSING PATHWAY WITH ANTHRAX TOXIN FUSION PROTEIN; WO09728191A1 MHC COMPLEXES AND USES THEREOF; U.S. Pat. No. 5,580,756 B71G fusion protein; U.S. Pat. No. 6,143,298 Soluble truncated forms of ICAM-1; U.S. Pat. No. 5,852,175 P-selectin glycoprotein ligand blocking antibodies; U.S. Pat. No. 5,800,815 Antibodies to P-selectin and their uses;

U.S. Pat. No. 6,037,454 Humanized anti-CD11a antibodies; U.S. Pat. No. 6,020,152 Lymphocyte-associated cell surface protein U.S. Pat. No. 5,807,734 Monoclonal antibodies and FV specific for CD2 antigen U.S. Pat. No. 5,622,701 Cross-reacting monoclonal antibodies specific for E- and P-selectin; U.S. Pat. No. 5,622,700 Method for treating a LFA-1-mediated disorder; JP06209788A2 IMMUNOASSAY OF HUMAN SOLUBLE ICAM-1, ANTIBODY AND KIT FOR MEASUREMENT THEREOF; JP03072430A2 ANTIVIRAL AGENT BY USING FUNCTIONAL DERIVATIVE OF INTERCELLULAR ADHESIVE MOLECULE; JP01135724A2 TREATMENT FOR NONSPECIFIC INFLAMMATION; U.S. Pat. No. 6,123,915 Methods for using agents that bind to VCAM-1; WO09929706A2 DISALICYLATE ANALOG BASED SIALYL LEWISx MIMETICS; WO09918442A1 DIAGNOSIS OF THROMBOTIC EVENTS BY DETECTING P-SELECTIN; U.S. Pat. No. 5,877,295 Antibodies which bind a subpopulation of Mac-1(CD11b/CD18) molecules which mediate neutrophil adhesion to ICAM-1 and fibrinogen; U.S. Pat. No. 5,869,460 Sulfated and phosphated saccharide derivatives, process for the preparation of the same and use thereof; U.S. Pat. No. 5,858,994 Carbohydrate conjugates as inhibitors of cell adhesion; U.S. Pat. No. 5,811,405 Multiply fucosylated dicarboxylic acids possessing antiadhesive properties; U.S. Pat. No. 5,654,282 Selectin binding glycopeptides; U.S. Pat. No. 5,632,991 Antibodies specific for E-selectin and the uses thereof; U.S. Pat. No. 5,599,676 Method for isolating a novel receptor for alpha4 integrins; U.S. Pat. No. 5,580,862 Sulfate ligands for L-selectins and methods of preventing sulfate addition; U.S. Pat. No. 5,508,387 Selectin binding glycopeptides; U.S. Pat. No. 6,177,547 Antibodies to P-selectin glycoprotein ligand; U.S. Pat. No. 5,827,670 Methods of isolating and detecting bone marrow stromal cells with VCAM-1-specific antibodies; U.S. Pat. No. 5,756,095 Antibodies with specificity for a common epitope on E-selectin and L-selectin; U.S. Pat. No. 5,565,550 Antibodies to ICAM-2, and fragments thereof; U.S. Pat. No. 6,099,838 Pharmaceutical compositions comprising anti-CD45RB antibodies for the inhibition of T-cell mediated immune responses; U.S. Pat. No. 5,595,737 Methods for using monoclonal antibodies specific for cell-surface bound LAM-1; U.S. Pat. No. 5,324,510, U.S. Pat. No. 6,183,988 Leukocyte-specific protein and gene, and methods of use thereof; U.S. Pat. No. 5,998,598; U.S. Pat. No. 5,997,865 Agonist antibodies against the flk2/flt3 receptor and uses thereof; U.S. Pat. No. 5,993,816 Methods to inhibit humoral immune responses, immunoglobulin production and B cell activation with 5c8-specific antibodies; U.S. Pat. No. 5,869,453 Cytotoxic T-cell epitopes; U.S. Pat. No. 5,861,151 Soluble fusion molecules with binding specificity for cell adhesion molecules; U.S. Pat. No. 5,843,441 Use of endothelial-leukocyte adhesion molecule-1 specific antibodies in the treatment of asthma; U.S. Pat. No. 5,821,332 Receptor on the surface of activated CD4+ T-cells: ACT-4; EP00868197A1 ANTI-SELECTIN ANTIBODIES FOR PREVENTION OF MULTIPLE ORGAN FAILURE AND ACUTE ORGAN DAMAGE; U.S. Pat. No. 5,817,515 Human B2 integrin alpha subunit antibodies; EP00528931B1 HUMANIZED CHIMERIC ANTI-ICAM-1 ANTIBODIES, METHODS OF PREPARATION AND USE; U.S. Pat. No. 5,776,775 Anti-LAM 1-3 antibody and hybridoma; U.S. Pat. No. 6,063,906 Antibodies to integrin alpha subunit; U.S. Pat. No. 5,997,865 Agonist antibodies against the flk2/flt3 receptor and uses thereof; U.S. Pat. No. 5,993,816 Methods to inhibit humoral immune responses, immunoglobulin production and B cell activation with 5c8-specific antibodies; U.S. Pat. No. 5,951,982 Methods to suppress an immune response with variant CD44-specific antibodies; U.S. Pat. No. 5,843,441 Use of endothelial-leukocyte adhesion molecule-1 specific antibodies in the treatment of asthma; U.S. Pat. No. 5,821,332 Receptor on the surface of activated CD4+ T-cells: ACT-4; U.S. Pat. No. 5,821,123 Modified antibody variable domains; EP00868197A1 ANTI-SELECTIN ANTIBODIES FOR PREVENTION OF MULTIPLE ORGAN FAILURE AND ACUTE ORGAN DAMAGE; U.S. Pat. No. 5,817,515 Human B2 integrin alpha subunit antibodies; EP00528931B1 HUMANIZED CHIMERIC ANTI-ICAM-1 ANTIBODIES, METHODS OF PREPARATION AND USE; U.S. Pat. No. 5,776,775 Anti-LAM 1-3 antibody and hybridoma; U.S. Pat. No. 5,776,755 FLT4, a receptor tyrosine kinase; U.S. Pat. No. 5,730,978 Inhibition of lymphocyte adherence with alpha.4β1-specific antibodies.

Examples of tumor specific antigens are numerous and are referred to in the hereinabove cited references and as well as the in the following references U.S. Pat. No. 6,132,980 Oct. 17, 2000 Antibodies specific for TRP-2 a human tumor antigen recognized by cytotoxic T lymphocytes; U.S. Pat. No. 6,165,464 Monoclonal antibodies directed to the HER2 receptor; U.S. Pat. No. 5,824,311 Treatment of tumors with monoclonal antibodies against oncogene antigens; U.S. Pat. No. 6,140,050 Oct. 31, 2000 Methods for determining breast cancer and melanoma by assaying for a plurality of antigens associated therewith; U.S. Pat. No. 6,051,226 MN-specific antibodies and their use in cancer treatment; U.S. Pat. No. 6,020,145 Methods for determining the presence of carcinoma using the antigen binding region of monoclonal antibody BR96; U.S. Pat. No. 5,980,896 Antibodies reactive with human carcinomas; U.S. Pat. No. 5,955,075-Method of inhibiting tumor growth using antibodies to MN protein; U.S. Pat. No. 5,917,124 Transgenic mouse model of prostate cancer; U.S. Pat. No. 5,914,389 Jun. 22, 1999 E6 associated protein; U.S. Pat. No. 5,912,143 Jun. 15, 1999 Polynucleotides encoding a human mage protein homolog; U.S. Pat. No. 5,910,626 Jun. 8, 1999 Acetyl-CoA carboxylase compositions and methods of use; U.S. Pat. No. 5,874,560 Feb. 23, 1999 Melanoma antigens and their use in diagnostic and therapeutic methods; U.S. Pat. No. 5,872,217 Feb. 16, 1999 Antibodies which specifically bind a cancerrelated antigen; U.S. Pat. No. 5,869,636 Feb. 9, 1999 Immunoreactive peptide sequence from a 43 kDhuman cancer antigen; U.S. Pat. No. 5,869,045 Feb. 9, 1999 Antibody conjugates reactive with human carcinomas; U.S. Pat. No. 5,866,124 Feb. 2, 1999 Antiidiotypic antibodies for high molecular weight-melanoma associated by same; U.S. Pat. No. 5,847,083 Dec. 8, 1998 Modified p53; U.S. Pat. No. 5,844,075 Melanoma antigens and their use in diagnostic and therapeutic methods; U.S. Pat. No. 5,843,685 Dec. 1, 1998 Production of chimeric mouse-human antibodies with specificity to human tumor antigens; U.S. Pat. No. 5,843,648 P15 and tyrosinase melanoma antigens and their use in diagnostic and therapeutic methods; U.S. Pat. No. 5,840,854, U.S. Pat. No. 5,830,470 Nov. 3, 1998 Humanized antibodies to ganglioside GM2; U.S. Pat. No. 5,830,464; U.S. Pat. No. 5,808,005 Human carcinoma antigen; U.S. Pat. No. 5,792,456 Mutant BR96 antibodies reactive with human carcinomas U.S. Pat. No. 5,78368 U.S. Pat. No. 5,773,579 Lung cancer marker; U.S. Pat. No. 5,772,997 Monoclonal antibodies directed to the HER2 receptor; U.S. Pat. No. 5,770,374; U.S. Pat. No. 5,705,157, Methods of treating cancerous cells with anti-receptor antibodies; U.S. Pat. No. 5,695,994 Dec. 9, 1997 Isolated cytolytic T cells specific for complexes of MAGE related peptides and HLA molecules; U.S. Pat. No. 5,693,763 Dec. 2, 1997 Antibodies to human carcinoma antigen; U.S. Pat. No. ?, Tumor rejection antigens which correspond to amino acid sequences in tumor rejection antigen precursor bage, and uses thereof; U.S. Pat. No. 5,681,701 Immortalized human fetal osteoblastic cells; U.S. Pat. No. 5,681,562 Oct. 28, 1997 U.S. Pat. No. 5,677,171 Oct. 14, 1997 Monoclonal antibodies directed to the HER2 receptor; U.S. Pat. No. 5,674,486 Oct. 7, 1997 U.S. Pat. No. 5,665,357 Sep. 9, 1997 Antibodies recognizing tumor associated antigen CA 55.1; Fonsatti E, et al Emerging role of protectin (CD59) in humoral immunotherapy of solid malignancies. Clin Ter. 2000 May-June; 151(3): 187-93. Knuth A, et al Cancer immunotherapy in clinical oncology. Cancer Chemother Pharmacol. 2000; 46 Suppl: S46-51. Sievers E L. Targeted therapy of acute myeloid leukemia with monoclonal antibodies and immunoconjugates. Cancer Chemother Pharmacol. 2000; 46 Suppl: S18-22; van Spriel A B, et al. Immunotherapeutic perspective for bispecific antibodies. Immunol Today. 2000 August; 21(8): 391-7; Green M C, et al. Monoclonal antibody therapy for solid tumors. Cancer Treat Rev. 2000 August; 26(4): 269-86. Xiang J Targeting cytokines to tumors to induce active antitumor immune responses by recombinant fusion proteins. Hum Antibodies. 1999; 9(1):23. Engberg J, et al. Recombinant antibodies with the antigen-specific, MHC restricted specificity of T cells: novel reagents for basic and clinical investigations and immunotherapy. Immunotechnology. 1999 March; 4(3-4): 273-8. O'Brien T J, T et al. More than 15 years of CA 125: what is known about the antigen, its structure and its function. Int J Biol Markers. 1998 October-December; 13(4): 188-95. Sharifi J, et al. Improving monoclonal antibody pharmacokinetics via chemical modification. Q J Nucl Med. 1998 December; 42(4): 242-9.

Ligands on immune or other cells which may be targeted with bispecific ligands in which one ligand of the pair dictates specificity for a population of cells or particular sub-population of those cells and a second ligand with reduced functional affinity is used to effect a specific immune function include those referenced in the following patents and publications therein referenced: U.S. Pat. No. 6,132,992 Expression vectors encoding bispecific fusion proteins and methods of producing biologically active bispecific fusion proteins in a mammalian cell; Antibody heteroconjugates and bispecific antibodies for use in regulation of lymphocyte activity; WO09942077 COMPOSITIONS AND METHODS FOR REGULATING LYMPHOCYTE ACTIVATION; U.S. Pat. No. 5,916,5600 Methods for inhibiting an immune response by blocking the GP39/CD40 and CTLA4/CD28/B7 pathways and compositions for use therewith; U.S. Pat. No. 5,876,718 Methods of inducing T cell non-responsiveness to transplanted tissues and of treating graft-versus-host-disease with anti-gp39 antibodies; EP00445228B IMMUNOTHERAPY INVOLVING CD28 STIMULATION; U.S. Pat. No. 5,709,859 Mixed specificity fusion proteins; U.S. Pat. No. 5,637,481 Expression vectors encoding bispecific fusion proteins and methods of producing biologically active bispecific fusion proteins in a mammalian cell; WO09720048 MODIFIED SFV MOLECULES WHICH MEDIATE ADHESION BETWEEN CELLS AND USES THEREOF; EP00336379 Antibody heteroconjugates for use in regulation of lymphocyte activity; EP00537293 LIGAND FOR CD28 RECEPTOR ON B CELLS AND METHODS; U.S. Pat. No. 5,182,368 Ligands and methods for augmenting B-cell proliferation; WO09300431 CTL4A RECEPTOR, FUSION PROTEINS CONTAINING IT AND USES THEREOF; EP00445228 IMMUNOTHERAPY INVOLVING CD28 STIMULATION; Role of cellular adhesion molecules in HIV type 1 infection and their impact on virus neutralization. AIDS Res Hum Retroviruses. 1998 October; 14 Suppl 3: S247-54. Cavenagh J D, et al Adhesion molecules in clinical medicine. Crit Rev Clin Lab Sci. 1998 September; 35(5): 415-59. Viney J L, Fong S. Beta 7 integrins and their ligands in lymphocyte migration to the gut. Chem. Immunol. 1998; 71:64-76. Aplin A E, et al. Signal transduction and signal modulation by cell adhesion receptors: the role of integrins, cadherins, immunoglobulin-cell adhesion molecules, and selectins. Pharmacol Rev. 1998 June; 50(2): 197-263.

With respect to ascertaining important amino acid residues for receptor activation or binding see also Zang, Q., Springer, T. A. (2001). Amino Acid Residues in the PSI Domain and Cysteine-rich Repeats of the Integrin beta 2Subunit That Restrain Activation of the Integrin alpha x beta 2. J. Biol. Chem. 276: 6922-6929; Binding site on the murine IFN-gamma receptor for IFN-gamma has been identified using the synthetic peptide approach, The Journal of Immunology, Vol 151, Issue 11 6206-6213; The Journal of Immunology, Vol, 143, Issue 11 3568-3579 The main immunogenic region of the nicotinic acetylcholine receptor identification of amino acid residues interacting with different antibodies, M Bellone et al.; Arend, W. P., Malyak, M., Guthridge, C. J. Gabay, C. (1998). INTERLEUKIN-1 RECEPTOR ANTAGONIST: Role in Biology. Annu. Rev. Immunol. 16: 27-55; The Journal of Immunology, Vol 155. Issue 10 4719-4725 Mapping of receptor binding sites on IL-1 beta by reconstruction of IL-1ra-like domains, The Journal of Immunology, 2000, 165: 6966-6974 Identification of Fetal Liver Tyrosine Kinase 3(Flt3) Ligand Domain Required for Receptor Binding and Function Using Naturally Occurring Ligand Isoforms Waithaka Mwangi³, Wendy, C Brown: and Guy H. Palmer.

The invention also contemplates multifunctional ligands comprising various combinations and permutations of such ligands including pairs and three different such ligands including multifunctional ligands including such combinations and a ligand which binding to a lymphatic vessel associated ligand. Additional pertinent references pertaining to formation of antibody dimers, microarrays of (and tissue microarrays) proteins including heterofunctional proteins and recombinant, ligands having application to the invention, and phage or ribosome display strategies having relevance herein include Zhu H. et al. Protein arrays and microarrays. Curr Opin Chem. Biol. 2001 February; 5(1): 40-5. references in IBC's conference on Protein Microarray Technology March 19-21 Santiago Calif.; WO 99/06834, WO 99/19506; WO 97/02342, WO 00/63701; WO 99/40434; U.S. Pat. No. 6,127,127; U.S. Pat. No. 6,146,830; WO 00/075298, U.S. Pat. No. 6,165,709 U.S. Pat. No. 6,204,023; Mar. 20, 2001 Modular assembly of antibody genes, antibodies prepared thereby and use; U.S. Pat. No. 5,846,818 Dec. 8, 1998 Pectate lyase signal sequence; U.S. Pat. No. 5,698,435 Dec. 16, 1997 Modular assembly of antibody genes, antibodies prepared thereby and use; U.S. Pat. No. 5,698,417 Dec. 16, 1997 Modular assembly of antibody genes, antibodies prepared thereby and use; U.S. Pat. No. 5,693,493 Dec. 2, 1997 Modular assembly of antibody genes, antibodies prepared thereby and use; U.S. Pat. No. 5,514,548 May 7, 1996 Method for in vivo selection of ligand-binding proteins; U.S. Pat. No. 5,648,237 Jul. 15, 1997 Expression of functional antibody fragments; U.S. Pat. No. 6,180,34 In vitro scanning saturation mutagenesis of proteins; U.S. Pat. No. 6,027,933 Surface expression libraries of heteromeric receptors; U.S. Pat. No. 5,910,573 Jun. 8, 1999 Monomeric and dimeric antibody-fragment fusion proteins; U.S. Pat. No. 6,150,58311 Transgenic animals expressing artificial epitope-tagged proteins; U.S. Pat. No. 6,132,992 Expression vectors encoding bispecific fusion proteins and methods of producing biologically active bispecific fusion proteins in a mammalian cell; U.S. Pat. No. 6,127,524 Binding molecules and computer-based methods of increasing the binding affinity thereof; U.S. Pat. No. 6,071,515 Dimer and multimer forms of single chain polypeptides; U.S. Pat. No. 6,054,297 Humanized antibodies and methods for making them; WO00004382 ARRAYS OF PROTEINS AND METHODS OF USE THEREOF; U.S. Pat. No. 6,008,023 Cytoplasmic expression of antibodies, antibody fragments and antibody fragment fusion proteins in E. coli; Phagemid for antibody screening; U.S. Pat. No. 5,980895 Nov. 9, 1999 Immunotoxin containing a disulfide-stabilized antibody fragment joined to a Pseudomonas exotoxin that does not require proteolytic activation; U.S. Pat. No. 5,962,255 Methods for producing recombinant vectors; U.S. Pat. No. 5,955,341 Heterodimeric receptor libraries using phagemids; WO09939210A1 HIGH DENSITY ARRAYS FOR PROTEOME ANALYSIS AND METHODS AND COMPOSITIONS THEREFOR; WO09931267 METHODS FOR THE SIMULTANEOUS IDENTIFICATION OF NOVEL BIOLOGICAL TARGETS AND LEAD STRUCTURES FOR DRUG DEVELOPMENT; U.S. Pat. No. 5,869,619 Modified antibody variable domains; U.S. Pat. No. 5,855,885 Isolation and production of catalytic antibodies using phage technology; U.S. Pat. No. 5,851,801 Dec. 22, 1998 Method of preparing polypeptide binding compositions derived from immunoglobulin variable regions; U.S. Pat. No. 5,849,500 Phagemid for antibody screening; binding composition; U.S. Pat. No. 5,837,846 Nov. 17, 1998 Biosynthetic binding proteins for immuno-targeting; U.S. Pat. No. 5,821,337 Oct. 13, 1998 Immunoglobulin variants; U.S. Pat. No. 5,821,123 Modified antibody variable domains; U.S. Pat. No. 5,789655 Aug. 4, 1998 Transgenic animals expressing artificial epitope-tagged proteins; U.S. Pat. No. 5,783,384 Selection of binding-molecules; U.S. Pat. No. 5,780,225 Method for generating libraries of antibody genes comprising amplification of diverse antibody DNAs and methods for using these libraries for the production of diverse antigen combining molecules; U.S. Pat. No. 5,770,356 Phagemids coexpressing a surface receptor and a surface heterologous protein; WO09808603 ISOLATION OF IMMUNOGLOBULINS; U.S. Pat. No. 5,716,805 Methods of preparing soluble, oligomeric proteins; U.S. Pat. No. 5,595,898 Modular assembly of antibody genes, antibodies prepared thereby and use; U.S. Pat. No. 5,582,996 Bifunctional antibodies and method of preparing same; U.S. Pat. No. 5,580,717 Recombinant library screening methods; Haab B B, Dunham M J, Brown P O. Protein microarrays for highly parallel detection and quantitation of specific proteins and antibodies in complex solutions. Genome Biol. 2001; 2(2): RESEARCH0004. Epub 2001 Jan. 22; Moch H, Kononen T, Kallioniemi O P, Sauter G. Tissue microarrays: what will they bring to molecular and anatomic pathology? Adv Anat Pathol. 2001 8:14-20; Borrebaeck C A. Antibodies in diagnostics—from immunoassays to protein chips. Immunol Today. 2000 August; 21(8): 379-82; Mendoza L G, et al. High-throughput microarray-based enzyme-linked immunosorbent assay (ELISA). Biotechniques. 1999 October; 27(4): 778-80, 782-6, 788; Morozov V N, Morozova TYa Electrospray deposition as a method for mass fabrication of mono- and multi-component microarrays of biological and biologically active substances. Anal Chem. 1999 Aug. 1; 71(15): 3110-7; Lueking A, et al. Protein microarrays for gene expression and antibody screening. Anal Biochem. 1999 May 15; 270(1): 103-11. Silzel J W, et al. Mass-sensing, multianalyte microarray immunoassay with imaging detection. Clin Chem. 1998 September; 44(9): 2036-43. Ekins R P. Ligand assays: from electrophoresis to miniaturized microarrays. Clin Chem. 1998 September; 44(9): 2015-30.

All publications referred to herein are indicative of the level of skill of those in the art to which the invention pertains.

With respect to lymphatic vessel associated ligands see also U.S. Pat. No. 5,776,755 (flt4). Mod Pathol 2000 February; 13(2): 180-5. EMBO J. 2001 Mar. 15; 20(6): 1223-1231. Nat Med 2001 February; 7(2): 199-205. Inhibition of lymphangiogenesis with resulting lymphedema in transgenic mice expressing soluble VEGF receptor-3 (VEGFR-3), J Pathol 2001 February; 193(2): 147-54 Localization of vascular endothelial growth factor-D in malignant melanoma suggests a role in tumour angiogenesis.

With respect to technologies having application herein see also Immunity 2001 April; 14(4): 437-46 The immunological barrier to xenotransplantation. Cascalho M, Platt J L.; WO 01/43779; WO 01/42285; WO 98/10795; WO 01/40803; WO 00/14212; Gastroenterology 2001 May; 120(6): 1330-8 An engineered human antibody to TNF (CDP571) for active Crohn's disease: a randomized double-blind placebo-controlled trial. Sandborn W J; WO 01/44282; WO 01/40309; WO 01/40274; WO 01/44300; Ann Rheum Dis 2001 May; 60(5): 433 Cancer and autoimmunity: autoimmune and rheumatic features in patients with malignancies, Abu-Shakra M, et al.; WO 01/40468; WO 01/40307; WO 01/42297; WO 01/42294; WO 01/42296; WO 01/40456; WO 01/40308; WO 01/42306; Curr Opin Immunol 2001 April; 13(2): 134-40 Immunity against cancer: lessons learned from melanoma. Houghton A N, Gold J S, Blachere N E.; WO 01/42288; WO 01/42288; WO 01/43771; WO 01/42308; WO 01/41804; WO 01/39722; WO 01/44808; WO 01/43770; WO 01/16166; WO 01/41803; WO 01/13110; WO 00/32752; WO 98/33528; WO 01/43695; J Am Pharm Assoc (Wash) 2001 May-June; 41(3): 383-91 Magic bullets finally find their mark.; Leukemia 2001 April; 15(4): 675-6; WO 01/44301; Anticancer Res 2001 January-February; 21(1B): 621-7 Immunotherapy for recurrent colorectal cancers with human monoclonal antibody SK-1. Koda K et al.; WO 01/10911; WO 01/42506; Int J Clin Pract 2001 April; 55(3): 211-6 Tumour necrosis factor as a therapeutic target in rheumatoid arthritis and other chronic inflammatory diseases: the clinical experience with infliximab; WO 01/44472; WO 01/40302; WO 01/40305)

With respect to surface plasmon resonance measurements of affinity see U.S. Pat. No. 6,111,652 High throughput surface plasmon resonance analysis system; U.S. Pat. No. 6,208,422 Surface plasmon sensor; EP01080365 SURFACE PLASMON RESONANCE SENSOR FOR THE SIMULTANEOUS MEASUREMENT OF A PLURALITY OF SAMPLES IN FLUID FORM; WO00106236A HIGH THROUGHPUT ANALYSIS OF MOLECULAR INTERACTION USING SURFACE PLASMON RESONANCE; High throughput surface plasmon resonance analysis system as well as Dimensions of antigen recognition and levels of immunological specificity. Adv Cancer Res. 2001; 80: 147-87. Use of optical biosensors for the study of mechanistically concerted surface adsorption processes. Anal Biochem. 2001 Jan. 15; 288(2): 109-25. Experimental design for analysis of complex kinetics using surface plasmon resonance. Methods. 2000 March; 20(3):310-8., and references cited in the foregoing references.

Skeie, G O, Lunde P K, Sejersted O M, Mygland A, Aarli J A, Gilhus N E. Autoimmunity against the ryanodine receptor in myasthenia gravis. Acta Physiol Scand. 2001 March; 171(3): 379-84. 28: Haufs M G, Haneke E. Epidermolysis bullosa acquisita treated with basiliximab, an interleukin-2 receptor antibody. Acta Derm Venereol. 2001 January-February; 81(1): 72. Woo E Y, et al Regulatory CD4(+)CD25(+) T cells in tumors from patients with early-stage non-small cell lung cancer and late-stage ovarian cancer. Cancer Res. 2001 Jun. 15; 61(12): 4766-72. Barrera P, Joosten L A, den Broeder A A, van De Putte L B, van Riel P L, van Den Berg W B. Effects of treatment with a fully human anti-tumour necrosis factor alpha monoclonal antibody on the local and systemic homeostasis of interleukin 1 and TNF alpha in patients with rheumatoid arthritis. Ann Rheum Dis. 2001 July; 60(7): 660-9. Nicholson J K, Browning S W, Hengel R L, Lew E, Gallagher L E, Rimland D, McDougal J S. CCR5 and CXCR4 expression on memory and naive T cells in HIV-1 infection and response to highly active antiretroviral therapy. J Acquir Immune Defic Syndr. 2001 Jun. 1; 27(2): 105-15. Kung S K, Su R C, Shannon J, Miller R G. Characterization of four new monoclonal antibodies that recognize mouse natural killer activation receptors. Hybridoma. 2001 April; 20(2): 91-101. 43: Bank I, Dardik R, Levy V, Goldstein I, Shoham J. Differential expression and regulation of CD6 on T-cell subsets revealed by monoclonal antibody (MAb) CH11. Hybridoma. 2001 April; 20(2): 75-84.

With respect to making multifunctional ligands see also U.S. Pat. Nos. 5,731,168 and 5,821,333.

With respect to TNF and TNFR variants, and functional fragments thereof, for use as antibody targets and binding moieties with respect to various aspects of the invention herein see WO 00/67793, WO 01/30300, WO 01/49321, WO 00/62790, WO 01/03720, WO 00/60079, WO 97/46686, WO 01/41803, WO 01/38526, WO 01/37874, WO 01/12812, WO 01/12671, WO 01/05834, WO 01/03720, WO 00/77191 WO 00/73321, WO 00/71150, WO 00/67793, WO 00/67034, WO 00/66608, WO 00/66156, WO 01/24811, as well as references cited therein. Many other TNFR variants and TNF analogs are known in the art.

With respect to cytokines and cytokine receptors see also the latest editions of Cytokine Reference: A Compendium of Cytokines and Other Mediators of Host Defense by Joost J. Oppenheim (Editor), Jan Vilcek, Nicos A Nicola (Editor); Cytokine Molecular Biology: A Practical Approach by Frances R. Balkwill (Editor), Fran Balkwill (Editor); Guidebook to Cytokines and Their Receptors by Nicos Nicola (Editor); The Cytokine Network and Immune Functions by Jaques Theze; Novel Cytokine Inhibitors by Gerry A Higgs (Editor), Brian Henderson (Editor); Homology Folding of Proteins: Application to Cytokine Engineering by Subahashini; Cytokines and Cytokine Receptors (2001); International Review of Experimental Pathology Cytokine-Induced Pathology, Part B: Inflammatory Cytokines, Receptors, and Disease by G. W. Richter, Kim Solez (Editor).

With respect to antibodies that bind to CCR5 see Mol Biol Cell 2002 February; 13(2): 723-737.

With respect to variations in chemokine receptors, cytokine and other receptors that can be exploited according to one or more aspects of the invention herein see 1: Csaszar A, Abel T. Receptor polymorphisms and diseases. Eur J. Pharmacol. 2001 Feb. 23; 414(1): 9-22. 2: Gibejova A. Chemokine receptors. Acta Univ Palacki Olomuc Fac Med. 2000; 143:9-18. 3: Nishimoto N, Kishimoto T, Yoshizaki K. Anti-interleukin 6 receptor antibody treatment in rheumatic disease. Ann Rheum Dis. 2000 November; 59 Suppl 1: i21-7. 4: Aggarwal B B. Tumour necrosis factors receptor associated signaling molecules and their role in activation of apoptosis, JNK and NF-kappa B. Ann Rheum Dis. 2000 November; 59 Suppl 1:i6-16. 5: Grignani G, Maiolo A. Cytokines and hemostasis. Haematologica. 2000 September; 85(9): 967-72. 6: Idriss H T, Naismith J H. TNF alpha and the TNF receptor superfamily: structure-function relationship(s). Microsc Res Tech. 2000 Aug. 1; 50(3):184-95. 7: van Deventer S J. Cytokine and cytokine receptor polymorphisms in infectious disease. Intensive Care Med. 2000; 26 Suppl 1:S98-102. 8: Gessner A, Rollinghoff M. Biologic functions and signaling of the interleukin-4 receptor complexes. Immunobiology. 2000 January; 201(3-4): 285-307. 9: Platanias L C, Fish E N. Signaling pathways activated by interferons. Exp Hematol. 1999 November; 27(11): 1583-92. 10: Schwertschlag U S, Trepicchio W L, Dykstra K H, Keith J C, Turner K J, Dorner A J. Hematopoietic, immunomodulatory and epithelial effects of interleukin-11. Leukemia. 1999 September; 13(9): 1307-15. 11: Blasi F. The urokinase receptor. A cell surface, regulated chemokine. APMIS. 1999 January; 107(1): 96-101. 12: Izuhara K, Shirakawa T. Signal transduction via the interleukin-4 receptor and its correlation with atopy. Int J Mol. Med. 1999 January; 3(1): 3-10. 13: Tsokos G C, Liossis S N. Lymphocytes, cytokines, inflammation, and immune trafficking. Curr Opin Rheumatol. 1998 September; 10(5): 417-25. 14: Morishita R, Nakamura S, Hayashi S, Aoki M, Matsushita H, Tomita N, Yamamoto K, Moriguchi A, Higaki J, Ogihara T. Contribution of a vascular modulator, hepatocyte growth factor (HGF), to the pathogenesis of cardiovascular disease. J Atheroscler Thromb. 1998; 4(3): 128-34. 15: Kashiwamura S, Okamura H. [IL-18 and IL-18 receptor] Nippon Rinsho. 1998 July; 56(7): 1798-806. [Japanese]0.16: Paxton W A, Kang S. Chemokine receptor allelic polymorphisms: relationships to HIV resistance and disease progression. Semin Immunol. 1998 June; 10(3): 187-94. 17: Arend W P, Malyak M, Guthridge C J, Gabay C. Interleukin-1 receptor antagonist: role in biology. Annu Rev Immunol. 1998; 16: 27-55. 18: Camussi G, Lupia E. The future role of anti-tumour necrosis factor (TNF) products in the treatment of rheumatoid arthritis. Drugs. 1998 May; 55(5): 613-20. 19: Taga T, Kishimoto T. Gp130 and the interleukin-6 family of cytokines. Annu Rev Immunol. 1997; 15: 797-819. 20: Paul W E. Interleukin 4: signaling mechanisms and control of T cell differentiation. Ciba Found Symp. 1997; 204:208-16; discussion 216-9. 1. (WO 01/49321) TNF INHIBITORS FOR THE TREATMENT OF NEUROLOGICAL, RETINAL AND MUSCULAR DISORDERS. 2. (WO 01/46261) METHOD FOR TREATING INFLAMMATION. 3. (WO 01/40464) INTERLEUKIN-1-RECEPTOR ASSOCIATED KINASE-3 (IRAK3) AND ITS USE IN PROMOTION OR INHIBITION OF ANGIOGENESIS AND CARDIOVASCULARIZATION. 4. WO 01/404648) INTERLEUKIN-1-RECEPTOR ASSOCIATED KINASE-3 (IRAK3) AND ITS USE IN PROMOTION OR INHIBITION: OF ANGIOGENESIS AND CARDIOVASCULARIZATION. 5. (WO 01/30850) UMLR POLYPEPTIDES. 6. (WO 00/77195) NUCLEIC ACID ENCODING NOVEL EGF-LIKE GROWTH FACTORS. 7. (WO 00/74719) METHOD OF TREATING CARCINOMA USING ANTIBODY THERAPY AND AMELIORATING SIDE EFFECTS ASSOCIATED WITH SUCH THERAPY. 8. (WO 00/02582) TREATMENT OF CELIAC DISEASE WITH INTERLEUKIN-15 ANTAGONISTS. 9. (WO 99/47170) PREVENTIVES OR REMEDIES FOR INFLAMMATORY INTESTINAL DISEASES CONTAINING AS THE ACTIVE INGREDIENT IL-6 ANTAGONISTS. 10. (WO 99/46376) RECEPTOR FROM THE SUPERFAMILY OF TNT-RECEPTORS FROM THE HUMAN LUNG. 11. (WO99/43809) PROTEASE-ACTIVATED RECEPTOR 4 AND USES THEREOF. 12. (WO 98/48017) FAMILY OF IMMUNOREGULATORS DESIGNATED LEUKOCYTE IMMUNOGLOBULIN-LIKE RECEPTORS (LIR). 13. (WO 98/47923) IL-5R ANTAGONISTS FOR TREATMENT OF INFLAMMATION, ASTHMA AND OTHER ALLERGIC DISEASES. 14. (WO 98/46620) A NOVEL HUMAN G-PROTEIN COUPLED RECEPTOR. 15. (WO 98/46265) METHODS FOR USING ANTAGONISTIC ANTI-AVB3 INTEGRIN ANTIBODIES. 16. (WO 98/36767) MODULATION OF THE HYPOTHALAMIC-PITUITARY-ADRENAL-ADIPOSE AXIS WITH LEPTIN RECEPTOR LIGANDS. 17. (WO 98/31809) HUMAN CC CHEMOKINE SLC18. (WO 98/30706) COMPOUNDS, COMPOSITIONS AND METHODS FOR THE ENDOCYTIC PRESENTATION OF IMMUNOSUPPRESSIVE FACTORS 19. (WO 98/24817) NOVEL DNA, NOVEL PROTEIN, AND NOVEL ANTIBODY 20. (WO 98/22499) NEURON AND NEURAL TUMOUR GROWTH REGULATORY SYSTEM, ANTIBODIES THERETO AND USES THEREOF 21. (WO 98/19706) IDENTIFICATION OF UNIQUE BINDING INTERACTIONS BETWEEN CERTAIN ANTIBODIES AND THE HUMAN B7.1 AND B7.2 CO-STIMULATORY ANTIGENS 22. (WO 98/18456) PROTEASE-ACTIVATED RECEPTOR 3 AND USES THEREOF. 23. (WO 98/14480) G PROTEIN-COUPLED RECEPTOR ANTAGONISTS. 24. (WO 98/02541) GAMMA-HEREGULIN. 25. (WO97/49818) G-BETA-GAMMA REGULATED PHOSPHATIDYLINOSITOL-3′ KINASE. 26. (WO 97/48804) TIE-2 RECEPTOR LIGANDS (TIE LIGAND-3; TIE LIGAND-4) AND THEIR USES. 27. (WO 97/41225) MAMMALIAN MIXED LYMPHOCYTE RECEPTORS, CHEMOKINE RECEPTORS [MMLR-CCR]. 28. (WO 97/24373) MONOCLONAL ANTIBODY ANTAGONISTS TO HAEMOPOIETIC GROWTH FACTORS. 29. (WO 97/21732) DESIGN OF HORMONE-LIKE ANTIBODIES WITH AGONISTIC AND ANTAGONISTIC FUNCTIONS. U.S. Pat. No. 6,235,880 Human sulfonylurea receptor U.S. Pat. No. 6,221,660 DNA encoding SNORF25 receptor. U.S. Pat. No. 6,214,797 Urocortin peptides, nucleic acid encoding same, methods for using same U.S. Pat. No. 6,214,344 Hepatocyte growth factor receptor antagonists and uses thereof. U.S. Pat. No. 6,210,904 Anticoagulant test. U.S. Pat. No. 6,207,152 Hepatocyte growth factor receptor antagonists and uses thereof. U.S. Pat. No. 6,204,017 Polynucleotide encoding a histamine receptor. U.S. Pat. No. 6,197,541 Recombinant thrombin receptors and assays using them. U.S. Pat. No. 6,184,358 IP-10/Mig receptor designated CXCR3, antibodies, nucleic acids, and methods of use therefore. U.S. Pat. No. 6,177,079 Antagonists of interleukin-15. U.S. Pat. No. 6,177,078 Monoclonal antibody antagonists to IL-3. U.S. Pat. No. 6,177,077 TNT inhibitors for the treatment of neurological disorders. U.S. Pat. No. 6,171,815 Human sulfonylurea receptor. U.S. Pat. No. 6,168,783 Antagonists of interleukin-15. U.S. Pat. No. 6,166,185 Antibodies to human TIE-2 ligands. U.S. Pat. No. 6,165,466 Antagonists of interleukin-15. U.S. Pat. No. 6,162,431 Serine/threonine protein kinase. U.S. Pat. No. 6,143,870 Thrombin receptor homolog. U.S. Pat. No. 6,136,957 Antibodies which bind granulocyte-macrophane colony-stimulating factor receptor. U.S. Pat. No. 6,124,101 Recombinant thrombin receptor and related pharmaceuticals. U.S. Pat. No. 6,111,075 Protese-activated receptor PAR4 (ZCHEMR2). U.S. Pat. No. 6,103,874 Human KDEL receptor. U.S. Pat. No. 6,096,873 Gamma-heregulin. U.S. Pat. No. 6,086,874 Antitumor agent effect enhancer containing IL-6 antagonist, U.S. Pat. No. 6,084,075 Agonist and antagonist antibodies to the chemokine receptor-2 (CCR2). U.S. Pat. No. 6,063,596 G-protein coupled receptors associated with immune response. U.S. Pat. No. 6,054,292 T-cell receptor protein. U.S. Pat. No. 6,043,212 Recombinant C140 receptor, its agonists and antagonists, and nucleic acids encoding the receptor. U.S. Pat. No. 6,033,869 Polynucleotide encoding a novel human cytokine/steroid receptor. U.S. Pat. No. 6,024,936 Antibody-based method of localizing activated thrombin receptors. U.S. Pat. No. 6,017,763 G-beta-gamma regulated phosphatidylinositol-3′ kinase. U.S. Pat. No. 6,013,480 Antagonists of interleukin-15. U.S. Pat. No. 6,013,479 Human Emr1-like G protein coupled receptor U.S. Pat. No. 5,994,097 Polynucleotide encoding human G-protein coupled receptor. U.S. Pat. No. 5,985,828 Mammalian receptors for interleukin-10 (IL-10). U.S. Pat. No. 5,985,583 Cloning and expression of gonadotropin-releasing hormonereceptor. U.S. Pat. No. 5,977,072 High affinity immunoglobulin E receptor-like protein. U.S. Pat. No. 5,976,852 K. kappa./.mu.-like protein tyrosine phosphatase, PTP lambda. U.S. Pat. No. 5,976,815 Bioassay using ALK-7, a novel serine threonine kinase receptor. U.S. Pat. No. 5,972,621 Methods of identifying compounds that modulate body weight using the OB receptor U.S. Pat. No. 5,965,709 IgE antagonists. U.S. Pat. No. 5,965,365 Serine/threonine protein kinase. U.S. Pat. No. 5,955,303 Human chemokine receptor-like protein. U.S. Pat. No. 5,952,175 DNA encoding a human progesterone receptor complex p23-like protein. U.S. Pat. No. 5,945,308 Human oxidized LDL receptor. U.S. Pat. No. 5,942,606 Viral receptor protein. U.S. Pat. No. 5,928,887.kappa./.mu.-Like protein tyrosine phosphatase, PTP lambda. U.S. Pat. No. 5,912,144 Edg-1-receptor homolog. U.S. Pat. No. 5,902,585 Methods of inducing T cell unresponsiveness to donor tissue or organ in a recipient with GP39 antagonists. U.S. Pat. No. 5,892,014 DNA encoding a protease-activated receptor 3. U.S. Pat. No. 5,891,720 Isolated DNA encoding a novel human G-protein coupled receptor. U.S. Pat. No. 5,891,674 Insulin receptor tyrosine kinase substrate. U.S. Pat. No. 5,891,638 Serine threonine kinase receptor, alk-7. U.S. Pat. No. 5,888,811 Corticotropin-releasing hormone receptor. U.S. Pat. No. 5,888,510 Chronic rheumatoid arthritis therapy containing IL-6 antagonists effective component. U.S. Pat. No. 5,886,148 Parathyroid hormone receptor. U.S. Pat. No. 5,874,400 Recombinant C140 receptor, its agonists and antagonists, and nucleic acids encoding the receptor U.S. Pat. No. 5,874,273 G-beta-gamma regulated phosphatidylinositol-3′ kinase. U.S. Pat. No. 5,874,224 Growth factor receptor binding protein. U.S. Pat. No. 5,871,930 High affinity immunoglobulin E receptor-like protein. U.S. Pat. No. 5,869,633 Thrombin receptor homolog polynucleotide. U.S. Pat. No. 5,869,609 G protein coupled glutamate receptors. U.S. Pat. No. 5,869,271 G-beta-gamma regulated phosphatidylinositol-3′ kinase. U.S. Pat. No. 5,869,049 Methods of inducing T cell unresponsiveness to bone marrow with gp39 antagonists. U.S. Pat. No. 5,863,796 Antibodies which specifically bind mammalian receptors for interleukin-10 (IL-10). U.S. Pat. No. 5,863,766 Human sigma receptor. U.S. Pat. No. 5,859,201 G-beta-gamma regulated phosphatidylinositol-3′ kinase. U.S. Pat. No. 5,856,448 Antibodies specifically reactive with thrombin receptor and its components. U.S. Pat. No. 5,856,133 G-beta-gamma regulated phosphatidylinositol-3′kinase. U.S. Pat. No. 5,856,132 G-beta-gamma regulated phosphatidylinositol-3′ kinase. U.S. Pat. No. 5,851,797 TIE ligand-3, methods of making and uses thereof. U.S. Pat. No. 5,840,853 Parathyroid hormone receptor and DNA encoding same. U.S. Pat. No. 5,837,499 DNA encoding C5A receptor antagonists having substantially no agonist activity and methods of expressing same. U.S. Pat. No. 5,834,240 DNA encoding a transforming growth factor-.beta receptor associated protein. U.S. Pat. No. 5,833,987 Treatment of T cell mediated autoimmune disorders. U.S. Pat. No. 5,831,047 Oligonucleotide probes to L-AP4 sensitive glutamate receptor sequences. U.S. Pat. No. 5,830,678 Method for identifying a target peptide that modulates the binding of epinectin ligand to integrin receptors. U.S. Pat. No. 5,824,500 Nucleic acid encoding novel human KDEL receptor. U.S. Pat. No. 5,817,480 DNA encoding a histamine H2 receptor. U.S. Pat. No. 5,814,507.kappa./.mu.-like protein tyrosine phosphatase, PTP lambda. U.S. Pat. No. 5,814,464 Nucleic acids encoding TIE-2 ligand-2. U.S. Pat. No. 5,811,245 Antibodies that specifically bind to ALK-7, a novel serine threonine kinase receptor. U.S. Pat. No. 5,807,824 C5 A receptor antagonists having substantially no agonist activity. U.S. Pat. No. 5,795,966 Antagonists of interleukin-1. U.S. Pat. No. 5,789,565 Serine threonine kinase receptor, ALK-7. U.S. Pat. No. 5,789,192 Mammalian receptors for interleukin-10 (IL-10). U.S. Pat. No. 5,763,575 Agonist and antagonist peptides of the C140 receptor. U.S. Pat. No. 5,759,994 Recombinant thrombin receptor and related pharmaceuticals. U.S. Pat. No. 5,750,366 Cloning and expression of gonadotropin-releasing hormone receptor. U.S. Pat. No. 5,747,279 Nucleic acid molecules encoding kappa₃ opioid receptors, receptors encoded thereby, and uses thereof. U.S. Pat. No. 5,747,267 Method for identifying a G protein coupled glutamate receptor agonist and antagonist. U.S. Pat. No. 5,738,999 L-AP4 sensitive glutamate receptors. U.S. Pat. No. 5,730,976 Method for treating macrophage pathogen infections by TGF-B antagonists. U.S. Pat. No. 5,726,036 Granulocyte-macrophage colony-stimulating factor receptor and derivatives thereof. U.S. Pat. No. 5,721,107 Antibodies to G protein coupled glutamate receptors. U.S. Pat. No. 5,716,804 Mammalian interleukin-10 (IL-10) super-activating receptors and variants. U.S. Pat. No. 5,716,789 Method to determine ligands, agonist and antagonist of C140 receptor. U.S. Pat. No. 5,707,632 Receptors for fibroblast growth factors. U.S. Pat. No. 5,688,768 Recombinant thrombin receptor and related pharmaceuticals. U.S. Pat. No. 5,686,597 Thrombin receptor homolog. U.S. Pat. No. 5,686,292 Hepatocyte growth factor receptor antagonist antibodies and uses thereof. U.S. Pat. No. 5,683,884 Methods for identifying modulators of human calcitonin mediated metabolism. U.S. Pat. No. 5,683,693 Method for inducing T cell unresponsiveness to a tissue or organ graft with anti-CD40 ligand antibody or soluble CD405,674,981 Human calcitonin receptor polypeptides. U.S. Pat. No. 5,674,689 Human calcitonin receptor polypeptides and methods of use. U.S. Pat. No. 5,646,036 Nucleic acids encoding hepatocyte growth factor receptor antagonist antibodies. U.S. Pat. No. 5,629,283 Granulocyte-macrophage colony-stimulating factor receptor and derivatives thereof. U.S. Pat. No. 5,622,839 Recombinant production of human calcitonin receptor polypeptides. U.S. Pat. No. 5,614,609 Serine threonine kinase receptor. U.S. Pat. No. 5,556,780 cDNAS encoding mouse and rat type-2 angiotensin II receptors and their expression in host cells. U.S. Pat. No. 5,543,143 Method for activating macrophages/monocytes. U.S. Pat. No. 5,516,894 A2b-adenosine receptors. U.S. Pat. No. 5,514,555 Assays and therapeutic methods based on lymphocyte chemoattractants. U.S. Pat. No. 5,506,107 Selecting ligand agonists and antagonists. U.S. Pat. No. 5,494,806 DNA and vectors encoding the parathyroid hormone receptor, transformed cells, and recombinant production of PTHR proteins and peptides. U.S. Pat. No. 5,451,658 Antagonists of human gamma interferon. U.S. Pat. No. 5,441,935 Growth factor receptors. U.S. Pat. No. 5,385,831 Method for producing a mammalian G protein coupled glutamate receptor. U.S. Pat. No. 5,334,380 Anti-endotoxin, interleukin-1 receptor antagonist and anti-tumor necrosis factor antibody with arginine-free formulations for the treatment of hypotension. U.S. Pat. No. 5,256,766 Recombinant thrombin receptor and related pharmaceuticals. U.S. Pat. No. 5,177,190 Purified C5a receptor from human polymorphonuclear leukocytes. U.S. Pat. No. 4,857,637 Methods and compositions for immunologically modulating growth hormone receptor activity; and references cited therein. (see also 1 (WO 01/49744) MOUSE G-PROTEIN COUPLED RECEPTOR MAS 2. (WO 01/49726) A NOVEL POLYPEPTIDE-HUMAN NATRIURETIC PEPTIDE RECEPTOR18 AND THE POLYNUCLEOTIDE ENCODING SAID POLYPEPTIDE 3. (WO 01/49321) TNF INHIBITORS FOR THE TREATMENT OF NEUROLOGICAL, RETINAL AND MUSCULAR DISORDERS 4. (WO 01/00657) NOVEL INDOLE PEPTIDOMIMETICS AS THROMBIN RECEPTOR ANTAGONISTS 5. (WO 00/62790) SOLUBLE TUMOR NECROSIS FACTOR RECEPTOR TREATMENT OF MEDICAL DISORDERS. 6. (WO 01/03720) PROMOTION OR INHIBITION OF ANGIOGENESIS AND CARDIOVASCULARIZATION BY TUMOR NECROSIS FACTOR LIGAND/RECEPTOR HOMOLOGS 7. (WO 01/46261) METHOD FOR TREATING INFLAMMATION 8. (WO 01/46191) 4-[ARYL(8-AZABICYCLO[3.2.1]OCTAN-3-YL)]AMINOBENZOIC ACID DERIVATIVES 9. (WO 01/46176) NON PEPTIDE TACHYKININ RECEPTOR ANTAGONISTS 10. (WO 01/45730) TWEAK RECEPTOR 11. (WO 01/45703) NITROSATED AND NITROSYLATED CYCLOOXYGENASE-2 INHIBITORS, COMPOSITIONS AND METHODS OF USE 12. (WO 01/40464) INTERLEUKIN-1-RECEPTOR ASSOCIATED KINASE-3 (IRAK3) AND ITS USE IN PROMOTION OR INHIBITION OF ANGIOGENESIS AND CARDIOVASCULARIZATION 13. (WO 01/44213) NEW P2X7 RECEPTOR ANTAGONISTS FOR USE IN THE TREATMENT OF INFLAMMATORY, IMMUNE OR CARDIOVASCULAR DISEASES 14. (WO 01/42268) DOG OREXIN 1 RECEPTOR 15. (WO 01/42208) CYCLOAMINE CCR5RECEPTOR ANTAGONISTS 16. (WO 01/41752) ISOFORM SPECIFIC INHIBITION FOR TREATMENT OF PAIN AND REDUCTION OF ANESTHETIC THRESHOLD 17. (WO 01/03720) PROMOTION OR INHIBITION OF ANGIOGENESIS AND CARDIOVASCULARIZATION BY TUMOR NECROSIS FACTOR LIGAND/RECEPTOR HOMOLOGS 18. (WO 01/40464) INTERLEUKIN-1-RECEPTOR ASSOCIATED KINASE-3 (IRAK3) AND ITS USE IN PROMOTION OR INHIBITION OF ANGIOGENESIS AND CARDIOVASCULARIZATION 19. (WO 01/40259) MONKEY OREXIN 1 RECEPTOR 20. (WO 01/40252) MONKEY CALCIUM SENSING RECEPTOR 21. (WO 01/04139) HUMAN AXOR29 RECEPTOR 22. (WO 01/36480) MOUSE 7-TRANSMEMBRANE RECEPTOR, AXOR45 23. (WO 01/00656) NOVEL INDAZOLE PEPTIDOMIMETICS AS THROMBIN RECEPTOR ANTAGONISTS 24. (WO 00/67793) DEATH DOMAIN CONTAINING RECEPTOR 4 25. (WO 01/34645) MODULATING IL-13 ACTIVITY USING MUTATED IL-13 MOLECULES THAT ARE ANTAGONISTS OR AGONISTS OF IL-13 26. (WO 01/34138) COMPOSITIONS AND METHODS FOR TREATMENT OF NEUROLOGICAL DISORDERS AND NEURODEGENERATIVE DISEASES 27. (WO 01/32656) POLYMORPHIC FORM OF A TACHYKININ RECEPTOR ANTAGONIST 28. (WO 01/32166) NEW COMBINATION COMPRISING A β₂-ADRENORECEPTOR AGONIST AND A LEUKOTRIENE RECEPTOR ANTAGONIST 29. (WO 01/32163) NEW COMBINATION COMPRISING A β₂-ADRENO RECEPTOR AGONIST AND A LEUKOTRIENE RECEPTOR ANTAGONIST 30. (WO 01/01922) USE OF SUBSTANCE P ANTAGONISTS FOR THE TREATMENT OF ADENOCARCINOMA 31. (WO 01/30850) UMLR POLYPEPTIDES 32. (WO 01/27153) A MURINE SEVEN-TRANSMEMBRANE RECEPTOR, MUS MUSCULUS MHNEAA81 33. (WO 01/25269) NOVEL HUMAN G-PROTEIN COUPLED RECEPTOR 34. (WO 01/24828) MODULATORS OF CYTOKINE MEDIATED SIGNALING PATHWAYS AND INTEGRIN β₂-RECEPTOR ANTAGONISTS FOR COMBINATION THERAPY 35. (WO 01/24798) USE OF CENTRAL CANNABINOID RECEPTOR ANTAGONIST FOR PREPARING MEDICINES 36. (WO 01/24797) INTEGRIN RECEPTOR ANTAGONISTS 37. (WO 00/68250) 7™ RECEPTOR RAT APJ 38. (WO 01/16121) HETEROCYCLIC COMPOUNDS AND METHODS OF USE THEREOF 39. (WO 01/14406) ANTIANDROGEN AGENTS 40. (WO 01/12671) HUMAN TUMOR NECROSIS FACTOR RECEPTOR TR16 41. (WO 01/10891) IL-16 ANTAGONISTS 42. (WO 01/10889) RAT-G-PROTEIN COUPLED RECEPTOR BRS3 43. (WO 01/10423) USE OF 5-HT3 RECEPTOR ANTAGONISTS FOR THE TREATMENT OF INFLAMMATIONS OF THE RESPIRATORY TRACT 44. (WO 01/07028) THE USE OF RETINOID RECEPTOR ANTAGONISTS IN THE TREATMENT OF PROSTATE CARCINOMA 45. (WO 01/05834) HUMAN TUMOR NECROSIS FACTOR RECEPTORS TR13AND TR14 46. (WO 01/05783) BRADYKININ B1 RECEPTOR ANTAGONISTS 47. (WO 01/04139) POLYNUCLEOTIDE AND POLYPEPTIDE SEQUENCES OF HUMAN AXOR29RECEPTOR AND METHODS OF SCREENING FOR AGONISTS AND ANTAGONISTS OF THE INTERACTION BETWEEN HUMAN AXOR29RECEPTOR AND ITS LIGANDS 48. (WO 01/03720) PROMOTION OR INHIBITION OF ANGIOGENESIS AND CARDIOVASCULARIZATION BY TUMOR NECROSIS FACTOR LIGAND/RECEPTOR HOMOLOGS 49. (WO 01/01922) USE OF SUBSTANCE P ANTAGONISTS IN THE TREATMENT OF THE ADENOCARCINOMAS 50. (WO 01/00659) BENZIMIDAZOLONE PEPTIDOMIMETICS AS THROMBIN RECEPTOR ANTAGONISTS 51. (WO 01/00657) NOVEL INDOLE PEPTIDOMIMETICS AS THROMBIN RECEPTOR ANTAGONISTS 52. (WO 01/00656) NOVEL INDAZOLE PEPTIDOMIMETICS AS THROMBIN RECEPTOR ANTAGONISTS 53. (WO 01/00576) INDOLE AND INDAZOLE UREA-PEPTOIDS AS THROMBIN RECEPTOR ANTAGONISTS 54. (WO 01/00198) COMPOSITIONS AND METHODS OF TREATING CANCER USING COMPOSITIONS COMPRISING AN INHIBITOR OF ENDOTHELIN RECEPTOR ACTIVITY 55. (WO 00/78317) INTEGRIN RECEPTOR ANTAGONISTS 56. (WO 00/77195) NUCLEIC ACID ENCODING NOVEL EGF-LIKE GROWTH FACTORS 57. (WO 00/76502) METHODS AND COMPOSITIONS FOR TREATING RAYNAUD'S PHENOMENON AND SCLERODERMA 58. (WO 00/74719) METHOD OF TREATING CARCINOMA USING ANTIBODY THERAPY AND AMELIORATING SIDE EFFECTS ASSOCIATED WITH SUCH THERAPY 59. (WO 00/73321) HUMAN TUMOR NECROSIS FACTOR RECEPTOR TR10 60. (WO 00/72801) ALPHA V INTEGRIN RECEPTOR ANTAGONISTS 61. (WO 00/71150) TUMOR NECROSIS FACTOR RECEPTOR 5 62. (WO 00/69831) SPIROIMIDAZOLIDINE DERIVATIVES, THEIR PREPARATION, THEIR USE AND PHARMACEUTICAL PREPARATIONS COMPRISING THEM 63. (WO 00/69820) CYCLIC AMINE DERIVATIVES AND THEIR USES 64. (WO 00/69463) COMPOSITIONS AND METHODS FOR TREATING CELL PROLIFERATION DISORDERS 65. (WO 00/69459) TREATMENT OF REFRACTORY HUMAN TUMORS WITH EPIDERMAL GROWTH FACTOR RECEPTOR ANTAGONISTS 66. (WO 00/68250) 7™ RECEPTOR RAT APJ 67. (WO 00/68244) 7™ RECEPTOR MOUSE APJ 68. (WO 00/67793) DEATH DOMAIN CONTAINING RECEPTOR 4 69. (WO 00/67034) METHODS OF USE OF THE TACI/TACI-L INTERACTION 70. (WO 00/67024) CANCER TREATMENT WITH ENDOTHELIN RECEPTOR ANTAGONISTS 71. (WO 00/66632) AGONISTS OR ANTAGONISTS FOR HAEMOPOIETIC GROWTH FACTORS 72. (WO 00/66522) GLUCOCORTICOID RECEPTOR MODULATORS 73. (WO 00/66156) DEATH DOMAIN CONTAINING RECEPTOR 5 74. (WO 00/64465) DEATH DOMAIN CONTAINING RECEPTORS 75. (WO 00/62790) SOLUBLE TUMOR NECROSIS FACTOR RECEPTOR TREATMENT OF MEDICAL DISORDERS 76. (WO 00/59532) THE USE OF DOMAINS OF TYPE IV COLLAGEN TO INHIBIT ANGIOGENESIS AND TUMOUR GROWTH 77. (WO 00/56862) HUMAN TUMOR NECROSIS FACTOR RECEPTOR TR9 78. (WO 00/56405) HUMAN TUMOR NECROSIS FACTOR RECEPTOR-LIKE 2 79. (WO 00/54772) AMYOTROPIC LATERAL SCLEROSIS TREATMENT WITH A COMBINATION OF RILUZOLE AND AN AMPA RECEPTOR ANTAGONIST 80. (WO 00/53596) IMIDAZOLE COMPOUNDS SUBSTITUTED WITH A SIX OR SEVEN MEMBERED HETEROCYCLIC RING CONTAINING TWO NITROGEN ATOMS 81. (WO 00/53175) COMPOUNDS AND METHODS 82. (WO 00/52028) TUMOR NECROSIS FACTOR RECEPTORS 6α; and 6β; 83. (WO 00/51974) ALPHA-AMINOACETIC ACID DERIVATIVES USEFUL AS ALPHA 4 BETA 7-RECEPTOR ANTAGONISTS 84. (WO 00/50459) HUMAN TUMOR NECROSIS FACTOR RECEPTOR-LIKE PROTEINS TR11, TR11SV1, AND TR11SV2 85. (WO 00/49170) MURINE 11cby RECEPTOR 86. (WO 00/48603) DIBENZO-AZEPINE DERIVATIVES AS αV INTEGRIN RECEPTOR ANTAGONISTS 87. (WO 00/48597) SYSTEMIC USE OF 5-HT 3 RECEPTOR ANTAGONISTS AGAINST RHEUMATIC INFLAMMATORY PROCESSES 88. (WO 00/48581) USE OF 5-HT3 RECEPTOR ANTAGONISTS 89. (WO 00/46343) SCREENING ASSAY FOR ANTAGONISTS OF FGFR-MEDIATED MALIGNANT CELL TRANSFORMATION AND TUMOR FORMATION 90. (WO 00/46215) BENZAZEPINE DERIVATIVES AS ALPHA-V INTEGRIN RECEPTOR ANTAGONISTS 91. (WO 00/46197) INDOLE DERIVATIVES AND THEIR USE AS MCP-1 RECEPTOR ANTAGONISTS 92. (WO 00/44763) COMPOSITIONS FOR TREATING INFLAMMATORY RESPONSE 93. (WO 00/43031) TUMOR NECROSIS FACTOR ANTAGONISTS AND THEIR USE IN ENDOMETRIOSIS 94. (WO 00/42852) COMPOUNDS AND METHODS 95. (WO 00/40716) SOLUBLE RECEPTOR BR43x2 AND METHODS OF USING 96. (WO 00/40239) COMPOUNDS AND METHODS 97. (WO 00/39166) NOVEL HYALURONAN-BINDING PROTEINS AND ENCODING GENES 98. (WO 00/37462) NON-PEPTIDE NK 1 RECEPTORS ANTAGONISTS 99. (WO 00/35887) VITRONECTIN RECEPTOR ANTAGONIST PHARMACEUTICALS 100. (WO 00/35492) VITRONECTIN RECEPTOR ANTAGONIST PHARMACEUTICALS 51. (WO 01/00657) NOVEL INDOLE PEPTIDOMIMETICS AS THROMBIN RECEPTOR ANTAGONISTS 52. (WO 01/00656) NOVEL INDAZOLE PEPTIDOMIMETICS AS THROMBIN RECEPTOR ANTAGONISTS 53. (WO 01/00576) INDOLE AND INDAZOLE UREA-PEPTOIDS AS THROMBIN RECEPTOR ANTAGONISTS 54. (WO 01/00198) COMPOSITIONS AND METHODS OF TREATING CANCER USING COMPOSITIONS COMPRISING AN INHIBITOR OF ENDOTHELIN RECEPTOR ACTIVITY 55. (WO 00/78317) INTEGRIN RECEPTOR ANTAGONISTS 56. (WO 00/77195) NUCLEIC ACID ENCODING NOVEL EGF-LIKE GROWTH FACTORS 57. (WO 00/76502) METHODS AND COMPOSITIONS FOR TREATING RAYNAUDS'S PHENOMENON AND SCHLERODERMA 58. (WO 00/74719) METHOD OF TREATING CARCINOMA USING ANTIBODY THERAPY AND AMELIORATING SIDE EFFECTS ASSOCIATED WITH SUCH THERAPY 59. (WO 00/73321) HUMAN TUMOR NECROSIS FACTOR RECEPTOR TR10 60. (WO 00/72801) ALPHA V INTEGRIN RECEPTOR ANTAGONISTS 61. (WO 00/71150) TUMOR NECROSIS FACTOR RECEPTOR 5 62. (WO 00/69831) SPIROIMIDAZOLIDINE DERIVATIVES, THEIR PREPARATION, THEIR USE AND PHARMACEUTICAL PREPARATIONS COMPRISING THEM 63. (WO 00/69820) CYCLIC AMINE DERIVATIVES AND THEIR USES 64. (WO 00/69463) TREATMENT METHODS FOR TREATING CELL PROLIFERATION DISORDERS 65. (WO 00/69459) TREATMENT OF REFRACTORY HUMAN TUMORS WITH EPIDERMAL GROWTH FACTOR RECEPTOR ANTAGONISTS 66. (WO 00/68250) 7TM RECEPTOR RAT APJ 67. (WO 00/68244) 7TM RECEPTOR MOUSE APJ 68. (WO 00/67793DEATH DOMAIN CONTAINING RECEPTOR 4 69. (WO 00/67034) METHODS OF USE OF THE TACI/TACI-L INTERACTION 70. (WO 00/67024) CANCER TREATMENT WITH ENDOTHELIN RECEPTOR ANTAGONISTS 71. (WO 00/66632) AGONISTS OR ANTAGONISTS FOR HAEMOPOETIC GROWTH FACTORS 72. (WO 00/66522) GLUCOCORTICOID RECEPTOR MODULATORS 73. (WO 00/66156) DEATH DOMAIN CONTAINING RECEPTOR 574. (WO 00/64465) DEATH DOMAIN CONTAINING RECEPTORS 75. (WO 00/62790) SOLUBLE TUMOR NECROSIS FACTOR RECEPTOR TREATMENT OF MEDICAL DISORDERS 76. (WO 00/59532) THE USE OF DOMAINS OF TYPE IV COLLAGEN TO INHIBIT ANGIOGENESIS AN TUMOUR GROWTH 77. (WO 00/56862) HUMAN TUMOR NECROSIS FACTOR RECEPTOR TR9 78. (WO 00/56405) HUMAN TUMOR NECROSIS FACTOR RECEPTOR-LIKE 2 79. (WO 00/54772) AMYOTROPIC LATERAL SCLEROSIS TREATMENT WITH A COMBINATION OF RILUZOLE AND AN AMPA RECEPTOR ANTAGONIST 80. (WO 00/53596) IMIDAZOLE COMPOUNDS SUBSTITUTED WITH A SIX OR SEVEN MEMBERED HETEROCYCLIC RING CONTAINING TWO NITROGEN ATOMS 81. (WO 00/53175) COMPOUNDS AND METHODS 82. (WO 00/52028) TUMOR NECROSIS FACTOR RECEPTORS 6&agr; 6&bgr; 83. (WO 00/51974) ALPHA-AMINOACETIC ACID DERIVATIVES USEFUL AS ALPHA 4 BETA 7-RECEPTOR ANTAGONISTS 84. (WO 00/50459) HUMAN TUMOR NECROSIS FACTOR RECEPTOR-LIKE PROTEINS TR11, TR11SV1, AND TR11SV2 85. (WO 00/49170) MURINE 11cby RECEPTOR 86. (WO 00/48603) SYSTEMIC USE OF 5-HT 3 RECEPTOR RECEPTOR ANTAGONISTS 87. (WO 00/48597) SYSTEMIC USE OF 5-HT 3 RECEPTOR ANTAGONISTS AGAINST RHEUMATIC INFLAMMATORY PROCESSES 88. (WO 00/48581) USE OF 5-HT3 RECEPTOR ANTAGONISTS 89. (WO 00/463343) SCREENING ASSAY FOR ANTAGONISTS OF FGFR-MEDIATED MALIGNANT CELL TRANSFORMATION AND TUMOR FORMATION 90. (WO 00/46215) BENZAZEPINE DERIVATIVES AS ALPHA-V INTEGRIN RECEPTOR ANTAGONISTS 91. (WO 00/46197) INDOLE DERIVATIVES AND THEIR USE AS MCP-1 RECEPTOR ANTAGONISTS 92. (WO 00/44763) COMPOSITIONS FOR TREATING INFLAMMATORY RESPONSE 93. (WO 00/43031) TUMOR NECROSIS FACTOR ANTAGONISTS AND THEIR USE IN ENDOMETRIOSIS 94. (WO 00/42852) COMPOUNDS AND METHODS 95. (WO 00/40716) SOLUBLE RECEPTOR BR43x2 AND METHODS OF USING 96. (WO 00/40239) COMPOUNDS AND METHODS 97. (WO 00/39166) NOVEL HYALURONAN-BINDING PROTEINS AND ENCODING GENES 98. (WO 00/37462) NON-PEPTIDE NK 1 RECEPTORS ANTAGONISTS 99. (WO 00/35887) VITRONECTIN RECEPTOR ANTAGONIST PHARMACEUTICALS 100. (WO 00/35492) VITRONECTIN RECEPTOR ANTAGONIST PHARMACEUTICALS 101. (WO 00/35488 VITRONECTIN RECEPTOR ANTAGONIST PHARMACEUTICALS 102. (WO 00/35455) HETEROARYL-ARYL UREAS AS IGF-1 RECEPTOR ANTAGONISTS 103. (WO 00/32578) BENZIMIDAZOLE COMPOUNDS THAT ARE VITRONECTIN RECEPTOR ANTAGONISTS 104. (WO 00/28988) NITROSATED AND NITROSYLATED H2 RECEPTOR ANTAGONIST COMPOUNDS, COMPOSITIONS AND METHODS OF USE 105. (WO 00/27421) LOCAL USE OF SOLUBLE TUMOR NECROSIS RECEPTOR I (sTNFRI) FOR PROPHYLAXIS AND TREATMENT OF CORNEAL TRANSPLANT REJECTION AND OTHER DISORDERS OF THE EYE 106. (WO 00/25805) VASCULAR ENDOTHELIAL GROWTH FACTOR-LIKE PROTEIN FROM ORF VIRUS NZ2 BINDS AND ACTIVATES MAMMALIAN VEGF RECEPTOR-2 107. (WO 00/25745) IRRIGATION SOLUTION AND METHOD FOR INHIBITION OF PAIN AND INFLAMMATION 108. (WO 00/24395) NEW USE OF GLUTAMATE ANTAGONISTS FOR THE TREATMENT OF CANCER 109. (WO 00/23471) USE OF A CYTOKINE-PRODUCING LACTOCOCCUS STRAIN TO TREAT COLITIS 110. (WO 00/23469) FRAGMENTS OF INSULIN-LIKE GROWTH FACTOR BINDING PROTEIN AND INSULIN-LIKE GROWTH FACTOR, AND USES THEREOF 111. (WO 00/23438) N-(IMIDAZOLYLALKYL) SUBSTITUTED CYCLIC AMINES AS HISTAMINE-H 3 AGONISTS OR ANTAGONISTS 112. (WO 00/23113) PEPTIDE-BASED CARRIER DEVICES FOR STELLATE CELLS 113. (WO 00/23066) IRRIGATION SOLUTION AND METHOD FOR INHIBITION OF PAIN AND INFLAMMATION 114. (WO 00/23062) IRRIGATION SOLUTION AND METHOD FOR INHIBITION OF PAIN AND INFLAMMATION 115. (WO 00/20578) A METHOD OF MODULATING CELL SURVIVAL AND REAGENTS USEFUL FOR SAME 116. (WO 00/20389) NAPHTHALENECARBOXAMIDES AS TACHYKININ RECEPTOR ANTAGONISTS 117. (WO 00/20371) PROSTAGLANDIN RECEPTOR LIGANDS 118. (WO 00/20003) NAPHTHALENECARBOXAMIDES AS TACHYKININ RECEPTOR ANTAGONISTS 119. (WO 00/14109) BASIC PRODUCTS HAVING ANTAGONISTIC ACTIVITY ON THE NK-1 RECEPTOR AND THEIR USE IN PHARMACEUTICAL COMPOSITIONS 120. (WO 00/10391) THE USE OF ADENOSINE A3 RECEPTOR ANTAGONISTS TO INHIBIT TUMOR GROWTH 121. (WO 00/09503) INTEGRIN RECEPTOR ANTAGONISTS 122. (WO 00/09152) THERAPEUTIC CHEMOKINE RECEPTOR ANTAGONISTS 123. (WO 00/08001) SUBSTITUTED ISOXAZOLE AS ESTROGEN RECEPTOR MODULATORS 124. (WO 00/06169) INTEGRIN RECEPTOR ANTAGONISTS 125. (WO 00/03716) TOPICAL COMPOSITIONS COMPRISING AN OPIOID ANALGESIC AND AN NMDA ANTAGONIST 126. (WO 00/02859) N-SUBSTITUTED NAPHTHALENE CARBOXAMIDES AS NEUROKININ-RECEPTOR ANTAGONISTS 127. (WO 00/02582) TREATMENT OF CELIAC DISEASE WITH INTERLEUKIN-15 ANTAGONISTS 128. (WO 00/01802) PEPTIDE ANTAGONISTS OF THE HUMAN UROKINASE RECEPTOR AND METHOD FOR SELECTING THEM 129. (WO 00/00194) OPHTHALMIC USES OF PPAR-GAMMA AGONISTS AND PPAR-GAMMA ANTAGONISTS 130. (WO 99/65944) PEPTIDE INHIBITORS OF αVβ3 AND αVβ5; 131. (WO 99/62955) METHOD OF DESIGNING AGONISTS AND ANTAGONISTS TO EGF RECEPTOR FAMILY 132. (WO 99/60015) IMIDAZOLIDINE DERIVATIVES, THE PRODUCTION THEREOF, THEIR USE AND PHARMACEUTICAL PREPARATIONS CONTAINING THE SAME 133. (WO 99/59635) USE OF A COX-2 INHIBITOR AND A NK-1 RECEPTOR ANTAGONIST FOR TREATING INFLAMMATION 134. (WO 99/58142) USE OF ANTI-PROLACTIN AGENTS TO TREAT PROLIFERATIVE CONDITIONS 135. (WO 99/58097) USE OF ANTI-PROLACTIN AGENTS TO TREAT PROLIFERATIVE CONDITIONS 136. (WO 99/57245) METHODS OF SCREENING FOR AGONISTS AND ANTAGONISTS OF THE INTERACTION BETWEEN THE HUMAN KIAA0001 RECEPTOR AND LIGANDS THEREOF 137. (WO 99/51245) NON-PEPTIDE BRADYKININ RECEPTOR ANTAGONISTS FOR USE IN TREATING OPHTHALMIC DISEASES AND DISORDERS 138. (WO 99/50249) INTEGRIN ANTAGONISTS 139. (WO 99/49856) ANTAGONISTS FOR TREATMENT OF CD11/CD18 ADHESION RECEPTOR MEDIATED DISORDERS 140. (WO 99/47170) PREVENTIVES OR REMEDIES FOR INFLAMMATORY INTESTINAL DISEASES CONTAINING AS THE ACTIVE INGREDIENT IL-6 ANTAGONISTS 141. (WO 99/47158) THERAPEUTIC CHEMOKINE RECEPTOR ANTAGONISTS 142. (WO 99/46376) RECEPTOR FROM THE SUPERFAMILY OF TNT-RECEPTORS FROM THE HUMAN LUNG 143 (WO 99/45927) VITRONECTIN RECEPTOR ANTAGONISTS 144. (WO 99/45905) PROPHYLAXIS AND TREATMENT OF MIGRAINE HEADACHES WITH THROMBOXANE SYNTHETASE INHIBITORS AND/OR RECEPTOR ANTAGONISTS 145. (WO 99/44612) SUBSTITUTED QUINAZOLINES AND ANALOGS AND THE USE THEREOF 146. (WO 99/43809) PROTEASE-ACTIVATED RECEPTOR 4 AND USES THEREOF 147. (WO 99/42464) SUBSTITUTED IMIDAZO[1,2-a; 3,4-a′]DIQUINOLINYLIUM INTERLEUKIN-8 RECEPTOR ANTAGONISTS 148. (WO 99/42463) SUBSTITUTED QUINOXALINE DERIVATIVES AS INTERLEUKIN-8 RECEPTOR ANTAGONISTS 149. (WO 99/42461) SUBSTITUTED QUINOXALINE DERIVATIVES AS INTERLEUKIN-8 RECEPTOR ANTAGONISTS 150. (WO 99/41257) GLUCOCORTICOID-SELECTIVE ANTI INFLAMMATORY AGENTS 151. (WO 99/41256) GLUCOCORTICOID-SELECTIVE ANTI-INFLAMMATORY AGENTS 152. (WO 99/40192) HUMAN RECEPTOR GPR14, AND A METHOD OF FINDING AGONIST AND ANTAGONIST TO HUMAN AND RAT GPR14 153. (WO 99/40091) BICYCLIC PYRIDINE AND PYRIMIDINE DERIVATIVES AS NEUROPEPTIDE Y RECEPTOR ANTAGONISTS 154. (WO 99/38532) METHODS FOR THE PREVENTION AND TREATMENT OF FIBROSIS AND SCLEROSIS 155. (WO 99/36541) INTERLEUKIN-1 RECEPTOR ANTAGONIST BETA (IL-1RAβ) 156. (WO 99/33806) 4-[ARYL(PIPERIDIN-4-YL)] AMINOBENZAMIDES WHICH BIND TO THE DELTA-OPIOID RECEPTOR 157. (WO 99/31099) INTEGRIN RECEPTOR ANTAGONISTS 158. (WO 99/31061) INTEGRIN RECEPTOR ANTAGONISTS 159. (WO 99/30713) INTEGRIN RECEPTOR ANTAGONISTS 160. (WO 99/30709) INTEGRIN RECEPTOR ANTAGONISTS 161. (WO 99/29729) ANTAGONISTS OF NEUROPILIN RECEPTOR FUNCTIONAL AND USE THEREOF 162. (WO 99/27962) USE OF A FIBRINOGEN RECEPTOR-ANTAGONIST FOR PREVENTING DISSEMINATED INTRAVASCULAR COAGULATION 163. (WO 99/26945) 1,3,4-THIADIAZOLES AND 1,3,4-OXADIAZOLES AS αvβ33 ANTAGONISTS 164. (WO 99/26943) THROMBIN RECEPTOR ANTAGONISTS 165. (WO 99/25857) TRANSGENIC MODELS OF INFLAMMATORY DISEASE 166. (WO 99/24471) OPIATE, CANNABINOID, AND ESTROGEN RECEPTORS 167. (WO 99/24423) PIPERIDINE DERIVATIVES AND THEIR USE AS TACHYKININ ANTAGONISTS 168. (WO 99/24421) IMIDAZOYLALKYL SUBSTITUTED WITH A FIVE, SIX OR SEVEN MEMBERED HETEROCYCLIC RING CONTAINING ONE NITROGEN ATOM 169. (WO 99/24406) PHENYL-ALKYL-IMIDAZOLES AS H3 RECEPTOR ANTAGONISTS 170. (WO 99/24405) H 3 RECEPTOR LIGANDS OF THE PHENYL-ALKYL-IMIDAZOLES TYPE 171. (WO 99/21555) ADENOSINE A3 RECEPTOR ANTAGONISTS 172. (WO 99/20758) HUMAN TUMOR NECROSIS FACTOR RECEPTOR-LIKE PROTEINS TR11, TR11SV1, AND TR11SV2 173. (WO 99/19462) ENHANCED IMMUNOGENIC CELL POPULATIONS PREPARED USING H2 RECEPTOR ANTAGONISTS 174. (WO 99/17773) COMPOUNDS AND METHODS 175. (WO 99/16465) METHOD FOR INHIBITING TUMOR ANGIOGENESIS IN A LIVING SUBJECT 176. (WO 99/11790) TUMOR NECROSIS FACTOR RECEPTOR ZTNFR-6 177. (WO 99/06049) INTEGRIN RECEPTOR ANTAGONISTS 178. (WO 99/04001) TUMOR NECROSIS FACTOR RECEPTOR ZTNFR-5 179. (WO 99/02499) QUINOLINE COMPOUNDS AND MEDICINAL USES THEREOF 180. (WO 99/01764) METHOD FOR RECOGNIZING AND DETERMINING GNRH RECEPTORS AND THE USE OF GNRH AGONISTS AND GNRH ANTAGONISTS AND OTHER GNRH RECEPTOR LIGANDS FOR THE TREATMENT WITH GNRH RECEPTORS OF TUMOURS ORIGINATING IN THE BRAIN AND/OR NERVOUS SYSTEM AND/OR MENINGES AND/OR OF KAPOSI SARCOMA 181. (WO 99/01444) POLYMORPHIC FORM OF THE TACHYKININ RECEPTOR ANTAGONIST 2-(R)-(1-(R)-(3,5-BIS(TRIFLUOROMETHYL)PHENYL)ETHOXY)-3-(S)-(4-FLUORO)PHENYL-4-(3-5 (—OXO-1H,4H-1,2,4,-TRIAZOLO) METHYLMORPHOLINE 182. (WO 99/01127) COMPOUNDS AND METHODS 183. (WO 99/00406) CYCLIC AGONISTS AND ANTAGONISTS OF C5a RECEPTORS AND G PROTEIN-COUPLED RECEPTORS 184. WO 98/58674) ANTI-TUMOUR PHARMACEUTICAL COMPOSITIONS CAPABLE OF REDUCING DRUG RESISTANCE IN TUMOUR CELLS 185. (WO 98/57647) COUP-TFII: AN ORPHAN NUCLEAR RECEPTOR REQUIRED FOR ANGIOGENESIS 186. (WO 98/56892) HUMAN TUMOR NECROSIS FACTOR RECEPTOR TR9187. (WO 98/56779) 4-SULFINYL BENZAMIDES AS CALCITONIN GENE-RELATED PEPTIDE RECEPTOR ANTAGONISTS 188. (WO 98/55153) NON-STEROIDAL RADIOLABELED AGONIST/ANTAGONIST COMPOUNDS AND THEIR USE IN PROSTATE CANCER IMAGING 189. (WO 98/54325) HUMAN FRP AND FRAGMENTS THEREOF INCLUDING METHODS FOR USING THEM 190. (WO 98/54202) HUMAN TUMOR NECROSIS FACTOR RECEPTOR TR10 191. (WO 98/54201) HUMAN TUMOR NECROSIS FACTOR RECEPTOR-LIKE PROTEIN 8 192. (WO 98/54187) SPIRO-AZACYCLIC DERIVATIVES AND THEIR USE AS THERAPEUTIC AGENTS 193. (WO 98/53069) GDNF RECEPTORS 194. (WO 98/49170) SPIRO-AZACYCLIC DERIVATIVES AND THEIR USE AS THERAPEUTIC AGENTS 195. (WO 98/48017) FAMILY OF IMMUNOREGULATORS DESIGNATED LEUKOCYTE IMMUNOGLOBULIN-LIKE RECEPTORS (LIR) 196. (WO 98/47923) IL-5R ANTAGONISTS FOR TREATMENT OF INFLAMMATION, ASTHMA AND OTHER ALLERGIC DISEASES 197. (WO 98/46751) OSTEOPROTEGERIN BINDING PROTEINS AND RECEPTORS 198. (WO 98/46620) A NOVEL HUMAN G-PROTEIN COUPLED RECEPTOR 199. (WO 98/46265) METHODS FOR USING ANTAGONISTIC ANTI-AVB3 INTEGRIN ANTIBODIES 200. (WO 98/43962) HETEROCYCLIC INTEGRIN INHIBITOR PRODRUGS 251. (WO 97/44333) 1,2,4-OXADIAZOLES AS ADHESION-RECEPTOR ANTAGONISTS 252. (WO 97/44329) DIARYLALKYL CYCLIC DIAMINE DERIVATIVES AS CHEMOKINE RECEPTOR ANTAGONISTS 253. (WO 97/41225) MAMMALIAN MIXED LYMPHOCYTE RECEPTORS, CHEMOKINE RECEPTORS [MMLR-CCR] 254. (WO 97/37655) αvβ3 ANTAGONISTS 255. (WO 97/35969) PEPTIDE LIGANDS OF THE UROKINASE RECEPTOR 256. (WO 97/34878) SUBSTITUTED 2,3-BENZODIAZEPIN-4-ONES AND THE USE THEREOF 257. (WO 97/33904) DEATH DOMAIN CONTAINING RECEPTORS 258. (WO 97/33887) SPIROCYCLE INTEGRIN INHIBITORS 259. (WO 97/33613) PARASITE-DERIVED ANTI-INFLAMMATORY IMMUNOMODULATORY PROTEIN 260. (WO 97/30991) NOVEL SUBSTITUTED N-METHYL-N-(4-(4-(1H-BENZIMIDAZOL-2-YL)[1,4]DIAZEPAN-1-YL)-2-(ARYL)BUTYL)BENZAMIDES USEFUL FOR THE TREATMENT OF ALLERGIC DISEASES 261. (WO 97/30990) NOVEL SUBSTITUTED N-METHYL-N-(4-(PIPERIDIN-1-YL)-2-(ARYL)BUTYL)BENZAMIDES USEFUL FOR THE TREATMENT OF ALLERGIC DISEASES 262. (WO 97/30989) NOVEL SUBSTITUTED N-METHYL-N-(4-(4-(1H-BENZIMIDAZOL-2-YL-AMINO) PIPERIDIN-1-YL)-2-(ARYL)BUTYL)BENZAMIDES USEFUL FOR THE TREATMENT OF ALLERGIC DISEASES 263. (WO 97/30079) PEPTIDE ANTAGONISTS OF CELLULAR MITOGENESIS AND MOTOGENESIS AND THEIR THERAPEUTIC USE 264. (WO 97/30069) 17-BETA-CYCLOPROPYL(AMINO/OXY) 4-AZA STEROIDS AS ACTIVE INHIBITORS OF TESTOSTERONE 5-ALPHA-REDUCTASE AND C17-20-LYASE 265. (WO 97/29775) COMPOSITIONS COMPRISING A CYCLOOXYGENASE-2 INHIBITOR AND A LEUKOTRIENE B 4 RECEPTOR ANTAGONIST 266. (WO 97/29079) NOVEL COMPOUNDS AND PHARMACEUTICAL USE THEREOF 267. (WO 97/28190) CYTOKINE ANTAGONISTS AND AGONISTS 268. (WO 97/24373) MONOCLONAL ANTIBODY ANTAGONISTS TO HAEMOPOIETIC GROWTH FACTORS 269. (WO 97/23480) NOVEL INTEGRIN RECEPTOR ANTAGONISTS 270. (WO 97/22604) NOVEL SUBSTITUTED 4-(1H-BENZIMIDAZOL-2-YL)[1,4]DIAZEPANES USEFUL FOR THE TREATMENT OF ALLERGIC DISEASES 271. (WO 97/21732) DESIGN OF HORMONE-LIKE ANTIBODIES WITH AGONISTIC AND ANTAGONISTIC FUNCTIONS 272. (WO 97/21702) 3-BENZYLAMINOPYRROLIDINES AND -PIPERIDINES AS TACHYKININ RECEPTOR ANTAGONISTS 273. (WO 97/21445) VASCULAR IRRIGATION SOLUTION AND METHOD FOR INHIBITION OF PAIN, INFLAMMATION, SPASM AND RESTENOSIS 274. (WO 97/20062) IL-12 P40 SUBUNIT FUSION POLYPEPTIDES AND USES THEREOF 275. (WO 97/19074) SUBSTITUTED 4-(1H-BENZIMIDAZOL-2-YL-AMINO)PIPERIDINES USEFUL FOR THE TREATMENT OF ALLERGIC DISEASES 276. (WO 97/19059) NOVEL SUBSTITUTED ARYL COMPOUNDS USEFUL AS MODULATORS OF ACETYLCHOLINE RECEPTORS 277. (WO 97/16442) SUBSTITUTED PYRIDYL PYRROLES, COMPOSITIONS CONTAINING SUCH COMPOUNDS AND METHODS OF USE 278. (WO 97/16202) CYTOKINES AND THEIR USE IN TREATMENT AND/OR PROPHYLAXIS OF BREAST CANCER 279. (WO 97/16159) ENHANCED ANTI-INFLAMMATORY ORAL COMPOSITION CONTAINING H 2 RECEPTOR ANTAGONIST AND ANTIMICROBIAL OILS 280. (WO 97/15298) COMBINATION OF LTD, RECEPTOR ANTAGONISTS WITH GLUCOCORTICOSTEROIDS 281. (WO 97/14671) CYCLOPENTYL TACHYKININ RECEPTOR ANTAGONISTS 282. (WO 97/13751) INDOLE CARBAMATES AS LEUKOTRIENE ANTAGONISTS 283. (WO 97/13514) NK-1 RECEPTOR ANTAGONISTS FOR PREVENTION OF NEUROGENIC INFLAMMATION IN GENE THERAPY 284. (WO 97/09046) COMPOUNDS AND METHODS 285. (WO 97/07135) BINDING OF OSTEOGENIC PROTEIN-I (OP-1) AND ANALOGS THEREOF TO THE CELL SURFACE RECEPTOR ALK-1 AND ANALOGS THEREOF)

With respect to clinical and pre-clinical trial development see Antibody Therapeutics Production, Clinical Trials, and Strategic Issues, By Rathin C. Das, Ph.D., M.B.A. & K. John Morrow, Jr., Ph.D., D&MD Publications October 2001, Chapter 6.

### Entity Associated and Entity Specific Markers

The term marker is used broadly to refer to any ligand or binding site for a “targeting” or an “effector” moiety of a multispecific ligand or antibody of the invention and is primarily used herein to refer to ligands which are the target of a “targeting” moiety (most often though not exclusively referred to herein as a first ligand binding moiety).

The literature is replete with examples of such markers, as well as antibodies which recognize them. With respect to cancer markers and immune cell markers, etc. many of these are referred to in Cancer: Principles and Practice of Oncology 6^{th} Ed. De Vita et al. Eds Lippincott 2001 and some are summarized at pages 309-311, 3197.

With respect to markers for osteoclasts and antibodies that bind thereto see for example Endocrinology 1989 August; 125(2): 630-7; Endocrinology 1990 December: 127(6): 3215-21; Lab Invest April; 60(4): 532-8; Calcif Tissue Int 1998 August; 63(2): 148-53. Such markers could be used for example to target RANK (associated with bone resorption etc.) on osteoclasts using a relatively low affinity second ligand binding moiety.

Other target applications of multispecific ligands of the invention include particularly receptors associated with angiogenesis (e.g. VEGFRs 1,2,3) such as KDR, FLK-1 and FLT-1, and various cancers cell types e.g. HER-2 and EGF-R, including FGF-R, PDGF-R, Tek and Tie2. Numerous other examples are referred to specifically and through references to the literature herein cited. Suitable markers for many types of target entities e.g. cells bearing such receptors are referred to or referenced herein or described in various subject reviews and texts herein cited, in connection with one or more aspects and embodiments of the invention described in this application, and many others are known to those skilled in the art and described in the literature including antibodies.

With respect to binding to biologic effector ligands the multispecific ligand may comprise a recombinantly produced receptor for such ligand.

As the invention contemplates that the multispecific ligands herein may be used for cancer, it is contemplated that combination therapies with chemotherapeutic and biotherapeutic agents may be used to advantage. Such agents are well known to those skilled in the art and include for example, alkylating agents, cisplatin and its analogues, antimetabolites, topoisomerase interactive agents, antimicrotubule agents, interferons, interleukins, hormonal therapeutics, differentiation agents, antiangiogenesis agents (see Cancer: Principles and Practice of Oncology 6^{th} Ed. De Vita et al. Eds Lippincott 2001 pp. 335-517).

With respect to ligands involved in mediating apoptosis see also WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) 1N SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034

Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WO0116170 NOVEL CARD PROTEINS INVOLVED IN CELL DEATH REGULATION

As stated above, in a related but also independent aspect, the invention contemplates a method of screening for an antibody which preferentially binds to a ligand when bound to a first receptor relative to another second receptor by screening for antibodies (e.g. by phage display, ribosome display, etc.) which bind to the ligand e.g. a cytokine, when bound in situ to the first receptor, and selecting among them those that bind to the ligand e.g. cytokine but do not bind (subtractive screening) or bind with lesser affinity when bound to the cytokine to the second receptor, as well as to antibodies and multifunctional ligands created by this method (see also U.S. Pat. No. 6,046,048 and WO 99/12973 and references cited therein with respect to TNF family of receptors). Variations in the extracellular domains of such receptors are known and can be ascertained by methods known to those skilled in the art. Accordingly the invention is directed to an antibody characterized in that it binds to an epitope on the ligand which permits the ligand to bind, while the antibody is bound to it, to a first receptor but not a second receptor. In a preferred embodiment both are cell surface receptors. In a preferred embodiment the ligand is a natural ligand, preferably a growth factor, cytokine or chemokine. In another embodiment one of the receptors is a soluble receptor. The invention is also directed to a method of evaluating the pleiotropic effects of a natural ligand by administering the said antibody including antigen binding fragments thereof and MRUs) and monitoring its effects. The invention contemplates that this antibody is a first or second moiety of a multifunctional ligand disclosed herein. Examples of receptors include the classes of VEGF receptors (see also 1: Sheppard D. Integrin-mediated activation of transforming growth factor-beta(1) in pulmonary fibrosis. Chest. 2001 July; 120(1 Suppl):S49-53. Chow D, Ho J, Nguyen Pham T L, Rose-John S, Garcia K C. In vitro reconstitution of recognition and activation complexes between interleukin-6 and gp130. Biochemistry. 2001 Jun. 26; 40(25): 7593-603. Kotenko S V, Izotova L S, Mirochnitchenko O V, Esterova E, Dickensheets H, Donnelly R P, Pestka S. Identification, cloning, and characterization of a novel soluble receptor that binds IL-22 and neutralizes its activity. J. Immunol. 2001 Jun. 15; 166(12): 7096-103. Gustin S E, Church A P, Ford S C, Mann D A, Carr P D, Ollis D L, Young I G. Expression, crystallization and derivatization of the complete extracellular domain of the beta(c) subunit of the human IL-5, IL-3 and GM-CSF receptors. Eur J. Biochem. 2001 May; 268(10): 2905-11. McCall A M, Shahied L, Amoroso A R, Horak E M, Simmons H H, Nielson U, Adams G P, Schier R, Marks J D, Weiner L M. Increasing the affinity for tumor antigen enhances bispecific antibody cytotoxicity. J. Immunol. 2001 May 15; 166(10): 6112-7. Piehler J, Roisman L C, Schreiber G. New structural and functional aspects of the type I interferon-receptor interaction revealed by comprehensive mutational analysis of the binding interface. J Biol. Chem. 2000 Dec. 22; 275(51): 40425-33. 7: Wiesmann C, Muller Y A, de Vos A M. Ligand-binding sites in Ig-like domains of receptor tyrosine kinases. J Mol. Med. 2000; 78(5): 247-60. 8: Born T L, Smith D E, Garka K E, Renshaw B R, Bertles J S, Sims J E., Protein, Nucleotide Identification and characterization of two members of a novel class of the interleukin-1 receptor (IL-1R) family. Delineation of a new class of IL-1R-related proteins based on signaling. J Biol. Chem. 2000 Sep. 29; 275(39): 29946-549: Xia X Z, Treanor J, Senaldi G, Khare S D, Boone T, Kelley M, Theill L E, Colombero A, Solovyev I, Lee F, McCabe S, Elliott R, Miner K, Hawkins N, Guo J, Stolina M, Yu G, Wang J, Delaney J, Meng S Y, Boyle W J, Hsu H., Protein, Nucleotide TACl is a TRAF-interacting receptor for TALL-1, a tumor necrosis factor family member involved in B cell regulation. J Exp Med. 2000 Jul. 3; 192(1): 137-43. 10: Kumaran J, Colamonici O R, Fish E N. Structure-function study of the extracellular domain of the human type I interferon receptor (IFNAR)-1 subunit. J Interferon Cytokine Res. 2000 May; 20(5): 479-85. 11: Lu D, Kussie P, Pytowski B, Persaud K, Bohlen P, Witte L, Zhu Z. Identification of the residues in the extracellular region of KDR important for interaction with vascular endothelial growth factor and neutralizing anti-KDR antibodies. J Biol. Chem. 2000 May 12; 275(19): 14321-30. 12: Bowie A, O'Neill L A. The interleukin-1 receptor/Toll-like receptor superfamily: signal generators for pro-inflammatory interleukins and microbial products. J Leukoc Biol. 2000 April; 67(4): 508-14. 13: Kumar S, McDonnell P C, Lehr R, Tierney L, Tzimas M N, Griswold D E, Capper E A, Tal-Singer R, Wells G I, Doyle M L, Young P R., Protein, Nucleotide, OMIM Identification and initial characterization of four novel members of the interleukin-1 family. J Biol. Chem. 2000 Apr. 7; 275(14): 10308-14. 14: Fujio K, Nosaka T, Kojima T, Kawashima T, Yahata T, Copeland N G, Gilbert D J, Jenkins N A, Yamamoto K, Nishimura T, Kitamura T., Protein, Nucleotide Molecular cloning of a novel type 1 cytokine receptor similar to the common gamma chain. Blood. 2000 Apr. 1; 95(7): 2204-10. 15: Touw I P, De Koning J P, Ward A C, Hermans M H. Signaling mechanisms of cytokine receptors and their perturbances in disease. Mol Cell Endocrinol. 2000 Feb. 25; 160(1-2): 1-9. Review. 16: Chritton S L, Sheng M.CYRL, a novel cytokine receptor-like protein expressed in testis, lung, and spleen. Biochem Biophys Res Commun. 2000 Jan. 27; 267(3):697-702. 17: Li H, Chen J, Huang A, Stinson J, Heldens S, Foster J, Dowd P, Gurney A L, Wood W I. Free in PMC, Protein, Nucleotide, OMIM Cloning and characterization of IL-17B and IL-17C, two new members of the IL-17 cytokine family. Proc Natl Acad Sci USA. 2000 Jan. 18; 97(2):773-18: Gary-Gouy H, Bruhns P, Schmitt C, Dalloul A, Daeron M, Bismuth G. The pseudo-immunoreceptor tyrosine-based activation motif of CD5 mediates its inhibitory action on B-cell receptor signaling. J Biol. Chem. 2000 Jan. 7; 275(1): 548-56. 19: Wiesmann C, Ultsch M H, Bass S H, de Vos A M., Protein, Structure Crystal structure of nerve growth factor in complex with the ligand-binding domain of the TrkA receptor. Nature. 1999 Sep. 9; 401(6749): 184-8. 20: Liu Y, Cruikshank W W, O'Loughlin T, O'Reilly P, Center D M, Kornfeld H. Identification of a CD4 domain required for interleukin-16 binding and lymphocyte activation. J Biol. Chem. 1999 Aug. 13; 274(33): 23387-95. 21: Donnelly R P, Dickensheets H, Finbloom D S. The interleukin-10 signal transduction pathway and regulation of gene expression in mononuclear phagocytes. J Interferon Cytokine Res. 1999 June; 19(6): 563-73. Review. 22: Kim H, Baumann H. Dual signaling role of the protein tyrosine phosphatase SHP-2 in regulating expression of acute-phase plasma proteins by interleukin-6 cytokine receptors in hepatic cells. Mol Cell Biol. 1999 August; 19(8): 5326-38. 23: Kernebeck T, Pflanz S, Muller-Newen G, Kurapkat G, Scheek R M, Dijkstra K, Heinrich P C, Wollmer A, Grzesiek S, Grotzinger J., Protein, Structure The signal transducer gp130: solution structure of the carboxy-terminal domain of the cytokine receptor homology region. Protein Sci. 1999 January; 8(1): 5-12. 24: Ernst M, Novak U, Nicholson S E, Layton J E, Dunn A R. The carboxyl-terminal domains of gp130-related cytokine receptors are necessary for suppressing embryonic stem cell differentiation. Involvement of STAT3. J Biol. Chem. 1999 Apr. 2; 274(14): 9729-37. 25: Hibi M, Hirano T. Signal transduction through cytokine receptors. Int Rev Immunol. 1998; 17(1-4): 75-102. Review. 26: Staunton D, Hudson K R, Heath J K. The interactions of the cytokine-binding homology region and immunoglobulin-like domains of gp130 with oncostatin M: implications for receptor complex formation. Protein Eng. 1998 November; 11(11): 1093-102).

With respect to markers that are useful for differentiating between various populations and sub-populations of cells, see also Human IL-18 Receptor and ST2L Are Stable and Selective Markers for the Respective Type 1 and Type 2 Circulating Lymphocytes, Woon Ling Chan, Nada Pejnovic, Christine A. Lee, and Nadia A. Al-Ali, J Immunol 2001; 167 1238-1244; CD4+ CD25 high Regulatory Cells in Human Peripheral Blood, Clare Baecher-Allan, Julia A. Brown, Gordon J. Freeman, and David A. Hafler, J Immunol 2001; 167 1245-1253.

In another aspect the invention is directed to multifunctional ligand comprising at least a first moiety which specifically binds to a ligand on the surface of a virus particle that is capable of infecting a mammalian and particularly a human cell including a cancer cell (excluding viruses which are known for use in gene therapy) and is preferably selected from the group consisting viruses which infect substantial populations of individuals including for example influenza virus and at least a second moiety which specifically recognizes a cancer cell, in one embodiment preferably a marker present on multiple different cancer types, especially cancer types that are individually or collectively most prevalent in the general population. In one embodiment such multifunctional ligand is a bispecific, trispecific or tetraspecific antibody. The invention contemplates that such a multifunctional ligand may be used to target such viruses to tumors in a manner which preferentially kills the cancer cells either through the action of the virus and/or by causing the immune response to the virus or virus infected cell to preferentially (relative to non-cancer cells) target the cancer cell for ablation. The invention is also directed to a method of treating cancer by retargeting virus with which an individual is otherwise infected to the cancer cell e.g. influenza. The invention also contemplates that the multifunctional ligand includes one or more effector moieties which assist in killing the virus and/or cancer cell or directing immune cells to the virus and/or cancer cell, if and when present in the individual, for example a moiety which specifically binds to such immune cell e.g. a T cell, as discussed above. Accordingly, the invention also contemplates that such multifunctional ligand may be used to treat influenza virus infections and secondarily to act prophylactically as a sentinel against any cancer cells which might develop during the course of the viral infection or a period of immune suppression or increased susceptibility to infection or cancer, including for example, as experienced by individuals with a particular immune suppressive disorder or condition or under treatment with immune suppressive drugs, individuals at risk for cancer or recurrence of a cancer, individuals of a particular age group, individuals experiencing a period of unusual stress which increases their susceptibility to disease or infection. The invention also contemplates that such a moiety is used in concert with prior immunization against the virus, so that the augmented immune response to the virus benefits the treatment of the cancer cells (see for example U.S. Pat. No. 6,169,175 and the art cited therein). The invention also directed to such a virus (excluding viruses known for use in gene therapy applications e.g. adenovirus) which is engineered to expresses on its surface a cancer targeting moiety such as a scFv (see for example EP 1038967, WO 94/10323 and the art cited therein). The invention is also directed to a method of identifying the expression or over-expression of cell surface markers associated with infection by such a virus, by subtractive screening relative to markers also expressed on non-infected such cells, for example using phage display or the like. Such markers may be used for vaccine-type or other immunotherapeutic strategies. Anti-virus markers including influenza virus markers and methods of identifying new such markers are well known in the art (see for example U.S. Pat. No. 5,589,174) (see also The role of the antibody response in influenza virus infection., Gerhard W., Curr Top Microbiol Immunol 2001; 260:171-90, Fernandez-Sesma A, Schulman J L, Moran T M. A bispecific antibody recognizing influenza A virus M2 protein redirects effector cells to inhibit virus replication in vitro. J Virol. 1996 July; 70(7): 4800-4; Todorovska A, Roovers R C, Dolezal O, Kortt A A, Hoogenboom H R, Hudson P J. Design and application of diabodies, triabodies and tetrabodies for cancer targeting. J Immunol Methods. 2001 Feb. 1; 248(1-2): 47-66., Staerz U D, Yewdell J W, Bevan M J. Hybrid antibody-mediated lysis of virus-infected cells. Eur J. Immunol. 1987 April; 17(4): 571-4; Fernandez-Sesma A, Schulman J L, Moran T M. A bispecific antibody recognizing influenza A virus M2 protein redirects effector cells to inhibit virus replication in vitro. J Virol. 1996 July; 70(7): 4800-4.)

The invention is also directed to a multifunctional ligand having at least a tumor cell targeting moiety and a moiety which binds to a tumor antigen which is shed from a cancer cell. In a preferred embodiment, the tumor antigen binding moiety preferably does not recognize the portion of the antigen which is most immunogenic and leaves that portion exposed for recognition by the immune system. The invention contemplates generating such preferred antibody or fragment thereof by using an immune complex between an antibody that binds to such immunogenic portion and the antigen as a target for phage display or generation or polyclonal sera. The invention also contemplates identifying antibodies which recognize immunogenic portions of the antigen by screening patient sera for antibodies which recognize the antigen. The invention also contemplates that such multifunctional ligand includes one or more effector moieties which assist in killing the cancer cell or directing immune cells to the cancer cell, for example a moiety which specifically binds to such immune cell e.g. a T cell receptor, as discussed above.

Without limiting the generality of or applicability of the foregoing, and without being limited by or limiting the scope of the claims, various embodiments of the invention may be summarized for ease of reference as follows: 1. A multispecific ligand comprising at least a first ligand binding moiety which preferentially binds with a first affinityⁱⁱⁱ to a first ligand having a first biodistribution* and at least a second ligand binding moiety which preferentially binds with a second affinity to a second ligand having a second biodistribution which is different^{iv} from that of the first ligand, and wherein the affinity of first and second ligand binding moieties are selected to bias the biodistribution of the multispecific ligand. 2. A multispecific ligand according to paragraph 1, wherein said multifunctional ligand comprises one or more ligand binding moieties which are antibodies. 3. A multispecific ligand according to paragraph 1 or 2, wherein the affinity of said first ligand binding moiety for the first ligand is higher than the affinity of the second ligand binding moiety for the second ligand so as to bias the biodistribution of the multispecific ligand in favor of the first ligand. 4. A multispecific ligand according to paragraph 3, wherein the first and second ligands have overlapping biodistributions.^{v} 5. A multispecific ligand according to paragraph 4, wherein the first ligand is a target cell population associated ligand and wherein said second ligand is present on a broader population of cells and wherein the biodistribution of the multispecific ligand is skewed in favour of the target cell population. 6. A multispecific ligand according to paragraph 1 or 5, wherein said first ligand is a marker associated with* one or more specific cell populations, infectious or parasitic agents, diseased cells, or disease associated* cells, optionally one of specific ligands herein mentioned or referenced or known to those skilled in the art. 7. A multispecific ligand according to paragraph 6, wherein said marker is a specific biological structure. 8. A multispecific ligand according to paragraph 6, wherein said marker is a specific receptor or receptor ligand. 9. A multispecific ligand according to paragraph 6, wherein said marker is a specific antigen. 10. A multispecific ligand according to paragraph 6, wherein said marker is a specific epitope. 11. A multispecific ligand according to paragraph 6, wherein said marker is a CD marker. 12. A multispecific ligand according to paragraph 6, wherein said marker is associated with a cancer cell or pre-cancerous cell. 13. A multispecific ligand according to paragraph 6, wherein said marker is associated with an autoimmune disorder or rheumatic disease. 14. A multispecific ligand according to paragraph 6, wherein said marker is associated with a specific tissue type. 15. A multispecific ligand according to paragraph 6, wherein said marker is associated with a specific organ. 16. A multispecific ligand according to paragraph 6, wherein said marker is associated with a cell or tissue of specific origin or class. 17. A multispecific ligand according to paragraph 6, wherein said marker is an MHC-peptide complex. 18. A multispecific ligand according to paragraph 6, wherein said marker is associated with a cell surface immunoglobulin. 19. A multispecific ligand according to paragraph 5 or 6, wherein said second ligand is a receptor, family of receptors or one or more particular receptor family members, optionally one of those specific receptors herein mentioned or referenced or known to those skilled in the art. The invention contemplates targeting any receptor present on any population of entities for which there is an entity-associated marker. 20. A multispecific ligand according to paragraph 19, wherein said second ligand is a cell surface receptor chosen from a group comprising tyrosine kinase type receptors, serine kinase type receptors, heterotrimeric G-protein coupled receptors, receptors bound to tyrosine kinase, TNF family receptors, notch family receptors, guanylate cyclase types, tyrosine phosphatase types, decoy receptors, and adhesion receptors, optionally one of the specific receptors herein mentioned or referenced or known to those skilled in the art. 21. A multispecific ligand according to paragraph 19, wherein said receptor requires cross-linking for biological activity. 22. A multispecific ligand according to paragraph 5 or 6, wherein said second ligand is a cell surface receptor and wherein said second ligand binding moiety blocks said receptor. 23. A multispecific ligand according to paragraph 1, 5 or 6, wherein said second ligand is a receptor ligand and wherein said second ligand binding moiety blocks interaction with the corresponding receptor. 24. A multispecific ligand according to paragraph 5 or 6, wherein said second ligand is a cell surface receptor which initiates a signal transduction and wherein said second ligand binding moiety effects a signal transduction. 25. A multispecific ligand according to paragraph 5, 6 or 19, wherein said antibody comprises a first VH* which preferentially recognizes said first ligand and a second VH which preferentially recognizes said second ligand. 26. A multispecific ligand according to paragraph 25, wherein at least one of said first and second VHs require the cooperation of a VL for binding to their respective ligands. 27. A multispecific ligand according to paragraph 25, comprising a first VL associated with said first VH and a second VL associated with said second VH and wherein both said first and second VHs require the cooperation of a VL for binding to the first and second ligands, respectively, and wherein said first and second VLs are the same^{vi} or functionally interchangeable*. 28. A multispecific ligand according to paragraph 25 or 27, wherein said bispecific antibody is a four chain antibody. 29. A multispecific ligand according to paragraph 28, wherein said bispecific antibody is a minibody, F(ab′)₂ or antibody devoid of a CH3 domain. 30. A multispecific ligand according to paragraph 25, wherein said bispecific antibody is a diabody. 31. A multispecific ligand according to paragraph 25 or 27, wherein said bispecific antibody is devoid of light chains. 32. A multispecific ligand according to paragraph 31, wherein said bispecific antibody comprises a pair of disulfide linked heavy chains or heavy chain portions each comprising at least a VH region, a hinge region and preferably, at least a portion of an Fc region at the carboxy terminus of the hinge region. 33. A multispecific ligand according to paragraph 31, wherein said bispecific antibody comprises a pair of VHs linked via a polypeptide linker. 34. A multispecific ligand according to paragraph 3, 5, 6 or 19 wherein the affinity of the first ligand binding moiety for the first ligand is at least approximately^{vii}, one, two, three, four, five, six, seven or eight orders of magnitude greater than the affinity of said second ligand binding moiety for the second ligand. 35. A composition comprising a multispecific ligand in a pre-determined dosage, said multispecific ligand comprising a first ligand binding moiety which preferentially binds with a pre-selected first affinity to a first ligand having a first biodistribution and a second ligand binding moiety which preferentially binds with a pre-selected affinity to a second ligand having a second biodistribution, which is different from that of the first ligand, and wherein the affinity of the first and second ligand binding moieties are selected to bias the biodistribution of the multispecific ligand in favor of a selected location of one or both of the ligands^{viii} such that a desired proportion of the dosage is delivered to the selected location. 36. A multispecific ligand according to paragraph 1 or 35, wherein the biodistributions of said first and second ligands overlap^{ix} and wherein the affinities of the first and second ligand binding moieties are selected to bias the biodistribution of the multispecific ligand in favour of a target cell population on which both first and second ligands are bioavailable for recognition by the first and second ligand binding moieties, relative to one or more non-target cell populations. 37. A multispecific ligand according to paragraph 36, wherein the affinity of first ligand binding moiety for the first ligand is at least, approximately, one, two, three, four, five, six, seven or eight orders of magnitude greater than the affinity of the second ligand binding moiety for the second ligand. 38. A multispecific ligand according to paragraph 36 or 37, wherein first and second ligands are bioavailable for contemporanous* recognition by the first and second ligand binding moieties. 39. A method of controlling the biodistribution of a ligand which interacts with a target ligand present on a heterogenous population of ligand bearing entities, said method comprising using a multispecific ligand comprising at least a first ligand binding moiety which preferentially* binds with a pre-selected* first affinity^{x} to at least a first ligand associated with a target sub-population of said heterogeneous population on which said first ligand and target (second) ligand are bioavaible for contemporaneous recognition and a second ligand binding moiety which preferentially binds with a pre-selected lesser affinity to said target ligand, and wherein the affinity of first and second ligand binding moieties are selected to bias the biodistribution of the multispecific ligand in favor of said target sub-population of ligand bearing entities. 40. A method of testing the biological effects of limiting the biodistribution of a ligand which interacts with a target ligand present on a heterogenous population of ligand bearing entities, said method comprising the step of administering a multispecific ligand comprising at least a first ligand binding moiety which preferentially* binds with a pre-selected* first affinity^{xi} to at least a first ligand associated with a target sub-population of said population of ligand bearing entities on which said first ligand and target (second) ligand are bioavaible for contemporanous recognition and a second ligand binding moiety which preferentially binds with a pre-selected lesser affinity to said target ligand, and wherein the affinity of first and second ligand binding moieties are selected to bias the biodistribution of the multispecific ligand in favor of said target sub-population of ligand bearing entities. 41. A multispecific ligand which preferentially binds to a target ligand on a selected sub-population of a heterogeneous population of cells bearing the target-ligand, the multispecific ligand comprising a first ligand binding moiety which preferentially binds to a cell sub-population associated ligand and a second ligand binding moiety which binds to the target-ligand, said first ligand binding moiety having an affinity for the sub-population associated ligand that is higher than the affinity of the second ligand binding moiety for the target ligand. 42. A multispecific ligand according to paragraph 41, wherein the affinity of said first ligand binding moiety for the cell sub-population associated ligand is approximately, one, two, three, four, five, six, seven or eight orders of magnitude greater than the affinity of said second ligand binding moiety for said target ligand. 43. A multispecific ligand according to paragraph 42, wherein said target ligand is a receptor or a receptor ligand^{xii}. 44. A multispecific ligand according to paragraph 42, wherein at least one of said first or second ligand binding moieties comprises an antibody heavy chain or functional portion(s) thereof including a VH or fragment thereof and an antibody light chain or functional portion(s) thereof including a VH or fragment thereof. 45. A method of selectively exerting a biological effect mediated through binding a target-ligand on a selected sub-population of a population of cells bearing the target-ligand, the method comprising the step of exposing the cells to a multispecific ligand comprising a first ligand binding moiety which preferentially binds to a cell sub-population associated ligand and a second ligand binding moiety which binds to the target ligand, said first ligand moiety having an affinity for the sub-population associated ligand that is higher than the affinity of the second ligand binding moiety for the target ligand. 46. A method according to paragraph 45, wherein the affinity of said first ligand binding moiety for the cell sub-population associated ligand is at least approximately, one, two, three, four, five, six, seven or eight orders of magnitude greater than the affinity of said second ligand binding moiety for said target ligand. 47. A method according to paragraph 45, wherein said target ligand is a receptor or a receptor ligand. 48. A method according to paragraph 45 wherein at least one of said first or second ligand binding moieties is an antigen binding fragment^{xiii} of an antibody. 49. A method of testing or controlling the biological effects of a ligand binding molecule by circumscribing its ability to bind to a diverse population of cells bearing a complementary target ligand, said method comprising using said ligand binding molecule together with a different ligand binding molecule which preferentially binds to another target ligand which is exclusively or preferentially associated with one or more sub-population(s) of said population of cells, and wherein the biological effects of said ligand binding molecule are controlled through prior association of said ligand binding molecule with said other ligand binding molecule to form a multispecific ligand and through the affinity of at least one of said ligand binding molecules being pre-selected to limit the propensity of said ligand binding molecule to bind to cells within said population of cells which do not preferentially express said other target ligand. 50. A method according to paragraph 49, wherein the affinity of said ligand binding molecule for said complementary target ligand is less than the affinity of the other ligand binding molecule for the other target ligand. 51. A method according to paragraph 49 or 50, wherein at least one of said first and second ligand binding molecules is an antibody*. 52. A method according to paragraph 49 wherein said first ligand binding molecule is an entity which exerts a biologic effect and said second ligand binding molecule is a multispecific ligand comprising a first ligand binding moiety that binds to a cell sub-population associated ligand and a second ligand binding moiety which binds to said entity. 53. A method according to paragraph 52 wherein said second ligand binging moiety is an antibody which binds to a pre-selected epitope on said entity and wherein the epitope of said second ligand binding moiety is selected on the basis of its proximity to a ligand binding portion of said entity such that the entity when bound to said multifunctional ligand has an affinity for said ligand which is less than the affinity of said first ligand binding moiety for said cell sub-population associated ligand. 54. A multispecific ligand according to paragraph 12, wherein said second ligand is a IL-8 receptor, a CCR7 receptor, a FAS receptor, or a CXCR4 receptor. 55. A multispecific ligand according to paragraph 6, wherein said marker is associated with an immune cell that is susceptible to viral infection. 56. A multispecific ligand according to paragraph 55, wherein said marker is CD4. 57. A multispecific ligand according to paragraph 55 or 56, wherein said second ligand is a CCR5 or CXCR4 receptor. 58. A multifunctional ligand according to paragraph 52, wherein said entity is a biologic effector ligand. 59. A multispecific ligand according to paragraph 36, wherein the affinities of said first and second ligand binding moieties are both selected to limit their individual ability to bind to the first and second ligands, respectively, and wherein their combined functional affinity biases the distribution of the multifunctional in favour of said target cell population. 60. An antibody which binds to an epitope on an entity which exerts a biologic effect via a binding interaction with a target ligand, said epitope being proximal to the binding site of said entity for the target ligand, such that the antibody bound to the entity reduces the affinity of the entity for its ligand without precluding its functional binding activity vis-à-vis said ligand. 61. A multispecific ligand comprising a first ligand binding moiety which binds with a pre-selected affinity to a target entity associated ligand and a second ligand binding moiety which binds with pre-selected affinity to an epitope on a biologic effector ligand which exerts a biologic effect via a binding interaction with a target ligand, said epitope being proximal to the binding site of said biologic effector ligand for the target ligand, such that the second ligand moiety bound to the biologic effector ligand reduces the affinity of the molecule for the target ligand without precluding its functional binding activity vis-à-vis said ligand and wherein said target ligand is present on a diverse population of entities consisting of the target entity and one or more non-target entities and wherein the affinity of the first ligand binding moiety for the target entity associated ligand is greater than that of the biologic effector ligand for the target ligand when bound to second ligand molecule, and wherein the affinity of the first ligand binding moiety is selected to bias the biodistribution of said biologic effector ligand in favour of the target entity relative to the non-target entit(ies). 62. A multispecific ligand according to paragraph 61, wherein first ligand binding moiety comprises a light chain linked to an antibody heavy chain portion comprising at least a VH, CH1 domain, hinge region and preferably at least a truncated Fc portion and said second ligand binding moiety comprises at least a VL linked to a heavy chain portion, optionally through a disulfide bond^{xiv} and wherein the heavy chain portion of said second ligand binding moiety is devoid of CH1 domain, and comprises a hinge region and preferably at least a truncated Fc portion, and wherein said heavy chain portions are linked via their respective hinge regions and optionally wherein the respective hinge regions are wholly or partially substituted or supplemented by a another linkage pair^{xv} e.g. a leucine zipper. 63. A multispecific ligand according to paragraph 12, wherein said second ligand is a marker associated with a lymphatic endothelial cell. 64. A multispecific ligand comprising a first ligand binding moiety which preferentially binds to a lymphatic endothelial cell associated marker and a second moiety which exerts a biologic function^{xvi}, optionally a therapeutic function, optionally an immune function*, optionally at least one of an immunizing^{xvii} function, a tolerizing function, a neutralizing function, an immune mediating function, and immune modulating function^{xix}, in relation* to an independent^{xx} entity, preferably within the lymphatic system. 65. A multispecific ligand according to paragraph 62, wherein the marker is selected to limit the ability of said endothelial cell to internalize said multispecific ligand. 66. A multifunctional ligand having, at least, a first portion which binds to a lymphatic vessel associated ligand and a second portion comprising an immune function exerting moiety. 67. A multifunctional ligand as defined in paragraph 66, wherein said first portion is an antibody. 68. A multifunctional ligand as defined in paragraph 66, wherein said immune function exerting moiety binds to a target ligand. 69. A multifunctional ligand as defined in paragraph 67, wherein said immune function exerting moiety comprises an antibody. 70. A multifunctional ligand as defined in paragraph 68, wherein said immune function exerting moiety comprises an antibody. 71. A multifunctional ligand as defined in paragraph 68, wherein said immune function exerting moiety binds to a ligand selected form the group consisting of CCR5, CTLA-4, LFA-1, ICAM-1, CD2, CD3, CD4, CD22, CD40, CD44; CD80, CD86, CD134 and CD154. 72. A multifunctional ligand as defined in paragraph 70, wherein said first portion binds to LYVE-1 or podoplantin. 73. A multifunctional ligand as defined in paragraph 70, wherein said immune function exerting moiety comprises an anti-idiotypic antibody. 74. A multifunctional ligand as defined in paragraph 73, wherein said anti-idiotypic antibody binds to an autoimmune antibody. 75. A multifunctional ligand as defined in paragraph 73, wherein said anti-idiotypic antibody mimics a cell surface expressed tumor antigen or a viral antigen. 76. A multifunctional ligand as defined in paragraph 70, wherein said immune function exerting moiety binds to a diseased cell. 77. A multifunctional ligand as defined in paragraph 70, wherein said immune function exerting moiety binds to an infectious agent or parasite. 78. A multifunctional ligand as defined in paragraph 76, wherein said diseased cell is a cancer cell. 79. A multifunctional ligand as defined in paragraph 76, wherein said diseased cell is a virally infected cell. 80. A multifunctional ligand as defined in paragraph 68, wherein said immune function exerting moiety binds to an immune cell. 81. A multifunctional ligand as defined in paragraph 69, 76 or 80 wherein said immune function exerting moiety binds with greater functional affinity to its target ligand than said first portion binds to its target ligand. 82. A multifunctional ligand as defined in paragraph 69, 76 or 80 wherein said immune function exerting moiety binds with greater affinity to its target ligand than said first portion binds to its target ligand. 83. A multifunctional ligand as defined in paragraph 69, 76 or 80, wherein said binds with greater avidity to its target ligand than said first portion binds to its target ligand. 84. A multifunctional ligand as defined in paragraph 80, wherein immune cell is associated with an autoimmune reaction. 85. A multifunctional ligand as defined in paragraph 80, wherein said immune cell is a CCR5-expressing cell. 86. A multifunctional ligand as defined in paragraph 78 or 80, wherein said second portion comprises an internalizing antibody and a cytotoxic component. 87. A multifunctional ligand as defined in paragraph 78 or 80, which is a bispecific antibody having a monovalent first portion and a monovalent second portion. 88. A multifunctional ligand as defined in paragraph 78 or 80, which is a bispecific antibody having a divalent first portion and a divalent second portion. 89. A multifunctional ligand as defined in paragraph 78 or 80, which is a trispecific antibody having a monovalent first portion and a second portion comprising a divalent immune function exerting moiety which binds to one or more target ligands on a target diseased cell or immune cell and a monovalent anti-CD3 or anti-CD28 antibody. 90. A multifunctional ligand as defined in paragraph 78 or 80, which is a trivalent trispecific antibody having a monovalent first portion and a second portion comprising a divalent immune function exerting moiety which binds to a target ligand on a target diseased or immune cell. 91. A multifunctional ligand as defined in paragraph 78, wherein said second portion comprises a cytokine component. 92. A multifunctional ligand as defined in paragraph 78, wherein said second portion comprises a cytotoxic component. 93. A multifunctional ligand as defined in paragraph 78, wherein said second portion comprises a ligand which is capable of binding to T cells. 94. A multifunctional ligand as defined in paragraph 87, wherein said ligand is an antibody which binds to T cells. 95. A multifunctional ligand as defined in paragraph 78, wherein said second portion comprises an anti-CD3 antibody or anti-CD28 antibody. 96. A multifunctional ligand as defined in paragraph 67, wherein second portion is a cytokine component. 97. A multifunctional ligand as defined in paragraph 67, wherein second portion is an anti-CD3 antibody or an anti-CD28 antibody. 98. A multifunctional ligand as defined in paragraph 13, wherein said second portion further comprises one or more components selected from the group consisting of a cytokine component, a cytotoxic component and an anti-CD3/CD28 component. 99. A multifunctional ligand as defined in paragraph 14, wherein said second portion further comprises one or more components selected from the group consisting of a cytokine component, a cytotoxic component and an anti-CD3/CD28 component. 100. A pharmaceutical composition comprising a multifunctional ligand as defined in paragraph 101. A pharmaceutical composition comprising a plurality of different multifunctional ligands. 102. A pharmaceutical composition as defined in paragraph 101, wherein said plurality of different multifunctional ligands exert a cooperative immune effect. 103. A pharmaceutical composition as defined in paragraph 101, wherein said plurality of different multifunctional ligands comprise a multifunctional ligand as described in paragraph 76 and at least one or both of the multifunctional ligands described in paragraph 95 or 96. 104. A method of inhibiting the formation of metastasis during the course of surgical removal of a tumor comprising administering to a patient prior to surgical treatment of the tumor site, a pharmaceutical composition comprising a multifunctional ligand as described in paragraph 78. 105. An immunocytokine comprising an anti-idiotypic antibody which recognizes the paratope of an antibody which binds to a lymphatic vessel associated ligand and a cytokine fused therewith or conjugated thereto. 106. An immunocytokine as defined in paragraph 105, wherein said cytokine component comprises IL-2 or a functional fragment thereof and/or IL-12 or a functional fragment thereof. 107. An immunocytokine as defined in paragraph 42, wherein said cytokine component comprises TNF-α or a functional fragment thereof. 108. A bispecific antibody comprising an anti-idiotypic antibody which recognizes the paratope of an antibody which binds specifically to a lymphatic vessel associated ligand and an anti-CD3 antibody or an anti-CD28 antibody. 109. A multifunctional ligand according to paragraph 66 comprising one or more amino acids that are substituted for amino acids that contribute to an immunogenic epitope. 110. A multifunctional ligand having, at least, a first portion which binds to a lymphatic vessel associated ligand and a second portion comprising an independent therapeutic function exerting moiety. 111. A bispecific ligand comprising a first ligand which binds to a first target ligand and a second ligand which binds to a second target ligand, and wherein the affinity of said first ligand is selected to enable binding to the first target ligand independently of the ability of said second ligand to bind to the second target ligand and wherein the affinity of said second ligand is selected to substantially reduce the probability of its binding to the second target ligand without the first ligand binding first or substantially contemporaneously to the first target ligand. 112. A bispecific antibody comprising a first antibody component which binds to a first target ligand and a second antibody component which binds to a second target ligand, and wherein the affinity or avidity or both the affinity and avidity of said first antibody component are selected to enable binding to the first target ligand independently of the ability of said second antibody component to bind to the second target ligand and wherein the avidity or affinity or both the affinity and avidity of said second ligand are selected to substantially reduce the probability of its binding to the second target ligand without the first ligand binding first or substantially contemporaneously to the first target ligand. 113. A multispecific ligand comprising a first moiety which binds to a first target ligand and a second moiety which binds to a second target ligand, and wherein the affinity or avidity or both the affinity and avidity of said first moiety are selected to enable the first moiety to bind to the first target ligand independently of the ability of said second moiety to bind to the second target ligand and wherein the avidity or affinity or both the affinity and avidity of said second moiety are selected to substantially reduce the probability of its binding to the second target ligand without the first moiety, first or substantially contemporaneously, binding to the first target ligand. 114. A multispecific ligand according to paragraph 113, wherein both moieties bind to different target ligands on the same cell. 115. A multispecific ligand comprising a first moiety which binds to a first target ligand and a second moiety which binds to a second target ligand, and wherein the affinity or avidity or both the affinity and avidity of said first moiety and the avidity or affinity or both the affinity and avidity of said second moiety are selected to substantially reduce the probability of either moiety binding for a sufficient duration or series of durations to its respective target ligand to a accomplish a therapeutic function without the other moiety, first or substantially contemporaneously, binding to its respective target ligand 116. A multispecific ligand comprising a first moiety which specifically binds to a first target ligand on a first entity and a second moiety which specifically binds to a second target ligand on a second entity, and wherein the affinity or avidity or both the affinity and avidity of said first moiety are selected to enable the first moiety to bind to the first target ligand independently of the ability of said second moiety to bind to the second target ligand and wherein the avidity or affinity or both the affinity and avidity of said second moiety are selected to enable the second moiety to bind to the second entity in preference to the first moiety binding to the first entity when both first and second moieties are substantially contemporaneously bound to the respective first and second entities. 117. A multispecific ligand comprising a first moiety which specifically binds to a first target ligand on a first entity and a second moiety which specifically binds to a second target ligand on a second entity, and wherein the second entity binds to a third target ligand, and wherein the affinity or avidity or both the affinity and avidity of said first moiety are selected to enable the first moiety to bind to the first target ligand independently of the ability of said second moiety to bind to the second target ligand and wherein the avidity or affinity or both the affinity and avidity of said first moiety are selected to enable the first moiety to bind to the first entity in preference to the second moiety binding to the second entity when both first and second moieties are substantially contemporaneously bound to the respective first and second entities, and wherein the avidity or affinity or both the affinity and avidity of said second moiety are selected to enable the third target ligand to bind to the second entity in preference to the second moiety binding to the second entity when both said third target ligand and the second moiety are substantially contemporaneously bound to the second entity. 118. A multispecific ligand comprising at least a first ligand binding moiety which specifically binds to a first ligand having a first biodistribution and a second ligand binding moiety which specifically binds to a second ligand having a second biodistribution, and wherein the affinity of the first and second ligand binding moieties are different and selected to bias the biodistribution of the multispecific ligand, and wherein the affinity of the first ligand binding moiety for the first ligand is at least, approximately, one order of magnitude greater than that of the second ligand binding moiety for the second ligand. The affinity of the first ligand binding moiety for the first ligand is optionally at least, approximately, two orders of magnitude greater than that of the second ligand binding moiety for the second ligand. The affinity of the first ligand binding moiety for the first ligand is optionally at least, approximately, three orders of magnitude greater than that of the second ligand binding moiety for the second ligand. The affinity of the first ligand binding moiety for the first ligand is optionally at least, approximately, four orders of magnitude greater than that of the second ligand binding moiety for the second ligand. The affinity of the first ligand binding moiety for the first ligand is optionally at least, approximately, five orders of magnitude greater than that of the second ligand binding moiety for the second ligand. The affinity of the first ligand binding moiety for the first ligand is optionally at least, approximately, six orders of magnitude greater than that of the second ligand binding moiety for the second ligand. The affinity of the first ligand binding moiety for the first ligand is optionally at least, approximately, seven orders of magnitude greater than that of the second ligand binding moiety for the second ligand. The affinity of the first ligand binding moiety for the first ligand is optionally at least, approximately, eight orders of magnitude greater than that of the second ligand binding moiety for the second ligand. 119. A multispecific ligand according to paragraph 118, wherein the first ligand is present on a first target cell population and wherein said second ligand is present on a second target cell population comprising the first target cell population and wherein the biodistribution of the multispecific ligand favours the first target cell population. 120. A multispecific ligand according to paragraph 119, wherein said multispecific ligand is capable of contemporaneously binding the first and second ligands on said target population. 121. A host cell or cell free expression medium comprising one or more polynucleotides, said one or more polynucleotides comprising one or more DNA sequences, said one or more DNA sequences comprising one or more polypeptides which are sufficient to constitute a multispecific ligand as defined in any of the preceding paragraphs. 122. A kit comprising one or more polynucleotides, said one or more polynucleotides comprising one or more DNA sequences, said one or more DNA sequences encoding one or more polypeptides which are sufficient to constitute a multispecific ligand as defined in any of the preceding paragraphs. 123. A liquid medium comprising comprising one or more polypeptides which are sufficient to constitute a multispecific ligand as defined in any of the preceding paragraphs. 124. A liquid medium comprising one or more host cells, said one or more host cells comprising one or more polynucleotides, said one or more polynucleotides comprising one or more DNA sequences, said one or more DNA sequences encoding one or more polypeptides which are sufficient to constitute a multispecific ligand as defined in any of the preceding paragraphs. 125. A substantially isolated polynucleotide comprising a DNA sequence encoding a polypeptide portion of a second ligand binding moiety as defined in any of the preceding claims, said polypeptide portion comprising a VH or VL, said second ligand binding moiety having a low affinity for said second ligand. 126. A substantially isolated polynucleotide according to paragraph 125, wherein said polynucleotide is a substantially isolated expression or cloning vector. 127. A method of making a multispecific ligand as defined in any of the preceding paragraphs comprising expressing at least one polynucleotide as defined in paragraph 122 or 125. 128. A pharmaceutical composition comprising a multispecific ligand as defined in any of the preceding paragraphs and a pharmaceutically acceptable excipient. 129. A therapeutic composition comprising a multispecific ligand as defined in any of the preceding paragraphs and a pharmaceutically acceptable excipient. 130. A method of treating a disease in a mammal comprising administering a therapeutically effective amount of a multispecific ligand according to any of the preceding claims. 131. A kit comprising a plurality of different multispecific ligands as defined herein. ⁱⁱⁱ Contrasted to functional affinity which may result from avidity^{iv} see fn 8^{v} The term epitope though technically understood to be specific for a given antibody, is used to refer to antigenic determinants that are situated proximally to one another so that two antibodies will be considered to bind to the same epitope if one competitively inhibits the binding of the other through any probative competitive inhibition experiment known to those skilled in the art.^{vi} have substantially the same amino acid composition i.e. with possible exception of one or more additions, deletions or substitutions including conservative amino acid substitutions which do not substantially affect the specificity and amino acid composition of the paratope^{vii} the term approximately in the context of orders of magnitude variations in affinity refers a variability that is up to a half an order or magnitude.^{viii} having regard to their respective bioavailabilities^{ix} The term “overlap” connotes that notwithstanding the difference in distributions of the first and second ligands the first and second ligands are bioavailable for recognition on the same entity. This term and related terms, exemplified below, are intended to exclude a situation where both ligands are preferentially expressed on substantially the same entity, for example two different tumor associated antigens associated differentially with a differentiated population of cells within a tumor, most particularly in the case where they are individually suitable targets for delivery of a toxic payload, and the terms “different” distributions and “heterogeneous” population are similarly understood to exclude such a common distribution, in the appreciation that the invention primarily represents an improved strategy for targeting two different ligands, in which one ligand has a broader distribution than the other or both have distributions that may overlap but are different from that of the target population. It will also be appreciated that the invention has particular application to a situation in which at least one of the non-target populations is one on which one of said first and second ligands is substantially represented (in contrast to one on which it simply enjoys limited expression).^{x} Contrasted to functional affinity which may result from avidity^{xi} Contrasted to functional affinity which may result from avidity^{xii} The term “receptor ligand” means a target ligand which is a ligand for a receptor, for example, a receptor on a cell or infectious agent or a receptor which circulates independently of another entity.^{xiii} The term “antigen binding fragment” refers to a polypeptide or a plurality of associated polypeptides comprising one or more portions of an antibody including at least one VH or VL or a functional fragment thereof.^{xiv} For example of the disulfide stabilized type developed by the NCl^{xv} e.g. fos-jun^{xvi} The moiety that exerts a biologic function is understood to be a biologic effector in the sense that its intended interaction with an entity in the lymphatic system or elsewhere in the organism has a biological consequence.^{xvii} For example using a toxin or immunogen fused or conjugated to (or having a corresponding ligand on the second binding moiety to which it binds) to an antibody which recognizes a lymphatic endothelial marker, for example an anthrax toxin fusion^{xviii} The term neutralizing is used broadly to refer to any interposition, interference or impediment which affects the function of the target entity^{xix} the terms modulating, mediating, neutralizing function etc. are not intended to be mutually exclusive and are each used broadly, for example the term modulating referring to effecting a change, and the term mediating preferably connoting an indirect effect achieved through the instrumentality of another entity, for example a cell, cytokine, chemokine etc.^{xx} I.e. an entity other than the lymphatic endothelial cell and other than any cell to which the first moiety is anchored.

Notwithstanding any indication to the contrary, it will be appreciated that the references herein cited have application to multiple different subjects and any qualifying remarks as to the applicability of the references is to be understood as relating to each of the subjects for which references are herein provided, as limited only by the title and subject matter of the reference.

All publications and references therein cited are herein incorporated by reference to the same extent as if each of the individual publications were specifically and individually indicated to be incorporated by reference in its entirety.

What is claimed is:

1. An isolated multispecific ligand which:
(A) has at least two binding specificities respectively for two different cell surface target ligands on the same cell;
(B) comprises:
(1) a first ligand binding moiety which preferentially recognizes a first cell surface target ligand that is specifically associated with a target cell population; and
(2) a second ligand binding moiety: (a) which serves as an effector by blocking or effecting signal transduction; (b) preferentially recognizes a second cell surface target ligand which is a cell surface receptor or receptor ligand expressed on the target cell population and a non-target cell population; (c) has an affinity for said second cell surface target ligand at least one order of magnitude lower than the affinity of the first ligand binding moiety for said first cell surface target ligand;
wherein
(i) the first ligand binding moiety and the second ligand binding moiety are each respectively, an antibody, an antibody binding fragment of said antibody, a natural ligand for the cell surface target ligand or mutants of said ligands modifying the affinity of said ligand;
(ii) the first ligand binding moiety binds to the first cell surface target ligand independently of the second binding moiety binding to the second cell surface target ligand and remains bound to the first cell surface target for a sufficient duration to assist the second ligand binding moiety to contemporaneously bind to the second cell surface target ligand on cells of the target cell population; and
(C) binds contemporaneously to the two different cell surface target ligands on cells of the target cell population that express both of said cell surface target ligands.

2. The multispecific ligand according to claim 1, wherein the affinity of the first ligand binding moiety for the first cell surface target ligand is at least approximately nanomolar affinity.

3. The multispecific ligand according to claim 1, wherein the affinity of the first ligand binding moiety for the first cell surface target ligand is at least approximately 0.1 nanomolar affinity.

4. The multispecific ligand according to claim 1, wherein the affinity of the first ligand binding moiety for the first cell surface target ligand is at least approximately two orders of magnitude higher than the affinity of the second ligand binding moiety for the second cell surface target ligand.

5. The multispecific ligand according to claim 1, wherein the affinity of the first ligand binding moiety for the first cell surface target ligand is at least approximately three orders of magnitude higher than the affinity of the second ligand binding moiety for the second cell surface target ligand.

6. The multispecific ligand according to claim 1, wherein the multispecific ligand is a bispecific antibody and wherein the affinity of the second ligand binding moiety for the second cell surface target ligand is between one and four orders magnitude lower than the affinity of the first ligand binding moiety for the first cell surface target ligand.

7. The multispecific ligand according to claim 1, wherein said second ligand is a serine kinase type receptor.

8. The multispecific ligand according to claim 1, wherein said second ligand is a heterotrimeric G-protein coupled receptor.

9. The multispecific ligand according to claim 1, wherein said second ligand is a TNF family receptor.

10. The multispecific ligand according to claim 1, wherein said second ligand is a notch family receptor.

11. The multispecific ligand according to claim 1, wherein said second ligand is a guanylate cyclase type receptor.

12. The multispecific ligand according to claim 1, wherein said second ligand is a tyrosine phosphatase type receptor.

13. The multispecific ligand according to claim 1, wherein said second ligand is an adhesion receptor.

13. The multispecific ligand according to claim 1, wherein said second ligand is a tyrosine kinase type receptor.

14. The multispecific ligand according to claim 1, wherein said second ligand is a receptor for a cytokine, chemokine, growth factor, colony stimulating factor or hormone.

15. The multispecific ligand according to claim 1, wherein the first ligand binding moiety is an effector.

16. The multispecific ligand according to claim 1, wherein said first ligand is a cell surface marker associated with one or more specific cell populations, diseased cells or disease-associated cells.

17. The multispecific ligand according to claim 16, wherein said disease-associated cells are one or more disease-mediating immune cells.

18. The multispecific ligand according to claim 16, wherein said cell surface marker is an antigen, an epitope, a CD marker, a MHC-peptide complex or a cell surface immunoglobulin or is associated with an activated immune cell.

19. The multispecific ligand according to claim 5, wherein said bispecific antibody comprises a pair of VHs linked through a flexible linker.

20. A composition comprising the multispecific ligand of claim 1 and an excipient or carrier.

22. A method of obtaining the multispecific ligand of claim 1 comprising:
(a) providing a first ligand binding moiety which binds to a first cell surface target ligand;
(b) providing a second ligand binding moiety which binds to a second cell surface target ligand;
wherein the affinity of the first ligand binding moiety for the first cell surface ligand is at least one order of magnitude higher than the affinity of the second ligand moiety for the second cell surface target ligand
and (c) linking said first ligand binding moiety to the second ligand binding moiety.

23. A method of identifying the multispecific ligand according to claim 1, (a) identifying a multispecific ligand having an improved or diminished ability to bind to or to block or effect a signal transduction on cells of a target cell population or a non-target cell population, relative to a control ligand.

24. A method according to claim 23, wherein the improved or diminished ability is demonstrated in a competition binding experiment.

25. A method according to claim 23, wherein the first ligand binding moiety or the second ligand binding moiety is selected from a library of variants having differing affinities.

26. A method according to claim 25, wherein the affinity of the first ligand binding moiety for the first cell surface ligand is pre-selected to be approximately nanomolar affinity.
